(19) United States
(12) Patent Application Publication
(10) Pub. No.: US 2022/0084645 A1
(43) Pub. Date: Mar. 17, 2022

(54) INTELLIGENT, INDIVIDUALIZED MEDICAL AND IMAGE MANAGEMENT SYSTEM

(71) Applicant: EHR Command Center, LLC, Merion Station, PA (US)

(72) Inventors: Leonard H. Ginsburg, Merion, PA (US); Nancy Wilson Crawford, Glen Mills, PA (US)

(21) Appl. No.: 17/035,648

(22) Filed: Sep. 28, 2020

Related U.S. Application Data
(60) Provisional application No. 62/907,410, filed on Sep. 27, 2019.

(51) Int. Cl. G16H 15/00; G16H 10/60; G16H 30/20

(52) U.S. Cl. CPC G16H 15/00; G16H 30/20; G16H 10/60

(57) ABSTRACT

A method, apparatus and system for in-context display of images and patient related information include retrieving patient related information from at least one patient database or server, displaying a medical record dashboard including one or more windows for displaying patient related information including at least one of medical services, clinical data, examination findings, diagnostic tests, and procedures performed on one or more patients, generating at least one thumbnail representation of at least one image related to the at least one of the medical services, the clinical data, the examination findings, the diagnostic tests, and the procedures performed on the one or more patients, and displaying at least one of the at least one generated thumbnail representations, such that when a thumbnail representation is selected, a respective image is displayed concurrently with the patient related information on the display.

### CROSS-REFERENCE TO RELATED APPLICATIONS

The present application is a continuation-in-part application of U.S. patent application Ser. No. 16/399,974 filed Apr. 30, 2019, which is a continuation of U.S. patent application Ser. No. 14/666,278 filed Mar. 23, 2015, which claims benefit of U.S. Provisional Patent Application Ser. No. 61/968,693 filed Mar. 21, 2014. The present application is also a non-provisional of U.S. Provisional Patent Application No. 62/893,688, filed Aug. 29, 2019 and a Provisional Patent Application No. 62/907,410, filed Sep. 27, 2019 and a Provisional Patent Application No. 62/983,350, filed Feb. 28, 2020 and a Provisional Patent Application No. 62/987,165, filed Mar. 9, 2020 and a Provisional Patent Application No. 63/026,547, filed May 18, 2020. The contents of these patent applications are hereby incorporated by reference in their entireties.

### BACKGROUND

Caregivers are often called upon to make rapid life and death decisions based on a patient's conditions in the context of a medical history as presented, for example, in an Electronic Medical Record (“EMR”). However, the visual display systems for conventional EMRs are often difficult to understand and require the user to move through multiple screens, interfaces, and menus to obtain the disparate information needed to make a care decision. This is problematic when caring for multiple patients in a busy practice and is particularly problematic in a critical care setting.

Conventional informational systems, such as EMR systems, provide computerized interfaces between medical professionals and their staff and patients and are designed to facilitate and streamline the business of medical care. Such systems enable a medical care provider to track the delivery of medical care, access a patient's medical records, track billing for services provided, and follow a patient's progress. However, such conventional informational systems, such as EMR systems, have mostly not met their promise because the systems include complex interfaces that require users to navigate through multiple layers, folders and/or windows to access even basic patient information. Recently, a Healthcare Information and Management Systems Society (HIMSS) survey showed that 40% of physicians would not recommend their EMR to a colleague, 63.9% said note writing took longer with electronic health records, and 32% were slower to read other clinician's notes. A recent study by Medical Economics indicated that 67% of physicians are displeased with their EMR systems.

Moreover, the complex interfaces associated with EMRs are particularly problematic at the point of care as they slow caregivers down and distract them from meaningful face-time, caring for patients. As a result, many caregivers defer their interaction with the EMR systems until after the patients have been treated. A recent study reported in the Annals of Internal Medicine reported that physicians are spending almost half of their time in the office on EMR and desk work and spend just 27% on face time with patients, which is what the vast majority of physicians went into medicine to do. Once the physician gets home, they average another one to two hours completing health records. Thus, the complex interfaces of current EMR systems have led to diminished quality of a caregiver's practice of medicine, diminished patient quality of care, and negatively impacted caregiver job satisfaction. More user-friendly interfaces enabling caregivers ready access to the information accessible through EMR systems at the point of care is needed to improve the caregiver-patient interactions and would be particularly useful in avoiding medical errors and missed diagnoses and increase compliance with insurance billing rules and regulations.

Communication of medical findings between caregivers seeing patients treated by multiple health care providers has also become more difficult. Now, rather than a phone call, simple fax or one page dictated medical summary, caregivers are now sending voluminous amounts of information as the EMR gets stuffed with insurance documentation requirements and cut and paste options from “previous visits.” Some medical conditions, such as diabetes, require multiple medical personnel to treat the patient. A single patient may have an eye doctor, family physician, endocrinologist, podiatrist, cardiologist, nephrologist, dietician/exercise physiologist, and diabetes education program coordinator. Primary care physicians can be audited and, if the annual report from a consultant is not in the chart, they can be financially penalized.

What is needed is a simple, elegant solution that enables caregivers to synthesize information and populate and document a chart when seeing a patient using a single presentation instance and enables a caregiver to identify medical problems through data visualization, where data is presented and displayed in an intuitive, easy to read manner and which enables the rapid identification of billing and collections and which enables easy sharing of medical findings, information and conclusions among multiple caregivers.

### SUMMARY

The above and other needs in the art are addressed by a data command center visual display system and associated methods for displaying data on a display screen from multiple data sources and allowing navigation amongst the data without leaving the display of the visual display system. Numerous technical issues rooted in computer technology must be solved for the data to be presented to the visual display system so that the data may be displayed in the command center using a single display interface. For example, the visual display system must provide access to the requisite health information systems and third-party support services whereby the data may be accessed, processed, and presented without unacceptable delay. Also, the display data must be collected and ordered to facilitate the various combinations of the data into respective display panels that may be navigated on the display screen. For example, it is desirable for the data to be configured in a task-based or specialty-specific display configuration for use by physicians, for example. To do this, various features in prior art systems needed to be acquired and combined in a new way to facilitate access to the features without having to navigate away from the display screen. For example, conventional EMR systems provide interfaces to third party prescription ordering systems but require the user the navigate to another system and away from the EMR interface. Accessing ordering screens without leaving the display screen becomes particularly difficult where the display screen space is limited as is the case for many physicians who use portable display devices and mobile computers. The structural embodiments described herein address these technical issues to generate the command center visual display system embodiments described herein.

In exemplary embodiments, such a data command center visual display system in accordance with the present principles includes a patient database that stores patient identification information, patient insurance information, patient medical history information, a computer readable storage medium having stored thereon instructions thereon, and a processor that executes the instructions to perform operations including creating a plurality of adjustable display panels configured to display predetermined combinations of the patient identification information, patient insurance information, patient medical history information, and creating a patient flowsheet that integrates the patient medical history information into a table that presents the patient's medical history by visit to at least one physician with respective procedures or actions performed during each visit represented as first icons identifying the procedure or action performed and second icons enabling selection of a new procedure or action, where the first and second icons provide links to associated patient medical information and ordering display panels that may be accessed without leaving the display screen. In response to selection of the second icon by a user of the visual display system, an ordering display panel is presented to the display screen in addition to the adjustable display panels and patient flowsheet. The desired procedures or actions may be ordered from the ordering display panels while relevant portions of the patient's medical history are still visible on the display screen. The scope of the claims also contemplates corresponding methods performed by the visual display system and users thereof.

In exemplary embodiments, the ordering display panel comprises an ePrescribing panel for ordering medication or a medical procedure ordering panel for ordering a medical procedure. By way of example, the medical procedure ordering panel for ordering a medical procedure may further provide a link to the quality reporting panel that displays quality reporting metrics and/or peer data related to the procedure that is being ordered. All of such ordering display panels are configured in the context of the screen display to conserve display space) so that the ordering display screen may be displayed while still being able to view the medical history data, for example.

In other exemplary embodiments, the ordering display panel comprises an imaging order panel for ordering a medical image of the patient or a lab order panel for ordering a lab test of the patient. In still other embodiments, instructions are provided that when executed create an image icon in an adjustable display panel and/or the patient flowsheet that, when selected by the user of the visual data system, opens a display window for viewing of one or more images without leaving the display screen.

In other exemplary embodiments, the visual display system incorporates financial data with the patient medical history data into the display panels. Such a visual display system includes a patient database that stores patient identification information, patient insurance information, patient medical history information, and patient payment information, a computer readable storage medium having stores thereon instructions thereon, and a processor that executes the instructions to perform operations including creating a plurality of adjustable display panels configured to display predetermined combinations of the patient identification information, patient insurance information, patient medical history information, and patient payment information, and creating a patient flowsheet that integrates the patient medical history information and patient payment information into a table that presents the patient's medical history by visit to at least one physician with respective procedures or actions performed during each visit represented as first icons identifying the procedure or action performed and second icons indicating whether the procedure or action has been paid for in part or in full, the first and second icons providing links to associated patient medical history information and/or patient payment information. In response to selection by a user of the visual display system, the adjustable display panels and patient flowsheet are moved into a task-based or specialty-specific display configuration such that the patient identification information, patient insurance information, patient medical history information, and patient payment information may be accessed without leaving the display screen. The task-based or specialty-specific display configuration is then presented to the display screen. In exemplary embodiments, selection of the first icons or second icons open display windows to associated medical history data and/or financial data and overlay a portion of the display screen with the display windows whereby the associated medical history data and/or financial data may be viewed by the user of the visual display system while the adjustable display panels and the patient flowsheet are displayed in a background on the display screen. Throughout this description, it will be appreciated that all financial data in the system, including costs to patient, is compartmentalized such that no user may see financial details for users or organizations not authorized in accordance with applicable policies and law. Also, the scope of the claims also contemplates corresponding methods performed by the visual display system and users thereof.

The visual display system includes a number of features that enable accessing information on the display screen. For example, third icons are provided in the patient flowsheet or display panels that include links to compliance information about compliance with insurance guidelines and/or good clinical practice guidelines for a procedure or action associated with each third icon. In exemplary embodiments, the compliance information includes aggregated medical treatment guidelines and an overview outlining similarities and differences amongst different medical treatment guidelines making up the aggregated medical treatment guidelines. The aggregated medical treatment guidelines may include information related to recommended follow-up with the patient, information related to procedures permitted or prevented by the patient's insurance or contra-indications, and information relating to proper billing for the procedure or action associated with a third icon selected from the patient flowsheet or display panels. In exemplary embodiments, the visual display system provides access to a clinical decision support system that uses a rules engine and/or natural language processing to aggregate the medical treatment guidelines and to generate the overview outlining similarities and differences amongst different medical treatment guidelines making up the aggregated medical treatment guidelines. The clinical decision support system and/or natural language processing system may further compare medical data to notice patterns, errors and anomalies in different entries or notes, find discrepancies in payments, alert the user of the visual display system about inconsistent medical documentation or improper orders, speed up the process of complying with regulations, alert the user of the visual display system that a plan or order is inconsistent with a preferred practice plan for a patient, or warn the user of the visual display system that billing certain procedures might not be covered. The natural language processing system may also be accessed parse notes in the patient flowsheet or display panels for potential ICD10 codes or alternative diagnosis.

The visual display system also includes a display configuration that enables users of the visual display system to order medications, diagnostic tests, images, procedures, and the like directly from the patient flowsheet or display panel. For example, an icon or link in the patient flowsheet or display panel may include an ePrescribing panel for ordering medication or a medical procedure ordering panel for ordering a medical procedure. The medical procedure ordering panel may further include a link to a quality reporting panel that displays quality reporting metrics and/or peer data related to the procedure that is being ordered. In other embodiments, an icon or link in the patient flowsheet or display panel may include an imaging order panel for ordering a medical image of the patient or a lab order panel for ordering a lab test of the patient. In still other embodiments, an image icon is provided in an adjustable display panel and/or the patient flowsheet that, when selected by the user of the visual data system, opens a display window for viewing of one or more images without leaving the display screen. In other embodiments, an alert icon is provided in an adjustable display panel and/or the patient flowsheet that, when selected by the user of the visual data system, opens an alert message without leaving the display screen. In still other embodiments, one of the display panels may be configured to accept today's visit notes from the user of the visual display system in connection with a patient visit for storage for access with other data of the one display panel.

Other novel features in exemplary embodiments include a moveable note icon for association with context information in a corresponding one of the adjustable display panels and/or the patient flowsheet. The note icon moves with the context information as the context information is moved on the display screen. When the note icon is selected, the user of the visual display system may enter a note relating to the context information.

In still other embodiments, data input by the user of the visual display system may trigger auto-population of information in the adjustable display panels and patient flowsheet and auto-population of the patient's medical record in an electronic medical record system. In the exemplary embodiments, the auto-population occurs without the user of the video display system leaving the display screen.

In other embodiments, new clinical information for the patient is provided to a diagnosis evaluation algorithm for comparison of the new clinical information with previous corresponding clinical information for the patient to determine whether the new clinical information is indicative of an improvement or worsening of the patient's medical condition. The visual display system further generates diagnosis indicators providing a visual representation of an improvement of a medical problem, disease, or symptom, or a worsening of a medical problem, disease, or symptom as a result of taking a particular medication or undergoing a particular medical procedure and displays the diagnosis indicators in the adjustable display panels and/or the patient flowsheet.

Other embodiments of the visual display system allow for increased speed of data presentation by a local database that stores a subset of patient identification information, patient insurance information, patient medical history information, and patient payment information, where the subset includes the patient identification information, patient insurance information, patient medical history information, and patient payment information for patients having an appointment within a predetermined time window.

The visual display system in exemplary embodiments includes interfaces to an external health information system and third party service systems. In exemplary embodiments, the external health information system includes at least one of an electronic medical records system, a practice management system, a health information exchange, a picture archive and communications system, a clearing house/billing system, and a laboratory system. On the other hand, the third party service systems may include one or more of an ePrescribing system, an insurance verification/referral/pre-authorization system, a system for establishing medical necessity by verifying that a procedure or medication is associated with a correct ICD10 code supporting its use, a clinical services pricing and location system, a claim status checking system, services in support of the National Correct Coding Initiative, services to proactively ensure claims are coded correctly to prevent issues in billing, claims compliance services that evaluate claims against National Coverage Determination (NCD) and Local Coverage Determination (LCD) guidelines as well as local insurance regulations to establish and document medical necessity, a natural language processing system, and artificial intelligence/cognitive systems that provide clinical decision support.

In exemplary embodiments, the patient identification information, patient insurance information, patient medical history information, and patient payment information is stored in the patient database in transactional tables that capture clinical and billing data and reporting tables where data is aggregated for a particular physician, practice, health system or other entity. Each table uses a surrogate primary key that is a unique value within the table used to identify a row that is not directly tied to data in that row. In the exemplary embodiments, XML code moves and stores different display panel and flowsheet views. The XML code further identifies a collection of panels and tabs, wherein within each panel is a panel ID that links the panel to a tab, the panel's position, and whether or not the panel is stacked with another panel. The XML code may also set up the display panels and patient flowsheet on the display screen by, for example, identifying a collection of columns and, for each column, a name of the column along with a data source. The display panels so configured are presented to the display screen for selection and display panel frames on the display screen are manipulated for receiving selected display panels.

In other exemplary embodiments, the patient flowsheet is organized around patient medical information corresponding to a particular disease state and/or procedures and/or insurance coverage and/or actions for treating the particular disease state.

The patient database may also be adapted to include patient medical history information from a plurality of medical care providers whereby the patient flowsheet may be adapted to include medical history information from more than one medical care provider in order to provide shared treatment of the patient in the patient flowsheet. In other embodiments, a summary table may be provided that illustrates everything the user of the visual display system has done for each patient in a particular time frame or for each patient having a particular disease state in a particular time frame. The summary table may also include information from other medical care providers who are providing shared treatment of the patient. If financial data, cost, charge, payment is on the summary table with the medical data, this data is compartmentalized such that no user may see financial details for users or organizations not authorized in accordance with applicable policies and law.

In yet other embodiments, a data command center visual display system is provided that presents dynamic data to a display screen. The command center visual display system includes a plurality of adjustable display panels configured to display predetermined combinations of patient identification information and patient medical information. A patient flowsheet is created that includes a table that presents the patient's medical information by medical service, medical procedure, diagnostic test, medication, and diagnosis that is prescribed, ordered, performed, or selected during respective encounters with at least one medical care provider. In response to selection by a user, at least two adjustable display panels containing medical information relating to one or more patients in the patient flowsheet are presented to the display in a single view. The user may edit or move the medical information or the patient identification information within the display panels while the display panels are simultaneously open.

In some embodiments, a method for rules-based data display in a data command center including a medical records dashboard including one or more windows including information received or derived from at least one patient database, the medical records dashboard comprising a display on a screen, using the one or more windows, of at least one of medical services, clinical data, examination findings, diagnostic tests, and the procedures performed on one or more patients, the one or more windows comprising a plurality of data entry fields, including at least one collapsible data entry field, for displaying the information received or derived from the at least one patient database, wherein the at least one of the medical services, the clinical data, the examination findings, the diagnostic tests, and the procedures are arranged in rows or columns on the screen according to at least one of a time and a date that the medical services, the clinical data, the examination findings, the diagnostic tests and the procedures were performed on the one or more patients, the method includes receiving patient-related data from the at least one patient database, comparing the received patient-related data with configuration rules to determine which portions of the received patient-related data are to be displayed in data entry fields of the medical records dashboard, identifying collapsible data entry fields of the at least one collapsible data entry field of the medical records dashboard that are determined to not have any patient-related data to display as collapsed data entry fields, displaying patient-related data in the data entry fields of the medical records dashboard in accordance with the configuration rules and collapsing data entry fields of the medical records dashboard identified as collapsed data entry fields.

In some embodiments, a data command center visual display system that displays data on a display screen includes a computing device comprising at least one processor, a non-transitory computer-readable medium, having stored thereon, software instructions that when executed by the at least one processor of the computing device, cause the computing device to perform operations comprising at least, linking to and receiving patient related medical records including patient data from at least one patient data source, and displaying a medical records dashboard including one or more windows, the medical record dashboard capable of displaying, using the one or more windows, patient data from at least one patient data source including at least one of medical services, clinical data, examination findings, diagnostic tests, and the procedures performed on one or more patients, the one or more windows comprising a plurality of data entry fields, including at least one collapsible data entry field, for displaying the information received or derived from the at least one patient database, wherein the at least one of the medical services, the clinical data, the examination findings, the diagnostic tests, and the procedures are arranged in rows or columns on the screen according to at least one of a time and a date that the medical services, the clinical data, the examination findings, the diagnostic tests and the procedures were performed on the one or more patients, wherein a display of patient data in the medical records dashboard is determined by: comparing the patient data with configuration rules to determine which portions of the patient data are to be displayed in the data entry fields of the medical records dashboard, identifying collapsible data entry fields of the at least one collapsible data entry field of the medical records dashboard that are determined to not have patient data to display as collapsed data entry fields, and displaying patient data in the data entry fields of the medical records dashboard in accordance with the configuration rules and collapsing data entry fields of the medical records dashboard identified as collapsed data entry fields.

In some embodiments, a method for unique patient identification of a subject patient in a data command center including patient-related data received or derived from at least one patient database includes collecting patient-related data having different data classifications from the at least one patient database, assigning a level of accuracy score for each of the patient-related data of the different classifications, adding, the level of accuracy scores for each of the patient-related data of the different classifications, comparing a total of the added level of accuracy scores to a previously determined matching threshold, if the total of the added level of accuracy scores exceeds the matching threshold, establishing an identification of the subject patient, and if the total of the added level of accuracy scores does not exceed the matching threshold, collecting additional patient-related data and returning to the assigning phase.

In some embodiments, a data command center visual display system for determining a unique patient identification includes a computing device comprising at least one processor, a non-transitory computer-readable medium, having stored thereon, software instructions that when executed by the at least one processor of the computing device, cause the computing device to perform operations comprising at least: linking to and receiving patient related medical records including patient data from at least one patient data source, collecting patient-related data having different data classifications from the at least one patient database, assigning a level of accuracy score for each of the patient-related data of the different classifications, adding, the level of accuracy scores for each of the patient-related data of the different classifications, comparing a total of the added level of accuracy scores to a previously determined matching threshold, if the total of the added level of accuracy scores exceeds the matching threshold, establishing an identification of the subject patient, and if the total of the added level of accuracy scores does not exceed the matching threshold, collecting additional patient-related data and returning to the assigning.

In some embodiments, a method for medication management and display in a data command center comprising one or more windows for display and including information received or derived from at least one patient database, the data command center displaying on a screen, using the one or more windows, at least one of medical services, clinical data, examination findings, diagnostic tests, and procedures performed on one or more patients, the one or more windows comprising a plurality of data entry fields for displaying the information received or derived from the at least one patient database, wherein the at least one of the medical services, the clinical data, the examination findings, the diagnostic tests, and the procedures are arranged in on the screen according to at least one of a time and a date that the medical services, the clinical data, the examination findings, the diagnostic tests and the procedures were performed on the one or more patients, includes determining, from at least one of the information received or derived from the at least one patient database and the at least one of the medical services, the clinical data, the examination findings, the diagnostic tests, and the procedures, medications administered to the one or more patients, generating a respective graphical representation for each of the determined medications administered to the one or more patients, and displaying at least one generated, respective graphical representation of at least one medication administered to a patient in the at least one or more windows in context with at least one of the information received or derived from the at least one patient database and the at least one of the medical services, the clinical data, the examination findings, the diagnostic tests, and the procedures, wherein the at least one generated, respective graphical representation of the at least one medication administered to the patient is arranged in on the screen according to at least one of the times and the dates that the at least one medication was being administered to the patient.

In some embodiments, a data command center visual display system that displays data on a display screen includes a computing device comprising at least one processor, a non-transitory computer-readable medium, having stored thereon, software instructions that when executed by the at least one processor of the computing device, cause the computing device to perform operations including at least, linking to and receiving patient related medical records including patient data from at least one patient data source, wherein the patient data includes at least one of medical services, clinical data, examination findings, diagnostic tests, and procedures performed on one or more patients, determining, from at least one of the patient data and the at least one of the medical services, the clinical data, the examination findings, the diagnostic tests, and the procedures, medications administered to the one or more patients, generating a respective graphical representation for each of the determined medications administered to the one or more patients, and displaying using the one or more windows, at least one of medical services, clinical data, examination findings, diagnostic tests, and procedures performed on one or more patients and at least one generated, respective graphical representation of at least one medication administered to a patient in context with at least one of the patient data and the at least one of the medical services, the clinical data, the examination findings, the diagnostic tests, and the procedures, wherein the at least one of the medical services, the clinical data, the examination findings, the diagnostic tests, and the procedures are arranged on the screen according to at least one of a time and a date that the medical services, the clinical data, the examination findings, the diagnostic tests and the procedures were performed on the one or more patients, and wherein the at least one generated, respective graphical representation of the at least one medication administered to the patient is arranged on the screen according to at least one of the times and the dates that the at least one medication was being administered to the patient.

In some embodiments, a method for a display of a graphical representation of complete medical history of a patient in a data command center comprising one or more windows for display and including patient-related data received or derived from at least one patient database, the method includes determining, from the patient-related data, a complete medical history of at least one patient including at least one of medical services, clinical data, examination findings, diagnostic tests, medications administered to and procedures performed on a patient, generating a graphical representation of the determined complete medical history of the patient including the at least one of medical services, clinical data, examination findings, diagnostic tests, medications administered to and procedures performed on the patient, and displaying the generated graphical representation in the at least one or more windows according to at least one of a time and a date that the at least one of the medical services, the clinical data, the examination findings, the diagnostic tests, and the procedures the medical services, the clinical data, the examination findings, the diagnostic tests and the procedures were performed on the one or more patients and at least one of the times and the dates that the medications were being administered to the patient, wherein a user is enabled to select a location in the displayed graphical representation and details regarding the at least one of medical services, clinical data, examination findings, diagnostic tests, medications administered to and procedures performed on the patient related to that selected location are presented to the user.

In some embodiments, a method for in-context display of images and patient related information includes retrieving patient related information from at least one patient database or server, displaying at least one medical record dashboard including one or more windows for displaying, using a single display interface, patient related information retrieved from or derived from the at least one patient database or server including at least one of medical services, clinical data, examination findings, diagnostic tests, and procedures performed on one or more patients. In some embodiments, the one or more windows include a plurality of data entry fields for displaying the patient related information received or derived from the at least one patient database, wherein the at least one of the medical services, the clinical data, the examination findings, the diagnostic tests, and the procedures are arranged on a display according to at least one of a time and a date that the medical services, the clinical data, the examination findings, the diagnostic tests and the procedures were performed on the one or more patients. In such embodiments, the method can further include generating at least one visual representation of at least one image related to the at least one of the medical services, the clinical data, the examination findings, the diagnostic tests, and the procedures performed on the one or more patients, and displaying at least one of the at least one generated visual representations on the display in at least one of the plurality of data entry fields, such that when a displayed visual representation is selected, a respective image is displayed concurrently with the patient related information on the display.

In some embodiments, a visual representation of a patient-related image in accordance with the present principles can include at least one thumbnail representation of an image.

In some embodiments, a user of an in-context image management system in accordance with the present principles is able to select an image to view by reviewing the thumbnail representations of the available patient-related images.

In some embodiments, a selection of a displayed visual representation of patient-related images can include clicking on the displayed visual representation of patient-related images using a pointing device.

In some embodiments, a selection of a displayed visual representation of patient-related images can include hovering over a displayed visual representation of patient-related images using a pointing device.

In some embodiment of the present principles, a system for in-context display of images and patient related information include a computing device comprising at least one processor and a non-transitory computer-readable medium, having stored thereon, software instructions. In such embodiments, when the software instructions are executed by the at least one processor of the computing device, the system is configured to perform operations including at least retrieving patient related information from at least one patient database or server, displaying at least one medical record dashboard including one or more windows for displaying, using a single display interface, patient related information retrieved from or derived from the at least one patient database or server including at least one of medical services, clinical data, examination findings, diagnostic tests, and procedures performed on one or more patients. In such embodiments, the one or more windows include a plurality of data entry fields for displaying the patient related information received or derived from the at least one patient database, and where the at least one of the medical services, the clinical data, the examination findings, the diagnostic tests, and the procedures are arranged on a display according to at least one of a time and a date that the medical services, the clinical data, the examination findings, the diagnostic tests and the procedures were performed on the one or more patients. In such embodiments the system is further configured to perform operations including generating at least one visual representation of at least one image related to the at least one of the medical services, the clinical data, the examination findings, the diagnostic tests, and the procedures performed on the one or more patients, and displaying at least one of the at least one generated graphical representation on the display in at least one of the plurality of data entry fields, such that when a displayed graphical representation is selected, a respective image is displayed concurrently with the patient related information on the display.

Other and further embodiments in accordance with the present principles are described below.

### DESCRIPTION OF THE DRAWINGS

FIG. 1 depicts a high-level block diagram of a Data Command Center in accordance with an embodiment of the present principles.

FIG. 2 depicts a high-level block diagram of a computing device 200 suitable for use with embodiments of a Data Command Center in accordance with the present principles.

FIG. 3 depicts a high-level diagram of a medical records dashboard selection window of, for example, a medical record system useful for selecting and launching at least a portion of a Data Command Center (CC) in accordance with an embodiment of the present principles.

FIG. 4A depicts an example of a medical records dashboard of the Data Command Center in accordance with an embodiment of the present principles.

FIG. 4B depicts a portion of the medical records dashboard of FIG. 4A in accordance with some embodiments of the present principles.

FIG. 4C depicts greater detail of at least some portions of the medical records dashboard of FIG. 4A in accordance with some embodiments of the present principles.

FIG. 4D depicts greater detail of at least some portions of the medical records dashboard of FIG. 4A in accordance with some embodiments of the present principles.

FIG. 5A depicts the medical records dashboard including a medical summary update process in accordance with some embodiments of the present principles.

FIG. 5B depicts a portion of the medical records dashboard including a notes update procedure in accordance with an embodiment of the present principles.

FIG. 6 depicts a user record access process of the medical records dashboard in accordance with an embodiment of the present principles.

FIG. 7A depicts a medical records access window of the medical records dashboard in accordance with an embodiment of the present principles.

FIG. 7B depicts the functionality of three specific rows in the user interface in accordance with an embodiment of the present principles.

FIG. 8 depicts a medical records and diagnosis update process of the medical records dashboard in accordance with an embodiment of the present principles.

FIG. 9 depicts a medical record update marker process of the medical records dashboard in accordance with an embodiment of the present principles.

FIG. 10 depicts a medical record update marker process of the medical records dashboard in accordance with an embodiment of the present principles.

FIG. 11 depicts a portion of a medical records dashboard in accordance with another embodiment of the present principles.

FIG. 12 depicts a portion of the medical records dashboard of FIG. 11 in accordance with an embodiment of the present principles.

FIG. 13 depicts a portion of a medical records dashboard configured for display as a function of disease or patient in accordance with another embodiment of the present principles.

FIG. 14 depicts a portion of a medical records dashboard configured for display as a function of disease of a patient and specifically configured to display data related to patients with diabetes in accordance with another embodiment of the present principles.

FIG. 15 depicts an embodiment of a medical records dashboard which can be displayed following a user's selection of at least one medical records dashboard from the medical records dashboard selection window in accordance with another embodiment of the present principles.

FIG. 16 depicts a ledger window accessible from the medical records dashboard of FIG. 15 in accordance with an embodiment of the present principles.

FIG. 17 depicts an embodiment of a Data Command Center menu including a medical records dashboard implemented as a data interface to a medical record system in accordance with an embodiment of the present principles.

FIG. 18 depicts an embodiment of a User View control panel that can be part of the View/Task menu of the medical records dashboard of the Data Command menu of the embodiment of FIG. 17 in accordance with an embodiment of the present principles.

FIG. 19 depicts an embodiment sticky note panel of the Data Command Center menu of FIG. 17, which is activated when the add sticky notes icon in FIG. 17 is selected in accordance with an embodiment of the present principles.

FIG. 20 depicts an embodiment of a Patient Information Panel of the Data Command Center menu, which can be activated when the Patient Information Bar is selected in accordance with an embodiment of the present principles.

FIG. 21 depicts an embodiment of a Patient Insurance Panel of the Data Command Center menu, which can be activated when the Patient Insurance Bar is selected in accordance with an embodiment of the present principles.

FIG. 22A depicts an embodiment of a Today's Visit Notes tab of the Data Command Center menu in accordance with an embodiment of the present principles.

FIG. 22B depicts an embodiment of a Surgeries tab of a medical records dashboard of a Data Command Center in accordance with an embodiment of the present principles.

FIG. 23 depicts an embodiment of a medical records dashboard in accordance with another embodiment of the present principles.

FIG. 24 depicts an embodiment of a co-managed medical records dashboard in the Data Command Center of the present principles in accordance with one embodiment.

FIG. 25A depicts a first portion of a medical records dashboard including an ability to launch a Co-Management process in accordance with an embodiment the present principles.

FIG. 25B depicts a second portion of a medical records dashboard including an ability to launch a Co-Management process in accordance with an embodiment the present principles.

FIG. 26A depicts a first portion of a medical records dashboard including a custom template creation process for co-management in accordance with an embodiment of the present principles.

FIG. 26B depicts a second portion of a medical records dashboard including a custom template creation process for co-management in accordance with an embodiment of the present principles.

FIG. 27A depicts a first portion of a workflow diagram of a Co Management process in accordance with an embodiment of the present principles.

FIG. 27B depicts a second portion of the workflow diagram of the Co Management process of FIG. 27A in accordance with an embodiment of the present principles.

FIG. 28 depicts a flow diagram of a method for Co-Management of patient information in a medical records dashboard in accordance with an embodiment of the present principles.

FIG. 29 depicts a flow diagram of a method for Unique Patient Identification in a Data Command Center in accordance with an embodiment of the present principles.

FIG. 30 depicts a first embodiment of a Medication Management chart that can be displayed in at least a portion of a medical records dashboard of the present principles in accordance with one embodiment.

FIG. 31 depicts an embodiment of the control panel #1 of the Medication Management chart of FIG. 30 in accordance with an embodiment of the present principles.

FIG. 32 depicts a Medication Management Chart that can be displayed as part of a medical records dashboard or as a stand-alone Medication Management tool in accordance with an embodiment of the present principles.

FIG. 33A depicts an example of how the Control Panel #1 of FIG. 30 can be implemented by a user to identify start and stop dates for the various medications taken by a user in accordance with an embodiment of the present principles.

FIG. 33B depicts an embodiment of a Medication Management Chart in which icons can be activated to bring up additional information in accordance with an embodiment of the present principles.

FIG. 33C depicts another embodiment of a Medication Management Chart in which icons can be activated to bring up additional information in accordance with another embodiment of the present principles.

FIG. 33D depicts an embodiment of the Medication Management Chart in which intraocular pressure, in addition to being listed by number, is also displayed as a vertical line graph, for example as depicted by element 1 in accordance with an embodiment of the present principles.

FIG. 33E depicts an embodiment of the Medication Management Chart of FIG. 33D in which the control panel can be used to input a reason that a medication has been started or stopped in accordance with an embodiment of the present principles.

FIG. 33F depicts an embodiment of the Medication Management Chart of FIG. 33D in which the control panel can be used to correct start and stop dates for a medication in accordance with an embodiment of the present principles.

FIG. 33G depicts an embodiment of the Medication Management Chart of FIG. 33D in which both corrected start and stop dates for a medication taken by a patient and incorrect start and stop dates for a medication taken by a patient and listed for example by a 3^{rd} party data provider such as an EMR can be displayed simultaneously in accordance with an embodiment of the present principles.

FIG. 33H depicts an embodiment of the Medication Management Chart of FIG. 33D in which a user is alerted that a medication being taken by a patient has changed, even if medications are being listed by class and the new medication is of the same class as the old medication in accordance with an embodiment of the present principles.

FIG. 33I depicts an embodiment of the Medication Management Chart of FIG. 33H in which a user is able to select a portion of a graph to bring up additional information associated with the graph in accordance with an embodiment of the present principles.

FIG. 34 depicts an illustration of a second embodiment of a Medication Management chart that can be displayed in at least a portion of the medical records dashboard of the present principles in accordance with one embodiment.

FIG. 35 depicts a medical records dashboard including a third embodiment of a Medication Management chart in accordance with an embodiment of the present principles.

FIG. 36 depicts a high-level workflow diagram of an embodiment of Medication Management in a Data Command Center in accordance with an embodiment of the present principles.

FIG. 37 depicts an exemplary embodiment of a Medications Management chart/tool which does not use rows or columns in accordance with an alternate embodiment of the present principles.

FIG. 38 depicts a whole view of an embodiment of a medical records dashboard of a Data Command Center in which a user/medical care provider is enabled to place orders in context with other relevant patient data/information.

FIG. 38A depicts a first enlarged portion of the Data Command Center of FIG. 38.

FIG. 38B depicts a second enlarged portion of the Data Command Center of FIG. 38.

FIG. 38C depicts a third enlarged portion of the Data Command Center of FIG. 38.

FIG. 38D depicts a fourth enlarged portion of the Data Command Center of FIG. 38.

FIG. 39 depicts an embodiment of a medical records dashboard of a Data Command Center in which a user/medical care provider is enabled to place orders in context with other relevant patient data/information, so as to enable the user/medical care provider to see the future orders in context in an embodiment not using rows and columns in accordance with the present principles.

FIG. 40A depicts a workflow diagram of a process for intelligently expanding, collapsing, displaying, and/or hiding columns, rows and/or any other portion of the medical records dashboard in accordance with an embodiment of the present principles.

FIG. 40B depicts a workflow diagram of a process for intelligently expanding, collapsing, displaying, and/or hiding columns, rows and/or any other portion of the medical records dashboard in accordance with an embodiment of the present principles.

FIG. 41 depicts a flow diagram of a method for rules-based data display in a data command center comprising a medical records dashboard in accordance with an embodiment of the present principles.

FIG. 42 depicts a graphical view of the entire medical history of a patient as a Whole Life tool in accordance with an embodiment of the present principles.

FIG. 43 depicts a post appointment summary chart of a Medical Guidance tool in accordance with an embodiment of the present principles.

FIG. 44 depicts an Evaluative Clinical Reporting (ECR) interface in accordance with an embodiment of the present principles.

FIG. 45A depicts a reporting architecture of a Data Command Center in accordance with an embodiment of the present principles.

FIG. 45B depicts an embodiment of the Orchestrator Master Pipeline of FIG. 45A in with an embodiment of the present principles.

FIG. 46A depicts a sequence diagram for executing a report in accordance with an embodiment of the present principles.

FIG. 46B depicts an embodiment of a medical records dashboard of a Data Command Center in which reports can be seen in context with an ability to order in accordance with an embodiment of the present principles.

FIG. 46C depicts an embodiment of a medical records dashboard of a Data Command Center in which reports can be seen in context with an ability to order in accordance with an embodiment of the present principles.

FIG. 46D depicts an embodiment of a medical records dashboard of a Data Command Center in which reports can be seen in context with an ability to order in accordance with an embodiment of the present principles.

FIG. 46E depicts an embodiment of a medical records dashboard of a Data Command Center in which reports can be seen in context with an ability to order and view orders in accordance with an embodiment of the present principles

FIG. 47 depicts an embodiment of a medical records dashboard of a Data Command Center in which alerts and tasks can be in accordance with an embodiment of the present principles.

FIG. 48 depicts a menu for pre-configuring alerts in accordance with an embodiment the present principles.

FIG. 49 depicts an embodiment of a medical records dashboard in which alerts are configured based on medication in accordance with the present principles.

FIG. 50 depicts three different representations of an intelligent alert configuration system overlayed upon several different aspects of an application in accordance with an embodiment of the present principles.

FIG. 51 depicts a view configuration page in accordance with an embodiment of the present principles.

FIG. 52 depicts a view configuration alerts and auto-tasks in accordance with another embodiment of the present principles.

FIG. 53 depicts a Data Command Center architecture and connectivity to external Health Information Technology systems and third-party services in accordance with an embodiment of the present principles.

FIG. 54 depicts examples of Health Information Technology systems in accordance with an embodiment of the present principles.

FIG. 55 depicts a medical records dashboard of a Data Command Center of the present principles that enables a healthcare provider while delivering medical care to a patient to participate in revenue cycle management in accordance with an embodiment of the present principles.

FIG. 56 depicts an embodiment of an in-context image management system of a Data Command Center of the present principles displaying thumbnailed images in rows and columns in accordance with an embodiment of the present principles.

FIG. 57 depicts an embodiment of the in-context image management system displaying images in the context of graphically visualized medical data modules in accordance with an embodiment of the present principles.

FIG. 58 depicts an embodiment of the in-context image management system displaying multiple patients and multiple images in accordance with an embodiment of the present principles.

FIG. 59 depicts an embodiment of a display of an in-context image management system enabling a direct edit of image information in accordance with an embodiment of the present principles.

FIG. 60 depicts a high level diagram of a Financial Flowsheet in accordance with an embodiment of the present principles.

FIG. 61 depicts a flow diagram of a method for in-context display of images and patient related information in accordance with an embodiment of the present principles.

The figures are not drawn to scale and may be simplified for clarity. It is contemplated that elements and features of one embodiment may be beneficially incorporated in other embodiments without further recitation.

### DETAILED DESCRIPTION

Embodiments of the present principles generally relate to a Data Command Center for displaying data on a display screen from multiple data sources and enabling navigation amongst the data on a single display. While the concepts of the present principles are susceptible to various modifications and alternative forms, specific embodiments thereof are shown by way of example in the drawings and are described in detail below. It should be understood that there is no intent to limit the concepts of the present principles to the particular forms disclosed. On the contrary, the intent is to cover all modifications, equivalents, and alternatives consistent with the present principles and the appended claims. For example, although embodiments of the present principles will be described primarily with respect to inter-function with an EMR system, such teachings should not be considered limiting. Embodiments in accordance with the present principles can inter-function with other informational systems such as Health Information Exchanges (HIEs), Billing Clearinghouses, Insurance Companies, Picture Archiving and Communication Systems (PACS) as well as third party services and the like.

In addition, the tool embodiments of the present principles are not limited in application to the details of construction and the arrangement of components set forth in the following description or illustrated in the following drawings. Embodiments of the present principles are capable of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, the term “medical care provider” is intended to represent any healthcare provider/clinical professional such as a doctor, physician, podiatrist, chiropractor, dentist, veterinarian, ancillary staff, nurses, physician's assistant, medical care provider, physical therapist, all allied health professionals, and/or hospital staff member. All such healthcare providers/clinical professional can implement embodiments of the present principles the tool as interchangeable users.

As used herein a row, column, or line of items (even a diagonal line) is intended to represent a sequencing or evaluation of information in any direction. In the embodiments depicted herein, information does not have to be depicted as having a visual or physical separation in the vertical or horizontal direction to be defined as being a row or column. In accordance with the present principles items next to each other horizontally, lined up in such a way that straight lines above and below can be drawn and items fall between those two horizontal lines, can be considered as being in a row. Items in rows can be related by similar time or other common or same denominator, such as a medical service, procedure, image or financial number, so that a user can quickly visualize trends or changes in those items. Similarly, items next to each other vertically, lined up in such a way that straight lines to the left and to the right can be drawn can be considered as being in a column. In some embodiments, items can be arranged diagonally and be considered to be in a row or a column.

As used herein, Practice Management Systems (PMs) are programs that perform the billing collection and reconciliation of payments as well as scheduling patients. PMs can also be referred to as Revenue Cycle Management (RCM) and have associated billing companies that use software to help practices and medical care providers get the bills out and collect money from insurance companies. In some embodiment, these entities can integrate with and work through clearing houses.

In the embodiments described herein, the terms window screen, scrolling screen, display. view, snapshot and the like can be used interchangeably and are intended to represent a single instance of the presentation of medical information associated with at least one patient. In the described embodiments, the single instance can be presented on one or more windows, in a single or multiple screens, a scrolling screen, in one or more views and using one or more snapshots. For example, in some embodiments in accordance with the present principles a user can access different panels from a scrolling screen and converge the panels into a single view or snapshot. That is, in accordance with the present principles, a user is able to compile data/information from various windows, screens, scrolling screens, displays, snapshots and the like and create a single instance presentation including the data/information of interest to the user for at least one patient. In accordance with the present principles, a single instance presentation can be presented on more than one monitor at a time. As used herein, the term single instance presentation is intended to describe a single display interface that is not limited to a single monitor. That is, in some embodiments, what defines a single instance presentation is the fact that there is a single interface, a single control that controls the presentation of the date/information, which can be then be viewed on one or more monitors or other means.

The term medical tests as described herein is intended to describe medical procedures performed for or on patients including but not limited to image or imaging, diagnostic tests, radiological tests or procedures, laboratories, chemistry and hematological tests, photography, genetic testing, nuclear scans, ultrasounds, x-rays, optical coherent tomography photographs and angiographies, assessments and plans, letters, examination findings and any medical testing or medical services that tests or screens patients for a medical condition, which in some instance can be identified by CPT codes. It should be further noted that in some instances, terms like diagnosis can be reflected by ICD 9 or 10 or similar identifying factors, and medications can be interchangeable.

As used herein, the terms icon, symbol, and indicator are all interchangeable and are intended to describe a visual element enabling the access of additional underlying information and having the ability to convey additional information simply by their presentation. That is, such visual elements can convey information by their display which can include such visual presentations including but not limited to words, numbers, blinking elements, flashing elements, color changing elements, elements in italics, underlined elements, and the like or any means that draws the attention of a user.

The reference to a medical records dashboard of the present principles described throughout the teachings herein is intended to refer to any embodiment of a medical records dashboard according to the present principles that is applicable to a currently described embodiment.

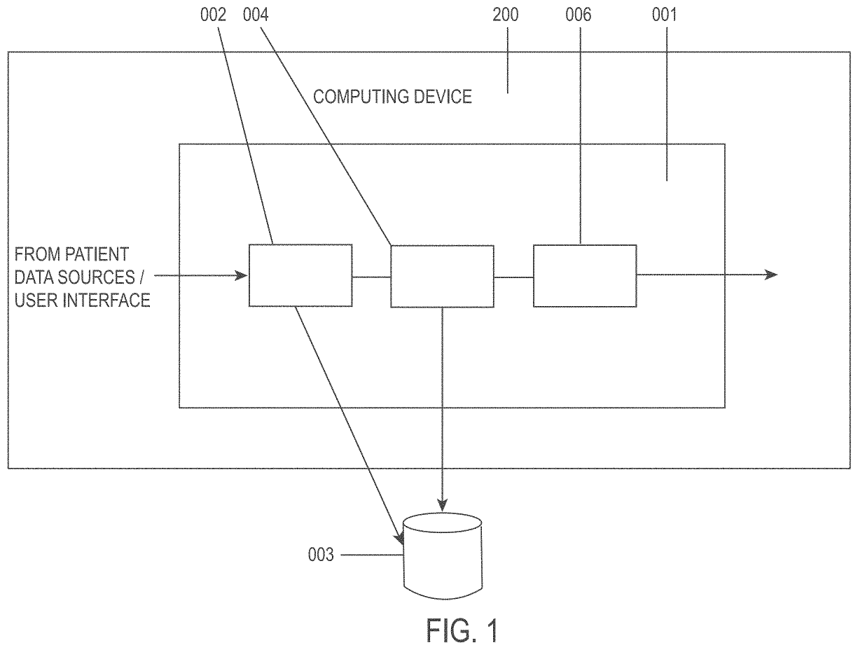

FIG. 1 depicts a high-level block diagram of a Data Command Center (DCC) 001 in accordance with an embodiment of the present principles. In the embodiment of FIG. 1, the Data Command Center 001 illustratively comprises an integration module 002 (i.e., to interface data between an EMR and the DCC), a Rules module 004 (i.e., to determine where and how the data is to be displayed), and a display module 006 (i.e., to display the data in the appropriate place). In the embodiment of FIG. 1, the integration module 002 and the rules module 004 can be in communication with a data storage 003. For example, the integration module 002 can store data from patient data sources in the data storage 003 and the rules module 004 can access the data storage 003 to retrieve data and/or information stored therein.

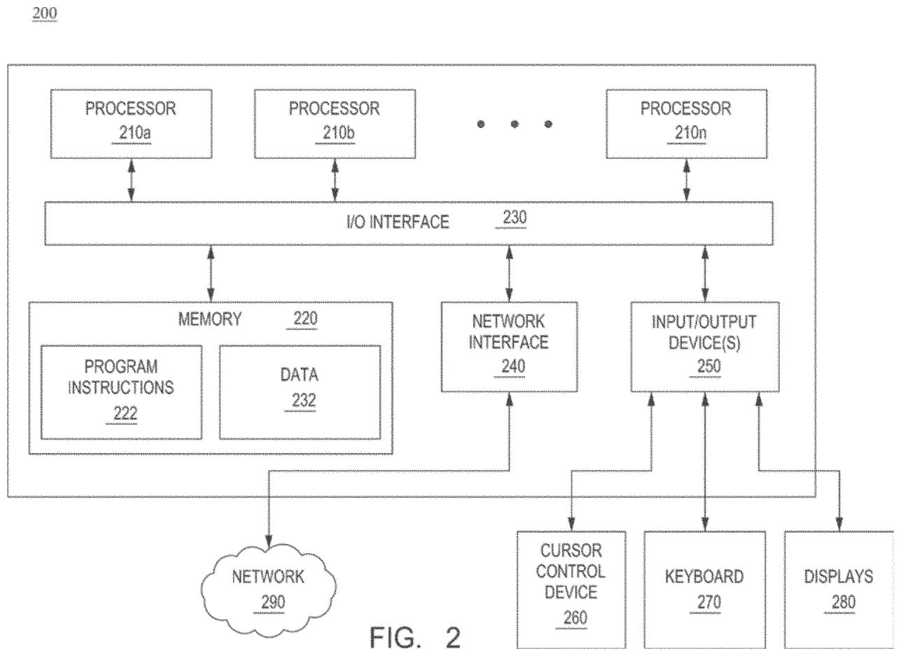

As depicted in FIG. 1, embodiments of a Data Command Center in accordance with the present principles, such as the Data Command Center 001 of FIG. 1, can be implemented in a computing device 200. FIG. 2 depicts a high-level block diagram of a computing device 200 suitable for use with embodiments of a Data Command Center in accordance with the present principles such as the user Data Command center 001 of FIG. 1. In some embodiments, the computing device 200 can be configured to implement methods of the present as processor-executable executable program instructions 222 (e.g., program instructions executable by processor(s) 210) in various embodiments.

In the embodiment of FIG. 2, the computing device 200 includes one or more processors 210a-210n coupled to a system memory 220 via an input/output (I/O) interface 230. The computing device 200 further includes a network interface 240 coupled to I/O interface 230, and one or more input/output devices 250, such as cursor control device 260, keyboard 270, and display(s) 280. In various embodiments, a user interface can be generated and displayed on display 280. In some cases, it is contemplated that embodiments can be implemented using a single instance of computing device 200, while in other embodiments multiple such systems, or multiple nodes making up the computing device 200, can be configured to host different portions or instances of various embodiments. For example, in one embodiment some elements can be implemented via one or more nodes of the computing device 200 that are distinct from those nodes implementing other elements. In another example, multiple nodes may implement the computing device 200 in a distributed manner.

In different embodiments, the computing device 200 can be any of various types of devices, including, but not limited to, a personal computer system, desktop computer, laptop, notebook, tablet or netbook computer, mainframe computer system, handheld computer, workstation, network computer, a camera, a set top box, a mobile device, a consumer device, video game console, handheld video game device, application server, storage device, a peripheral device such as a switch, modem, router, or in general any type of computing or electronic device.

In various embodiments, the computing device 200 can be a uniprocessor system including one processor 210, or a multiprocessor system including several processors 210 (e.g., two, four, eight, or another suitable number). Processors 210 can be any suitable processor capable of executing instructions. For example, in various embodiments processors 210 may be general-purpose or embedded processors implementing any of a variety of instruction set architectures (ISAs). In multiprocessor systems, each of processors 210 may commonly, but not necessarily, implement the same ISA.

System memory 220 can be configured to store program instructions 222 and/or data 232 accessible by processor 210. In various embodiments, system memory 220 can be implemented using any suitable memory technology, such as static random-access memory (SRAM), synchronous dynamic RAM (SDRAM), nonvolatile/Flash-type memory, or any other type of memory. In the illustrated embodiment, program instructions and data implementing any of the elements of the embodiments described above can be stored within system memory 220. In other embodiments, program instructions and/or data can be received, sent or stored upon different types of computer-accessible media or on similar media separate from system memory 220 or computing device 200.

In one embodiment, I/O interface 230 can be configured to coordinate I/O traffic between processor 210, system memory 220, and any peripheral devices in the device, including network interface 240 or other peripheral interfaces, such as input/output devices 250. In some embodiments, I/O interface 230 can perform any necessary protocol, timing or other data transformations to convert data signals from one component (e.g., system memory 220) into a format suitable for use by another component (e.g., processor 210). In some embodiments, I/O interface 230 can include support for devices attached through various types of peripheral buses, such as a variant of the Peripheral Component Interconnect (PCI) bus standard or the Universal Serial Bus (USB) standard, for example. In some embodiments, the function of I/O interface 230 can be split into two or more separate components, such as a north bridge and a south bridge, for example. Also, in some embodiments some or all of the functionality of I/O interface 230, such as an interface to system memory 220, can be incorporated directly into processor 210.

Network interface 240 can be configured to allow data to be exchanged between the computing device 200 and other devices attached to a network (e.g., network 290), such as one or more external systems or between nodes of the computing device 200. In various embodiments, network 290 can include one or more networks including but not limited to Local Area Networks (LANs) (e.g., an Ethernet or corporate network), Wide Area Networks (WANs) (e.g., the Internet), wireless data networks, some other electronic data network, or some combination thereof. In various embodiments, network interface 240 can support communication via wired or wireless general data networks, such as any suitable type of Ethernet network, for example; via digital fiber communications networks; via storage area networks such as Fiber Channel SANs, or via any other suitable type of network and/or protocol.

Input/output devices 250 can, in some embodiments, include one or more display terminals, keyboards, keypads, touchpads, scanning devices, voice or optical recognition devices, or any other devices suitable for entering or accessing data by one or more computer systems. Multiple input/output devices 250 can be present in computer system or can be distributed on various nodes of the computing device 200. In some embodiments, similar input/output devices can be separate from the computing device 200 and can interact with one or more nodes of the computing device 200 through a wired or wireless connection, such as over network interface 240.

Those skilled in the art will appreciate that the computing device 200 is merely illustrative and is not intended to limit the scope of embodiments. In particular, the computer system and devices can include any combination of hardware or software that can perform the indicated functions of various embodiments, including computers, network devices, Internet appliances, PDAs, wireless phones, pagers, and the like. The computing device 200 can also be connected to other devices that are not illustrated, or instead can operate as a stand-alone system. In addition, the functionality provided by the illustrated components can in some embodiments be combined in fewer components or distributed in additional components. Similarly, in some embodiments, the functionality of some of the illustrated components may not be provided and/or other additional functionality can be available.

The computing device 200 can communicate with other computing devices based on various computer communication protocols such a Wi-Fi, Bluetooth® (and/or other standards for exchanging data over short distances includes protocols using short-wavelength radio transmissions), USB, Ethernet, cellular, an ultrasonic local area communication protocol, etc. The computing device 200 can further include a web browser.

Although the computing device 200 is depicted as a general purpose computer, the computing device 200 is programmed to perform various specialized control functions and is configured to act as a specialized, specific computer in accordance with the present principles, and embodiments can be implemented in hardware, for example, as an application specified integrated circuit (ASIC). As such, the process steps described herein are intended to be broadly interpreted as being equivalently performed by software, hardware, or a combination thereof.

Those skilled in the art will also appreciate that, while various items are illustrated as being stored in memory or on storage while being used, these items or portions of them can be transferred between memory and other storage devices for purposes of memory management and data integrity. Alternatively, in other embodiments some or all of the software components can execute in memory on another device and communicate with the illustrated computer system via inter-computer communication. Some or all of the system components or data structures can also be stored (e.g., as instructions or structured data) on a computer-accessible medium or a portable article to be read by an appropriate drive, various examples of which are described above. In some embodiments, instructions stored on a computer-accessible medium separate from the computing device 200 can be transmitted to the computing device 200 via transmission media or signals such as electrical, electromagnetic, or digital signals, conveyed via a communication medium such as a network and/or a wireless link. Various embodiments can further include receiving, sending or storing instructions and/or data implemented in accordance with the foregoing description upon a computer-accessible medium or via a communication medium. In general, a computer-accessible medium can include a storage medium or memory medium such as magnetic or optical media, e.g., disk or DVD/CD-ROM, volatile or non-volatile media such as RAM (e.g., SDRAM, DDR, RDRAM, SRAM, and the like), ROM, and the like.

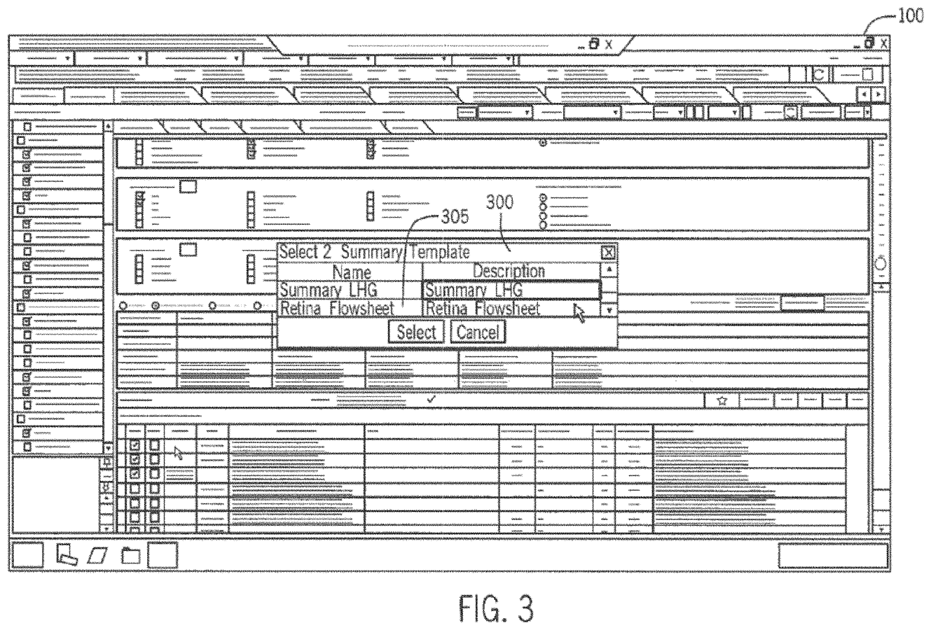

In some embodiments, a Data Command Center (CC) in accordance with the present principles is implemented as a data interface to a medical record system (e.g., EMR). In such embodiments a medical care provider can utilize a conventional medical record system to launch or enter a Data Command Center (CC) in accordance with the present principles including a medical-services tracking system that can display information dashboards, tables, charts, windows, as will be described herein. For example, FIG. 3 depicts a high-level diagram of a medical records dashboard selection window of, for example, a medical record system useful for selecting and launching at least a portion of a Data Command Center (CC) in accordance with an embodiment of the present principles. For example, as depicted in FIG. 3, a medical records dashboard selection window 300 can include one or more selectable medical records dashboards from which a user can select to access at least one medical records dashboard. For example, in one non-limiting example embodiment, the user can select a “Retina Flowsheet” 305 to access and/or launch a medical records dashboard including a retina flowsheet in a Data Command Center (CC) in accordance with the present principles. In some further embodiments, the at least one medical records dashboard can include any number of selectable medical records for any medical condition, and/or any medical diagnosis, and/or any medical treatment.

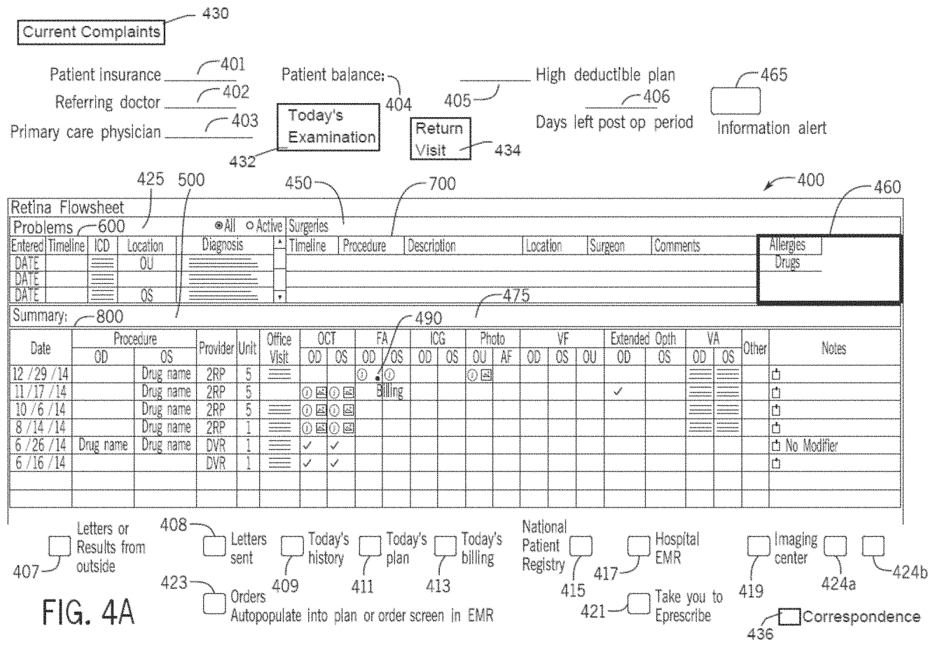

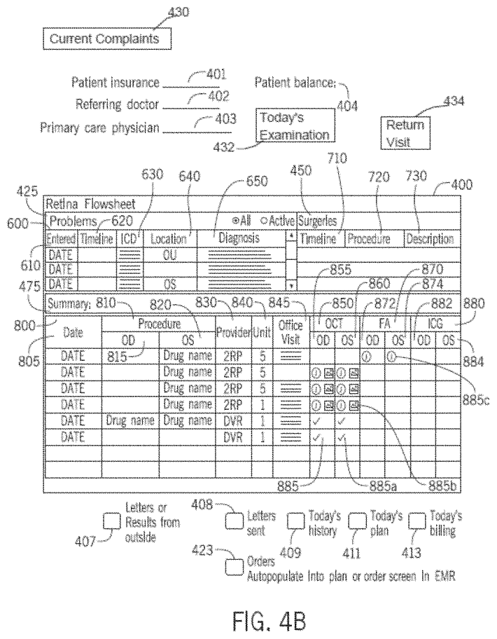

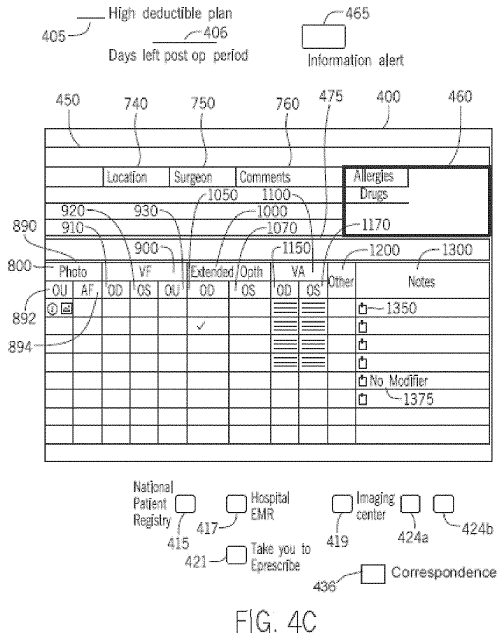

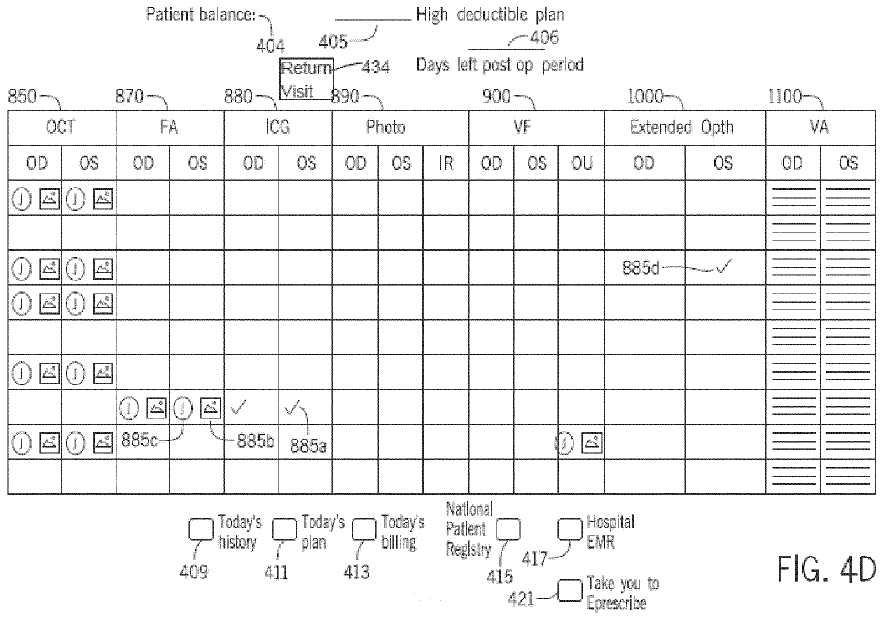

For example, FIG. 4A depicts a medical records dashboard 400 of the Data Command center 001 in accordance with an embodiment of the present principles. The medical records dashboard 400 is capable of displaying data from one or more medical records, and/or track medical procedures and services based on claims made or billing signed off by a physician for one or more delivered medical procedures or services. For example, in some embodiments the integration module 002 of the Data Command center 001 can dynamically link to various external databases comprising patient information that can be displayed in the medical records dashboard 400 by the display module 006 in accordance with rules for display in the rules module 004. For example, in some embodiments, the Data Command center 001 can function as a portal to patient information prepared by the user or patient information from other sources.

In some embodiments, the medical records dashboard 400 can be auto-populated by the display module 006 in accordance with rules in the rules module 004 as a function of claims made or billing signed off by a physician. In such embodiments, any data displayed within the medical records dashboard 400 is derived from one or more claim records that have been billed for one or more procedures or services have previously been provided to the patient. In some other embodiments, auto-population can be enabled in both directions interacting as a switchboard between the entire EMR and the medical records dashboard 400 along with what is added to any window, sub-window, column or entry in the medical records dashboard 400 being automatically added to the appropriate part of the chart for documentation before finalizing the encounter.

The medical records dashboard 400 can display information related to any medical procedures or services in relation to care of a patient. For example, in some embodiments, the medical records dashboard 400 can display information related to medical procedures or services in relation to retinal eye medical care of a patient. In some embodiments, the medical records dashboard 400 can display information including components where there is a summary of the patient's problem list that a user can input patient information and constantly update and change. Further, this information can be auto-populated with the touch of a button into a designated location such as the current plan documenting the patient's current visit (thus aiding documentation for the current visit). Further, whatever is important for a user to input into the day's visits for documentation can be initially inputted in the table, and then permanently into the day's patient visits. Further, a summary section of the medical records dashboard 400 can be dynamic and can be changed at every visit rather than being written to an unchangeable document or file (e.g., such as a PDF). Further, any patient data that is input, received, analyzed, or created can be auto-populated into any portion of the dashboard 400, and/or can form a dataflow out of the medical records dashboard 400 to another electronic system or server, or another user, observer, or other third-party.

In some embodiments, the medical records dashboard 400 can display various windows and sub-windows based on a user preference and/or current or previous user interaction with the medical records dashboard 400. For example and with reference to FIG. 4A, in some embodiments, the medical records dashboard 400 can display a problems window 425 and/or a surgeries window 450 where information related to a patient's medical problems and surgeries can be displayed in information columns 600, 700 respectively. Further, in some embodiments, patient information related to allergies and drugs can be displayed within the allergies/drug section 460. This information can be auto-populated from a variety of sources, or inputted by a user.

In some embodiments, the medical records dashboard 400 can include a summary window 475 enabling a user to view and edit summary information related to the patient, any details of care provided to the patient, and/or and any medical diagnosis information prepared by a medical practitioner. Further, in some embodiments, the medical records dashboard 400 can also display detailed information related to any medical procedures or services provided to the patient, including procedures or services that are auto-populated by claims made, or billings or payments including billing signed off by a physician as detailed above. For example, in some embodiments, the medical records dashboard 400 can display visual display window 500 including information columns 800 that can be auto-populated by claims made or billings signed off by a physician. The auto-population can include billings, payments, or other information from anywhere in the EMR chart. For example in some embodiments, the information that is auto-populated can include treatment summaries, and/or diagnosis summaries, and/or patient feedback summaries, and/or other physician summaries, and so on. For example, in some embodiments, the Data Command center 001 can display and/or auto-populate at least one field, table, or window with at least one of a patient's prior medical procedures, diagnostic tests, surgeries, current medications, current illnesses, treated illnesses, and so on. The Data Command center 001 can auto-populate various data fields via an electronic dataflow established between the Data Command center 001 and one or more computer systems of servers that comprise patient information (e.g., such as electronic medical records). The dataflow can comprise a two-way flow from the source of patient data to the Data Command center 001 and from the Data Command center 001 to the source. In some embodiments, this information can be any medical diagnosis information, any medical procedures or services provided to the patient, procedures or services by claims made, or billings or payments including billing signed off by a physician as detailed earlier, any information from anywhere in the EMR chart including treatment summaries, and/or diagnosis summaries, the patient's prior medical procedures, diagnostic tests, surgeries, current medications, current illnesses, treated illnesses, and/or patient feedback summaries, and/or other physician summaries, patient outcome summaries, treatment summaries, and/or diagnosis summaries, and/or patient feedback summaries, and/or other physician summaries or treatments. Further, in some embodiments, the information that is auto-populated can include patient outcome summaries. For example, in some embodiments, the Data Command center 001 processes a plurality of patient outcomes and displays an analysis of patient outcomes based at least in part on patient information from treatment summaries, and/or diagnosis summaries, and/or patient feedback summaries, and/or other physician summaries or treatments. In some embodiments, the patient outcomes can include or comprise physician quality reporting system (PQRS) quality measures. In some embodiments, calculated or reported patient outcomes can include or comprise at least one PQRS measures code.

As depicted in FIG. 4A, the medical records dashboard 400 can include miscellaneous information identifying the patient, information related to the patient's insurance plan, physicians and referring physicians, and the patient's current balance. Other information can relate to the patient's prior visit, prior diagnosis or procedure and any important information relevant to the next visit. Additional information can relate to the current visit, including history of illness and chief or current medical complaint, billing information, and retrievable medical information including pharmacy information. For example and as depicted in FIG. 4A, in some embodiments, the medical records dashboard 400 can include a patient insurance entry 401, referring physician entry 402, and primary care physician entry 403. The medical records dashboard 400 can also include patient balance entry 404, and a high deductible plan entry 405. Important patient information related to a pending or current visit can include a “days left post-op period” entry 406 and/or an information alert 465. In some embodiments, the information alert 465 can be auto-populated based on other information or entries in the medical records dashboard 400. In other embodiments, the information alert 465 can be set by any user to alert the user or other user of information relevant to the patient. In some embodiments, the information alert 465 can comprise a daily technician update, including information to medical information such as blood pressure, or whether the patient is pregnant, or any other urgent information with which a member of a health care team can alert another member. Further, this information can become permanent or can be deleted from the medical records dashboard 400, and from any record or table accessible from the medical records dashboard 400, including any medical record. Further, this information can serve as or be configured as a “sticky note” that can be removed from any of the above-mentioned records. For example, the “sticky note” can be an electronic sticky note riding on the dashboard or any record accessible from the dashboard.

Furthermore, the medical records dashboard 400 can provide improvement as described where test interpretations and evaluation of patients, once documented and billed, usually become date stamped, and cannot be easily amended without applying a new date of amendment. In some embodiments, the Data Command center 001 can improve and follow care that will not necessarily be used as part of a particular day's medical record. Therefore, months or years apart, physicians can add notes into the table when new findings, discoveries, or realizations warrant it without feeling encumbered that they are “changing past medical record” and a disclosure of such can be at the bottom of the medical records dashboard 400. Allowing physicians and technicians to add and change notes within the medical records dashboard 400 (rather than changing a patient's EMR chart) can enable a user to summarize critically important health/history/treatment data, which can then be used as a faster point of reference while examining the patient. Notes that exist on the medical records dashboard 400 can flag or alert a user to an important medical change, and can be used as an additional form of communication to strengthen lines of communication between technicians/clinic staff and physicians to better ensure that a medical care provider is quickly directed to important medical information.

In some embodiments, a daily technician update can be accessed or otherwise made visible to the user in at least one portion of the dashboard 400. In some embodiments, the information alert 465 can be displayed in a specific color and/or with a specific graphic and/or animation. For example, in some embodiments, the information alert 465 can comprise a flashing red animation. To protect the medical care provider during an audit, a statement on the medical records dashboard 400 can be added that “notes on this table” are not necessarily added at the time listed as the date and not for documentation in a medical record, but as a rapid reminder medical decision making and cliff note reference tool. As another example, if this patient's records were ever sent to another medical care provider or insurance company or were audited, this is critical information that a medical care provider is often not privy to and an icon on the table will alert the physician of this fact. By selecting this or another icon, the history of this audit or records release request or other occurrence can be seen. So, if an insurance company is requesting a medical necessity report or other information that is needed by a billing office or anyone else, the medical care provider can be informed on the medical records dashboard 400 so that the medical care provider can instantly decide what is needed.

As depicted in FIG. 4A, the medical records dashboard 400 can include an icon 407 enabling access to one or more letters or results from external data sources or third parties. In some embodiments, the medical records dashboard 400 can further include an icon 408 enabling access to letters sent 408, which can be written, typed, and/or dictated from the user and/or another medical care provider. In some embodiments, the medical records dashboard 400 includes an entry or access to the current day's history, the current day's plan, and/or to the current day's billing. For example, as depicted in FIG. 4A, the medical records dashboard 400 includes a “Today's history” button or icon 409, a “Today's plan” button or icon 411, and a “Todays billing” button or icon 413. In some embodiments, the medical records dashboard 400 can include a correspondence button or icon 436, which can be used to view, access, enter, and/or auto-populate correspondence related to a patient's care. Such correspondence can include any medical record and/or any correspondence generated while the patient is under care by the user and/or any other physician or medical practitioner, medical services provider, and/or medical insurance company.

In some embodiments, the medical records dashboard 400 can display a summary of the patient's problem list in which a user can input patient information and constantly update and change. For example, the medical records dashboard 400 of FIG. 4A includes an icon/button 430 for enabling the entry of or access to current complaints of the patient. In some embodiments, if a user activates (e.g., by clicking using a cursor) the button 430, information related to the patient's current medical problems or complaints can be shown and/or displayed and/or updated by a user. In some embodiments, the information can be auto-populated into the medical records dashboard 400.

In some embodiments, the medical records dashboard 400 can include a today's examination access icon/button 432 enabling a user to access, view and/or input patient information, patient examination results, tests, notes, or any information relevant to the medical care of the patient. By activating (e.g., by clicking using a cursor) today's examination access icon/button 432, information related to the patient's examination including medical problems or complaints patient information, patient examination results, tests, notes, etc., can be presented and/or displayed and/or updated using one or more windows and the like. In some embodiments, the information associated with the today's examination access icon/button 432 can be auto-populated into the medical records dashboard 400 by, for example, the display module 006 following auto-population rules of the rules module 004.

In some embodiments, any stored or displayed patient's examination records/data can be cleared from the medical records dashboard 400 following some time period once a patient visit is complete. In some embodiments, the medical records dashboard 400 can remove the display of or access to a previous patient's examination details once the patient visit has ended. In some embodiments, the medical records dashboard 400 can remove display or access to a previous patient's examination details later in the day of the patient's visit, or before the following day, or at any time selected by the user. In some embodiments, the information can be auto-populated into any EMR system for recordation into one or more EMR's of the patient. In some embodiments, for any auto-populated information that includes technical information without any associated professional interpretation, the Data Command center 001 via the medical records dashboard 400 can provide a visual and/or audible alert to enable a user to provide an update for auto-population to an EMR system.

In some embodiments, the medical records dashboard 400 can include at least one link to information from external databases, providers, hospitals (e.g., such as a discharge summary), clinics and/or testing laboratories, etc., (e.g., where the information can include the overall diagnostic imaging center of the practice for certain pieces of equipment and into the machine to actually see all of the study). In the latter example, the medical records dashboard 400 can receive information from at least one coupled database and/or server and/or controller. For example, as depicted in the embodiment of FIG. 4A, the Data Command center 001 via the medical records dashboard 400 can have entry or access to third party data sources via icons/buttons, including but not limited to, the National Patient Registry icon/button 415, the hospital EMR icon/button 417, the imaging center icon/button 419 and the ePrescribe icon/button 421.

In some embodiments, orders can be auto-populated into the medical records dashboard 400 or order screen of an EMR using, for example, the Orders icon/button 423. For example, in some embodiments, during or after completion of a patient examination, any medical service, medical test or diagnostic, or other medical service can be auto-populated into an order section of the medical records dashboard 400. Any recommendation for a return visit can be viewed, accessed, and/or auto-populated using the return visit icon/button 434 of the medical records dashboard 400. For example, in some embodiments, the recommendations can be any advised next steps in the patient's care, any diagnosis, prescriptions, tests, etc. In some embodiments, the aforementioned “Today's plan” icon/button 411 can be used to view, access, and/or auto-populate details including for a day's activities for the patient examination.

In some embodiments, an “Imaging Center” icon/button 424a of the medical records dashboard 400 enables a user access to the piece or pieces of diagnostic equipment that were used that or another day for performing tests on a patient(s) so the user can now measure and/or access the test results. Such functionality can be internal to the user's practice so that any diagnostic equipment can be accessed. The ability to access the diagnostic equipment and data directly, in accordance with the present principles, enables a user access to not just one single piece of diagnostic equipment but all equipment available and all tests available can be evaluated and the evaluation of changes of such tests over time can be made.

In the embodiment of the medical records dashboard 400 of FIG. 4A, the icon/button 424b enables a user access to a company sponsoring a clinical research website (i.e., sometimes a pharmaceutical company and other times a company that invented a device). Such functionality enables a clinical researcher access to such a website and input any data that was obtained from a patient visit using the access provided using the access provided. In a research application in accordance with the present principles, a researcher is provided access to diagnostic equipment and/or to a research spreadsheet where the researcher can access and input data.

Further details of the problems window 425, surgeries window 450, and command center visual display window 500 are provided in FIGS. 4B-4D illustrating enlarged views of portions of the medical records dashboard 400. For example, FIG. 4B depicts a portion of the medical records dashboard 400 of FIG. 4A in accordance with some embodiments of the present principles. As illustrated in FIG. 4B, in some embodiments, the information columns 600 of the problems window 425 can include a date column 610, a timeline column 620, an “ICD” column 630 for international classification of disease codes including international classification of disease codes, such as version 9 or version 10, (hereinafter collectively referred to as “ICD code” information), location of the problem or disorder (shown as “OD”, “OS”, “OU” identifying right eye, left eye, both eyes), or from any part of the body, and a diagnosis column 650 for detailing information related to an initial diagnosis or final diagnosis of a patient's problem or disorder that can be auto-populated or input manually. Further, in some embodiments, the information columns 700 of the surgeries window 450 can include information related to services or procedures that were provided to the patient (procedure columns 720), a description of the services or procedures performed (description columns 730), and when the services or procedures were provided (timeline columns 710).

FIG. 4C depicts greater detail of at least some portions of the medical records dashboard 400 of FIG. 4A in accordance with some embodiments of the present principles. Referring to FIG. 4C, in some embodiments, the surgeries window 450 can include location information 740, surgeon or physician information 750, and a comments section 760. Referring to the display window 500 of FIG. 4C, the information columns 800 can include a date column 805, and a procedure column 810 illustrating or providing access to information detailing one or more procedures performed on the patient. Further, the procedure column 810 can include an “OD” column 815, and “OS” column 820 providing right and left eye procedure information, or could be a body part (i.e., orthopedic surgery limb versus spine). In some embodiments, information related to the medical care provider, the location where the procedure was performed, and office visit information can be provided to the user in column 830, and unit column 840, and office visit column 845.

FIG. 4D depicts greater detail of at least some portions of the medical records dashboard 400 of FIG. 4A in accordance with some embodiments of the present principles. Referring to FIG. 4D, in some embodiments the user can view information related to tests and procedures performed on the patient. For example, in some embodiments, such information can include information related to one or more medical imaging procedures such as an optical coherence tomography (“OCT”), or fluorescein angiography (“FA”), and/or indocyanine green chorioangiography (“ICG”), or any current procedural terminology code (hereinafter “CPT code”), including any CPT code found in the American Medical Association CPT 2015 professional edition or other edition, the entire contents of which is incorporated by reference. Moreover, the user can view information related to tests and procedures performed on the patient based on an ICD code. Other clinical vocabularies such as Systematized Nomenclature of Medicine (hereinafter “SNOMED codes”) can be used in other embodiments as the system is not limited.

In some embodiments, the user can compare patient clinical information, such as labs and vitals using the medical records dashboard 400, before and after a selected medication has been prescribed or a procedure has been performed to better understand the effect of the medication or procedure on the patient. Similarly, in other embodiments, the user can examine how the current patient compares against other patients in the practice and population in general using the medical records dashboard 400 to better understand outcomes.

As depicted in FIGS. 4A-4D, in some embodiments, medical procedures performed (including any of the aforementioned medical imaging procedures) that have been billed and claimed can be viewed or accessed by a user within any of the “OCT” column 850 (split as an “OD” column 855 and “OS” column 860), an “FA” column 870 (split as an “OD” column 872 and “OS” column 874), and/or “ICG” column 880 (split as “OD” column 882 and “OS” column 884).

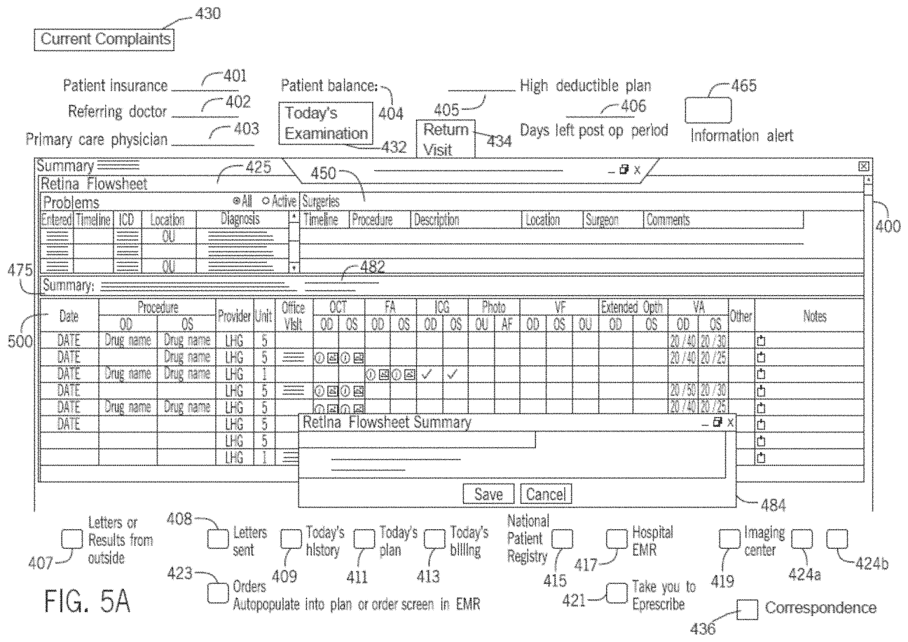

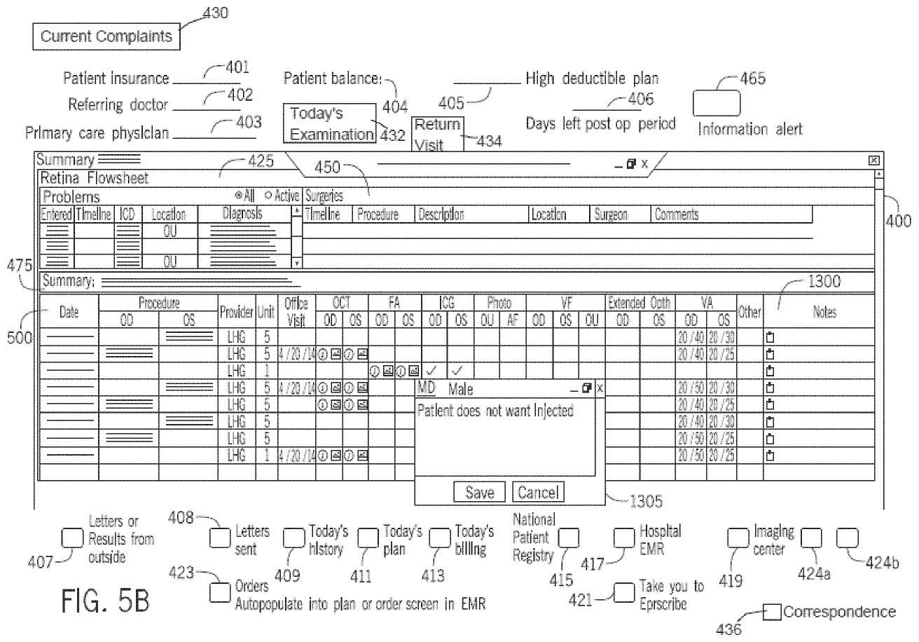

Referring back to FIG. 4C, the information columns 800 can include a photo column 890 configured to enable a user to access any photographic images of the patients eyes including optical and auto-fluorescent images of the eyes (“OU” column 892 and “AF” column 894). In some embodiments, if visual function tests were performed, information can be viewed or accessed in the visual field “VF” column 900 (including an “OD” column 910, “OS” column 920, and/or “OU” column 930). Some embodiments also include an extended ophthalmology column 1000 (including “OD” column 1050 and “OS” column 1070), and a visual acuity column (“VA” column 1100, including “OD” column 1150, and “OS” column 1170). In some embodiments, as described earlier, other details of various tests, procedures or services can be viewed or accessed in the other column 1200. Further, information associated with any of the user-accessible tests, procedures or services or other notes provided by the user and/or medical care provider can be viewed or accessed in the notes column 1300 using one or more notes access icons/buttons 1350 and/or by viewing a note entry 1375 (e.g., and/or any note entered using the note entry window 1375. . . . The functionality of the notes column 1300 is further discussed below with reference to FIG. 5B). In accordance with embodiments of the present principles, the information can be auto-populated into the medical records dashboard 400 or into EMR plan pages as described above with respect to different embodiments. That is, as described above, various data fields/entries of the medical records dashboard 400 of the Data Command center 001 of, for example, FIG. 1, can be auto-populated via an electronic dataflow established between the Data Command center 001 and one or more computer systems of servers that comprise patient information (e.g., such as electronic medical records). The dataflow to and from the medical records dashboard 400 can comprise a two-way flow from the source of patient data to the Data Command center 001, and from the Data Command center 001 to the data source.

In some embodiments, the medical records dashboard 400 can include visual cues, icons, or markers representing and/or enabling access to detailed information related to medical services, procedures or tests provided to the patient. Further, by employing data visualization techniques, a user's eye can be trained to quickly identify these icons or markers and increase the efficiency of user accessing key medical indicators such as test results and surgical histories. For example, in some embodiments, medical services, procedures or tests performed or provided can be assigned a visual code, icon, or graphical marker. For example, the embodiment of the medical records dashboard 400 of FIG. 4B depicts visual cues, icons, or markers 885 representing medical services, procedures or tests performed or provided to a patient. In some embodiments, the information columns 800 within the display window 500 can include at least one “test done, no image attached” icon 885a, one or more “see image in order viewer” icon 885b, at least one “view order interpretation” icon 885c, and/or at least one “procedure billed or claims made” icon 885d, where an appearance in the medical records dashboard 400 can indicate that a claim was made, and a change in color or other method (italics, bold, etc.) can represent whether the bill was paid. Further, FIG. 4D depicts another example of “test done, no image attached” icon 885a, “see image in order viewer” icon 885b, “view order interpretation” icon 885c, and “procedure billed or claims made” icon 885d, in which an appearance in the medical records dashboard 400 represents a claim was made, and a change in color or other notification method can represent whether the bill was paid. Data visualization icons and markers located in the medical records dashboard 400 can be used to quickly identify billing or coding errors by enabling a user to determine inconsistencies among various entries in the medical records dashboard 400, and thus can empower the physician to be proactive and thorough in areas of compliance with insurance guidelines. The use of these icons to identify potential errors in coding can provide an additional level of protection and proofing to reduce and prevent potential billing and/or malpractice errors.

In some embodiments, the medical records dashboard 400 can provide a text summary of any entry within the medical records dashboard 400. As described earlier, the summary window 475 can enable a user to view and edit summary information related to the patient, any details of care provided to the patient, and/or and any medical diagnosis information prepared by a medical practitioner. In some embodiments, the user can add and/or edit the summary information. For example, FIG. 5A depicts the medical records dashboard 400 including a medical summary update process in accordance with some embodiments of the present principles. In the embodiment depicted in FIG. 5A, the medical records dashboard 400, including the problems window 425, surgeries window 450, summary window 475, and command center visual display window 500, can further include summary comments 482 that can be entered, updated, expanded using the summary input window 484. In some embodiments, a user can enter information within the summary input window 484 for entry into the summary window 475.

FIG. 5B depicts a portion of the medical records dashboard 400 including a notes update procedure in accordance with an embodiment of the present principles. For example, using a notes update procedure, a user can add or update information associated with any of the user-accessible tests, procedures or services or other notes provided by the user and/or medical care provider in the notes column 1300. As depicted in the embodiment of FIG. 5B, the medical records dashboard 400 can include the problems window 425, the surgeries window 450, and the summary window 475, and the visual display window 500 of with notes column 1300 of the medical records dashboard 400 can be updated with one or more notes using the note entry window 1305.

In some embodiments, placement or viewing functions of the medical records dashboard 400 can be toggled using a left or right mouse click function. For example, in some embodiments, following an initial impression or diagnosis, a right click can bring up a note function (e.g., through note entry window 1305), and/or a left click can bring up the summary function (e.g., through summary window 475 as summary comments 482).

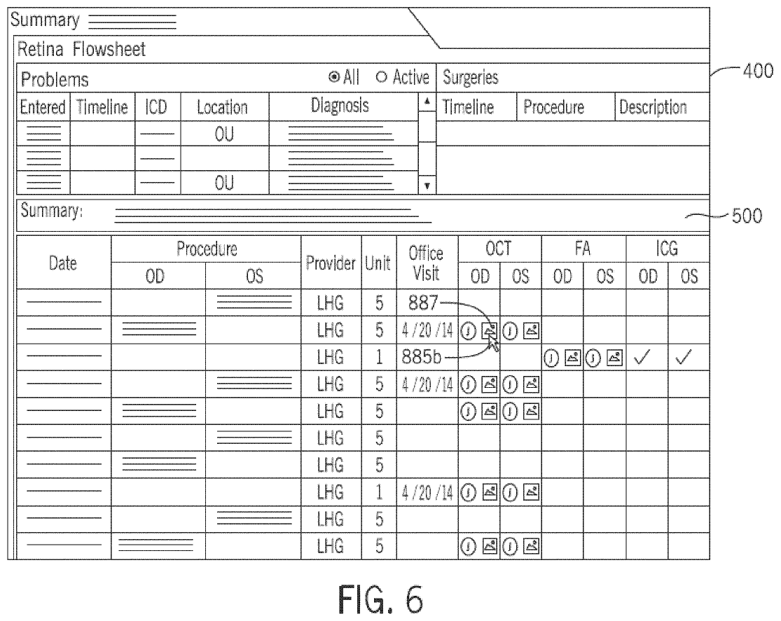

Referring to at least FIG. 4B, in some embodiments, a user can access underlying information linked to visual cues, icons, or markers 885 by, for example, using a single click or mouse-over. That is, in accordance with the present principles, in some embodiments a user can use the visual display window 500 of the medical records dashboard 400 to access and view any information auto-populated within the visual display window 500 and/or other windows or sub-windows of the medical records dashboard 400. For example, FIG. 6 depicts a user record access process of the medical records dashboard 400 in accordance with an embodiment of the present principles. In some embodiments, a user action 887 (depicting a user click or mouse-over of a cursor) can enable a user to access and view information (in this example, information linked to “see image in order viewer” icon 885b). In some further embodiments, a user can use a single click of or mouse-over of a portion of the medical records dashboard 400 to access and view any information within any portion of the medical records dashboard 400. Further, in some embodiments, a user can use left and right mouse clicks to navigate from one portion of the medical records dashboard 400 to another. Furthermore, in some embodiments, a right-click mouse function can be used to bring up and update a an portion of the medical records dashboard 400 and/or display any important information in the medical record dashboard 400, and a left-click can bring the user back to another portion of the medical records dashboard 400.

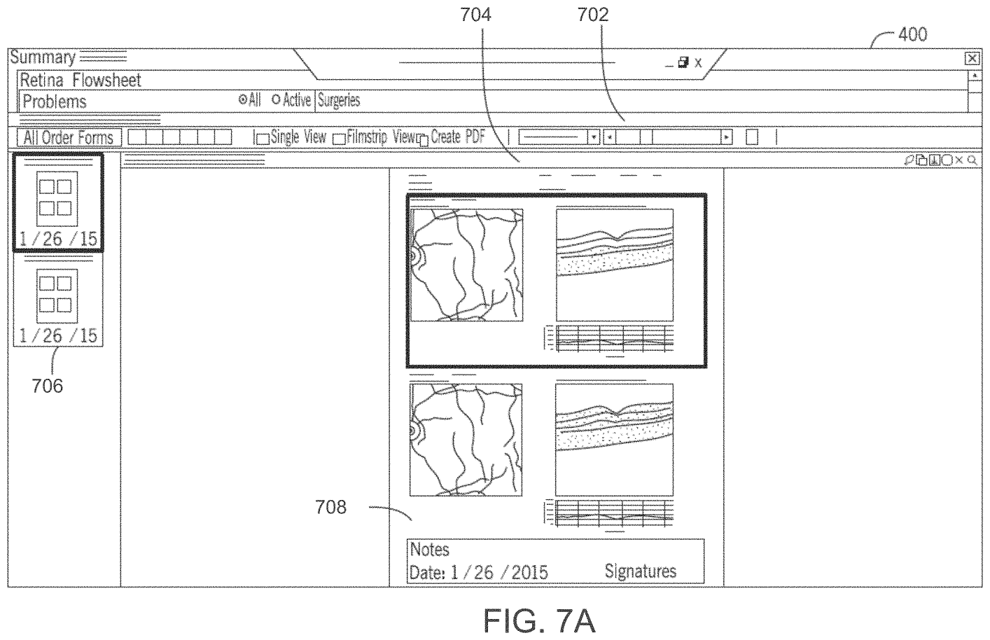

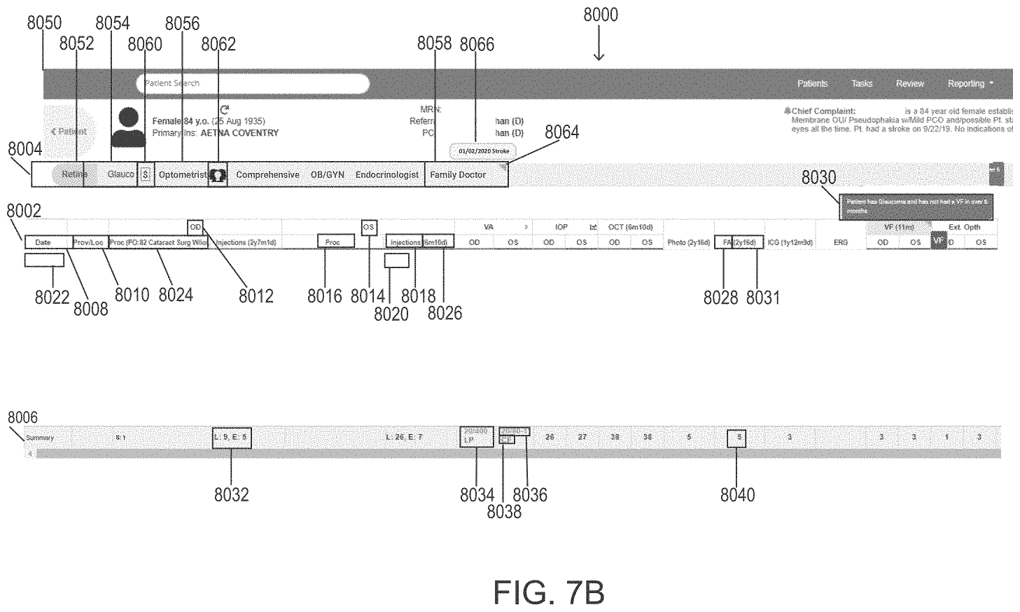

In some embodiments, the Data Command center 001 via the medical records dashboard 400 can display at least one medical record as a result of the user action 887. For example, FIG. 7A depicts a medical records access window 702 of the medical records dashboard 400 in accordance with an embodiment of the present principles. In some embodiments, the user's action (represented by user action 887) can cause a display of the medical record access window 702 including a medical record display 704. Further, in some embodiments, at least one medical record 708 can be selected from the medical record list 706 for viewing in the medical record display 704. As illustrated in FIG. 7A, in some embodiments, the at least one medical record 708 can comprise an image or photograph such as an optical and/or fluorescein angiogram image. In other embodiments, the at least one medical record 708 can comprise an X-ray image. In some further embodiments, the at least one medical record 708 can include an MRI scan or any report or anything ordered or performed by medical care providers. In some embodiments, the at least one medical record 708 can comprise one or more dictated letters from the user or another medical care provider. Further, in some embodiments, the at least one medical record 708 can comprise a record or any portion of a correspondence from another medical care provider.

A unique aspect of the medical records dashboard 400 of the Data Command center 001 in accordance with the present principles is that so much relevant patient information can be viewed in context to the procedures, the clinical information and/or the medical services provided over time while having direct one click access to any image and diagnostic test or plan. In addition, in embodiments of the present principles all of the patient studies can be accessed in context of all other patient data.

In some embodiments, images related to patient treatment can be viewed as thumbnails in one or more windows being visible and accessible along with at least portions of all other data available on the medical records dashboard 400. In some embodiments, a user is able to manipulate and modify an image with the ability to store and recall the modified image. For example, a user, while looking at an image in context, can mark and make notations, draw on the image. Such modifications can be stored with the image or with a copy of the image.

In some embodiments, thumbnails of respective images can be displayed with an ability to see the full-size image including relevant information. For example, in some embodiments, thumbnails can be displayed across the bottom of a display of all images in a column of the medical records dashboard 400, one per image, such that a user is able to pull up all visual fields of a column, such as the OCT column. Further embodiments describing the display of patient care related images are described further below. In accordance with the present principles, what is critical is not that the Data Command center 001 via the medical records dashboard 400 can display images related to patient care, but instead that all of the images can be looked at in context with clinical and exam findings and or procedural information and dates of other medical services lined up in an intuitive way that enables a user to quickly decide which image or test to review, and in some embodiments, receive guidance from the Data Command center 001 on how to proceed with treatment using various tools and functionality of the Data Command center 001 described herein.

In some embodiments, a user is able to assign a respective icon for accessing and representing images in the medical records dashboard 400. In some such embodiments, the assigned icon can visually represent information related to the image, including whether or not the image indicates that a patient's condition has gotten better, worse or has remained the same.

In some embodiments, the Data Command center 001 via the medical records dashboard 400 can enable a user to access underlying information linked or related to diagnostic codes listed in the medical records dashboard 400. In some embodiments the Data Command center 001 via the medical records dashboard 400 can enable a user to access underlying information linked or related to billing codes. For example, in some embodiments, using a single click or mouse-over, a user can use information made available via the visual display window 500 of the medical records dashboard 400 to access and view any information related to diagnostic and/or billing codes. In some embodiments, the diagnostic and/or billing code information and payment history can be displayed in a separate document or window. In some other embodiments, diagnostic and/or billing code information can be display overlaid onto the medical records dashboard 400 (e.g., as a pop-up window or transient text and/or graphics).

FIG. 7B depicts the functionality of three specific rows in the user interface in accordance with an embodiment of the present principles. That is, FIG. 7B illustrates how three rows or panels in the user interface 8000 can convey a plethora of information for a healthcare professional in some embodiments. Header 8002 is an example of a header that can appear on each individualized specialty-based provider's actionable dashboard. As illustrated at 8004, different actionable dashboards that have been particularly designed for different providers of different specialties can be accessed. In the embodiment of FIG. 7B, a summary row 8006 can be provided on each individualized dashboard for each doctor, specialist, or other user and can be specialized for each user.

In the header 8002, the date is represented at 8008. The date can include a time, day, and/or date of a patient visit or the visit of a group of patients. 8010 can include the initials or name of the provider who cared for the patient or if just a location of testing, can include an indication of the location of the test performed. That is, in some embodiments, a medical care provider's initials can be presented at 8010, which can also include a location, as providers can have multiple offices. In some embodiments, the information in 8010 can include an abbreviation, description, or identifying factor of which office a patient visited. In FIG. 7B 8022 shows an example of one of many patient encounters over time.

In FIG. 7B, 8012 depicts an example of a column that includes a procedure and, in the embodiment of FIG. 7B, is divided into two different sides of a patient's body. In 8012 OD is displayed, which in eye care refers to a patient's right eye. In the case of orthopedics, 8012 could reference a patient's right knee. Similarly, an OS in 8014 represents the left column or in the case of eye care, the left eye, and in the case of orthopedics can refer to a left knee. As illustrated, the OS or left column of the header can be a procedure 8016. Under the column of the left eye is listed, by encounter, identifying procedures such as injections 8018. Item 8020 depicts a focal procedure that has been performed (e.g., injection of Eylea), while 8022 shows the date of service being Mar. 21, 2017.

As illustrated at 8024, the header 8002 is able to display that a cataract surgery has been performed and that a postoperative period is counting down. The header 8002 can also display that injections 8026 were last performed six months and ten days ago. FIG. 7B depicts that another data element 8028, such as FA, can be displayed over time. In addition, 8030 can display a last time this test or item was performed on a patient. In the embodiment of FIG. 7B, 8030 depicts that a test was performed 2 years and 16 days ago. Header 8002 also includes a pop-up 8030 of underlying information. In the embodiment of FIG. 7B, the patient has a diagnosis of glaucoma and has not had a visual field in over six months.

The summary row 8006 of FIG. 7B shows how not only is the total number of something that had occurred in the rows above counted, but it can be divided according to what was performed. That is, in the embodiment of FIG. 7B, summary row 8006 is a smart summary column. In the embodiment of FIG. 7B, 8032 demonstrates that there were twenty-six Ls and seven Es of which one E (Eylea) is shown at 8020. The embodiment of FIG. 7B depicts an example of a retina doctor who performs injections in the right eye in this case, and used “L,” which stands for Lucentis times and “E,” which stands for Eylea. 8034 similarly demonstrates the summary cell in a column of the left eye. 8036 shows the vision in the left eye is 20/80-1 and could also reflect the best number or event that occurred in the entire row overall of dates of service, and can be highlighted to inform the user that it is the best value. 8038 shows CF. In this illustration of a retina surgeon, CF means count fingers, which is very bad vision and is, therefore, red. For the first time in this illustration, a retina surgeon can know, over the time that the doctor has been delivering services, or any doctor, what is being reflected in encounters and rows above. The best vision was 20/80 (8036). The worst was count fingers (8038). This can also be the best blood pressure and the worst blood pressure. Every different specialty in medicine has different ways that it would like to measure the highs and lows in a column. 8040 simply shows the counting of the number that had occurred in all of the encounters. In this case five FAs 8028.

It is important to note that the tool can measure anything in the row and display it in multiple different ways. The choice could be just to see the high and low as in 8036 and 8038 over a short period of one year or over as many years as there have been encounters. It can also be set to show percentage changes over time. In any case, this summary provides a tremendous amount of information to the provider for enabling rapid decisions.

A panel 8050 may be located at the top, side, or bottom of the display in FIG. 7B and may provide access for each specialist to different types of healthcare providers or different doctors who want to customize the display. Any type of doctor or dentist or other health care provider of any specialty can be listed. As few or as many as have actionable dashboards that can be accessed immediately with direct access by simply clicking on the specialist's name. For instance, the specialists may be retina doctors 8052, glaucoma doctors 8054, or an optometrist 8056. All three happen to be types of eye doctors. All three could be in the same practice, separate practices, or even in different countries. Each, when clicked on, pulls up an actionable dashboard specially designed for them or their practice in that specialty. 8058 provides an example of a non-eye doctor, in this case, the family doctor.

It is important to note that any health care provider, if given permission by the patient, and each specialty noted in FIG. 7B could see the actionable dashboard of the other specialists for as little or as much as each would allow. There is some information in an actionable dashboard to each specialist, practice, or doctor that they might not want others to see, which can be hidden (e.g., payments and costs). In addition, next to each actionable dashboard can also be additional information that can also be pulled up instead of the actionable dashboard itself. For instance, a dollar sign 8060 could be for providing for each practice or actionable dashboard, payments, costs or any financial matter that can pop-up to show a different type of financial dashboard. 8062 shows an example that can pull up any type of additional information, such as a shared care dashboard between different providers.

8064 illustrates that an entire cell can alert all of the other providers of something important. It can be a color change, or flash, or blink. When activated, it represents that there is some type of important event, for instance, that all providers should know. A pop-up 8066 also may be shown at all times or by hovering over 8064. The popup could represent whatever the important item is to be alerted. For instance, a new diagnosis like the patient had a stroke on Jan. 2, 2020, which all providers would like to know. It can also inform all providers that the patient missed an appointment that was very important with that doctor. So, that all specialist would know that and be able to remind the patient.

It will be further appreciated that the actionable dashboard may further include a communication center where users can send messages to each other in a HIPAA compliant way. In other words, a physician, while seeing a patient, can send a message to their chief technician or the office manager to talk about following up on a patient or also to the billing office that there is a billing problem. Then, staff can report back to the doctor and this message can be imbedded into the smart actual dashboard so that the next time a doctor sees the patient through icons and columns of correspondence of communication within the practice, the doctor can pull up what was the response to a message they had sent earlier. This response can be read live while treating the patient so that the doctor can take it into perspective while making decisions. The messaging system, attachments or anything else can be sent to the doctor or health care provider in any way that they would want. Whether through email or the internal messaging system or as a tickler system within the EMR system that automatically toggles back and forth to the actionable dashboard, so the doctors can see their messages at the end of the day or the end of the week, or while seeing the patient. It really helps organize the doctor's life, so this actionable dashboard becomes the communication hub, the switchboard, for the entire practice, while communicating with the health care provider.

With reference to FIG. 7A, in some embodiments, at least one medical record 708 can comprise a transition of care document (hereinafter “CCD”). In some embodiments, the Data Command center 001 via the medical records dashboard 400 can be configured to receive one or more CCDs from one or more medical care providers for display to the user. In some embodiments, the Data Command center 001 via the medical records dashboard 400 can be configured to extract information from the CCD for display to the user. For example, in some embodiments, information from a received CCD can be extracted and used to populate one or more data columns or fields of the medical records dashboard 400 and/or one or more linked data columns or fields of the medical records dashboard 400. In some embodiments, the Data Command center 001 via the medical records dashboard 400 can be configured to receive direct messaging information exchange with other healthcare organizations such as IHE profiles, CDA and CCD, NwHIN Direct, HL7v2, HL7v3, DICOM, X12, ITK (UK), DMP (France), and NEHTA (Australia), etc. For example, in some embodiments, the integration module 002 of the Data Command center 001 can include an HL7 message router and schemas for exchange of direct messages including a graphical editor for transforming messages and data.

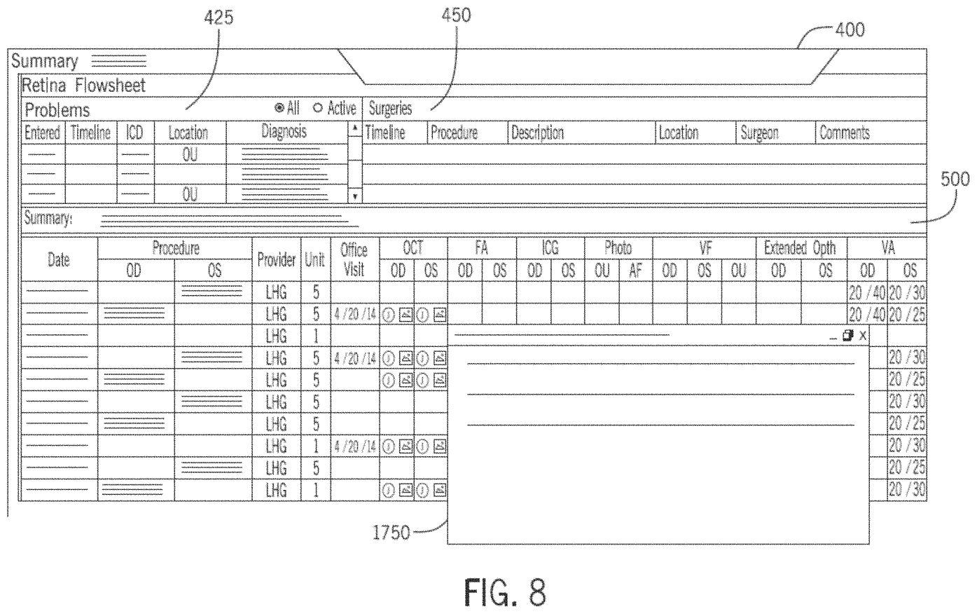

In some embodiments of the present principles, a user via the medical records dashboard 400 of the Data Command center 001 can retrieve and/or update information related to a medical diagnosis. For example, FIG. 8 depicts a medical records and diagnosis update process of the medical records dashboard in accordance with an embodiment of the present principles. In some embodiments, the medical records dashboard 400 including problems window 425, surgeries window 450, summary window 475, and command center visual display window 500 can include an option to enable a user to update or enter at least one medical diagnosis using a medical record/diagnosis window 1450. In some embodiments, multiple medical diagnoses can be provided or updated by a user. In some embodiments, the user providing the medical diagnosis can be any medical practitioner providing the service or procedure to the patient. In some other embodiments, the medical record/diagnosis window 1450 can be updated by a user other than the medical practitioner providing the service or procedure to the patient.

Further, in some embodiments, information can also be auto-populated into the EMR plan pages. A Data Command Center in accordance with the present principles via the medical records dashboard 400 can auto-populate various data fields related to information in any one of the problems window 425, surgeries window 450, summary window 475, and record/diagnosis window 1450 via an electronic dataflow established between the Data Command Center and one or more computer systems of servers that comprise patient information (e.g., such as electronic medical records). The dataflow can comprise a two-way flow from the source of patient data to the Data Command Center, and from the Data Command Center to the source.

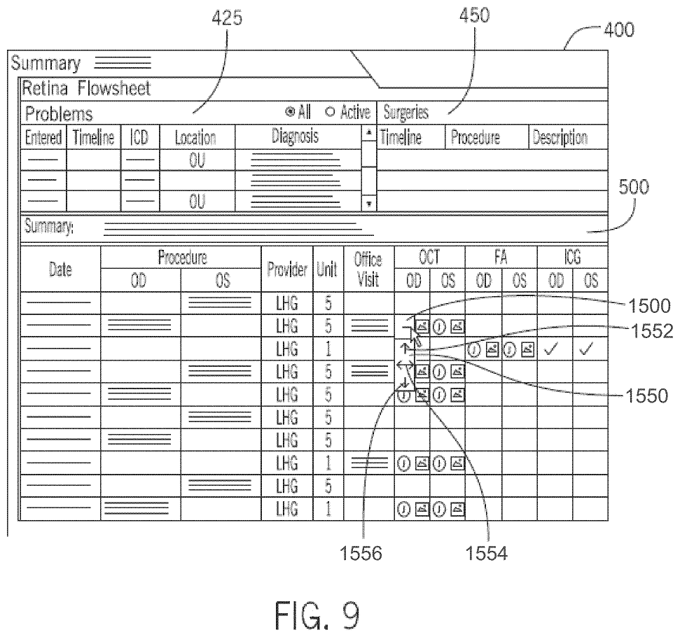

In some embodiments, the Data Command center 001 via the medical records dashboard 400 can enable a user to update information displayed in the visual display window 500. For example, in some embodiments, a user can update information related to a medical diagnosis and/or information related to a medical test or other service or procedure. For example, FIG. 9 depicts a medical record update marker process of the medical records dashboard in accordance with an embodiment of the present principles. That is, in FIG. 9 the medical records dashboard 400, including problems window 425, surgeries window 450, and summary window 475 is depicted with a record update marker 1500 being accessed by a user and displaying an update marker selection tab 1550. In some embodiments, the update marker selection tab 1550 can include a user-selectable marker or icon. For example, in some embodiments, update marker selection tab 1550 can include a selectable diagnosis indicator 1552, a selectable diagnosis indicator 1554, and/or a selectable diagnosis indicator 1556. In some embodiments, the selectable diagnosis indicators 1552, 1554, 1556 can provide a graphical representation of a medical diagnosis, outcome, or test. For example, in some embodiments, the diagnosis indicators 1552, 1554, 1556 can provide a visual representation of an improvement of a medical problem, disease, or symptom, or a worsening of a medical problem, disease, or symptom. Further, in some embodiments, the diagnosis indicators 1552, 1554, 1556 can provide a visual representation of a medical problem, disease, or symptom that is stable or substantially unchanged. In some embodiments, the diagnosis indicators 1552, 1554, 1556 can provide a visual representation directly related to one or more variables of a physical test. For example, in the field of ophthalmology, some imaging tests can provide an analysis of the thickness of the retina related to an eye disease such as macular degeneration. In some embodiments, an increase in thickness can represent a worsening of the condition, whereas a decrease in thickness can represent an improvement. A stable or unchanged thickness can indicate the disease is responding to treatment or is in remission. Further, by using data visualization techniques such as by using a color change or other method (e.g., such as using italics, bold text, and/or underlined text), a particular important change in a test can be marked for internal reference alerting a physician to the tests or procedures that are important and to take note for future reference. Further, in some embodiments, the diagnosis indicators 1552, 1554, 1556 can comprise a color and/or graphical change providing a visual representation of items billed, items not billed, or tests needing reports or interpretations are required. A color change or data visualization method (e.g., such as using italics, bold text, and/or underlined text) can also tell a physician if a test or procedure was billed, rejected, or if an interpretation needs to be made.

As an example embodiment, the diagnosis indicators 1552, 1554, 1556 can provide a visual representation of the status of a patient with an eye disease such as macular degeneration. For example, in some embodiments, the diagnosis indicators 1552, 1554, 1556 can be selected from the update marker selection tab 1550 when the user intends to indicate a worsening of the condition (e.g., where the thickness of the retina is increasing). In some embodiments, any of the diagnosis indicators 1552, 1554, 1556 can be color-coded to represent a status or provide a visual indicator of a medical condition, test, or diagnosis linked to the diagnosis indicators 1550. For example, in some embodiments, the diagnosis indicator 1552 can be color coded red and the diagnosis indicator 1556 can be color-coded green. Further, the diagnosis indicator 1554 can be color-coded blue or black. In some other embodiments, the diagnosis indicator 1552 can be color coded green and the diagnosis indicator 1556 can be color-coded red. In other embodiments, other graphical markers or icons can be used, and/or other colors can be used to differentiate the diagnosis indicators 1552, 1554, 1556. Further, in some embodiments, in addition to or in place of using a color differentiation between the diagnosis indicators 1552, 1554, 1556, one or more of the diagnosis indicators 1552, 1554, 1556 can flash or pulsate.

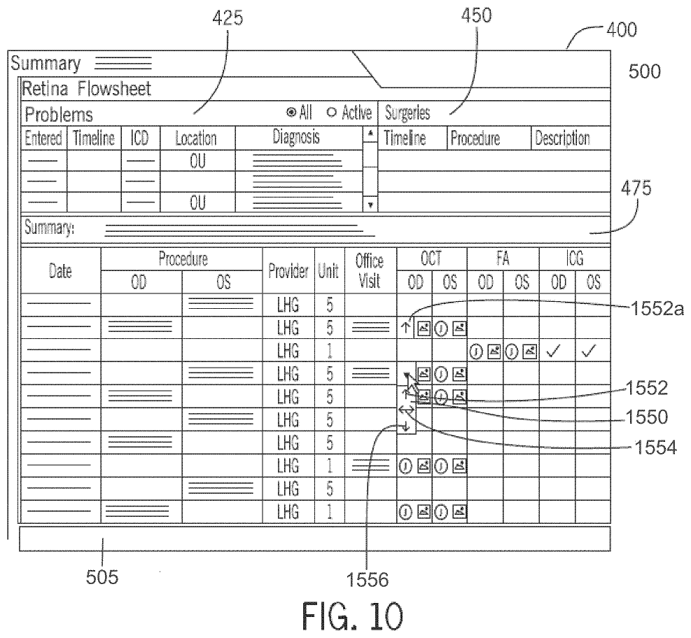

In some embodiments, the Data Command center 001 via a medical records dashboard of the present principles, such as the medical records dashboard 400, can enable a user to provide a plurality of updates to information displayed in the visual display window 500. For example, in some embodiments, a user can update information related to a medical diagnosis and/or information related to a medical test or other service or procedure, and subsequently provide further updates to the same information or to other information. For example, FIG. 10 depicts a medical record update marker process of the medical records dashboard 400 in accordance with an embodiment of the present principles. The embodiment of the medical records dashboard 400 of FIG. 10 includes a problems window 425, surgeries window 450, summary window 475, and command center visual display window 500. The command center visual display window 500 depicts diagnosis indicator 1552a representing previously updated information. The visual display window 500 also illustrates a user updating information with a process described above using the update marker selection tab 1550 comprising a selection of diagnosis indicator 1552, diagnosis indicator 1554, or diagnosis indicator 1556. In some embodiments, the diagnosis indicator 1156 can be modified to be indicative of updated information or status of a patient and/or a patient's disease, test, or medical condition. Further, any ICD, SNOMED or similar code can be inserted.

In addition, FIG. 10 illustrates a portion of the medical records dashboard 400 including a scrolled display in accordance with some embodiments. In some embodiments, the medical records dashboard 400 including problems window 425, surgeries window 450, summary window 475 can include a command center visual display window 500 that comprises a scroll display 505. In some embodiments, any information displayed in the command center visual display window 500 can be scrolled by the user to bring non-visible portions of the command center visual display window 500 into view. Such capability can enable the user to view the entire history of the patient independent of the number of years of history that is on record.

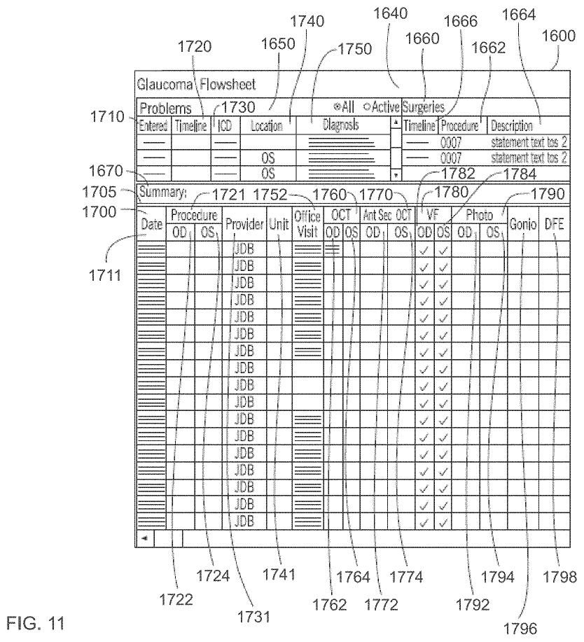

FIG. 11 depicts a portion of a medical records dashboard 1600 in accordance with another embodiment of the present principles. In some embodiments, the medical records dashboard 1600 can display data from one or more medical records, and/or track medical procedures and services based on claims made or billing signed off by a physician for one or more delivered medical procedures or services. Further, in some embodiments, the medical records dashboard 1600 can be auto-populated as a function of claims made or billing signed off by a physician, auto-populated from any portion of a selected chart. In this instance, any data displayed within the medical records dashboard 1600 can be derived from one or more claim records that have been billed for one or more procedures or services have previously been provided to the patient. In reference to the medical records dashboard 1600 and/or the previously described medical records dashboard 400, in some embodiments, auto-populating visits by actual claims made or billings signed off by a physician, by definition occurs after the visit with a patient.

In some embodiments, the Data Command Center of the present principles can auto-populate some information of the medical records dashboard at the time the patient is seen, or shortly thereafter, or even before in preparation for a visit (i.e., lab results), so that even if a patient is not seen on a particular day, the user (e.g., medical care provider) can view the displayed information in the table for information. For example, in some embodiments, information related to vision can be made with the current date at the time patient is seen. In some embodiments, a user or user's assistant can update the Data Command Center with medical tests or test results (e.g., a vision test) as they are performed or shortly thereafter (i.e., on the same day). In this example, this information can immediately trigger the current date and auto-populate the vision column. This information can then be immediately viewed by a user and/or medical care provider and can be updated with notes or comments or other information as the user and/or medical care provider is attending to the patient. Further, after the claim has been made for any diagnostic tests or examinations or procedures that have not yet been billed, the date will then auto-populate in the future with the other related columns. In some embodiments, while examining a patient, important information and/or certain parameters that are critical to follow can be immediately updated to the Data Command Center. Using these procedures, the Data Command Center of the present principles can enable a medical care provider to review the patient's medical history, treatment history, and instantly see items of importance on the day they're examining a patient. For example, the user and/or medical care provider can be enabled by the Data Command Center, on the day the patient is examined, to review information such as a vision or glaucoma table, intraocular pressure, blood pressure, blood sugar, etc. When billing claims are made, further information is filled to complete the billed claims record. As a further example, a patient may be seen a few days apart and the diagnostic tests etc. and claims have not yet been made, however the Data Command Center can be configured to show that the patient was seen that day (e.g., with a vision, pressure test, etc.), and the Data Command Center can enable a user (such as a physician) to interpret and/or add special notes on the day they see a patient or before they see the patient rather than waiting to make some notes when a claim is actually generated.

If a medical office wishes to communicate results or a test (e.g., a pathology result or test) to a user, in some embodiments, a blinking cursor can appear to alert the user. Also any written or typed correspondence or any links to dictated information using voice recognition can be coupled to or integrated with a medical records dashboard via the Data Command Center of the present principles. For example, in some embodiments, the integration module 002 of the Data Command center 001 of FIG. 1 can integrate such information into the medical records dashboard. In some embodiments, information can be auto-populated into the medical records dashboard with the touch of a button into a designated location such as the current plan documenting the patient's current visit (thus aiding documentation for the current visit). Further, whatever is important for a user to input into the day's visits for documentation can be initially inputted in the table, and then permanently into the day's patient visits. Further, a summary section of a medical records dashboard can be constantly fluid, and can be changed at every visit rather than being written to an unchangeable document or file (e.g., such as a PDF). Any patient data that is inputted, received, analyzed, or created can be auto-populated into any portion of a medical records dashboard. The Data Command Center can auto-populate in a one-way or two-way direction in various data fields related to information in any patient information via an electronic dataflow established between the Data Command Center and one or more computer systems of servers that comprise patient information (e.g., such as electronic medical records). The dataflow can comprise a two-way flow from the source of patient data to the Data Command Center, and from the Data Command Center to the source including another electronic system or server, or another user, observer, or other 3rd party.

By following a patient on the day of delivery (e.g., for a vision intraocular pressure or anything else) the Data Command Center can enable the user and/or medical care provider via the medical records dashboard to see the diagnostic test on same day even though it has not been billed. Further, this procedure can enable the medical care provider to optionally add a note and allow free hand typing at the end of the line.

In some embodiments, medical information populated within medical records dashboard (e.g., shown as visual cues, icons, or markers 885 representing medical services, procedures or tests performed or provided to the patient) can include a visual marker such as a red dot. In some embodiments, the Data Command Center can display the red dot until a claim is actually made at which time the Data Command Center can display a green dot (i.e., the Data Command Center can convert the red dot to a green dot). In some embodiments, by clicking on the dot, the user can toggle between the payment screen and the command center visual display window 500, 1700. This can allow medical care providers to improve patient care, to review the actual picture of a diagnostic test that is displayed within the command center visual display window 500, 1700, to review other diagnostic tests results, and to compare to what happened on other days. In some embodiments, at any time, a medical care provider can click on the dot to access a display where the claim is billed, and any payment that was made can be displayed. This process can help to reduce medical errors enabling medical care providers to quickly review the billings and claims made or billings signed off by a physician and payment portions of the Data Command Center. Further, this procedure serves as an additional tool to minimize coding, compliance with insurance guidelines, and medical treatment errors, as the Data Command Center can provide a quick reference tool that can pull all critical medical and procedure data from the patients EMR chart into a concise and clear table.

In some embodiments, a medical records dashboard of the present principles can display information related to medical procedures or services in relation to care of a patient with glaucoma. In some embodiments, the medical records dashboard can display various windows and sub-windows based on a user preference and/or current or previous user interaction with the medical records dashboard. As depicted in FIG. 11, in some embodiments a medical records dashboard 1600 can include information columns 1640 including a problems window 1650 and/or a surgeries window 1660 where information related to a patient's medical problems and surgeries can be displayed. In some embodiments, the medical records dashboard 1600 can include a summary window 1670 enabling a user to view and edit summary information related to the patient, any details of care provided to the patient, and/or and any medical diagnosis information prepared by a medical practitioner. Further, the medical records dashboard 1600 can also display detailed information related to any medical procedures or services provided to the patient, including procedures or services that are auto-populated by claims made or billing signed off by a physician as detailed above or other method. For example, in some embodiments, the medical records dashboard 1600 can display a visual display window 1700 including a plurality of information columns 1705. In some embodiments, the visual display window 1700 can be scrolled by the user to display other portions of the visual display window 500.

In some embodiments, the medical records dashboards of the present principles can also display detailed information related to notification of payment of any medical procedures or services provided to the patient, including procedures or services that are auto-populated by claims made or billing signed off by a physician as detailed above or other method. Moreover, the medical records dashboards can enable a user to access and/or track the status of the billing and payment process at any point in time. For example, in some embodiments, the medical records dashboards can access and view any patient encounter form (i.e. a superbill), any claims made to a clearing house, any updates on accepted or rejected bills from the clearing house, any claims made to an insurance company, and/or any payments received for any claims made.

As depicted in FIG. 11, in some embodiments of a medical records dashboard, the problems window 1650 can include a date and time information in entered date column 1711, a timeline column 1720, an “ICD” column 1730 for ICD code information, location of the problem or disorder (shown as “OD”, “OS”, “OU” identifying right eye, left eye, both eyes) (column 1740), and a diagnosis column 1750 for detailing information related to an initial diagnosis or final diagnosis of a patients problem or disorder. Further, the surgeries window 1660 can include information related to services or procedures were provided to the patient (procedure columns 1662), a description of the services or procedures performed (description columns 1664), and when the services or procedures were provided to the patient (shown as timeline columns 1666), and can include a surgical report that can be brought up and viewed by the user.

Referring to the visual display window 1700 of the medical records dashboard 1600 of FIG. 11, the information columns 1705 can include a date column 1711, and a procedure column 1721 illustrating or providing access to information detailing one or more procedures performed on the patient. Further, the procedure column 1721 can include an “OD” column 1722, and “OS” column 1724 providing right and left eye procedure information. In some embodiments, information related to the medical care provider, location where the procedure was performed, and office visit information can be provided to the user in the provider column 1731, and unit column 1741, and office visit column 1752. In some embodiments, the visual display window 1700 can enable a user to view information related to tests and procedures performed on the patient including, but not limited to one or more medical imaging procedures such as an optical coherence tomography (“OCT”), or fluorescein angiography (“FA”), and/or indocyanine green chorioangiography (“ICG”). In some embodiments, medical procedures performed (including any of the aforementioned medical imaging procedures) that have been billed and claimed can be viewed or accessed by a user within any of the “OCT” column 1760 (shown split as an “OD” column 1762 and “OS” column 1764), an “Ant Seg OCT” column 1770 (split as an “OD” column 1772 and “OS” column 1774).

In some embodiments, if visual function tests were performed, information can be viewed or accessed in the “VF” column 1780 (including an “OD” column 1782, and/or an “OS” column 1784. Some embodiments include a photo column 1790 configured to enable a user to access any photographic images of the patient's eyes including optical and/or auto-fluorescent images of the eyes (“OD” column 1792 and “OS” column 1794). Further, the embodiment of the medical records dashboard 1600 of FIG. 11 includes a Gonio column 1796 providing access to gonioscopy data and/or information related to a dilated fundus examination (“DFE” column 1798). In some embodiments, the surgeries window 1660, can include a location column, a surgeon column, and a comments column (not shown).

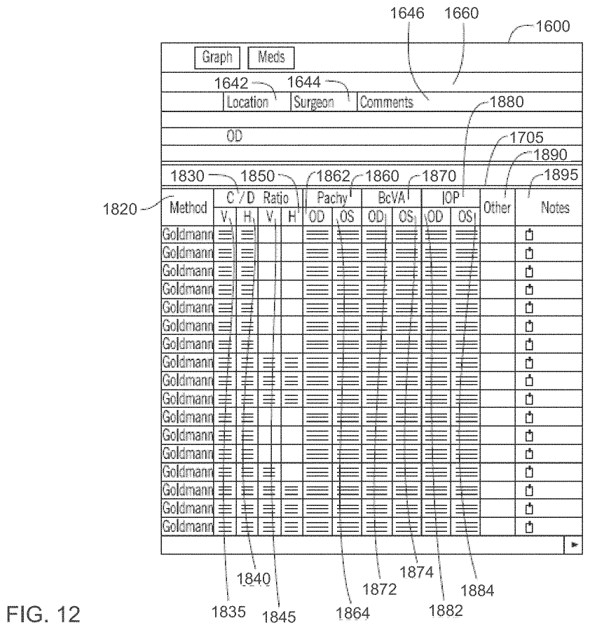

In some embodiments, the visual display window can enable a user to view information related to tests and procedures performed on the patient including a cup-to-disc ratio (“C/D”) to assess the progression of glaucoma, Pachymetry data (“Pachy”), refraction test information such as best-corrected visual acuity (“BCVA”), and/or intraocular pressure (IOP) data. For example, FIG. 12 depicts a portion of the medical records dashboard 1600 of FIG. 11 including column 1820, “C/D ratio” column 1830, “Pachy” columns 1860, “BcVA” columns 1870, and “IOP” columns 1880. In some embodiments, the “C/D ratio” column 1830 includes “V” column 1835, “H” column 1840, “V” column 1850, and “H” column 1850. Further, in some embodiments, the “Pachy” columns 1860 includes “OD” column 1862, and “OS” column 1864. In some embodiments, the “BcVA” columns 1870 includes “OD” columns 1872, and “OS” columns 1874. Some embodiments include “TOP” columns 1880 including “OD” columns 1882, and “OS” columns 1884. In some embodiments, other columns 1890 can be used to add additional test information. Further, the visual display window 1700 can also include a notes column 1895 for accessing and updating notes related to tests and medical diagnosis. In some embodiments, the tracking display window 1700 can be updated with comments and notes as described earlier.

In some embodiments, the Data Command Center can display and auto-populate a medical records dashboard of the present principles, such as the medical records dashboard 400 and/or the medical records dashboard 1600, with more than one patient information. For example, in some embodiments, any windows, sections, or columns of the medical records dashboard can display information related to a plurality of patients. Any patient data that is inputted, received, analyzed, or created can be auto-populated into any portion of the dashboard, where the Data Command Center can auto-populate in either a one-way or a two-way direction. Thus, data fields related to information in any patient information can be communicated via an electronic dataflow established between the Data Command Center and one or more computer systems of servers comprising patient information (e.g., such as electronic medical records). Further, in some embodiments, any information displayed by Data Command Center can display and auto-populate the medical records dashboard as a function of patients seen during a specified time period. In some other embodiments, the Data Command Center can display and auto-populate the medical records dashboard as a function of a specified disease and/or diagnosis. For example, in some embodiments, the Data Command Center can display and auto-populate the medical records dashboard as a function of a diagnosis or procedure or prescribed medication or lab or imaging test from input received from a physician or other medical practitioner or provider. For instance, every patient who has the diagnosis of diabetes with their name and the date last scene is auto-populated. Certain parameters that may need to be followed by the user from all of their patients with this condition can be auto-populated. For example, in the case of patients with diabetes, parameters can include how often they've missed appointments, blood sugar, hemoglobin A1C, medications, major new medical complications such as heart attack, stroke, amputations, blindness, each of which can be auto-populated and followed to enable the user to see how all their patients are doing. In some embodiments, input and the ability to display data can be based on single values or on complex multi-variate input (i.e. patients with diabetes, taking metformin and seen by the practice in the last 30 days).

In some embodiments, a user(s) can receive, via the medical records dashboard, a daily report on all the patients they have seen, what the diagnosis codes are and what CPT, ICD, or office visit billing codes were done whether they have been billed or not. In some embodiments, the user can view a report of patients for a specific day or week based on appointments or other data such as referrals. With this functionality, at the end of the day physicians can see all of their activity or the practice's activity on the medical records dashboard of the present principles and using data visualization techniques can realize what activity still needs to be completed or was not entered properly. The user can then review the same information in a few days' time and weeks or months later to ensure that the proper billing and collections has occurred. Additionally, the medical records dashboard enables direct one click access to underlying data, so without leaving the screen, providers can make medical decisions. For instance, an icon displayed on the medical records dashboard can represent that a test was performed on a particular patient or group of patients and that test can be directly accessed by activating that icon. If more information is needed, the entire individual flowsheet of the patient can be, with no more than one click, brought up, decisions made, and then with one click, return to viewing the entire medical records dashboard.

In some embodiments, two monitors can be implemented during which a first monitor can display the medical records dashboard and the second monitor, controlled by the first, can display the data from a selected patient. In such embodiments, even a single click to return to the medical records dashboard is not needed as information can be displayed on both monitors at once. In some embodiments, a portion or all of the data of a medical records dashboard, as well as the diagnostic tests, can be sent to a patient portal, to an email server, and/or as a fax. Further, in some embodiments, a user can be alerted via the medical records dashboard, when claims are sent out for payment and when claims are actually paid. For example, in some embodiments, the above described methods of display can provide a mechanism for displaying payments to the user, and if claims are being made for each patient seen in any particular day, week or month.

In some embodiments, any report, note, letter, referral or diagnostic test can be sent from a medical records dashboard of the present principles to an EMR, patient portal, a messaging platform to an email server, and/or as a fax. Messages can be transmitted to a patient or another practice focused on appointment reminders, medication prescriptions and/or refills, and good care management guidelines. It should be recognized that data interoperability and messaging are not limited to the examples provided but apply to any information within the Data Command Center.

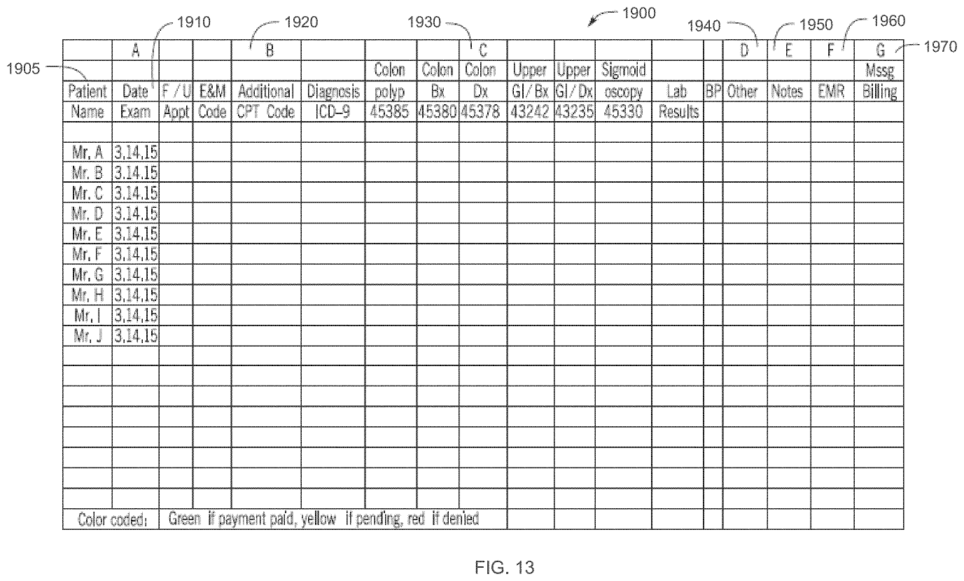

FIG. 13 depicts a portion of a medical records dashboard 1900 configured for display as a function of disease or patient in accordance with another embodiment of the present principles. In some embodiments, the medical records dashboard can be displayed overlaid on a previously viewed dashboard such as the medical records dashboard. For example, in some embodiments, the medical records dashboard can be displayed in the visual display window 500. In other embodiments, the medical records dashboards can be displayed independently from the medical records dashboard, and the user can toggle a display of any of the medical records dashboards.

The medical records dashboard 1900 of FIG. 13 provides a list of patients 1905, and within column 1910, an entire day of patients listed by date or by insurance coverage can be provided or the list can comprise a single patient with multiple visits. For example, in some embodiments, within column 1920 of the medical records dashboard 1900, an office visit and any items billed for a routine examination day and any other CPT codes billed that day can be displayed. In some embodiments, some specialties will have many CPT codes during an office visit (e.g. Ophthalmologists), whereas others (e.g., Gastroenterologists) can have four during an office visit. In some embodiments, column 1930 of the medical records dashboard 1900 can include the procedures that a physician can perform and are usually not on the same day as the exam (these can be GI physician examples). In some further embodiments, the column 1940 of the medical records dashboard 1900 can include various important parameters that can be followed for a specific patient. Some embodiments of a medical records dashboard, such as the medical records dashboard 1900, include column 1950 that enables a physician to write notes about patient care issues and column 1960, which enables a user to access that patient's personal EMR or review table and can also send a message to the patient. In some embodiments, the column 1970 of the medical records dashboard 1900 can enable a user access to the charge payment history of the patient and also enable a message to be sent to the billing department from this table. In some embodiments, columns can be reconfigured such that all patients with a particular insurance company that have a particular diagnosis on any given date or over a prolonged period of time can have tests and procedure results, as well as payments compared, allowing on one screen anyone to rapidly move through this visual display system to enable rapid comparison of results and potential payment anomalies for the same insurance company for the same CPT codes but perhaps different ICD9 or 10 diagnosis and to see if there is a mismatch (e.g., through the use of artificial intelligence systems discovering insurance payment irregularities). In some embodiments, columns 1920, 1930 of the medical records dashboard 1900 can be colored ‘black’ when a claim is made, and can be colored ‘green’ if paid, and can be colored ‘yellow’ if a payment is pending, and can be colored ‘red’ if payment denied by one rendition, e.g., physician reconciliation report of messages sent individuals for follow up, and/or a report of all the message activity from any given day.

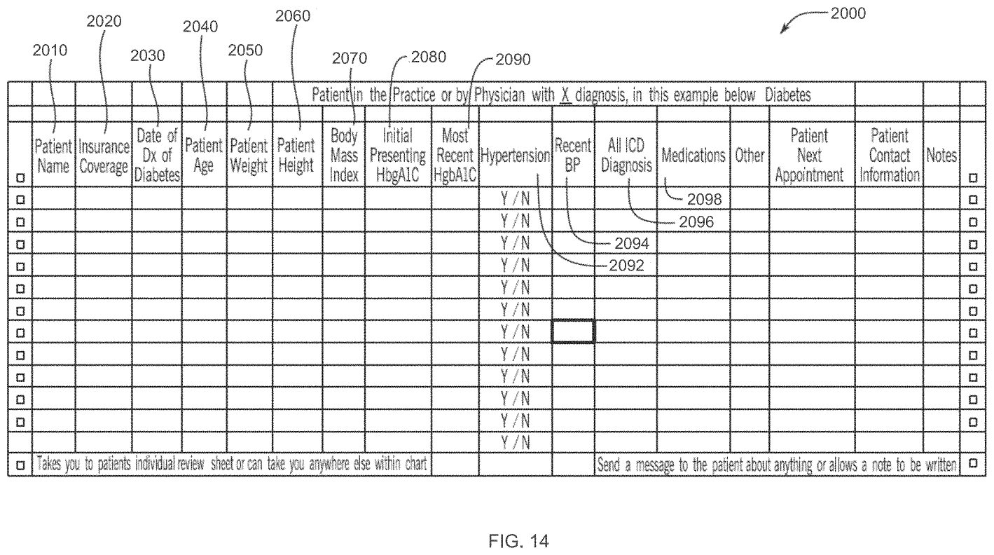

FIG. 14 depicts a portion of a medical records dashboard 2000 configured for display as a function of disease of a patient and specifically configured to display data related to patients with diabetes in accordance with another embodiment of the present principles. In the medical records dashboard 2000, the display for patients 2010 can include a variety of medical, billing, and insurance related information. FIG. 14 illustrates a portion of a medical records dashboard for display as a function of patients with a specific disease ICD, such that many patients may be compared at the same time, which can be useful for clinical research or for tracking clinical outcomes. The embodiment of FIG. 14 tracks all patients in a practice or a subset of patients and can compare, for example, particular diagnostic test results in patients with a particular condition, the results of a particular medication, how all the patients did who received a particular intraocular lens into the eye, etc. The information is put into each individual patient's table, but all patients with that issue can also be called up on a single table such as illustrated in FIG. 14. In some embodiments, such functionality can compile and compare all patients with a particular insurance company that have a particular diagnosis and compare tests and procedure results, as well as payments, allowing a user on one screen to rapidly see results, changes, patterns and payment anomalies. This feature also allows the extraction of patients with similar conditions for referrals and clinical research, batch generation for cross-referrals (e.g. optometry for an ophthalmologist), etc. In some embodiments, the medical records dashboard 2000 can be displayed as shown or can be sorted based on any of the data columns. For example, the patients 2010 can be shown including information displaying insurance coverage 2020, date of diagnosis of diabetes 2030, the patient's age 2040, the patient's weight 2050, the patient's height 2060, the body mass index 2070, the initial presenting HbgA1C 2080, the most recent HbgA1C 2090, the hypertension status 2092, the recent blood pressure 2094, the All ICD diagnosis 2096 and the current or past medications 2098. In some embodiments, the medical records dashboard 2000 can be reconfigured to display patients 2010 sorted by any of the columns 2020, 2030, 2040, 2050, 2060, 2070, 2080, 2090, 2092, 2094, 2096, 2098.

The medical records dashboard 2000 of FIG. 14 enables a user to present on one display everything the physician has done in a particular time frame, such as a day. For instance, a line of information for the information entered into the chart for every patient for today's visit, can be presented in the dashboard 2000. Whatever the user records, like the patient's vision and any diagnostic tests or laser procedures or injections in the eyes that were actually done that day, can be displayed on the medical records dashboard 2000 for interpretation. A medical records dashboard of the present principles can also enable a user to configure follow-up enabling a user to check again (e.g. 48 hours later) when everything for that day should have already been billed or 60 days later when everything is paid and all dates queried can be compared. Also, the user is able to review all patients having a common insurance carrier to facilitate satisfactory payments from the insurance company.

In some embodiments, a Data Command Center of the present principles can enable an addition of a date alert or self-destruction of any information or data entered or auto-populated in the medical records dashboard 400. For example, in some embodiments, any message, or note, or summary, or any medical data can include a date alert and/or a self-destruct function that can remove and/or delete information from the medical records dashboard 400. In other embodiments, the historical date and/or an alert or warning can be provided with any auto-populated or user-summoned information to assist the user with an assignment of relevancy to any data being reviewed prior to, during, or after a patient visit or examination. In some embodiments, this feature can optimize the standard of care being delivered by the user. For instance, this feature can help monitor preferred practice patterns or serve as a reminder on information needed for clinical review.

In some embodiments, a Data Command Center of the present principles enables the prioritization of relevant data in at least portions, columns and rows of a medical records dashboard, while minimizing less important values. This functionality enables a user to focus on the most important data pertinent to the current use case (i.e., with a patient that has a certain diagnosis, several preferred diagnostic test results and data are germane). In some embodiments, such display capabilities can be applied to data that originates from additional users/EHR deemed important and which can be rendered in chronological order. Utilizing Artificial Intelligence (AI), Natural Language Processing (NLP), and/or conventional business logic, a Data Command Center of the present principles can programmatically filter out unnecessary information and queries for display.

In some embodiments, a medical records dashboard of the present principles can be configured based on key events, results, date/time, and/or logical parameters which can include, but are not limited to Diagnoses, Medication Start/End Dates, Allergy Start/End Dates, Billing History, Demographic Data, Observations/Plans, and Life Events. In accordance with the present principles, the format and display of rendered data in the medical records dashboard will make maximum usage of space by shrinking less relevant rows, auto-sizing of columns, and automatically collapsing less relevant data. The intention of this functionality is to provide the most efficient view in the medical records dashboard of relevant data, while not overloading the provider with information not germane to the current configuration. An example would be if a patient has glaucoma, there are specific columns in the medical records dashboard that are highly important to monitoring the chronic condition but some which have no relevance. In this example, the patient that has a diagnosis of glaucoma will have Intra-Ocular Pressure (TOP), Glaucoma medications, etc. displayed prominently while the other less relevant columns are masked.

In some embodiments, the Rules module 004 of the Data Command Center of the present principles can include a Flowsheet Editor Interface that provides a method by which a user/medical care provider can configure the formatting and display of data intended for the medical records dashboard. This simplified interface editor embodies “What You See Is What You Get” (WYSIWYG) methodology, in some embodiments including drag and drop of Flowsheet elements. Upon completion of a Template for the medical records dashboard, associated parameters can be defined. The Flowsheet Editor enables a user to define how columns and rows will be displayed in the medical records dashboard. While users have the ability to only view predefined data, filtered data may trigger a rule to display in lieu of predefined filters. Preconditioned upon required contract/agreement between users, data can display from multiple, disparate sources to display continuity of care.

In some embodiments, the Flowsheet Editor Interface of the Data Command Center also enables an end-user to configure how summary rows are presented within the medical records dashboard. A user can choose to discard certain edge-cases from the summary calculation, take the highest and lowest values, take an average, or some other logical calculation to determine how the individual columns summary row data presents itself. In addition to enabling changes which reflect the display of the data, it is possible for the user to program alerts and auto-tasks which are sent as a result of the rule threshold being exceeded. For example, if a user/medical care provider determines that a new alert rule must be created, they are able to select the column, or columns, apply logical rules to the column or columns being analyzed, and set the task associated with rule which will be sent to the user-defined staff member or groups of staff members. As another example, a user can choose to add a new alert for those patients which have a diagnosis of Glaucoma and have not had a required diagnostic test, a Visual Field, in 365 days. The user is then able to set a task that will be sent to staff to schedule the diagnostic test that will automatically be sent when the system and user-defined auto task and alerts are processed. The editor interface also enables a user to configure a manner in which the alert is displayed in the medical records dashboard. For example, a user can set the display of an alert to any of the following, but not limited to, headers, within the rows and columns of the flowsheet, on the patient demographic panel, etc.

Pre-defined display rules can override a user-defined configuration of a medical records dashboard when the rule is prioritized, in some embodiments, for patient safety reasons. These overrides can display information regarding a subject visit in a prominent color. For example, if a patient had recently found out that she was pregnant, it becomes very important that she does not have certain diagnostic tests performed as such tests can endanger the viability/health of the fetus. For example, a Fluorescein Angiogram should not be performed on a patient to monitor the progress of a patient if she is pregnant. Due to the potential life altering consequences, the Data Command Center, through the use of, for example AI, is smart enough to override a medical records dashboard template, and prominently display the visit from the OBGYN on the medical records dashboard, in an instance in which the patient was confirmed to be pregnant.

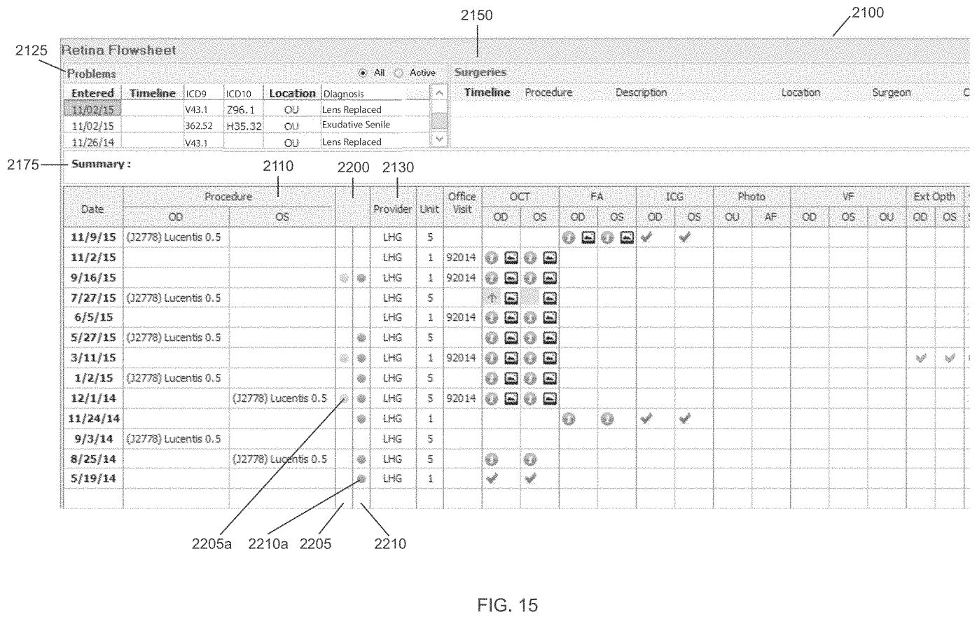

In some embodiments, a Data Command Center of the present principles can enable a user to access a detailed ledger comprising patient financial information from a medical records dashboard. In some embodiments, the medical records dashboard can include at least one visual indication of a payment for services provided, where detailed information of the charges, payments, write-offs, adjustments, and balances can be accessed and displayed. For example, FIG. 15 depicts an embodiment of a medical records dashboard 2100 which can be displayed following a user's selection of at least one medical records dashboard from the medical records dashboard selection window in accordance with another embodiment of the present principles. For example, in some embodiments, the user can make a selection of “Retina Flowsheet” to access and/or launch the medical records dashboard 2100. In some embodiments, the medical records dashboard 2100 can include a display of data from one or more medical records and can track medical procedures and services based on claims made or billing signed off by a physician for one or more delivered medical procedures or services. Some embodiments include a Data Command Center that can dynamically link to various external databases comprising patient information that can be displayed in the medical records dashboard 2100. For example, in some embodiments, the Data Command Center can function as a portal to patient information prepared by the user or patient information from other sources. Further, in some embodiments, the medical records dashboard 2100 can be auto-populated as a function of claims made or billing signed off by a physician. In this instance, any data displayed within the medical records dashboard 2100 is derived from one or more claim records that have been billed for one or more procedures or services have previously been provided to the patient. In some other embodiments, auto-population can be enabled in both directions interacting as a switchboard between the entire EMR and the medical records dashboard 2100.

In some embodiments, the medical records dashboard 2100 can display information related to medical procedures or services in relation to retinal eye care of a patient. In other embodiments, a medical records dashboard can display information related to medical procedures or services in relation to any kind of medical care of a patient. In some embodiments, the medical records dashboard 2100 can display various windows and sub-windows based on a user preference and/or current or previous user interaction with the medical records dashboard 2100. For example, in some embodiments, the medical records dashboard 2100 can display a problems window 2125 and/or a surgeries window 2150 where information related to a patient's medical problems and surgeries can be displayed.

In some embodiments, the medical records dashboard 2100 of FIG. 15 can display information including components where there is a summary of the patient's problem list in which a user can input patient information and constantly update and change. Further, this information can be auto-populated with the touch of a button into a designated location such as the current plan documenting the patient's current visit (thus aiding documentation for the current visit). Further, whatever is important for a user to input into the day's visits for documentation can be initially inputted in the table, and then permanently into the day's patient visits. Further, the summary section of the medical records dashboard 2100 can be constantly fluid and can be changed at every visit rather than being written to an unchangeable document or file (e.g., such as a PDF). For example and as depicted in FIG. 15, the medical records dashboard 2100 can include a summary window enabling a user to view and edit summary information related to the patient, any details of care provided to the patient, and/or and any medical diagnosis information prepared by a medical practitioner. Further, the medical records dashboard 2100 can also display detailed information related to any medical procedures or services provided to the patient, including procedures or services that are auto-populated by claims made, or billings or payments including billing signed off by a physician as detailed above. Additionally, all of the features of the previously described medical records dashboards of the present principles can be provided in the medical records dashboard 2100.

Some additional features of a medical records dashboard of the present principles, such as the medical records dashboard 2100 of FIG. 15, include displaying at least one visual indication of a payment for services provided. Further, the user can be provided with access to a detailed ledger comprising financial information related to one or more procedures. For example and as depicted in FIG. 15, the medical records dashboard 2100 can comprise a payment indicator column 2200 including one or more indicator and/or access icons. For example, in some embodiments, the payment indicator column 2200 can comprise a column or columns 2205/2210 that can be populated with one or more indicator or access icons 2205a/2210a. In some embodiments, the one or more indicator or access icons can comprise icons of color such as yellow or green to indicate a status of payment. The payment indicator column 2200 can be located anywhere on the of the medical records dashboard 2100. In the embodiment of FIG. 15, the payment indicator column 2200 is positioned between the procedure column 2110, illustrating or providing access to information detailing one or more procedures performed on the patient and information related to the medical care provider, and the provider column 2130, that can display the location where the procedure was performed, and office visit information.

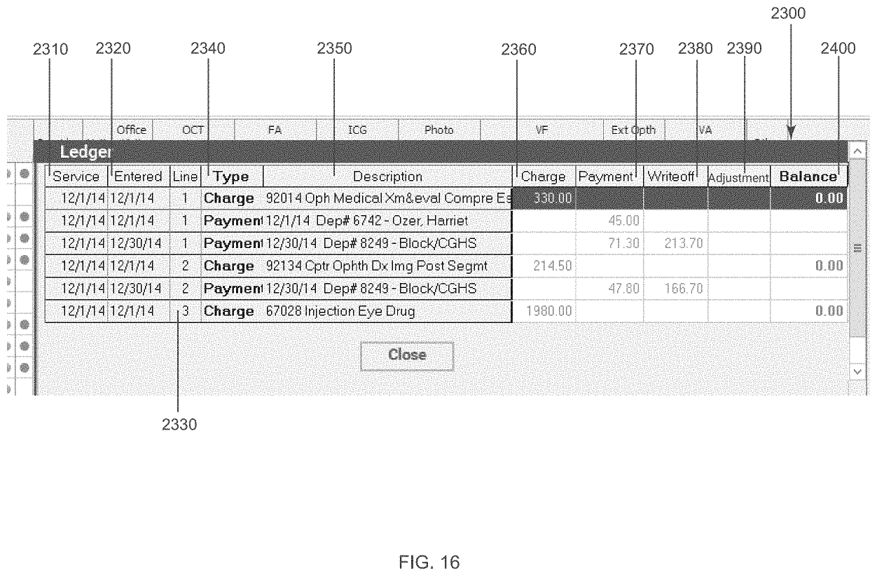

In some embodiments, one or more of the icons of the payment indicator column 2200 can be accessed by the user to initiate the display of more detailed financial information. For example, FIG. 16 depicts a ledger window 2300 accessible from the medical records dashboard 2100 of FIG. 15 in accordance with an embodiment of the present principles. In some embodiments, the Data Command Center of the present principles can display the ledger window 2300 overlaid onto the medical records dashboard 2100. In other embodiments, the ledger window 2300 can be displayed in place of the medical records dashboard 2100. In other embodiments, the ledger window 2300 can be displayed with the medical records dashboard 2100. In some embodiments, the ledger window 2300 can include information processed by the Data Command Center, which includes information related to the date of procedure, description of the procedure, dates entered, a charge type, etc. For example and as depicted in the embodiment of FIG. 16, the ledger window 2300 can include the service to column 2310, entered column 2320, line column 2330, type column 2340, and description column 2350. Further, in some embodiments, the ledger window 2300 can include information related to payments and billing. For example, in some embodiments, the ledger window 2300 can include a display of a charge column 2360, payment column 2370, write-off column 2380, adjustment column 2390, and a balance column 2400. In some embodiments, the user can close the ledger window 2300 and return to the medical records dashboard 2100 at any time. In other embodiments, more than one ledger window 2300 can be displayed based on selections made by the user in the medical records dashboard 2100.

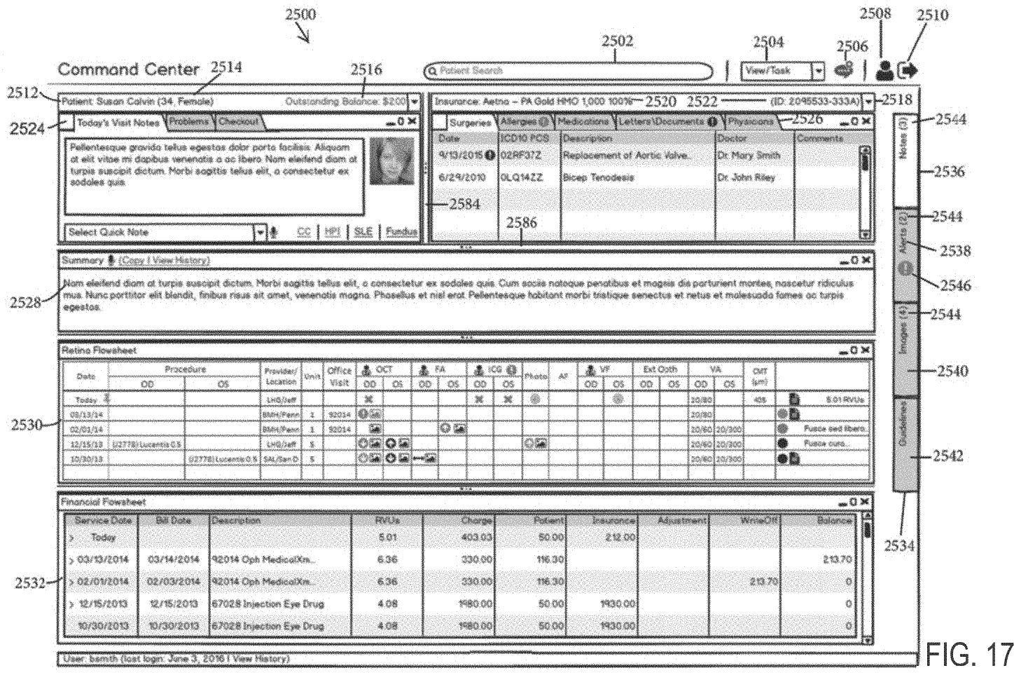

FIG. 17 depicts an embodiment of a Data Command Center menu 2500 including a medical records dashboard 2530 implemented as a data interface to a medical record system in accordance with an embodiment of the present principles. The Data Command Center menu 2500 in the embodiment of FIG. 17 is designed to interact with a conventional EMR system although, as noted above, the Data Command Center menu 2500 of FIG. 17 can be used with other large data systems to present data to users in a meaningful way. In addition, the exemplary embodiment illustrates a Data Command Center menu 2500 for implementation in an Ophthalmology practice. Those skilled in the art will appreciate that the interface can be readily configured for other medical specialties. The Data Command Center menu 2500 is able to display data from multiple data sources in multiple different panels on a single interface. In the exemplary embodiment of FIG. 17, the Data Command Center menu 2500 provides a comprehensive overview of the patient's clinical and financial history as well as providing a means to quickly order tests while retaining the ability to see previous medical history. Clinical and insurance guidelines as well as preferred practices can be quickly accessible based on the patient's conditions, medications and procedures so that a medical care provider/user can readily provide optimal care and be compliant with medical and billing requirements. The medical care provider thus becomes a part of revenue cycle management for each patient in the medical care provider's practice.

A Data Command Center and medical records dashboard in accordance with the present principles can incorporate self-deleting staff messages that are presented to the Data Command Center. For example, a staff person can send a message about a patient to the medical care provider that appears in a display window in either of the Data Command Center menu 2500 and the medical records dashboard 2530 with a message such as “the patient has been waiting over an hour and is upset” or the “patient has previously filed a malpractice complaint” that do not become part of the patient's medical record. The message can be programmed to be deleted once the patient's visit is billed.

As depicted in the embodiment of FIG. 17, a user has the ability to search for patients at 2502, select different views of the data at 2504, add sticky notes at 2506, access user information at 2508, and logout at 2510. Immediately below that on the upper left-hand side of the Data Command Center menu 2500 is the Patient Information Bar 2512, which contains the patient's identifying information 2514 so the user knows they are looking at the correct patient record. The Patient Information Bar 2512 also notifies the user of patient's outstanding balance 2516. To the right of the Patient Information Bar 2512 is the Patient Insurance Bar 2518, which provides the patient's insurance information 2520, including the ID number 2522 for the patient's primary insurance. Below the Patient Information Bar 2512 is a collection of tabs 2524 displaying different sets of information about the selected patient. A different collection of tabs 2526 is found underneath the Patient Insurance Bar 2518. Under tabs 2524 is the Summary panel 2528 where the user can enter notes about the patient (e.g. patient did not show up for missed appointments).

The medical records dashboard 2530 (illustratively depicted as a Retina Flowsheet), is an encounter driven panel that summarizes key clinical and financial information in chronological or reverse chronological order at a glance, allows the user to order new Procedures and Imaging tests, and provides assistance complying with insurance regulations, as will be described in more detail below. Below the medical records dashboard 2530 is the Financial Flowsheet 2532 providing a summary of the financial information related to the patient and this is adapted to provide the user with the ability to drill down into individual transactions. On the right side of the medical Data Command Center menu 2500 are a series of vertical tabs 2534 that when individually clicked slide out to provide more information to the user. The Notes Tab 2536 expands to display patient notes, while the Alerts Tab 2538 expands to display patient alerts (e.g. patient's chart was requested by insurance company or sent for a second opinion). The Images Tab 2540 expands to display images for the patient and the Guidelines Tab 2542 expands to display clinical practice and insurance guidelines along with preferred practice patterns where applicable. On each tab, displayed next to the tab title is the count of the new items 2544 since the last time the user accessed the patient record. Once the tab is expanded, the new count will be removed because the user has seen the information. If important or critical data exists within the tab, a special alert icon 2546 is displayed on the tab (describe modules and details of this function). Once viewed, the alert icon 2546 is removed. As will become apparent from the following description, the layout of the Data Command Center menu 2500 permits access by the user to all of the relevant information within one click of the mouse and without having to steer to other screens that would take the user away from the Data Command Center menu 2500. For example, the information is either available in a display window, behind a tab, or available via a pop-up window; accordingly, the user does not have to leave the display screen to access the information (describe modules and details of this function).

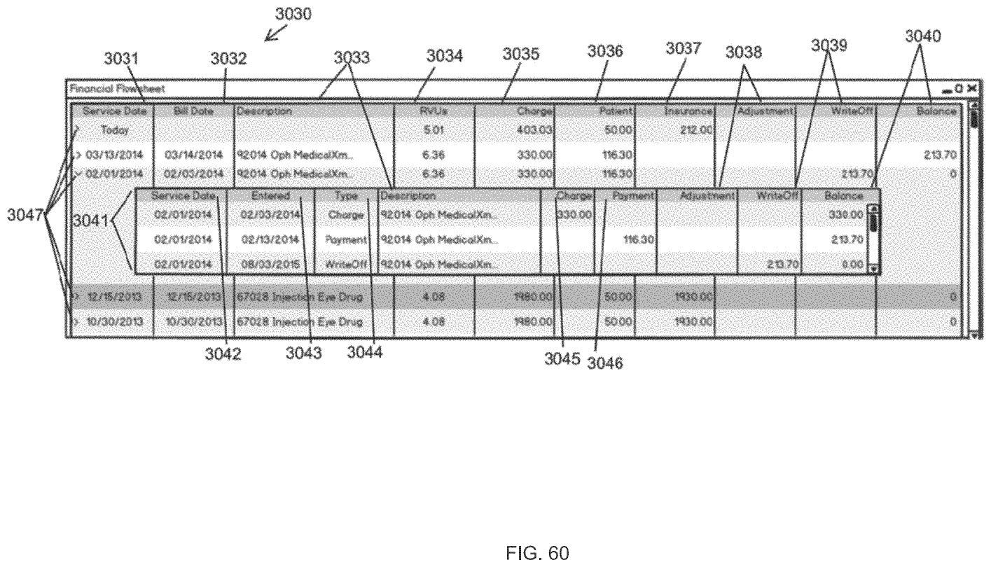

The Financial Flowsheet 2532 is illustrated in FIG. 60. The Financial Flowsheet 2532 contains a dynamic table 3030 of financial activity for the patient, as opposed to the physician's practice, as is usually the case with conventional EMR systems. As illustrated, an exemplary embodiment of the Financial Flowsheet 2532 includes the following fields: the Service Date 3031, Bill Date 3032, Service Description 3033, Relative Value Units (RVUs) 3034 that determine how a physician is paid in accordance with work effort, Gross Charges 3035, the Amount Due from the Patient 3036, the Amount Due from Insurance 3037, any Adjustments 3038, Write Offs 3039, and the outstanding balance 3040. An intelligent alert configuration system can be launched to configure rules, alerts, notifications, and tasks based on clinical and/or financial parameters denoted in FIG. 60. The rules, alerts, notifications, and tasks may be applied to one or a group of patients. Selection of the Encounter expansion control 3047 produces a second dynamic table 3041 within the first dynamic table 3030. The second table 3041 presents individual financial transactions associated with the encounter. In an exemplary embodiment, the second dynamic table 3041 contains: the Service Date 3042, Date Entered 3043, the Type of financial activity entered 3044, the Service Description 3033, the Charge or Amount Billed 3045, Payments 3046, Adjustments 3038, Write Offs 3039, and the outstanding balance 3040. The intelligent alert configuration system illustrated may be launched to configure rules, alerts, notifications, and tasks based on clinical and/or financial parameters at a more granular level, which may then be applied to one or a group of patients.

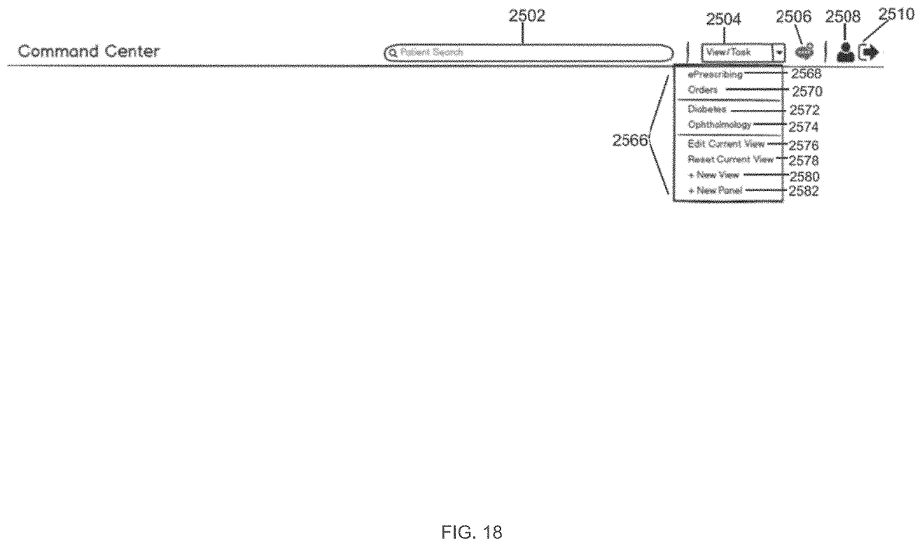

FIG. 18 depicts a User View control panel 2566 that can be part of the View/Task menu 2504 of the medical records dashboard of the Data Command menu of the embodiment of FIG. 17 in accordance with an embodiment of the present principles. The user View control panel 2566 of FIG. 18 displays views for selection by the user. As illustrated, the user can select one of several Views 2568-2574. Views ePrescribing 2568 and Orders 2570 are examples of task-based views, while Diabetes 2572 and Ophthalmology 2574 are examples of condition or specialty specific views. In an exemplary embodiment, the system has several pre-configured views but more can be added over time by deploying new versions of the system or by user modification. The user can edit the Current View 2576, Reset the Current View 2578 to its default configuration, create a new View 2580, or create a new Panel 2582. If the user selects a new view from options 2568-2574, the screen layout is changed to reflect the selected view for the current patient. (describe modules and details of this function). Referring back to FIG. 17, it should be noted the dimensions of the different panels can be resized by changing the location of the vertical sliders 2584 and horizontal sliders 2586. This enables the user to control how space/area on the display is used for each panel. As the user adjusts the sliders 2584 and 2586, the dimensions are remembered so when the user returns to the view at a later time the system remembers the dimensions. The user also can reset the view to its default dimensions by clicking the Reset Current View 2578 option in the view control panel 2566. The entire view also resizes based on the dimensions of the user's monitor and the size of the browser display.

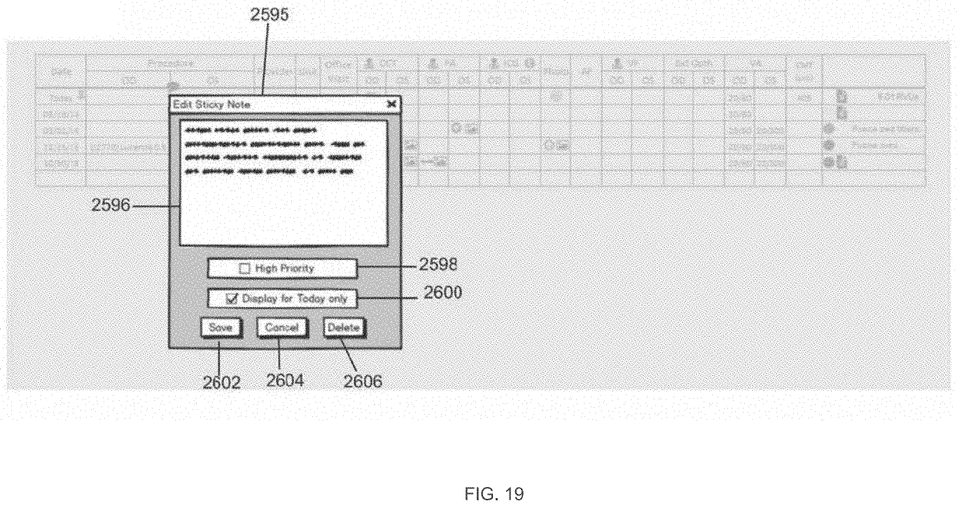

FIG. 19 depicts a sticky note panel 2595 of the Data Command Center menu 2500 of FIG. 17, which is activated when the add sticky notes icon 2506 in FIG. 17 is selected in accordance with an embodiment of the present principles. As illustrated, the user may click and drag the icon 2506 to any location on the page and drop it where they want it placed. This allows the user to place the note in context of the information to which it refers. The icon 2506 then stays in place and is associated with dynamic data until deleted by a user or it expires based on the note settings. (describe modules and details of this function). When the icon 2506 is placed, the Edit Sticky Note control panel 2595 is displayed. The Edit Sticky Note control panel 2595 can also be displayed if a user selects the icon 2506. Once the Edit Sticky Note control panel 2595 is opened, the user can enter a note in field 2596, select if the note is high priority 2598, select if the note should only be displayed today 2600, in which case after the date it is entered the note will no longer display. The user can save the note by clicking the Save Button 2602 or cancel the action by clicking the Cancel Button 2604. The user can delete the note by clicking the Delete button 2606 at which point the action is confirmed before deleting. When a user moves the mouse over the note icon 2506, the note text is displayed next to it in a tooltip. The note icon is colored, e.g., black if it is not high priority and, e.g., red if it is high priority or can flash or be highlighted in any other manner. All deleted or expired Sticky Notes along with the location and duration where they are displayed can be preserved for purposes of legal discovery but may not be accessible to the user as a general practice.

Referring back to FIG. 17, when the user selects the user profile icon 2508, a panel (not shown) is presented to enable the user to edit typical user information including their name, address, phone numbers, email address and password. The user can also select their preferred email or phone number or other method for communications sent by the Data Command Center of the present principles. When the user selects the Logout icon 2510, if any data is not saved, the user can be prompted to save data before closing the system. If the user answers positively that they want to save unsaved data, the user is not logged out. Once all data is saved, the user is prompted to confirm their desire to logout. If confirmed, the user is logged out.

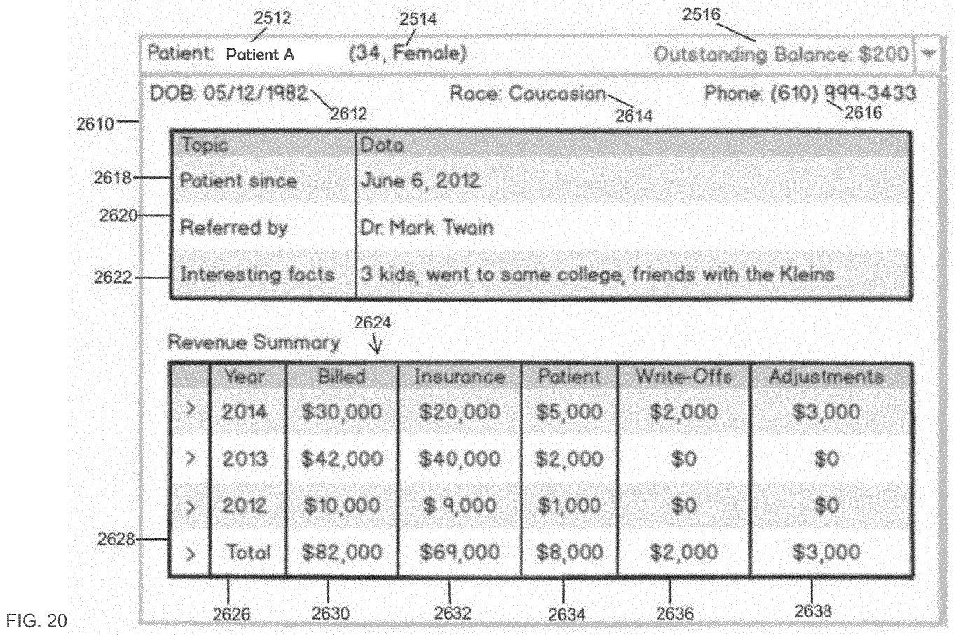

The Patient Information Bar 2512 illustrated in FIG. 17 displays high level information about the patient. The user can click on the Patient Information Bar 2512 and the Patient Information Panel 2610 shown in FIG. 20 is displayed underneath the Patient Information Bar 2512. Clicking the bar a second time or anywhere else on the page will close the Patient Information Panel 2610. The Patient Information Panel 2610 contains the patient's date of birth 2612, race 2614, phone number 2616, the date when the patient was first seen 2618, the referring physician 2620, and interesting facts to remember 2622. Information in the Patient Information Panel 2610 can be edited in place by clicking on the item to be changed. When clicked, the field will change to a text field allowing the value to be changed with a Save button displayed next to it (not depicted). Clicking Save will save the data. Clicking a Cancel button (not shown) will not save the data leaving the value unchanged and the control will revert to static text. Below these fields is a Revenue Summary 2624 for the patient. The Revenue Summary 2624 displays patient totals for each year 2626 as well as grand totals 2628 for the total amount billed 2630, amount paid by insurance 2632, amount paid by the patient 2634, amount written off 2636, and the adjustments 2638 for each year.

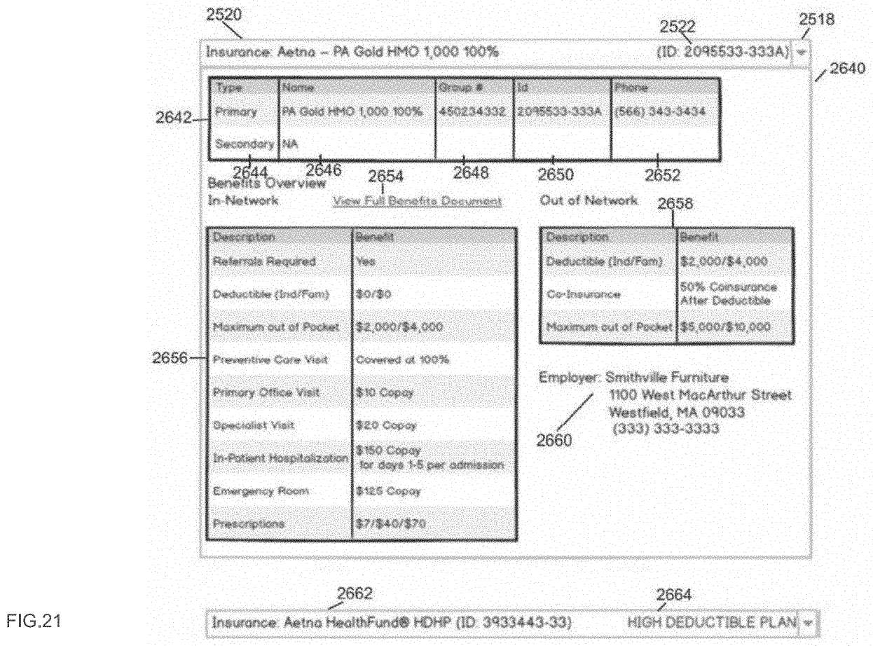

To the right of the Patient Information Bar 2512 in FIG. 17 is the Patient Insurance Bar 2518. The user can click on the bar and the Patient Insurance Panel 2640 illustrated FIG. 21 is displayed underneath the Patient Insurance Bar 2518. Clicking the Patient Insurance Bar 2518 a second time or anywhere else on the page will close the Patient Insurance Panel 2640. The Patient Insurance Panel 2640 contains information about the patient's insurance 2642 including the type 2644, name 2646, group number 2648, insurance ID number 2650, and phone number 2652 for each insurance company. A link to the patient's benefit document 2654 is provided as well as an overview of the patient's in-network benefits 2656 and out of network benefits 2658 as provided by the patient's insurance company. The values illustrated as 2656 and 2658 are provided for example purposes and will vary based on the data provided by insurance companies. The patient's employer's address and contact information 2660 is also displayed for convenience. Item 2662 displays an alternative embodiment of the Patient Insurance Bar 2520. In this case, the patient has a high deductible plan and this fact is displayed at 2664 in red in the Patient Insurance Bar 2520 to be sure the physician is aware that, in this case, the patient has a high deductible plan.

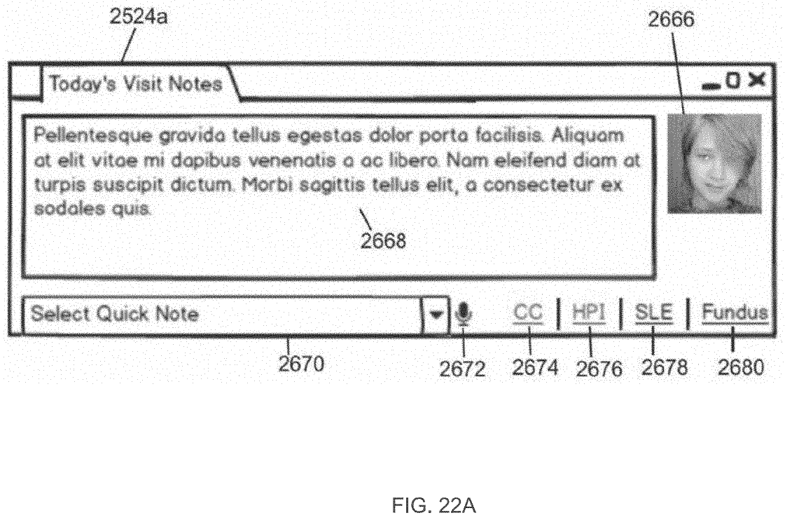

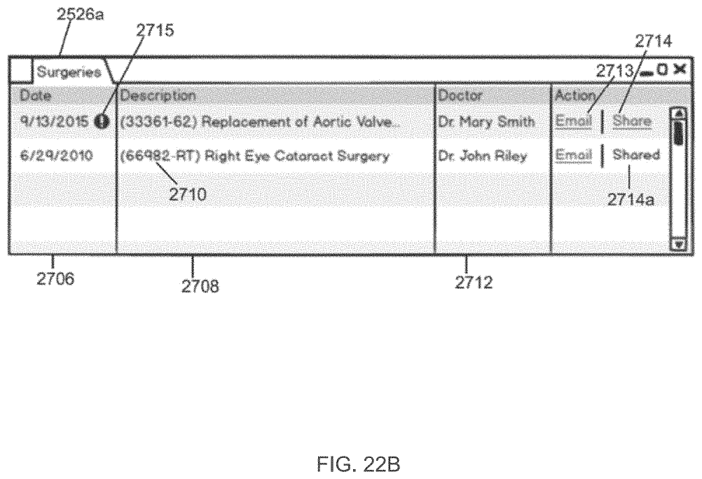

FIG. 22A depicts an embodiment of a Today's Visit Notes tab 2524a of the Data Command Center menu 2500 in accordance with an embodiment of the present principles. The Today's Visit Notes tab 2524a as illustrated in FIG. 22A contains elements related to capturing information about notes specific to today's visit. The tab 2524 contains a photo 2666 of the patient, free form text notes 2668, a control allowing the user to select pre-configured notes 2670, an icon 2672 that triggers a dictation feature allowing text entry into free form text notes 2668 via voice recognition, and a set of links 2674-2680 that are reminders to complete important aspects of an encounter in the EMR. The patient image 2666 is imported from the EMR and text notes 2668 can also be imported from the EMR through the Command Center CCOW Implementation described below. Items 2674-2680 display the status of the chief compliant (CC) 2674, history of present illness (HPI) 2676, slit lamp exam (SLE) 2678, and Fundus photograph 2680. This status is also provided to the Command Center via the CCOW implementation. Items 2674-2680 are displayed in red until complete at which time they are displayed in black. Based on EMR access and functionality, items 2674-2680 are links back to the specific area in the EMR. In an exemplary embodiment, the physician may dictate or type notes into the Today's Visit Notes tab 2524a that automatically generates a letter to a referring physician or another physician alerting that physician about something important in the patient's medical history. Beneficially, the referring letter may be generated while the patient's medical history is on the display screen in the Data Command Center interface.

Embodiments of the present principles can further include a Problems tab, which displays a patient's problem list as imported from the EMR. The following fields can be displayed, including but not limited to; date entered, associated ICD10 code, body location, and diagnosis. The user can manually order the list in order of severity or importance by clicking and dragging the rows. A Sort by Date can sort the list in reverse chronological order and Sort by Importance can sort the list using the user's manual ordering. If the user has not adjusted the order of Problems, it will display in reverse chronological order. A default sort order can be by date, but, in some embodiments, the user's last selection is remembered and automatically selected when the user returns to the application.

Embodiments of the present principles can further include a Checkout tab used to determine when a patient should return to the practice. This can also be used for a return visit to a shared physician's office which would then also in some embodiments populate a shared care medical table that can be given to a patient for a future reminder of appointment. The Checkout tab can be configured to display a recommended clinical guideline based on Clinical Decision Support algorithms of the Data Command Center. A user can select a count and a period to generate a time period in which the patient should return. A search feature can implement basic type-ahead search and results listing enabling the user to select an item. In the case of either the search or a drop-down menu the selected item can be listed underneath. The user has the ability to delete the item by clicking an associated delete icon. The user can also enter a free form text note or use dictation by selecting a dictation icon 2702. When complete, the user can click a Save button to save the Checkout information and send it to the EMR or clear the information by clicking a Clear button.

FIG. 22B depicts an embodiment of a Surgeries tab of a medical records dashboard of a Data Command Center in accordance with an embodiment of the present principles. The Surgeries tab 2526a as illustrated in FIG. 22B displays information about the patient's surgeries. The Surgeries tab 2526a displays the date of surgery 2706, the description 2708 including the billing (ICD10) code 2710, the primary physician 2712, and several actions including the ability to email 2713 or share 2714 the patient record with another physician. The shared notation 2714a signifies that the patient record has already been shared with the other physician. A notes column (not shown) displays the first few characters or words based on available space of an associated note. Moving the mouse over the specific note displays it in a pop up. In the case of some surgeries, a hospital or physician will not be paid if readmitted in 30 days. In these cases, if a surgery has been performed in the last 30 days a black circle with an exclamation point 2715 can be displayed next to the date. Moving the mouse over the icon displays a message stating how many days are left until the patient can be readmitted. An associated note can also indicate that the patient is participating in a capitated plan where anything the physician orders for the patient will not be reimbursed.

When a patient record is shared with another medical professional, if the professional does not have access to the D at a Command Center of the present principles, the other medical professional can receive an email to register for access to the Data Command Center. In some embodiments, if the professional does have an account but a new patient is being shared, the physician can receive an email notification. The new external user will only have access to the specific patients that are shared. Such sharing of patient medical records amongst the patient's physicians better enables the physicians to work together to follow preferred practice patterns for patient treatment as may be required by insurance companies and/or the government. This process is particularly helpful for managing patients with certain chronic diseases like diabetes in which a nephrologist, podiatrist, ophthalmologist, endocrinologist, and family physician need to see each other's results. Another example is shared care before and after cataract surgery where optometrists and ophthalmologist need to see each other's results.

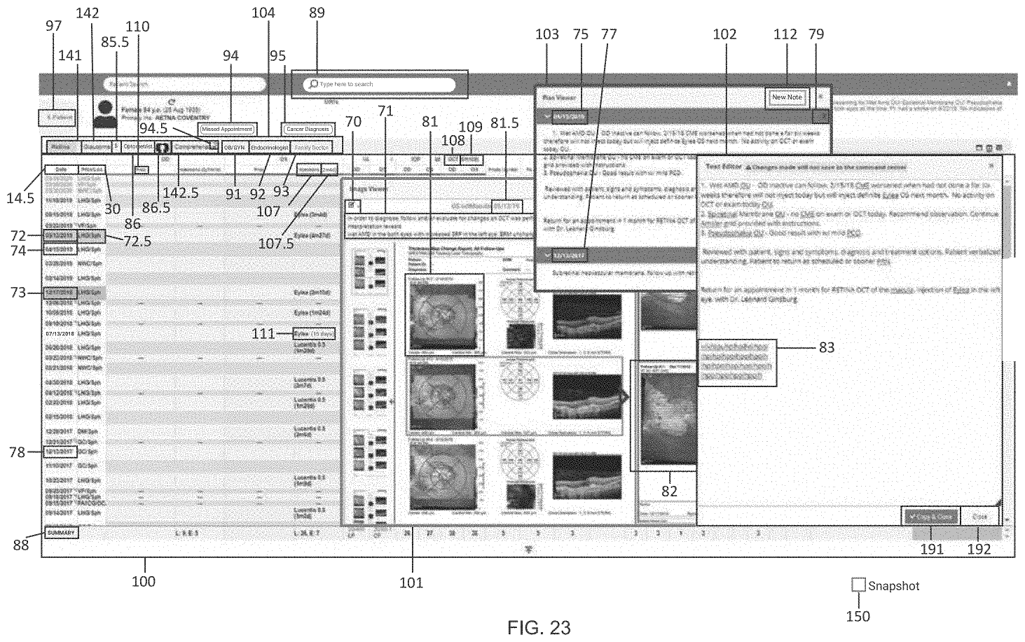

FIG. 23 depicts an embodiment of a medical records dashboard in accordance with another embodiment of the present principles. In accordance with the present principles, the medical records dashboard of FIG. 23 is intended to provide and display to a user/medical care provider with all patient data/information necessary to perform accurate and efficient patient care using a single display. In the embodiment of the medical records dashboard of FIG. 23, panels 100, 101, 102, 103, and 104 are some examples of different panels that can be moved around, toggled, simultaneously active (i.e., information from each panel can be assessed interchangeably without changing views) and displayed while critical information is viewed. In each column, what is an important data element over time can be followed as noted in column 14.5. This enables a user to view the information vital to evaluation of their patients. In addition, in some embodiments, the medical records dashboard of FIG. 23 enables, direct access to patient data/information (no more than one click, one hover or selected directly in any manner). Some embodiments enable toggling by a mechanism such as alt-tab to gain access to underlying patient data/information or associated screen, tab or window. A user/medical care provider is able to decide what is important to pull up, directly to view, and can move the separate windows or other pop-ups out of the way to view important patient data/information underneath. In one embodiment, a Rules module, such as the Rules module 004 of the Data Command center 001 of FIG. 1, can be configured to know what information for the patient is important, what information must not be blocked, and when information is directly clicked and displayed, enables the movement of a needed columns into a set area on the screen where critical information remains in view. In the embodiment of FIG. 23, an example of two data sets that remain in view is depicted by column 14.5, which includes the date of service when an encounter occurred with a patient, and column 106, which displays the provider and location of encounter. In the embodiment of the medical records dashboard of FIG. 23, all of the other columns, such as column 107, which depicts injections performed on a patient and/or procedures column 110 can be moved or at least partially covered from display.

Alternatively or in addition, in some embodiments none of the patient data/information is completely blocked from view through the use of transparency viewing. In FIG. 23, block 81 displays an image of an OCT that displays to a user/medical care provider if injections of the left eye are working. In the embodiment of the medical records dashboard of FIG. 23, column 107 is viewed, not blocked, so the user can correlate when the injection (or any procedure of clinical information or diagnostic test) was performed and how it relates to the information that was pulled up, with direct access to any additional information. In some embodiments of a medical records dashboard of the present principles, columns/windows/pop-ups of interest to a user can be moved to another portion of the medical records dashboard where no patient data/information or patient data/information of little or no interest to a user, exists. For example, if the user would also like to compare OCT data (108) and in particular the left eye, as this example shows injections of certain medications (i.e. Eylea, Lucentis) and column 107 over time, the user could simply drag 108 or just 109 (left eye) over to column 110, because no data is present in that area of the medical records dashboard. Now all in one view and in a particular section of the medical records dashboard, exists all information that user would need to compare OCTs (108) over time with injections (107). In another example, when an OCT of left eye (109) is being compared to when an injection is done in the left eye 107, then 109 (OS), can be moved, dragged or automatically be placed in location for example next to or in place of 110. A user remains in control and able to move items out of view and by activating icon 150 can take a snapshot (record) of a current arrangement of the medical records dashboard such that a record of the arrangement can be stored.

Simultaneously, a medical records dashboard of the present principles enables a user/medical care provider to recall and view plans of the past by activating a plan or A&P column or a particular plan in a column. The medical records dashboard of FIG. 23 enables current and past plans to be simultaneously displayed. As such, in context, a new note could be created in block 112. A medical records dashboard of the present principles, such as the medical records dashboard of FIG. 23, enables images, procedures, dates of service, plan, or any other patient-related data/information, such as clinical measurement, i.e. VA (vision OD 121.5—right or OS 122.5—left), to be compared in context. By way of example, how a treatment is working as measured by an image, clinical parameter, or any other related data set can be interpreted and noted in the medical records dashboard in at least block 71, which can be a new interpretation and can be edited by activating icon 70. In one embodiment a plan viewer can be accessed by activating block 112 and a new note or the editing of an old exiting note 79 can be accomplished via a text editor window 102. In the embodiment of the medical records dashboard of FIG. 23, a user/medical record provider is enabled to type or dictate a note 83 accurately while relevant information is viewed in for example a window. Although in the embodiment of FIG. 23 the medical records dashboard only provides a user/medical care provider one means for editing notes, in some embodiments, a medical records dashboard of the present principles can provide a user/medical care provider many ways to edit notes.

In the medical records dashboard of FIG. 23, panel 104 enables a user/medical care provider to select to view patient-related data/information from a number of different health care providers, such that patient-related data/information from every medical care provider that has ever cared for a patient can be viewed by, for example, all other specialties who provide care for that patient. For example in FIG. 23, a user/medical care provider can select to see patient care data/information related to a retina specialist 141 and/or a glaucoma specialist 142. In some embodiments, sharing of patient-related data/information from other users/medical care providers can require permission from at least one of the patient and the other user/medical care provider.

In the medical records dashboard of FIG. 23, panel, arranges patient data/information displayed in rows and columns. Users/medical care providers can have dashboards that are similar in display because the users/medical care providers charge, order, or perform similar CPT codes and often treat similar ICD diagnostic codes. Type of eye doctors are listed in order in this example #141 (retina), 142 (glaucoma), 86 (optometrist), and 142.5 comprehensive eye doctor.

In the embodiment of the medical records dashboard of FIG. 23, the different users/medical care providers can let all the other providers know something is important by highlighting the tab 141, 142, 91, 92, and 93 in the medical records dashboard view of other users/medical care providers. In such embodiments, a user/medical care provider is able to hover or otherwise active the highlighted tab to bring into view a message 94 that can detail an important aspect of patient care for the corresponding other user/medical care provider. As depicted in FIG. 23, a current user/medical care provider is alerted that a patient has missed appointments with a corresponding user/medical care provider. In another example, a tab to a family doctor 93 could light up or blink or in any way get a user's attention to indicate that an event is particularly important. In another example and as depicted in FIG. 23, when activated by a user/medical care provider, over a blinking endocrinologists tab 92 can appear an alert window 95 that can inform a user/medical care provider that a patient has received a diagnosis of cancer. In some embodiments, such important messages can be caused to display without requiring a user to activate or hover over a blinking or colored specialist tab.

There are situations where doctors, even if in separate practices and separate specialties, what they do can impact what another doctor does. By way of example, a retina surgeon injects many times in an eye, up to 12 times a year. But, clearly, if a family doctor discovers cancer that might change the frequency a retina doctor may want to inject. If a patient has a stroke, there are some research studies that suggest the medication that one doctor is using, in this case displayed 107 injections in the eye, by a retina surgeon might increase the risk of another stroke. In some embodiments, a Rules module of the present principles, such as the Rules module 004 of the Data Command center 001 of FIG. 1, is configured to recognize such situations in which treatment by one doctor can effect a treatment by another doctor and, in such instances, the Rules module 004 is configured to generate an alert to be displayed to all users/medical care providers of such situations.

There are many different ways that embodiments of a medical records dashboard of the present principles can display important information. By way of another example, at any time, if an important event occurs in any encounter of any provider, the information can be inserted into a row in chronological order, where it makes sense, to show on a timeline that the event occurred. So, if it was discovered that the patient had a stroke on May 25, 2019, as depicted by number 72 in FIG. 23, the initials of a caring provider can be displayed under the provider instead of a current provider as depicted in FIG. 23 by the intersection of 106 and 72 marked as 72.5. The difference between providers can be highlighted in many different ways. If it's a provider that is not normally on a row on clinical panel 105 or for example in this case, illustrated as an example of a retina doctor provider, then this new provider with a row can be highlighted or be a smaller row or a larger row. Also, instead of having the normal information in columns, because the other provider might not perform similar CPTs, instead in some embodiments there can be displayed, at the end of the row in a specially designated area for outside attachments or notes, information and it can be identified if the information is from a different provider.

In the embodiment of the medical records dashboard of FIG. 23, 85.5 can include financial data, and in this example shows ‘$’ sign. In such embodiments, access to financial data can be limited to only user/medical care providers credentialed to have access for instance only the users/medical care providers and colleagues in their practice can have access. In the embodiment of FIG. 23, icon 86.5 can be activated to enable access to financial data to different users/medical care providers. For example, in FIG. 23 86 is an example of an optometrist and 86.5 depicts an icon with appearance of two faces which can represent sharing access.

In the embodiment of the medical records dashboard of FIG. 23, the glaucoma specialists has 85.5 next to it, which can be used to launch a revenue cycle management (RCM), which is just one mechanism that any user/medical care provider can use to get more information in regards to their own practice's billing or any other information. By way of example, in the embodiment of FIG. 23, activating icon 85.5 can enable access to a user/medical care provider to cost, charges, any financial information payments, rejections, to which the user/medical care provider has access. In one embodiment, the financial information can comprise a mirror-image of the clinical dashboard, so a doctor, by toggling back and forth, a transparency or overlay can be used to determine what was charged, paid, rejected, or authorized for every service performed. Alternatively or in addition, clicking on RCM on the same view or on the same scanning screen the information that is financial in nature can be displayed under, over, above, or superimposed, similar to transparent paper, with one embodiment, the billing function, being behind or lighter and clinical being darker or vice versa. In some embodiments, each row of panel 104 can have 85.5 or 86.5 next to every one of the tabs (actionable dashboards of different providers).

In some embodiments of the present principles, a user of a medical records dashboard is identified upon use. For example, in some embodiments, a user/medical care provider is required to provide identifying information when the user/medical care provider wants to use a medical records dashboard of the present principles. In some embodiments, a user/medical care provider can provide predetermined configuration information to identify how a medical records dashboard should be displayed for that particular user. For example, in some embodiments a Rules module, such as the Rules module 004 of the Data Command center 001 of FIG. 1 can have access to configuration information for a medical records dashboard provided by a user. In such embodiments, the Rules module 004 can be configured to arrange and cause a display of the medical records dashboard in accordance with the predetermined configuration information provided by the user, for example, upon initiation of the medical records dashboard by the user.

Alternatively or in addition, in some embodiments, a user/medical care provider can drag and drop portions of a medical records dashboard to arrange the medical records dashboard into an arrangement that is best for the user and/or the user's practice or in some embodiments, into an arrangement that is best for a particular patient. For example, an eye doctors might care more about a condition like diabetes, so any doctor that takes care of diabetes, endocrinologists, family doctors, kidney specialists, urologists tend to have more patients and procedures related to diabetes than other specialists, like a radiologist.

In the embodiment of the medical records dashboard of FIG. 23, when a user selects 79, window 83 is displayed for inserting notes, which can then be saved and closed by selecting 191, or just closed by selecting 192.

Tab 107 of FIG. 23 is a tab for providing a user information regarding injections given to a patient, and tab 107.5 of FIG. 23 can provide quick information about the injections including a number of injection or a type of the injections. In FIG. 23, 111 depicts the identification of an example of an Eylea injection having been performed on Jul. 13, 2018, and it is red but can be highlighted in many different ways. In 111 adjacent to Eylea it says 15 days which in this example count from the last time an injection in the eye was done. In the embodiment of FIG. 23, the medical records dashboard depicts that Lucentis was injected Jun. 28, 2018 which is only days earlier from a Jul. 13, 2018 injection of Eylea and the column counts in the embodiment from one to the other. In some instances, procedures of Eylea or Lucentis injections are allowed only every 28 days from each other. In embodiments of the present principles, a Rules module, such as the Rules module 004 of the Data Command center 001 of FIG. 1, can be configured to have access to information, including but not limited to, rules regarding how frequent or far apart medications can be given, and in some embodiments, the Rules module 004 is configured to cause the display of an alert if a user/medical care provider is attempting to order a procedure improperly or if procedures have already been performed improperly.

In the embodiment of FIG. 23, panel 101 can be used to display diagnostic test and images. In the embodiment of FIG. 23, when tab 70 is selected an interpretation panel 71 is opened, which can display notes of an interpretation of patient care that could be actually written on the day of treatment. Element 81 of FIG. 23 is an image of a test performed on the patient.

In some embodiments, image icons, representative of results of test performed on a patient, can be selected to cause a display of an underlying corresponding image, such that a user/medical care provider can, in context, make a determination of the test and see the actual test while knowing whether there was a procedure or in this example a medication injection done, as depicted in 107.5

The embodiment of the medical records dashboard of the present principles of FIG. 23 illustratively includes a search box 89. The search box of the medical records dashboard of FIG. 23 can be used to search for a doctor, a date, an image, particular procedures, a particular diagnosis, payment rejections and payments and substantially any other patient related data/information related to the medical records dashboard. In some embodiment, the medical records dashboard can instantly reconfigure based on what is searched and can be configured to display only the portions of the medical records dashboard for which search results are returned. Combinations of queries can be searched. For instance, show only the rows and dates of service with the diagnosis of diabetes that had injections of a particular medication, column 107. Instantly, only the rows with injections with the patient having a diagnosis of a certain ICD like diabetes or if comparing a particular diagnostic test with a procedure and trying to correlate it, along with a clinical finding, the user could search “show me only the rows and dates of service where the vision was between 20/20 and 20/80” or “the pressure of 16 to 20 that also had the same date of service, a procedure in 107 of Eylea and also had an OCT. The patient data/information associated with the medical records dashboard can then be searched and in some embodiments, only rows and columns of the medical records dashboard related to the search can be searched.

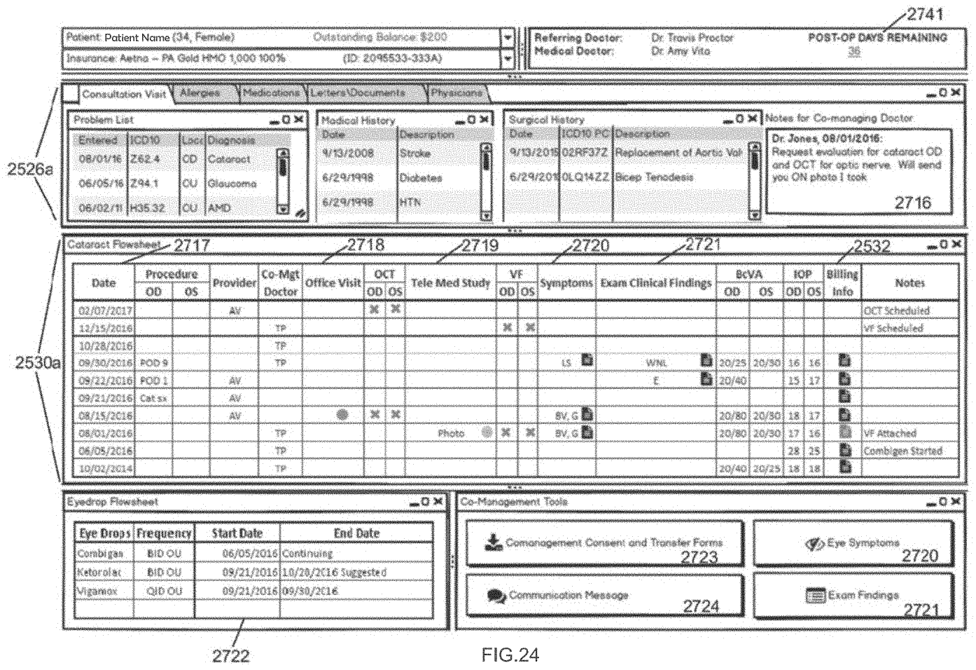

FIG. 24 depicts an embodiment of a co-managed medical records dashboard in the Data Command Center of the present principles in accordance with one embodiment. In the embodiment of FIG. 24, an optometrist and an ophthalmologist share (co-manage) a patient's cataract surgery then share treatment of the patient's glaucoma. A notes field 2716 in the Consultation Visit 2526a panel presents a mechanism to facilitate contextual content surrounding the co-managed procedure(s). A Cataract Flowsheet 2530a (purpose optimized dynamic panel) is presented with structured data elements designed to facilitate the identified procedure as conducted by multiple care givers. The Cataract Flowsheet 2530a (purpose optimized dynamic panel) is presented with structured data elements designed to facilitate the identified procedure as conducted by multiple care givers. The Cataract Flowsheet 2530a is arranged by interaction dates 2717 and tracks office visits 2718 (both scheduled and realized) including sending reminders to patients and alerts when an appointment is missed (not shown), provides means to review and issue concurrence or dissent with diagnostic tests 2719, a summary of symptoms 2720, and a summary of exam findings. The Data Command Center keeps track of appointments between comanaging providers and when an appointment is not kept. The Data Command Center enables messaging to both providers as well as reminders through patient portal for patient to schedule appointment. (describe modules and details of this function). Where available, billing summaries 2532 are presented in the Cataract Flowsheet 2530a as well. Clicking the billing summary 2532 can open a new billing window to show billing details. Eye drops after cataract surgery and/or glaucoma treatment can be tracked on the Eye Drop Flowsheet 2722 (another purpose optimized dynamic panel).

In the embodiment of FIG. 24, there is panel of co-management tools that provide the user with a means to download relevant forms 2723, and to send direct messages to the co-managing physician using button 2724 to access a co-management message center. An indication of the number of postop days remaining 2741 may also be provided. All financial data in the system, including costs to patient, is compartmentalized such that no user can see financial details for users or organizations not authorized in accordance with applicable policies and law. In addition, any rows and columns of information can be programmed to include or exclude those data fields from either provider.

To co-manage a patient using the interface embodiment illustrated in FIG. 24, when a referring medical care provider outside the practice wants a consultation, he or she can connect to the practice they are referring to and send information by opening the Cataract Flowsheet 2530a. The referring physician can manually insert or auto-populate information from any previous visit of the patient and provide an annotation 2717 giving the reason for the consultation. When the receiving consultation medical care provider examines the patient, the Cataract Flowsheet 2530a is auto-populated with the medical care provider's findings. Co-management forms can be downloaded from the table at 2718 either at the time of the referral or after the consulting medical care provider fills out the paperwork and the patient signs a consent form by selecting co-management forms or by visiting the referring medical care provider's website. A message is sent to the referring medical care provider to open the Cataract Flowsheet 2530a and the consent or other forms can be clicked upon and the referring medical care provider can read or sign any forms needed. The “co-management consent” can change color or be distinguished in some other fashion when received back from the referring physician. Every time the surgeon sees the patient, the Cataract Flowsheet 2530a automatically includes the date and findings. Then, post-operatively the co-managing physician, or consultation physician or optometrist, when they see the patient in their office, auto-populates or fills out on the Cataract Flowsheet 2530a and shares any results.

In the embodiment of FIG. 24, notes may be communicated between the medical care provider by selecting “communication message” 2724 to determine if there is any information that needs to be shared for office visits. The date column 2719 and office visit column 2720 are tied together. Some of the columns are left blank until the patient actually shows up for a future visit. For instance, after a surgery or consultation, the consulting medical care provider, just as they would normally give an appointment card to a patient can actually give a co-management medical summary table where it shows the date of the future appointment at the referring or sharing medical care provider's office, and when that date arrives the patient is seen and everything is auto populated so the surgeon can see the results that the co-managing medical care provider found. The findings are auto populated by the optometrist/referring medical care provider/co-managing sharing medical care provider. If the appointment date is missed, the table can link up with the missing ticket report or send an alert to the patient themselves, the surgeon, the referring medical care provider, business managers or anyone else as appropriate.

In accordance with the present principles, shared medical care may be provided in management of common eye conditions besides cataracts, such as glaucoma. For example, an optometrist/general ophthalmologist can manage interval visits after the glaucoma specialist establishes a plan of care. That is, after initial consultation, the plan can be shared with the referring or co-managing medical care provider. At a subsequent examination, the referring medical care provider accesses patient data, executes the plan and enters the data into a Cataract Flowsheet and/or a Glaucoma Flowsheet. An alert can then be sent to the glaucoma specialist confirming that the action plan is being carried out. This facilitates can care for the patient according to the plan. The glaucoma specialist can follow up every year or two while sharing interval visits with the referring optometrist/general ophthalmologist. Multiple benefits of the concepts of the present principles include excellent care, appropriate supervision, reduced cost, improved quality of care of the patient without undue distance traveled. At any point of execution of the treatment plan, treatment can be altered based on clinical data available to the patient, glaucoma specialist as well as the referring medical care provider at all times. Of course, other fields of medicine and industry have similar examples. For example, orthopedic surgeons share care with podiatrists and family physicians share care with all medical specialists. A prime example is shared care with multiple healthcare providers caring for a patient with a chronic disease, state such as diabetes. One patient can have an eye doctor, podiatrist, primary care doctor, endocrinologist, nephrologist, dietician, exercise physiologist, all who need to share care. Different medical care providers can order the same or different tests. If they are in separate health systems, they may not know each other's diagnostic tests, but through the shared medical records dashboard of the present principles, medical care providers can avoid duplication of ordering tests, thereby, reducing costs and delivering better care. In some embodiments, different practices can identify what is important for them to know about a patient and information from the various respective medical records dashboards can be combined so that the identified important information can populate into a single dashboard.

For instance, a general ophthalmologist can have a complex case, for instance neovascular glaucoma, which can sometimes be associated with carotid disease. In some instances the ophthalmologist can send the patient to a glaucoma surgeon. In some embodiments, the pertinent portions of the medical records dashboard of the general ophthalmologist's can be displayed to the glaucoma surgeon, who now has the necessary information to care for the patient. The general ophthalmologist's medical records dashboard can be automatically populated to include the encounters between the patient(s) and the general ophthalmologist, so that medical care providers can, in real time, see what the changes in the patient's treatment are made. In some embodiments, other specialist can become involved in the treatment of a patient and can also have respective medical records dashboards that can share information with some or all of the other medical records dashboards of already involved medical care providers.

In addition, embodiments of the present principles as described above can be implemented to track laboratory tests. For example, every day a family physician and the patients they see can schedule radiological or diagnostic tests to be performed on a patient. A difficulty arises in keeping track of all the different referrals and/or the medications that are prescribed. A medical records dashboard of a Data Command Center of the present principles is able to keep track of every single diagnostic test, medication, or consultation that medical care providers prescribe. Using a medical records dashboard of the present principles, a medical care provider can sort a patient's medical history by date ordered, date performed, or by patient. The results can be automatically collated in rows and columns or in other orientations on a single display. As a patient's laboratory results come back, an entire group of patients that were seen in any time period or for a particular diagnostic test can be displayed in red on a medical records dashboard until the results are received. Upon receiving the test results, the test results can turn another color to indicate the receipt of the results. In such a way, a medical care provider is able to track all of their practice's patients and what the results are, when they are received. In some embodiments, a medical care provider can be alerted to abnormal results.

In embodiments in which the Data Command Center of the present principles, such as the Data Command center 001 of FIG. 1, enables the Co-Management of patient information available via a medical records dashboard of the present principles and as described above, Co-Management is meant to refer to referrals, transfers of care, and any instance of the sharing of patient data either unidirectionally, bidirectionally, or multi-directionally between a Data Command Center of the present principles and any source of patient data and the management of such data via, for example, a medical records dashboard in accordance with the present principles. In some embodiments, a Co-Management process of the present principles can be accessed utilizing a button, keystroke, or series of keystrokes, to initiate the Co-Management workflow.

In some embodiments, upon initiation of a Co-Management process of the present principles, a user can be given the option (i.e., via a prompt on a display) to select a predetermined template for performing Co-Management, to select to determine a custom configuration for performing Co-Management, or to select a hybrid configuration for performing Co-Management. For example, in some embodiments, a template or set of templates can be preconfigured and stored and accessible to at least one of the Rules module 004 and the Display module 006 of the Data Command center 001 for configuring the medical records dashboard and displaying the medical records dashboard in accordance with a selected, preconfigured template. In some embodiments, a predetermined templates can be preconfigured based upon conditions including but not limited to a specialty of at least one medical care provider/user, practice location of at least one medical care provider/user, the identity of at least one medical care provider/user and/or at least one patient, at least one patient's conditions, procedures performed on at least one patient, risk factors for at least one patient, diagnostic results of at least one patient, future orders for at least one patient, future appointments for at least one patient, data values recorded for at least one patient, data values not recorded for at least one patient, calculated data values for at least one patient and absolute values for display. That is in some embodiments, portions, columns, and/or rows of a medical records dashboard to be displayed or hidden can be determined based on a selected preconfigured template of a Co-Management process in accordance with the present principles.

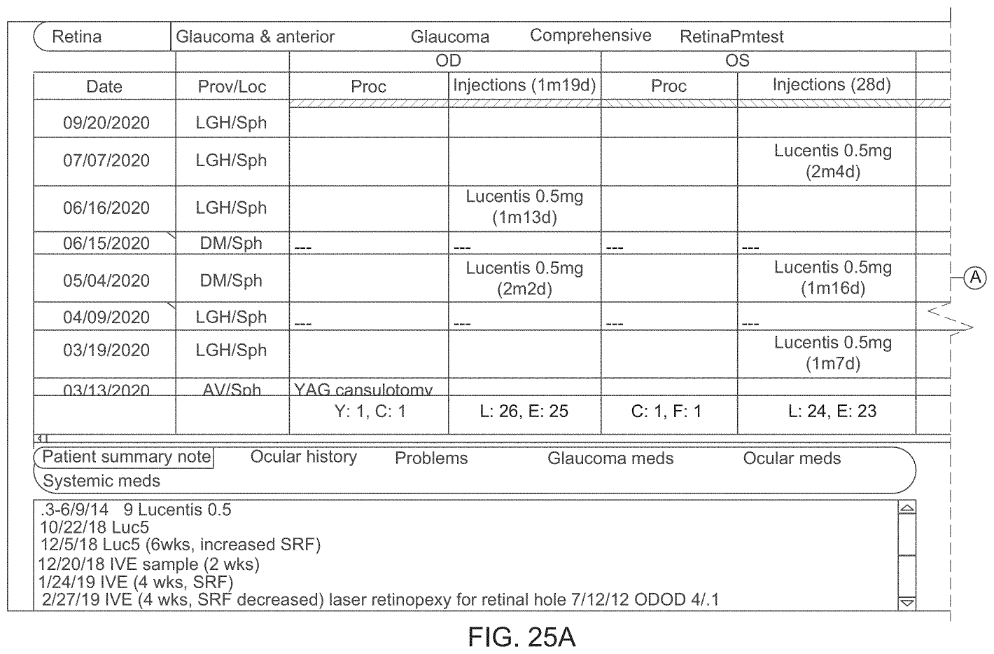

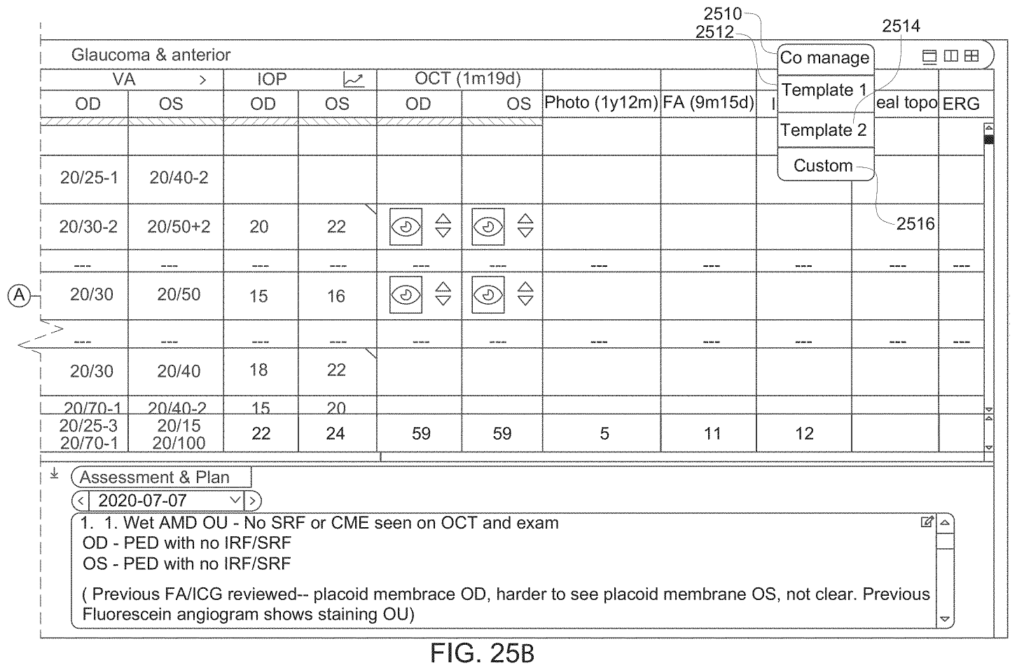

Alternatively or in addition, in some embodiments portions, columns, and/or rows of a medical records dashboard to be displayed or hidden can be determined based on a custom template of a Co-Management process in accordance with the present principles. In some embodiments a Co-management template of the present principles can be determined using, for example, a user interface of the computing device 200 of FIGS. 1 and 2. That is, in some embodiments a user interface can be implemented to create a custom Co-Management template in accordance with the present principles. For example, FIG. 25A and FIG. 25B, collectively referred to as FIG. 25, depicts a medical records dashboard including an ability to launch a Co-Management process in accordance with an embodiment the present principles. In the embodiment of FIG. 25, the medical records dashboard includes a Con-Manage icon/button 2510 for launching a Co-Management process. Upon selection of the Co-Manage icon/button 2510, a menu is displayed on the medical records dashboard enabling a user to select between preconfigured templates, illustratively preconfigured template 1, 2512, and preconfigured template 2, 2514. In the embodiment of FIG. 25, the displayed menu further enables a user to select the ability to create a custom template 2516.

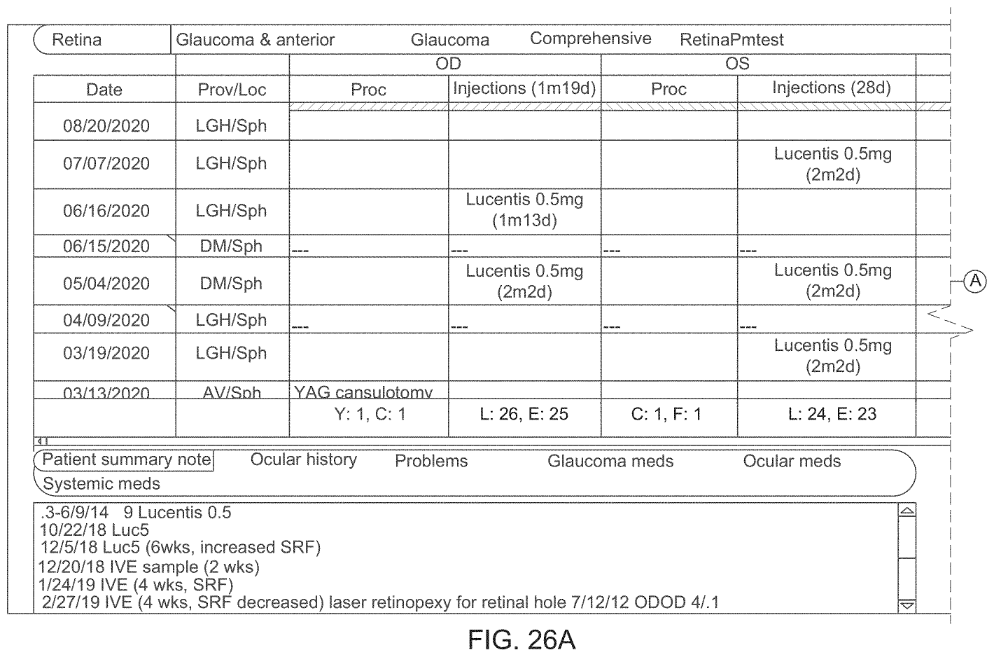

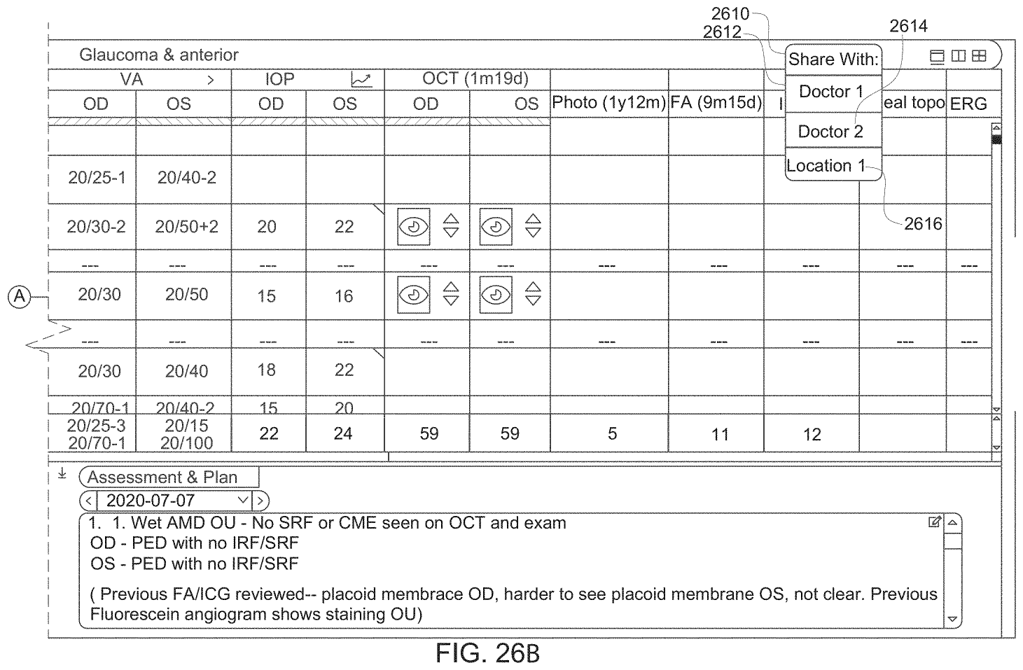

Upon selection by a user of the custom template 2516, a process is initiated that, in some embodiments, enables a user to select portions, columns and/or rows of the medical records dashboard to display or hide. For example, FIG. 26A and FIG. 26B, collectively referred to as FIG. 26, depicts a medical records dashboard including a custom template creation process for co-management in accordance with an embodiment of the present principles. In the embodiment depicted in FIG. 26, a user is given the ability to select, for example using a user interface (i.e., mouse, keyboard, etc.), portions, columns, and/or rows of the medical records dashboard to be accessible to (i.e., displayed to) a shared user(s) of a Data Command Center of the present principles, such as the Data Command center 001 depicted in FIG. 1, via a medical records dashboard in accordance with an embodiment of the present principles. Alternatively, a user can select, via the process described above, portions, columns, and/or rows of the medical records dashboard to be hidden from (i.e., not displayed to) a shared user(s) of the Data Command Center.

In some embodiments, information regarding preconfigured templates and custom templates for a Co-Management process in accordance with the present principles can be associated with at least one of the Rules module 004 and the Display module 006 of the Data Command center 001 of FIG. 1. For example, in some embodiments, information regarding preconfigured templates can be stored in a storage means accessible to the Rules module 004. As such, during a Co-Management process in accordance with the present principles, when a preconfigured template is selected by a user, the Rules module 004 can configure what portions, columns, and/or rows of the medical records dashboard are to be hidden or displayed based on the preconfigured template selected by the user. Such information can then be made available to the Display module 006, which causes the display or lack of display of portions, columns, and/or rows of the medical records dashboard based on the determinations and information associated with a selected, preconfigured template.

In some embodiments in which a user selects to create a custom template, upon selection of the creation of a custom template, the Rules module 004 can initiate a process, for example as described above with reference to FIG. 26, for enabling a user(s) to select to which to portions, columns, and/or rows of the medical records dashboard a user(s) is to be allowed or denied access. In some embodiments the Rules module 004 stores such custom template configuration selected by the user(s) in a storage means accessible to the Rules module 004 and the Display module 006. Upon creation of a custom template for Co-Management in accordance with the present principles, the Display module 006 can cause the display or lack of display of portions, columns, and/or rows of the medical records dashboard based on the determinations and information associated with a created, custom template.

In addition to the selection of a preconfigured template, for example preconfigured template 1, 2512, and preconfigured template 2, 2514, and/or the creation of a custom template, for example custom template 8716, in some embodiments, a Data Command Center of the present principles, such as the Data Command Center 100 depicted in FIG. 1, via a medical records dashboard, can enable a user(s) to select parameters that decide to whom/what/where to enable access or deny access to portions, columns, and/or rows of the medical records dashboard. For example, in the embodiment of FIG. 26 the medical records dashboard comprises a Share With menu 2610 enabling a user(s) to select to whom/what to enable access or deny access to portions, columns, and/or rows of the medical records dashboard. In some embodiments, the Share With menu 2610 can include predetermined selections such as a first doctor, Doctor 1 2612, a second doctor, Doctor 2 2614, and a location, such as the location of a medical practice, Location 1 2616. Alternatively or in addition, in some embodiments the medical records dashboard can enable a user(s) to input identifying information including but not limited to a specialty of at least one medical care provider/user, practice location of at least one medical care provider/user, the identity of at least one medical care provider/user and/or at least one patient, at least one patient's conditions, procedures performed on at least one patient, risk factors for at least one patient, diagnostic results of at least one patient, future orders for at least one patient, future appointments for at least one patient, data values recorded for at least one patient, data values not recorded for at least one patient, calculated data values for at least one patient and absolute values for display to identify to whom/what to enable access or deny access to portions, columns, and/or rows of the medical records dashboard.

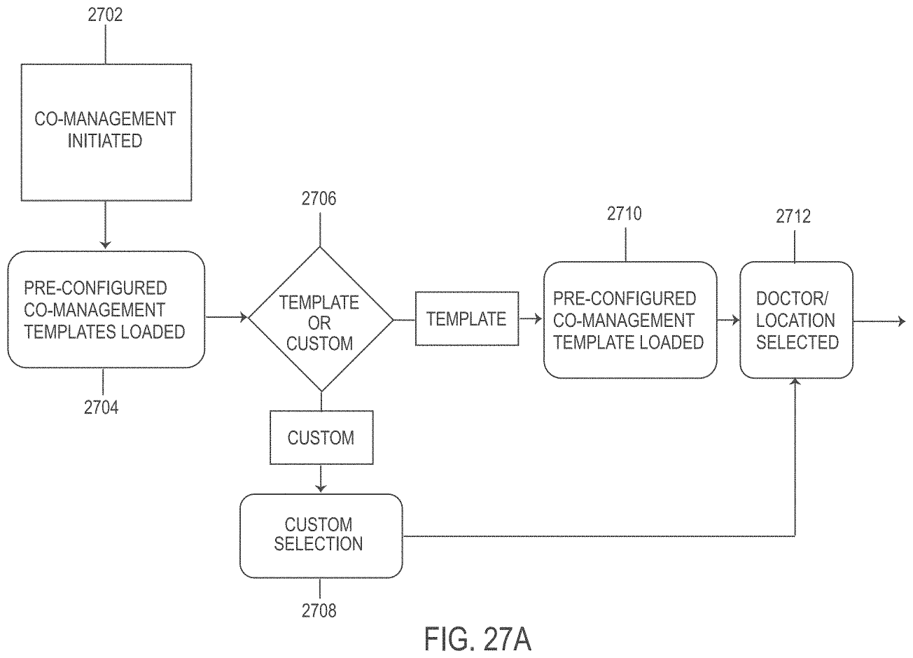

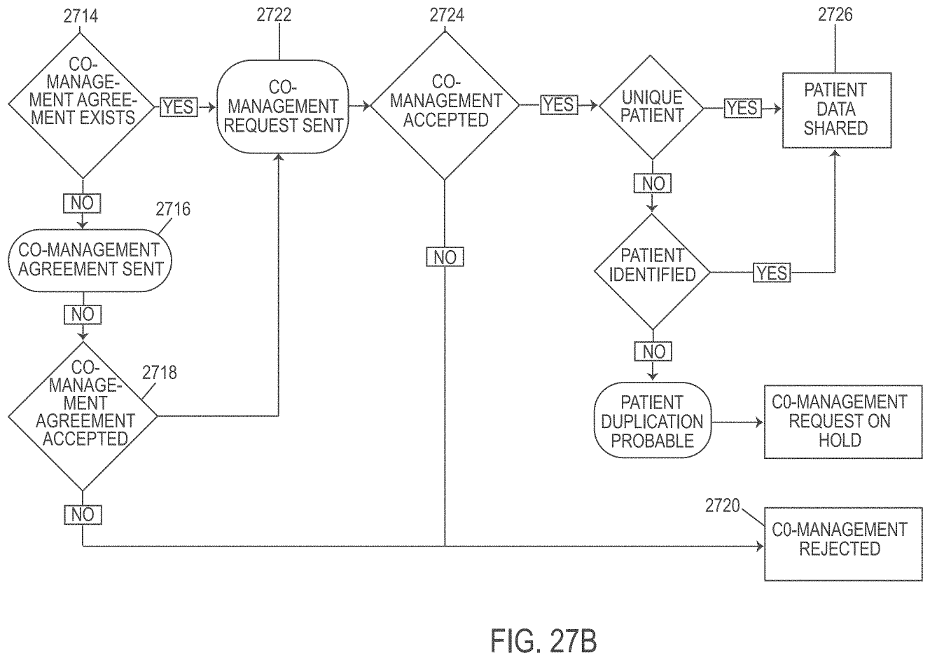

FIG. 27A depicts a first portion of a workflow diagram of a Co-Management process and FIG. 27B depicts a second portion of the workflow diagram of the Co Management process of FIG. 27A in accordance with an embodiment of the present principles (referred to collectively as FIG. 27 herein). In the embodiment of FIG. 27A and FIG. 27B, the Co-Management process is initiated at 2702. At 2704 preconfigured Co-Management templates are all loaded. A selection is then made by a user(s) to use a pre-configured template(s) or to use the Custom option to create a Custom Co-Management configuration at 2706. If a user selects to use a pre-configured template, the selected pre-configured template is loaded at 2708. If a user selects to create a Custom Co-Management configuration, user selections for creating the Custom Co-Management configuration and determining which portions, columns, and/or rows of the medical records dashboard to which to grant or deny access are made at 2710. In the embodiment of FIG. 27A and FIG. 27B, at 2712, a user selects select to whom/what/where to enable access or deny access to portions, columns, and/or rows of the medical records dashboard.

At 2714 it is determined if a Co-Management agreement exists. If no Co-Management agreement exists a Co-Management agreement is communicated to at least one other user at 2716. At 2718 it is determined if the communicated Co-Management agreement was accepted by another user. If the communicated Co-Management agreement was not accepted by another user, the Co-Management agreement is cancelled at 2720. If at 2718 it is determined that the communicated Co-Management agreement was accepted by at least one other user, a Co-Management request is communicated to an accepting user at 2722.

Referring back to 2714, if it is determined that a Co-Management agreement does exist, the process also proceeds to 2722 during which a Co-Management request is communicated to at least one user with which the Co-Management agreement exists. At 2724 it is determined if the Co-Management request was accepted. If at 2722 it is determined that the Co-Management agreement is not accepted, the Co-Management agreement is cancelled at 2720. If at 2722 it is determined that the Co-Management request has been accepted by at least one user, the patient data is shared at 2726 in the medical records dashboard in accordance with the pre-configured template selected or the custom configuration created and the whom/what/where selections made by a user(s).

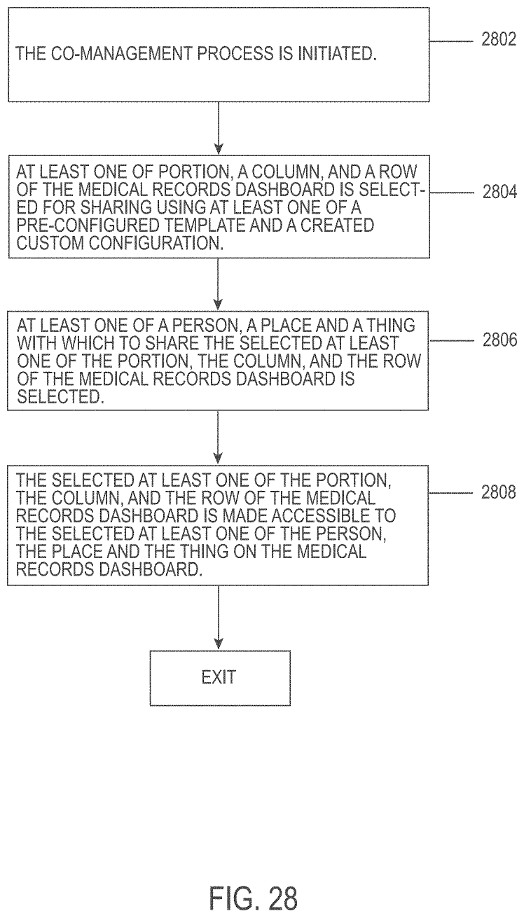

FIG. 28 depicts a flow diagram of a method for Co-Management of patient information in a medical records dashboard in accordance with an embodiment of the present principles. In the embodiment of FIG. 28, the method begins at 2802 during which the Co-Management process is initiated. For example and as described above, in some embodiments the medical records dashboard can include a Co-Management icon/button for initiating a Co-Management process in accordance with the present principles. The method can proceed to 2804.

At 2804, at least one of a portion, a column, and a row of the medical records dashboard is selected for sharing using at least one of a pre-configured template and a created custom configuration. The method can proceed to 2806.

At 2806, at least one of a person, a place and a thing with which to share the selected at least one of the portion, the column, and the row of the medical records dashboard is selected.

At 2808, the selected at least one of the portion, the column, and the row of the medical records dashboard is made accessible to the selected at least one of the person, the place and the thing on the medical records dashboard. The method can then be exited.

In some embodiments, the Co-Management Workflow can exist in a single, unidirectional state, whereby the party that initiates the Co-Management request shares data with the recipient, but the recipient does not reciprocate sharing of patient data. In another embodiment, the party that initiates the Co-Management request shares patient data with the recipient, and the recipient initiates a Co-Management request to the party that initiated the initial request, thus data is shared bidirectionally. In another embodiment, several parties initiate Co-Management requests, and each party shares data with each other party, in a multi-directional state. At any point, a Co-Management participant my opt to no longer share data with one or more recipients, at which point data sharing and the Co-Management workflow reaches a logical end.

In some embodiments, upon initiation of Co-Management, a record of the Co-Managed patient is recorded, including all relevant Patient Identifiers from all parties involved in Co-Management. Alternatively or in addition, upon initiation of Co-Management, shared configurations are recorded. Shared configurations can be used to determine what data from each party can be viewed within a recipient's medical records dashboard in accordance with the present principles.

In some embodiments, a source of patient data can exist within storage means associated with respective Data Command Centers of users participating in the Co-Management of the present principles. In such embodiments, shared data can consist of a series of links or cached data in the respective Co-Management databases. Links or cached data can be updated upon any change in source. Additionally in some embodiments, data can be recorded within a Co-Management database as well as a database/storage means associated with a participating user's respective Data Command Center, the data including, but not limited to, audit logs of Co-Management Workflow interactions, Messaging between users, file and document sharing between users, and notifications and/or triggers for automated tasks. It should be noted that, in some embodiments, a Co-Management Workflow in accordance with the present principles can be non-linear, can be automated in whole or in individual or groups of steps, and algorithms can intelligently update, flag, or otherwise override certain steps of the Co-Management Workflow.

In one example of a Co-Management Workflow in accordance with the present principles, a primary care physician (PCP) can initiate the Co-Management Workflow for a single patient having multiple Specialists. Each Co-Managing Specialist can opt to Co-Manage with one of more PCPs and Specialists. In some embodiments, the Co-Managed patient data would not be shared further than one logical step, thus a PCP can share their patient data with Specialist 1, who then shares their patient data with Specialist 2, but the PCP's patient data would not be shared with Specialist 2 unless the PCP takes action to initiate Co-Management with Specialist 2.

In a second example, a doctor can initiate a Co-Management Workflow of the present principles with a patient during a Transfer of Care, in which case, the patient's data is shared unidirectionally, and the recipient is not expected to share data back with the initiating doctor, nor is there an expectation that the patient would return to the transferring doctor.

In a third example of a Co-Management Workflow of the present principles and with the context of a hospital and several physicians, as is normally the case in patient care, any number of Co-Management Agreements and Workflows can be in place to allow for patient data sharing between any to all recipients of a Co-Management Request. This configuration can include unidirectional sharing, bidirectional sharing, and multi-directional sharing of patient data in accordance with the present principles.

In co-management, where different practices share information about the same patient, it is critical to identify that the patient that is being shared is in fact the same person. There can be dozens of John Smiths and systems cross-reference by looking at the last name, the age, the gender, the zip code and perhaps the home address. But still, there can be confusion between patients. In medicine you can take no chances that you confuse one patient with the other and when patients travel from different offices or different EMRs and computer systems, the possibility of confusion is present.

In some embodiments, the Data Command Center of the present principles, such as the Data Command center 001 of FIG. 1, enables unique patient identification by incorporating patient medical history information. Current methods for identifying patients include matching Social Security Numbers (SSN) and Driver's License Numbers, where available. However, as privacy became more of a concern in the modern digital age, such data is becoming less available to medical care providers and their Practices. In addition, other methods for identifying patients can include identifying patients via First Name, Middle Name or Initial, Last Name, Age, Sex, Address, City, State, and Zip Code. Such information, however, is subject to flaws of human error, such as typos, human choice, such as a patient offering a nickname instead of the accurate name on a birth certificate or other identification. In addition, even having accurate patient information, it can still be difficult to distinguish between two people having the same name. Using such current methods, multiple systems are only able to match patients whose information is listed exactly the same in the multiple systems, a limitation which requires human intervention and prevents full automation of the process.

A subset of data exists within the Medical Community, as mandated by Meaningful Use 2014 and 2015 EHR Certification requirements specified in 45 CFR § 170.102, known as the Common Clinical Data Set (CCDS). The CCDS consists of patient information including, Patient Name, Sex, Date of birth, Race, Ethnicity, Preferred language, Smoking status, Medical Problems, Medications being taken, Medication allergies, Laboratory test(s) having been performed on the patient, values of the Laboratory result(s), Vital signs, Procedures, Care team member(s), Immunizations, Unique device identifier(s) for a patient's implantable device(s), Assessment and plan of treatment, Treatment Goals, Health concerns and the like.

CCDS was developed to encourage interoperability through the exchange of a common data set and is routinely shared between practices by means of the Direct Messaging Exchange, a secure messaging system by which Continuity of Care Document (CCD) or other document conforming to the Clinical Document Architecture (CDA) as defined in the 2014 and 2015 Certified EHR requirements. This is the current standard for Clinical Data transport between EHRs, thus between practices. The future requirement, Fast Healthcare Interoperability Resources (FHIR), expands on the clinical data set to include more discrete data points.

In accordance the present principles, the inventors propose to incorporate such additional data, such as the data supplied through the CCDS, to accurately identify unique patients using a combination of techniques including but not limited to a Common PII Matching technique, a Problems, Allergies, and Medications technique, a Doctors, Locations, and Procedures technique, and CCDS data technique.

In a Common PII Matching technique, none of the PII data may be valid given name changes, nicknames, and misspellings, as well as marriage and legal name changes, addresses and phone numbers change over time, and the increasing reluctance of patient and practice alike to maintain or share key identification numbers. At best, every data point would need to match exactly to ensure the closest match, but can still fall short in the cases of same names such as in the case of George Forman's eight sons all named George Edward Foreman, if date of birth and suffix data was not present. Twins could make identification even more difficult. As evident, the Common PII Matching technique may not be reliable on its own for identifying unique patients.

In a Problems, Allergies, and Medications technique, a commonly shared data set which includes key conditions (Problems), allergies to certain medicines (Allergies), and specific medications (Medications), is compared to determine a profile of a patient which offers an additional level of accuracy by taking a loose match from PII and determining if that patient also has the same list of Medical Problems, Allergies, and Medications in a system for comparison. The likelihood that two people within similar PII, or lacking key aspects of PII, would also share the same Problems, Allergies, and Medications is a significant reduction in ambiguity. For instance, George Foreman's 3rd son may share certain genetic predispositions to Medical Problems and even share Allergies with a 1^{st} son, but the likelihood that George Foreman's two sons would have been prescribed the same exact Medications for these and any other Problems they have is minimal.

In a Doctors, Locations, and Procedures technique, information from a document complying with the CCDA can be used for identifying a unique patient. For example, each CCD, or document complying with the CCDA, is required to have specific information in the Header of the document denoting the Care Provider, Date, and Location. The body of the document contains Procedures and relative Dates. The high accuracy enabled when comparing patients' Doctors, Locations, and Procedures is a product of the inability for a Doctor to see more than one patient at the exact same time, the unlikelihood of that even if the doctor saw more than one patient at the same time, and at the same location, the Doctor still would have little ability to perform the same procedure at the same time on more than one patient.

In a CCDS data technique, additional Data from the CCDS, when available, offers increased accuracy in patient identification and matching. That is, comparing patient information including at least Patient Name, Sex, Date of birth, Race, Ethnicity, Preferred language, Smoking status, Medical Problems, Medications being taken, Medication allergies, Laboratory test(s) having been performed on the patient, values of the Laboratory result(s), Vital signs, Procedures, Care team member(s), Immunizations, Unique device identifier(s) for a patient's implantable device(s), Assessment and plan of treatment, Treatment Goals, Health concerns and the like, among different patients, greatly increases the accuracy of unique patient identification.

In some embodiments of a Unique Patient Identification method of a Data Command Center in accordance with the present principles, a Unique Patient Identification algorithm collects every available Identification Point, validates the points for presence of data, and assigns each Identification point a level of accuracy as it pertains to Patient Matching. Presence of data points with High Accuracy are prioritized and validated. Each Exact match is scored for accuracy. Each Likely Match is appropriately scored for accuracy. Each data point with no matching counterpart is negatively scored. Presence of data points with Moderate Accuracy are then prioritized and validated. Each Exact match is scored for accuracy. Each Likely Match is appropriately scored for accuracy. Each data point with no matching counterpart is negatively scored. Moderate accuracy data points are scored lower than High accuracy data points. Presence of data points with Low Accuracy are then prioritized and validated. Each Exact match is scored for accuracy. Each Likely Match is appropriately scored for accuracy. Each data point with no matching counterpart is negatively scored. Low accuracy data points are score lower than Moderate accuracy data points.

Upon gathering and analyzing all available data for Unique Patient Identification, scores are tallied and compared to an acceptable Matching Threshold. In some embodiments of the present principles, the Matching Threshold is configured to clearly exceed a matching accuracy of current patient identification techniques with the inclusion of far more points of identification to compare. In some embodiments, the matching of the present principles can occur without the requirement of matching on current PII data. For example, George Edward Foreman IV may have been staying with a friend in Florida when he visited a doctor. Not wanting to be identified as the son of the famous boxer, he purposely listed his name as G. Foreman and address as the place he was staying. Date of birth may have been left blank. A positive identification can still be made, in accordance with the present principles, if the clinical data supplied matches with a high enough degree of accuracy clinical data stored for George Edward Foreman IV, such as the unique identifier on his knee replacement or the fact that a large number of Doctors, Locations, Procedures, Problems, Allergies, Medications, and Lab Results are found to be matching, while the name, address, and date of birth have non-matching counterparts.

A Unique Patient Identification algorithm of the present principles can reach a logical end when a positive match is determined, or no positive match can be made. In some embodiments, should no positive match be made, the patient and possible matches can be flagged for human intervention.

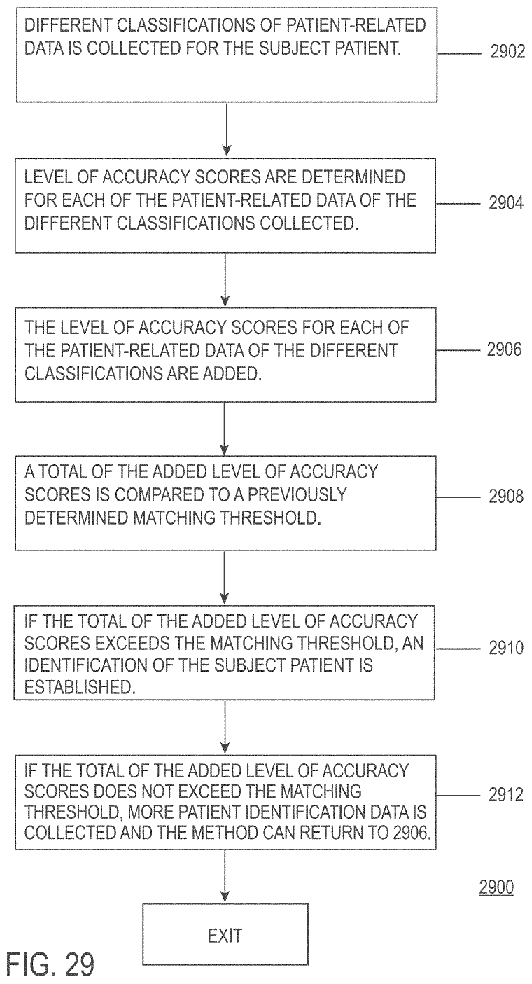

FIG. 29 depicts a flow diagram of a method for Unique Patient Identification for a subject patient in a Data Command Center including patient-related data received or derived from at least one patient database in accordance with an embodiment of the present principles. The method 2900 of FIG. 29 illustratively begins at 2902 during which different classifications of patient-related data is collected for the subject patient. For example and as described above, in some embodiments, data from the Common Clinical Data Set and other sources can be collected to be used in patient identification techniques of the present principles. The method 2900 can proceed to 2904.

At 2904, level of accuracy scores are given for each of the patient-related data of the different classifications collected. The method 2900 can proceed to 2906.

At 2906, the level of accuracy scores for each of the patient-related data of the different classifications are added. The method 2900 can proceed to 2908.

At 2908, a total of the added level of accuracy scores is compared to a previously determined matching threshold. The method 2900 can proceed to 2910.

At 2910, if the total of the added level of accuracy scores exceeds the matching threshold, an identification of the subject patient is established. The method 2900 can proceed to 2912.

At 2912, if the total of the added level of accuracy scores does not exceed the matching threshold, more patient identification data is collected and the method 2900 can return to 2906. The method 2900 can then be exited.

It is critical for a medical care provider to know what medications a patient has ever taken or is currently taking, what the frequency is, why the medication was taken or discontinued and reasons for switching to another medication. There is currently no medication management tool that visually correlates the clinical parameters or disease state findings that the medication is prescribed to have an impact on. A Data Command Center of the present principles via at least one of a medical records dashboard and a Medications Management chart or tool in accordance with the present principles enables a user to correlate frequency, amount and types of medications taken to enable the user to visualize how that medication affects the parameters reviewing modulation such as blood pressure, eye pressure, weight, heart rate, etc. and corresponding it to when the medications were taken to see if there is a cause and effect. There is no system that can also correlate and display on a view surgical intervention, an injection or any other intervention and see how these additional factors correlate with timing of medication taken and how all this impacts clinical finding, measurements, disease progression and symptoms. A Data Command Center of the present principles enables a user to visually correlate diagnostic tests and images that may show how all these treatment modalities result in changes or lack thereof on lab results, imaging, etc. For example and as enabled by embodiments of the present principles, if a patient is being treated for cancer and chemotherapeutic medication can be seen with direct access on one screen with x-rays taken over time showing changes in size of a tumor or mass along with the labs or clinical symptom changes all in context of when surgical or radiation therapy intervention was performed, enables medical care providers to efficiently and accurately make medical decisions.

Embodiments of a Data Command Center of the present principles can also be linked to a Pharmaceutical system or other provider of prescribed medication (i.e., E-prescribe or a similar system) such that a medical care provider is enabled to accurately track when medication was actually received by a patient. It can be very difficult if not impossible with current systems for a medical care provider to know when a medication was actually received by a patient. That is, medical care providers often rely on scribes to write prescriptions and when patients call to refill the medication, often it is not the medical care provider who prescribes the refills of medication but an assistant who does so. Even further, just because a medical care provider orders a drug for a patient that does not mean the patient actually went and got it filled or that the medication was taken as prescribed. To further complicate matter, patients can be given different medication than prescribed by the medical care provider because a generic drug instead of a brand drug could have been given.

Embodiments of a Data Command Center of the present principles can also be linked to home monitoring devices or system for being able to more accurately determine when medication was actually taken by a patient. That is, just because medications are prescribed and received by a patient does not mean that the patient has started taking the medication or even taking it as prescribed. A patient may also misunderstand what the doctor actually wants the patient to do and is actually taking the medication incorrectly. Embodiments of a Data Command Center via, for example, a medical records dashboard of the present principles enable medical care providers to more accurately track medications and how they are being taken by patients, which improves quality of care. More specifically, in accordance with the present principles, a medical care provider is enabled to visualize the medications, the start and stop dates, reasons for discontinuation, and is enabled to manage and change the display based on reality they confirm with the patient at point of care and via the pharmaceutical and home monitoring devices that can be linked into the Data Command Center of the present principles.

As described above, embodiments of a Data Command Center via, for example, at least one of a medical records dashboard and a Medication Management chart/tool of the present principles enables medical care providers to more accurately track medications and dates associated with the medications, for example in rows and columns. In some embodiments a Data Command Center via, for example, at least one of a medical records dashboard and a Medication Management chart/tool of the present principles can display tracked medication information in graph form. In some embodiments, each medication or class of medications associated with a patient can be represented by a bar graph or a linear graph or other visual method or means that in either the vertical direction or in a horizontal direction the doctor can visualize the actual start and stop dates of all relevant medications for a patient, which can all be seen simultaneously with any other relevant data that the medications can impact. More specifically, in some embodiments, a Data Command Center in accordance with the present principles, such as the Data Command center 001 of FIG. 1, can further include the ability to intelligently display medications in context (referred to by the inventors in some embodiments as Medication Management), by grouping, categorizing, expanding, contracting, displaying, hiding, and highlighting or flagging medications to visually present medications to a user of the Data Command Center (e.g., medical care provider) in a medical records dashboard in a manner that makes such medication more easily identifiable by the user. In one embodiment, Medication Management exists as a series of intelligent vertical columns representing individual medications, classes of medications, categories of medications, or logical groupings of medications, differentiating medications by color or combinations of colors, symbols, and/or text, graphing start and stop dates and times or individual doses correlated to relevant values and relevant events. In accordance with the present principles, graphical differentiation between medications can consist of individual colors for individual medications, combinations of colors for medications including more than one component, or complex graphical representations. In some embodiments, color standards, such as defined by the American Academy of Ophthalmology, can be used for color coding the medications and/or custom colors can be used. For example, in ophthalmology and with respect to eye care, medications have been assigned in the industry to have a certain color on the eye drop bottle or cap. In some embodiments, these colors can be displayed allowing recognition by the user of the class of medication. For instance, yellow is a beta blocker one of which is Timoptic. In accordance with the present principles, medical care providers who have memorized the color caps can instantly recognize, by viewing a medical records dashboard of the present principles, the class of medication without even seeing the name.

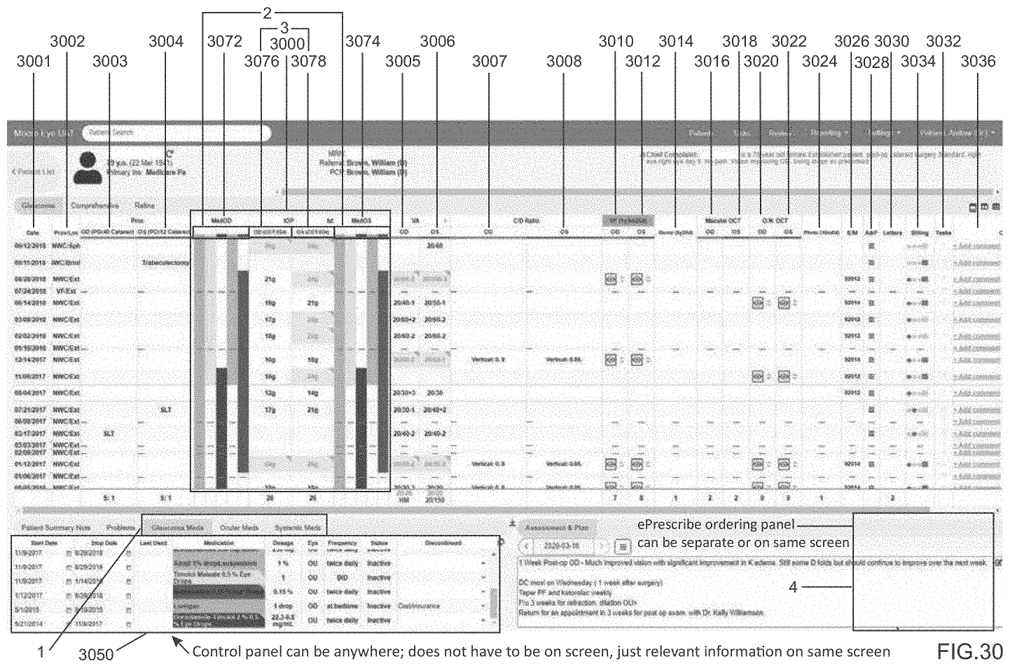

For example, FIG. 30 depicts a first embodiment of a Medication Management chart 3000 that can be displayed in at least a portion of a medical records dashboard of the present principles in accordance with one embodiment. The medical records dashboard of FIG. 30 illustratively comprises a patients Glaucoma chart including a date column 3001, a Provider/Location column 3000, a Procedures column for a right eye 3003 and for a left eye 3004, the Medications Management Chart 3000, a VA column for the right eye 3005 and for the left eye 3006, a C/D Ratio column for the right eye 3007 and for the left eye 3008, a VF column for the right eye 3010 and for the left eye 3012 including a Gonio column 3014, a Macular OCT column for the right eye 3016 and for the left eye 3018, an O.N. OCT column for the right eye 3020 and for the left eye 3022, a Photo column 3024, an E/M column 3026, an A&P column 3028, a Letters column 3030, a Tasks column 3032, a Billing column 3034, and a Comments column 3036 all arranged to depict information in rows of the medical records dashboard of FIG. 30 by date.

The Medications Management Chart 3000 of FIG. 30 includes a Medication column for the right eye 3072 and the left eye 3074, illustratively on either side of an IOP column for a right eye 3076 and the left eye 3078. all arranged to depict information in rows of the medical records dashboard by date. In the Medications Management Chart 300 of FIG. 30, the Medication column for the right eye 3072 and the left eye 3074 are illustratively separated into sections for separately displaying bars for each of a plurality of available medications. The embodiment of FIG. 30 depicts an example of a medical records dashboard including medication management in the field of eye care, however embodiments of the present principles can be applied to substantially any medical specialty and the like.

In the embodiment of FIG. 30, the pressure of each eye of a patient is measured from 0 to 50. In addition, each of the medications taken associated with each respective eye of the patient are depicted in bar graph form and distinguished by color according to the dates taken. In the embodiment of the medical records dashboard of FIG. 30, the color bars representing the medications administered to the patient are displayed adjacent to respective pressure data points for each eye according to a date administered to allow the user to directly correlate the effect of the medication on a respective eye. In the embodiment of FIG. 30, section #2 depicts the medication bar graphs, section #3 depicts clinical measurements of eye pressures that are affected by the medications, and window #4 depicts an ordering panel enabling the ordering of medication through, for example, E-prescribe, DoctorFirst, or other methods. In the embodiment of FIG. 30, window #1 depicts an embodiment and location of a control panel 3050 of the medical records dashboard, which identifies which medications are represented by which colors and identified a dosage, a frequency and a status of the medications being administered to a patient.

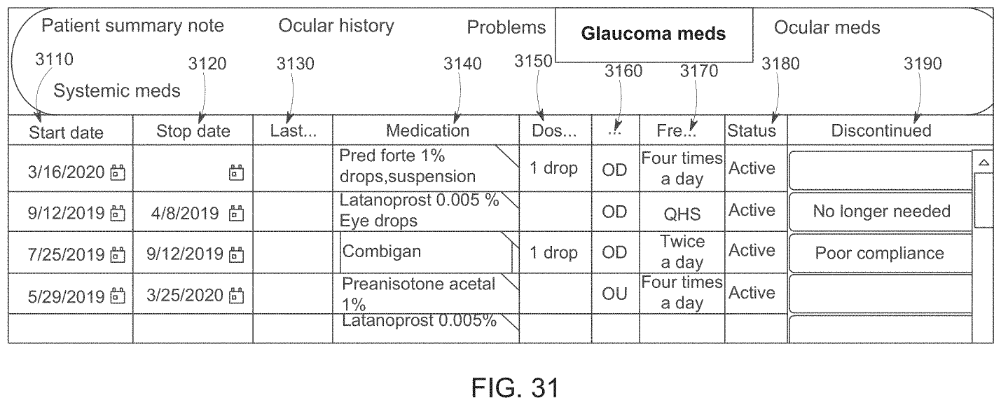

For example, FIG. 31 depicts an embodiment of the control panel #1 of the Medication Management chart of FIG. 30 in accordance with an embodiment of the present principles. The control panel of FIG. 31 illustratively includes a Start Date Column 3110 depicting a start date of a medication in a respective row, a Stop Date Column 3120 depicting a stop date (if any) of the medication in the respective row, a Last Column 3130 depicting a date when the medication in the respective row was last taken, a Medications Column 3140 depicting medications taken by the patient, a Dosage Column 3150 depicting a dosage amount of the medication taken by the patient, a Location Column 3160 indicating in what part of the patient's body the medication was applied, a Frequency Column 3170 indicating how often the medication is being applied, a Status Column 3180 depicting if the medications are or are not currently being applied, and a Discontinued Column 3190 depicting a reason for discontinuance of the medication (if a reasons exists). As depicted in FIG. 31, in accordance with some embodiments of the present principles, the Medications Column 3140 can be color coded such that each medication comprises a respective color.

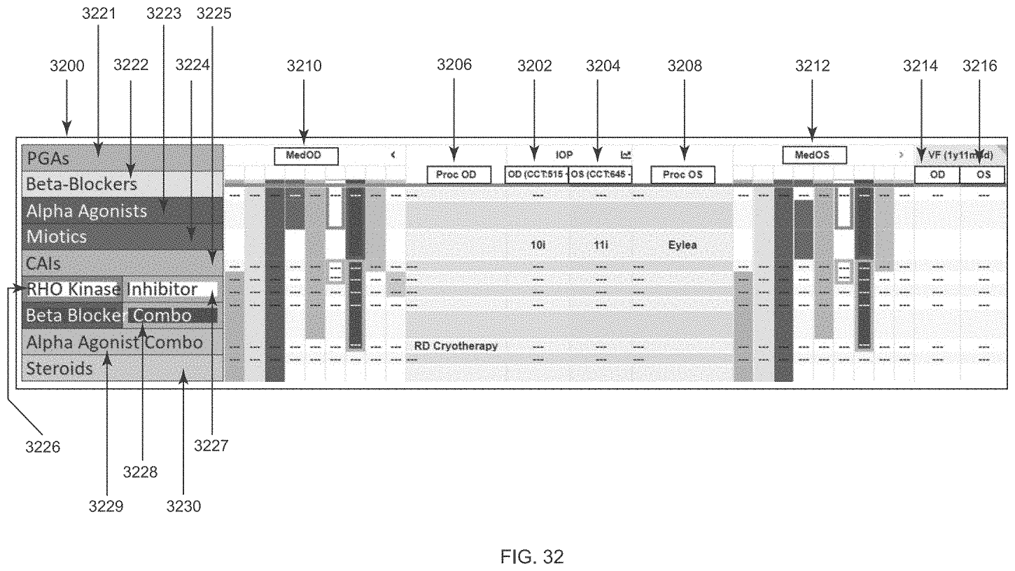

For example, FIG. 32 depicts a Medication Management Chart 3200 that can be displayed as part of a medical records dashboard or as a stand-alone Medication Management tool in accordance with an embodiment of the present principles. In the embodiment of FIG. 32, the Medication Management Chart 3200 includes a center section including respective columns depicting an intraocular pressure (IOP) for a patient's right eye 3202 and an intraocular pressure (IOP) for the patient's left eye 3204 on various different dates. In FIG. 32, next to the respective pressure columns for the patient's right eye 3202 and the patient's left eye are respective columns depicting respective procedures performed on the patient's right eye 3206 and the patient's left eye 3208 on the different dates. The Medication Management Chart 3200 of FIG. 32 further includes respective columns depicting respective medications administered to the patient right eye 3210 and the patient's left eye 3212 on the different dates. The Medication Management Chart 3200 of FIG. 32 further includes a visual field (VF) column for the right eye 3214 and a VF column for the left eye 3216.

The Medication Management Chart 3200 of FIG. 32 further illustratively includes a color-coded key identifying medications present in the Medication Management Chart 3200. In the embodiment of FIG. 32, medications administered to the patient's eyes include PGAs 3221, Beta-Blockers 3222, Alpha Agonists 3223, Miotics 3224, CAIs 3225, Rho Kinase 3226 and Inhibitor 3227, Beha-Blocker Combo 3228, Alpha Agonist Combo 3229, and Steroids 3230. As depicted in the Medication Management Chart 3200 of FIG. 32, in some embodiments, combinations of drugs can exist and can be depicted as a combination of the colors of the drugs that make-up the drug combination. Although in the embodiment of FIG. 32, the Medication Management Chart 3200 illustratively comprises a color-coded key for identifying the medications, in other embodiments of a Medication Management Chart of the present principles, a color-coded key does not have to be included. In addition, although in the embodiment of the present principles depicted in FIG. 32, the Medication Management Chart 3200 depicts a combination of drugs as a bar having one color representing a first drug and a box around the bar in a second color representing a second drug of the combination, in some embodiments a drug combination can be represented using a cross-hatch method, in which stripes in a bar are a first color representing a first drug and the rest of the bar is as second color representing the second drug of the combination. In accordance with the present principles, a drug combination can include more than two drugs and drugs and drug combinations can be represented by assigning a color to each drug and if a medication has more than one drug in it then all colors can be displayed by any means in, on or around the bar representing that medication.

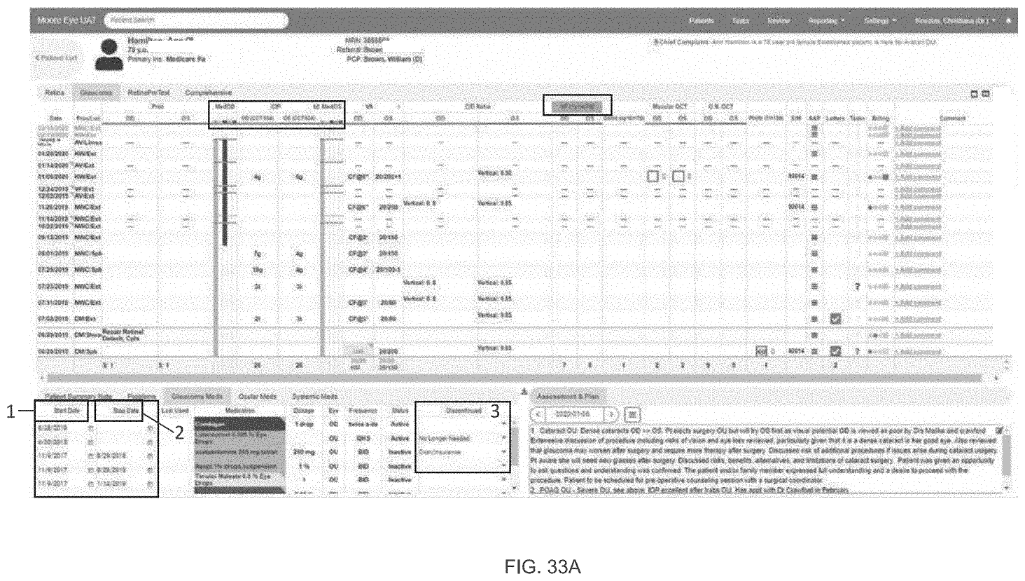

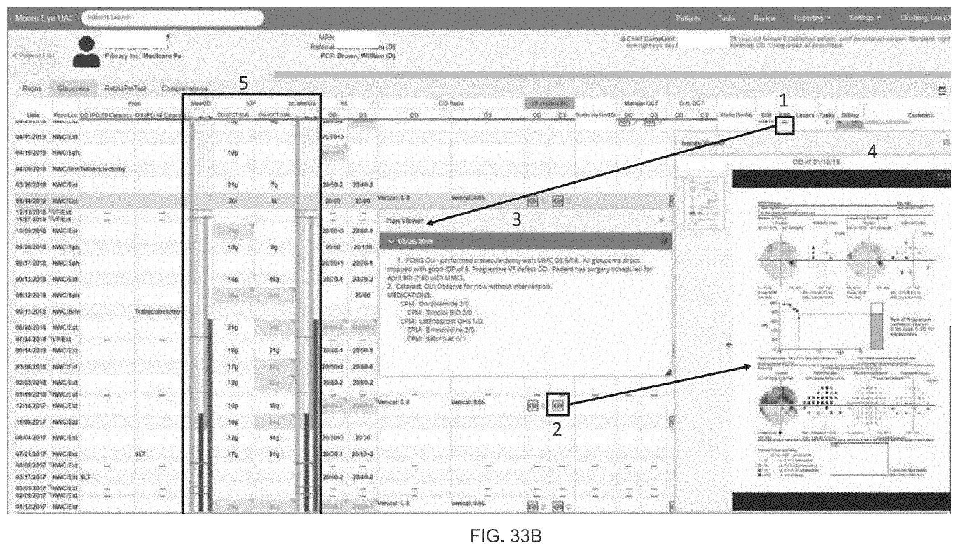

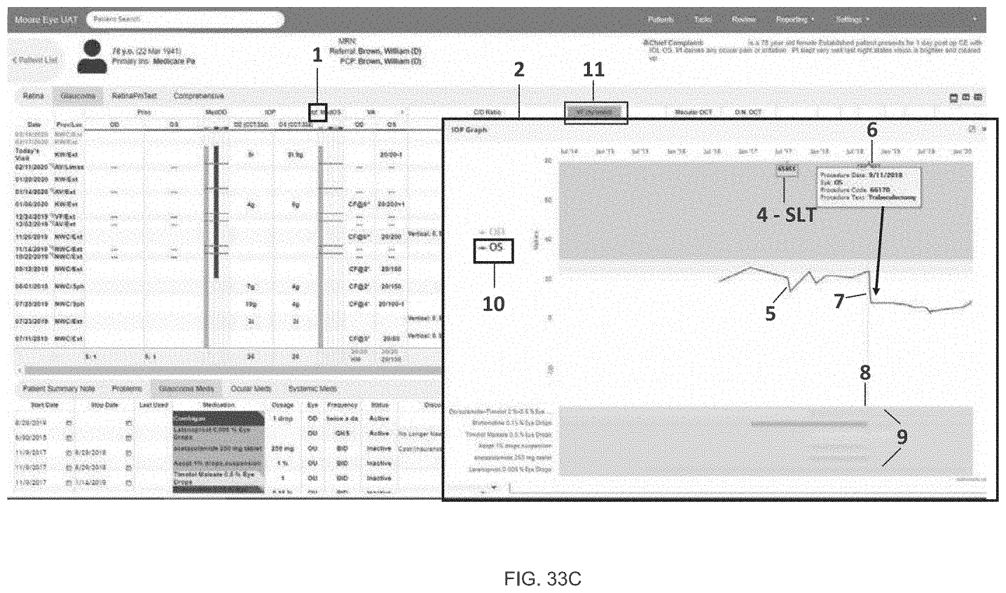

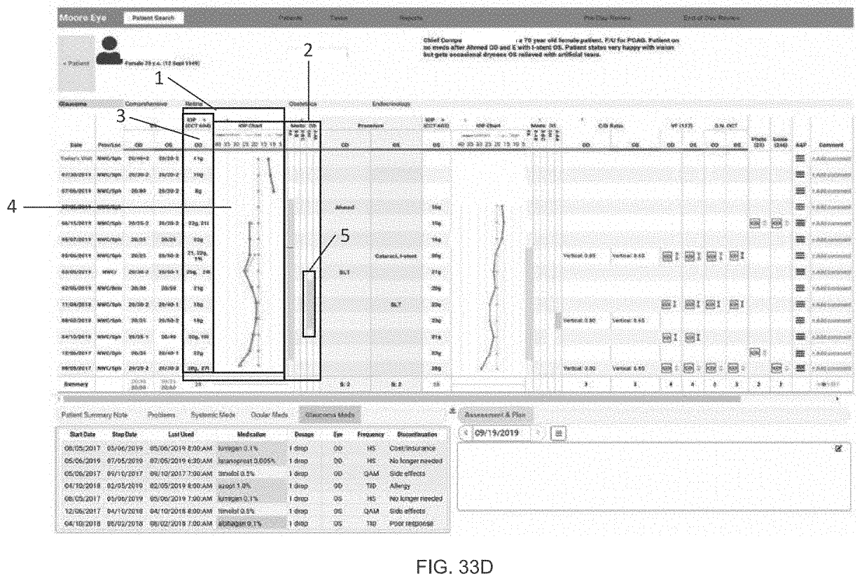

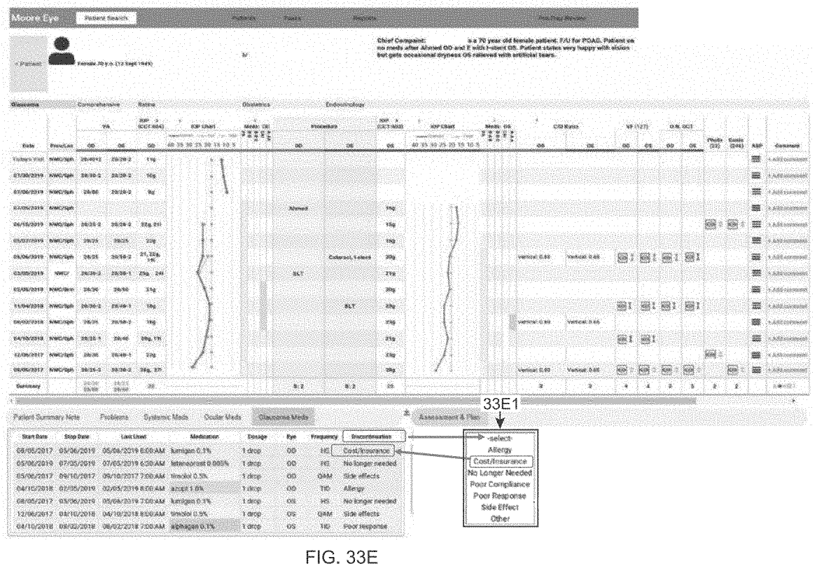

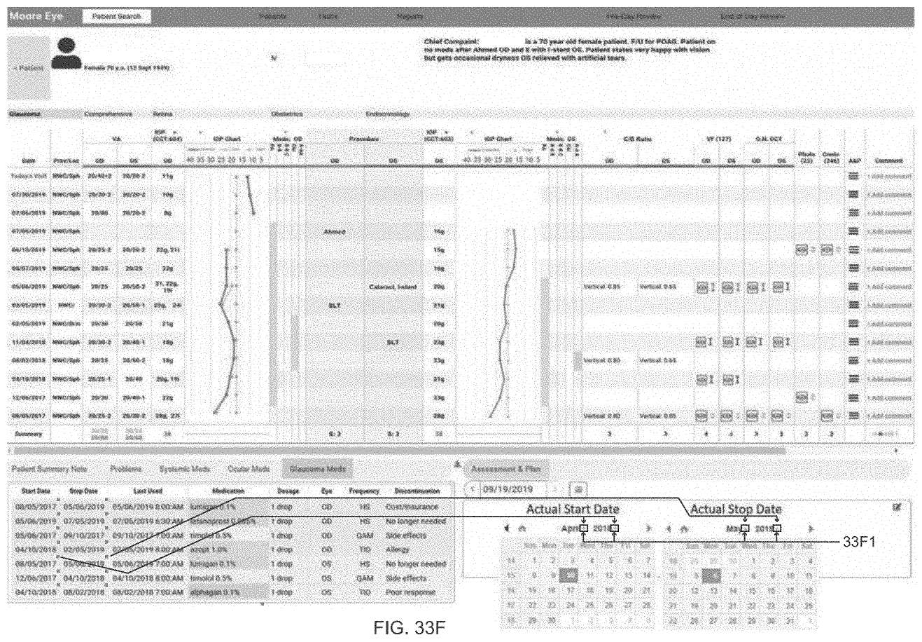

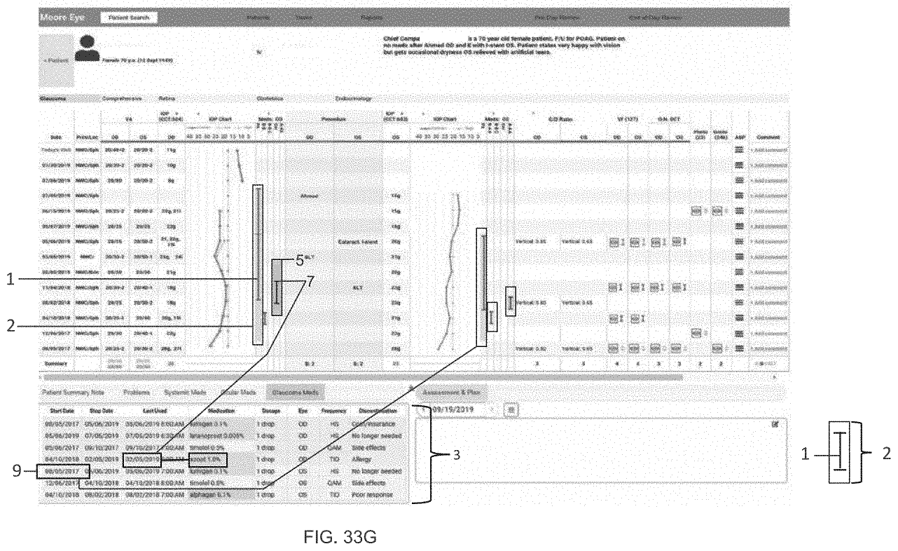

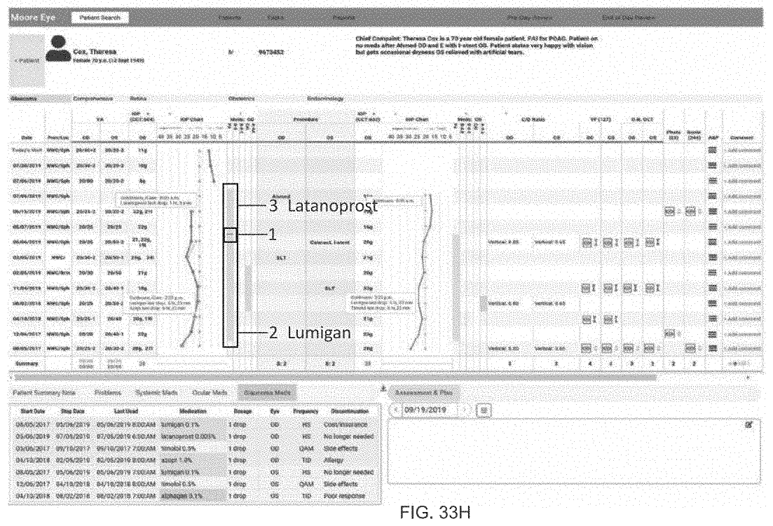

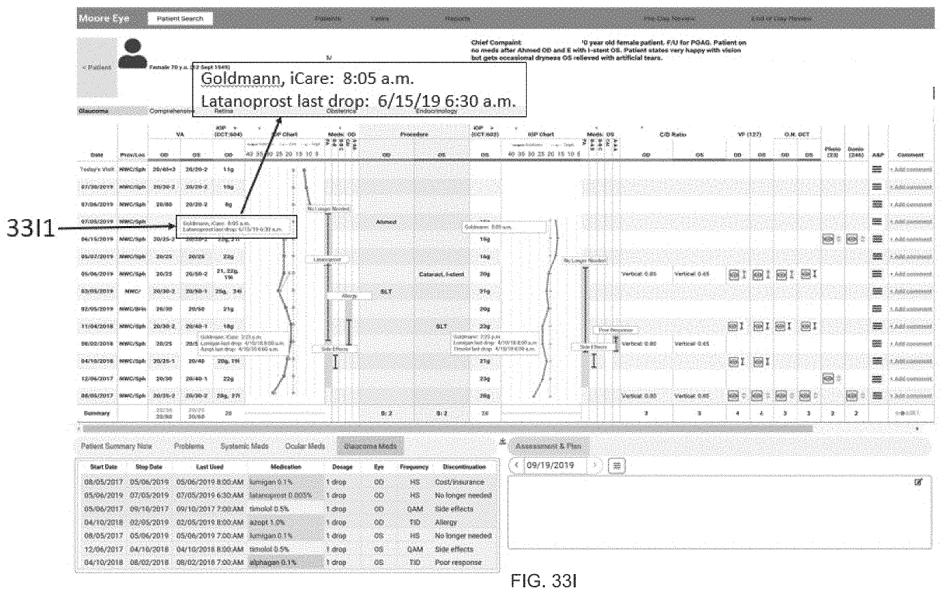

FIGS. 33A-33I depict embodiments of a Medication Management Chart having different features in accordance with the present principles and will be described with reference to the medical records dashboard and the Medications Management Chart 3000 of FIG. 30. FIG. 33A depicts an example of how the Control Panel #1 of FIG. 30 can be implemented by a user to identify start and stop dates for the various medications taken by a user in accordance with an embodiment of the present principles. FIG. 33A further depicts how section 2 of the Control Panel #1 can be implemented to identify and assign colors for the medications and section 3 of the Control Panel #1 can be implemented to note reasons for a patient discontinuing a medication.

FIG. 33B depicts an embodiment of a Medication Management Chart in which icons can be activated to bring up additional information in accordance with an embodiment of the present principles. In FIG. 33B, element 1 depicts how by clicking on a column heading, information available under the column heading, such as a note inserted by a user, can be accessed, for example, in a pop-up window, element 3. In FIG. 33B, a note regarding a treatment plan for the patient was accessed via a pop-up window (element 3) when an icon under the column heading was activated. As depicted in the embodiment of FIG. 33B, the column heading can contain an icon indicating that a note exists. Element 2 of FIG. 33B depicts an icon in the OS column of the VF column that when activated can cause a display of a pop-up window (element 4), which displays a visual field image performed on the patient's left eye. Element 5 of FIG. 33B depicts how all of the medications being taken by a patient can be simultaneously displayed using bar graphs and color coding of the present principles.

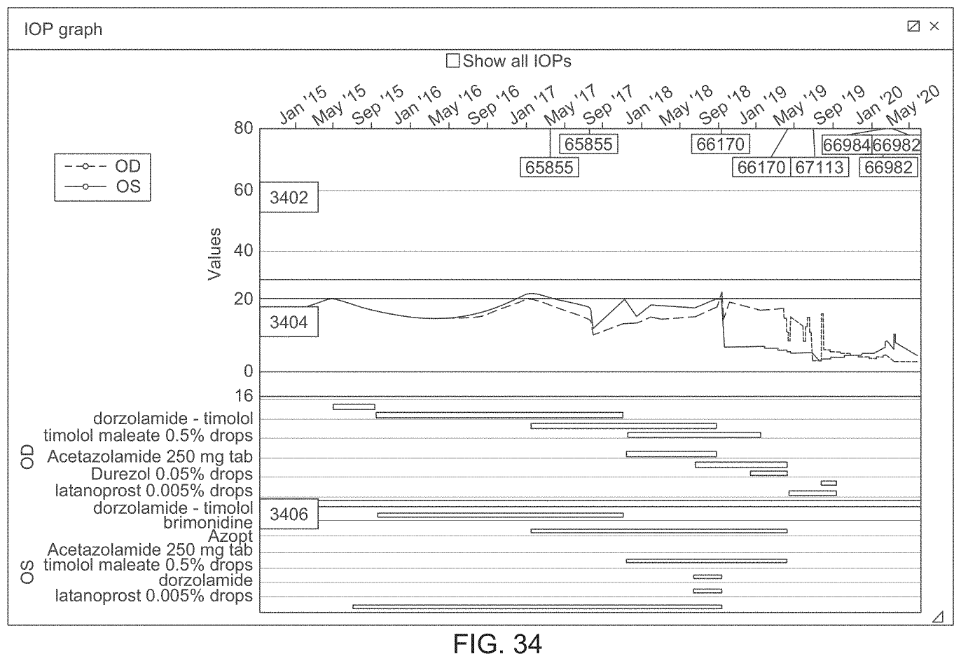

FIG. 33C depicts another embodiment of a Medication Management Chart in which icons can be activated to bring up additional information in accordance with another embodiment of the present principles. In FIG. 33C, element 1 depicts how activating an icon in, for example, a column heading of the Medications Management chart can cause a display of a pop-up window (element 2), which in some embodiments can display another embodiment of a Medication Management chart which displays mediations using horizontal lines and correlates patient well-being data/information (i.e., intraocular pressure) with events that occurred to the patient that would affect the patient's well-being (i.e., the application of medications, surgery, etc.) and with a medication timeline (described in greater detail with respect to FIG. 34). As depicted in FIG. 33C, using such Medication Management charts of the present principles, a user can make a reasoned estimation of what caused a decline or an improvement in the patient's well-being. As further depicted in FIG. 33C, a user can select to display information for one eye at a time or for both eyes simultaneously.

FIG. 33D depicts an embodiment of the Medication Management Chart in which intraocular pressure, in addition to being listed by number, is also displayed as a vertical line graph, for example as depicted by element 1 in accordance with an embodiment of the present principles. In the embodiment of FIG. 33D, element 2 displays bar graphs of the medications being taken by the patient. element 3 of FIG. 33D depicts values of intraocular pressures of a right eye and element 4 displays the corresponding vertical line graphs of the values pointed out by element 3.

FIG. 33E depicts an embodiment of the Medication Management Chart of FIG. 33D in which the control panel can be used to input a reason that a medication has been started or stopped in accordance with an embodiment of the present principles. In the embodiment of FIG. 33E, a drop down menu 33E1 can be used to enable a user to select a reason that a medication has been started or stopped.

FIG. 33F depicts an embodiment of the Medication Management Chart of FIG. 33D in which the control panel can be used to correct start and stop dates for a medication in accordance with an embodiment of the present principles. In the embodiment of FIG. 33F, a drop down menu 33F1 can be used to enable a user to correct/input a date that a medication has been started or stopped.

FIG. 33G depicts an embodiment of the Medication Management Chart of FIG. 33D in which both corrected start and stop dates for a medication taken by a patient and incorrect start and stop dates for a medication taken by a patient and listed for example by a 3^{rd} party data provider such as an EMR can be displayed simultaneously in accordance with an embodiment of the present principles. In the embodiment of FIG. 33G, element 1 depicts a line depicting a start and stop date of a medication being taken by a patient as listed in an EMR. In FIG. 33G the line pointed out by element 1 is displayed within a bar pointed out by element 2, which depicts start and stop dates of a medication being taken by a patient as identified by a user. FIG. 33G also depicts an alternative embodiment. That is, FIG. 33G depicts an orange bar depicting a medication being taken by the patient. The orange bar depicts start and stop dates of the medication as listed in an EMR and a black line within the orange bar which depicts start and stop dates of the medication as determined by the user. Importantly and in accordance with the present principles, FIG. 33G depicts that more than one stop and stop date can be depicted for each medication in a Medication Management Chart of the present principles.

FIG. 33H depicts an embodiment of the Medication Management Chart of FIG. 33D in which a user is alerted that a medication being taken by a patient has changed, even if medications are being listed by class and the new medication is of the same class as the old medication in accordance with an embodiment of the present principles. For example, in the embodiment of FIG. 33H, element 1 depicts a horizontal line in the bar of the medication that is being taken by the patient and that is being changed. element 2 of FIG. 33H depicts that before a change the medication being taken by the patient is Lumigan. The line pointed out by element 1 depicts a change in medication and element 3 depicts that the medication being taken after the change is Latanoprost, which is in the same class of medications as Lumigan.

FIG. 33I depicts an embodiment of the Medication Management Chart of FIG. 33H in which a user is able to select a portion of a graph to bring up additional information associated with the graph in accordance with an embodiment of the present principles. For example, in the embodiment of FIG. 33I, when a user hovers a selection tool (e.g., mouse) over a specific date portion of an IOP graph, a window 3311 appears displaying to the user information detailing, for example, when and/or where on that particular day an intraocular pressure was measured. Similarly and as depicted in FIG. 33I, when a user hovers over a specific date portion of a medications graph, the window 3311 appears displaying to the user information detailing, for example, at what time or how long ago the medication was taken by the patient. In some embodiments, a time between the measurement in the office, for instance, a blood pressure or a pressure of the eye, and how long ago the patient actually took the medication can be measured and displayed, since some medications have a short duration of action and such information would be useful to the user.

In another embodiment and as briefly described above, Medication Management in a Data Command Center in accordance with the present principles exists as a series of intelligent horizontal rows within a correlative graph representing individual medications, classes of medications, categories of medications, or logical groupings of medications, differentiating medications by color or combinations of colors, symbols, and/or text, graphing start and stop dates and times or individual doses, correlated to relevant values and relevant events. In accordance with the present principles, graphical differentiation between medications can consist of individual colors for individual medications, combinations of colors for medications including more than one component, or complex graphical representations. In some embodiments, color standards, such as defined by the American Academy of Ophthalmology, can be used for color coding the medications and/or custom colors can be used. For example, in ophthalmology and with respect to eye care, medications have been assigned in the industry to have a certain color on the eye drop bottle or cap. In some embodiments, these colors can be displayed allowing recognition by the user of the class of medication. For instance, yellow is a beta blocker one of which is Timoptic. In accordance with the present principles, medical care providers who have memorized the color caps can instantly recognize, by viewing a medical records dashboard of the present principles, the class of medication without even seeing the name. Alternatively or in addition, in some embodiments of the present principles a user can identify which generic or brand medication the patient is taking by any means including rolling over the graph and seeing the name of the medication pop up.

FIG. 34 depicts an illustration of a second embodiment of a Medication Management chart that can be displayed in at least a portion of the medical records dashboard of the present principles in accordance with one embodiment. In the embodiment of the present principles depicted in FIG. 34, the medications in the Medication Management chart are color-coded. Illustratively, in the Medication Management chart of FIG. 34, a top section 3402 illustrates dates of relevant events. In some embodiments, such events can include but are not limited to applied medications which can be taken by mouth (orally), given by injection into a vein (intravenously, IV), into a muscle (intramuscularly, IM), into the space around the spinal cord (intrathecally), or beneath the skin (subcutaneously, sc), placed under the tongue (sublingually) or between the gums and cheek (buccally), inserted in the rectum (rectally) or vagina (vaginally), placed in the eye (by the ocular route) or the ear (by the otic route), sprayed into the nose and absorbed through the nasal membranes (nasally), breathed into the lungs, usually through the mouth (by inhalation) or mouth and nose (by nebulization), applied to the skin (cutaneously) for a local (topical) or bodywide (systemic) effect, and/or delivered through the skin by a patch (transdermally) for a systemic effect, surgeries and any other procedures that can affect a patient's well-being.

A second, lower section 3404 of the Medication Management chart of the embodiment of FIG. 34 depicts a line graph correlating the relevant events that can affect a patient's well-being (i.e., the application of medications, surgery, etc.) of the top section 3402 to relevant values of patient well-being data/information (i.e., intraocular pressure) for each of a right eye and a left eye. In the Medication Management chart of FIG. 34, a third, lower section 3406 depicts a horizontal view of medications, which no longer spans a column of appointments, but denotes start/stop dates/times across a linear model. The linear model accounts for dates and key events in the top section of the diagram, such as the application of medications and major surgeries that may also have an effect on the results displayed in the middle section of the diagram. The third lower section 3406 displays an array of medications horizontally in context of the events and factors which can affect results, clearly showing the effect of medications and events on a single, or combination of multiple, tracked values.

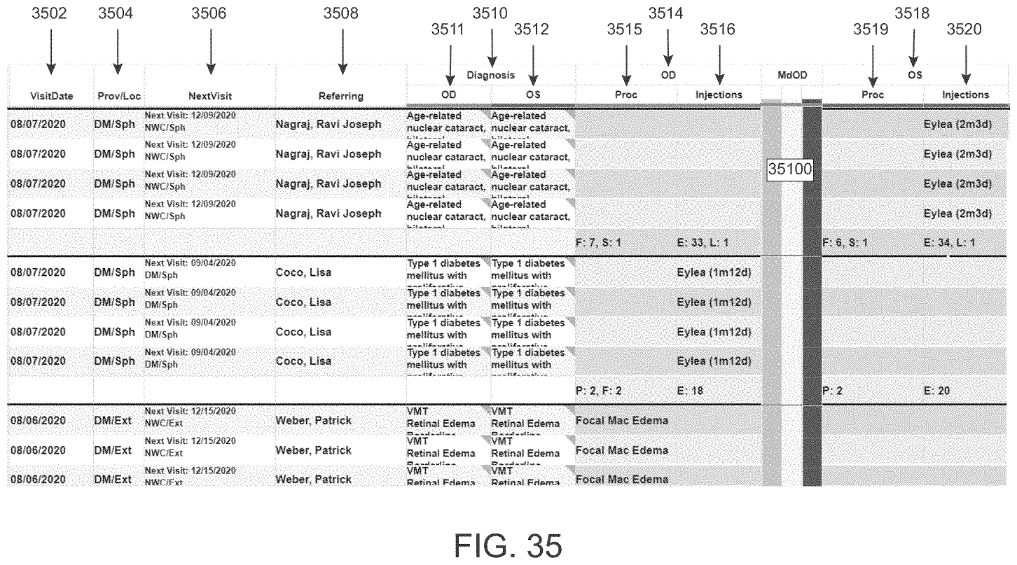

In another embodiment, Medication Management in a Data Command Center in accordance with the present principles exists as a series of intelligent vertical columns representing individual medications, classes of medications, categories of medications, or logical groupings of medications, differentiating medications by color or combinations of colors, symbols, and/or text, graphing start and stop dates and times or individual doses. For example, FIG. 35 depicts a medical records dashboard including a third embodiment of a Medication Management chart in accordance with an embodiment of the present principles. That is, the medical records dashboard of FIG. 35 includes a plurality of rows and columns and a Medication Management chart 35100 in accordance with the present principles. In the embodiment of FIG. 35, the columns of the medical records dashboard include a VisitDate Column 3502 listing the visit date of a patient, a Provider/Location Column 3504, a NextVisit Column 3506 listing a next visit date for the patient, a Referring provider Column 3508 listing the name of, for example, a referring doctor, a Diagnosis Column 3510 including an OD column 3511 and an OS column 3512 including a diagnosis for each of a right and a left eye, a separate OD Column 3514 including a Procedure column 3515 listing procedures performed on a patient's right eye and an Injections column 3516 listing injections performed on the patient's right eye, and a separate OS Column 3518 including a Procedure column 3519 listing procedures performed on a patient's left eye and an Injections column 3520 listing injections performed on the patient's left eye.

In the medical records dashboard of FIG. 35, the Medication Management chart 35100 depicts a representation of color-coded vertical medication columns as described above with respect to the embodiments of FIGS. 30, 32 and 33.

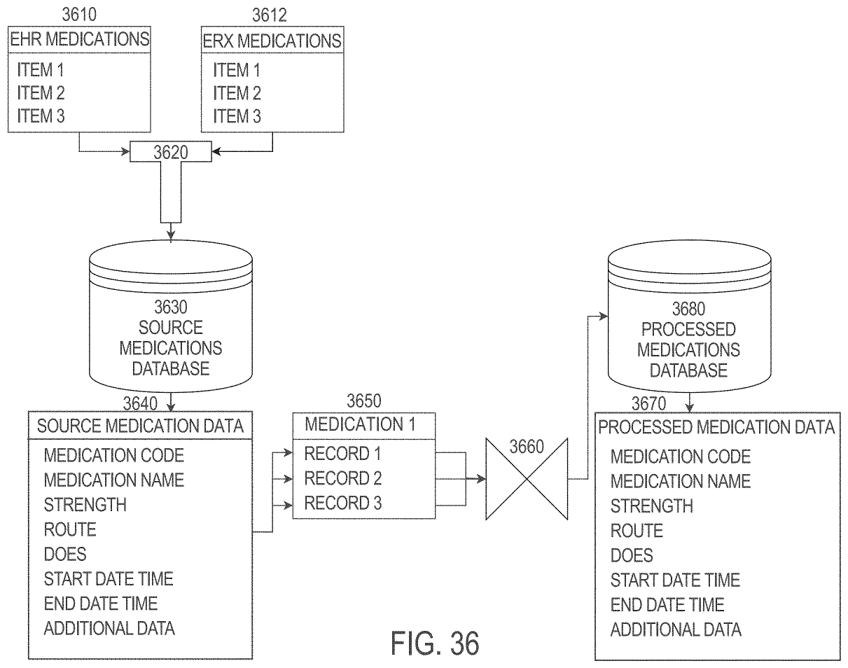

FIG. 36 depicts a high-level workflow diagram of an embodiment of Medication Management in a Data Command Center in accordance with an embodiment of the present principles. In the embodiment of FIG. 36, Medication source data can be stored within an EHR 3610 or eRx platform 3612. In some embodiments, as depicted in FIG. 36, medication data can be imported by an API or other means of digital communication, for example in one embodiment by the integration module 002 of the Data Command center 001 of FIG. 1, and can be compiled into a table 3620 which can be stored in a database 3630. Data from a relevant source stored in the database 3630 can be extracted. Block 3640 depicts an accurate representation of extracted data from the relevant source. The data from the relevant source can then be isolated to at least one specific medication from the source data. Block 3650 of FIG. 36 represents records isolated for a specific medication from the source data. The isolated data can then be processed at 3660 through several intelligent algorithms to surmise a final representation of the view of the specific medication, in some embodiments a longitudinal view. For example, in some embodiments, source data disparities and variance of medication data between sources can be addressed by intelligent algorithms which acquire available data about the medications and data sources and process toward a desired result. Algorithms account for presence if codified data, non-codified data, null values, and other datatypes. Such algorithms can be directed to exporting consistent representations of source data. In some embodiments of the present principles, such algorithms can be applied by the Rules module 004 of the Data Command center 001 of FIG. 1 and can be stored in a means for storage accessible to at least the Rules module 004.

Each medication column or row in a medical records dashboard of the present principles can consist of one or more individual medications as depicted by processed medications data as listed in block 3670, which can be stored in the Processed Medications Database 3680.

In some embodiments, the Display module 006 of the Data Command center 001 of FIG. 1 in accordance with the present principles causes the display of the Medication Management data in a medical records dashboard of the present principles as described above, and specifically in at least one of the vertical, horizontal, and textual embodiments described above and in accordance with individual medications, classes of medications, categories of medications, or logical groupings of medications, differentiating medications by color and/or combinations of colors, symbols, and/or text, graphing start and stop dates and times or individual doses, correlated to relevant values and relevant events as described above.

In some embodiments of at least one of a medical records dashboard and a Medication Management chart in accordance with the present principles, Medication columns and rows can expand, contract, hide, or be display based on a medical care provider's specialty, the identity of a medical care provider and/or a patient, patient conditions, patient procedures, risk factors, diagnostic results, future orders, future appointments, values recorded, values not recorded, calculated values, and absolute values for display, unless otherwise disallowed in accordance with Collapsible Columns and/or Rows that can Collapse and Expand.

In some instances, medication data can be sourced from misleading, unreliable, or inconsistent records reflecting multiple start and stop dates and times for a single medication due to each individual reorder of a medication stopping a prior prescription and starting a new one, or not stopping but adding a new start date and time, and may not reflect actual patient usage of said medication. As such, in some embodiments a Data Command Center via at least of a medical records dashboard and a Medication Management chart in accordance with an embodiment of the present principles enables a user to manually override misleading, unreliable, or inconsistent records to accurately represent medication usage. In such embodiments, each instance of source medication data being altered can be recorded in an audit log to account for data integrity as well as data accuracy. In some embodiments, the source medication data itself is never altered, updated, added, or removed. In some embodiments, updating medication data in any instance of Medication Management in accordance with the present principles reflects in every instance of the Medication Management. For example, editing a stop date and time in a list view of a medical records dashboard can also update the stop date and time in all graphical views. In some embodiments, medication updates can be stored separately from source medication data.

In general, in accordance with the present principles, embodiments of a Data Command Center via at least one of a medical records dashboard and a Medication Management chart of the present principles enable medical care providers to visualize medications, respective start and stop dates, reasons for discontinuation, and enables medical care providers to manage and change a display based on facts able to be confirmed with a patient at a point of care and even with home monitoring devices that can be linked. As described above, in some embodiments each medication can be represented by a bar graph or a linear graph or other visual method or means that in either the vertical direction or in a horizontal direction, a medical care provider can visualize the actual start and stop dates of all relevant medications for their specialty or for that patient all seen simultaneously with any other relevant data that the medications can impact. The medications and any encounters or clinical services or measurements that the patient takes at home or home monitoring devices can all be automatically or manually inputted. The Medication Management chart/Medication Management tool of the present principles can initially be populated by information in the EMR, which may or may not be accurate, or from E-prescribe systems. A medical care provider using, for example a medical records dashboard, can make changes and through a linear bar graph or other means, each column or row can represent a particular medication or class of medication. With all of the patient's medications that are relevant to that medical care provider or the condition being treated, all medications that the patient is taking now or in the past, can be displayed so that medical care providers will know all the medications that the patient has ever taken.

Embodiments of the present principles provide access to whatever information is relevant to the treatment of a patient and is enabled to share this information with all other medical care providers. All medication that can be used to manage a particular condition can all be displayed on a single screen if there is room or collapsed so doctors can visualize other options. In some embodiments, just the columns and/or rows are automatically displayed and other medication alternatives hidden until, through any means, a user accesses hidden patient-related information. In some embodiments, a Data Command Center via, for example at least one of a medical records dashboard and a Medication Management chart of the present principles, can offer clinical decision support in that if there is a set preferred treatment plan or the Data Command Center has programmed proper alternatives that a medical care provider should consider, the medical care provider can start the patient on a particular medication that can be suggested in a blank row or column next to other medications with the name of the suggested medicine.

In some embodiments, each user can move the columns and rows on which the medications are on to a particular section while being able to collapse and expand the entire history of every medication that the patient has taken. Each column or row, depending on whether a horizontal or vertical display is preferable, would be displayed from a start to a stop date and each corresponding date can be listed by office visit of encounter with different medical care providers and or by month, by day, by year, by hour or even minutes especially useful if the patient is hospitalized. In some embodiments of the present principles, a Data Command Center can receive inputs from a user via a user interface on how at least one of a medical records dashboard and a Medication Management chart should be configured to display patient related information from outside sources. For example, in some embodiments patient-related data/information from outside sources can be integrated into the Data Command center 001 via the Integration module 002 of the Data Command center 001 of FIG. 1. Once patient-related data/information is received by the Data Command center 001, the data can be compared to rules to be executed by the Rules module 004, which determine how and if received patient-related data should be displayed. As described above, in some embodiments, at least some of the rules for handling patient-related data/information can be provided to the Rules module 004 of the Data Command center 001 using a user interface. Patient-related data/information can then be caused to be displayed by the Display module 006 on at least the medical records dashboard of the present principles in accordance with the rules of the Rules module 004.

In some embodiments, multiple start and stop dates can exist for a medication based on when a patient admits that they really took the medication. As such, a medication bar graph might appear interrupted because, for example, the same medication might have been taken in 1993 and then re-started again in 2003 or the patient only took the medication for 10 months out of 12 months in a particular year. Such findings can be critical to patient care because if a patient does not take the medication as prescribed it can have an impact on a clinical finding or symptom or disease progression such as high blood pressure. Should a patient have blood pressure measured and suddenly the blood pressure is high, a medical care provider needs to know if it is not that the medication did not work, but perhaps that the patient did not take the medication.

An onset of other medical conditions or interventions such as surgeries or other life events like a death in the family can also be displayed in at least one of a medical records dashboard and a Medication Management chart of the present principles so a medical care provider can determine and take into all the information that can impact the well-being of a patient. As such in some embodiments, a medical records dashboard and a Medication Management chart of the present principles can display clinical findings, measurements, the laboratory findings, and/or whatever the medication impacts a patient's well-being such that a true change in a patient's well-being can be measured accurately and a medical care provider can see visualize the true effects of medications along with other medical services, interventions and life events. By way of example, in the field of ophthalmology there are glaucoma medications, which are pressure medications for the eye. Sometimes just one eye drop will make the pressure go down, sometimes two, three and four different types of drops are needed. Usually medical care providers add a medication if an eye pressure is not controlled to the level desired or if the medical care provider wants to replace one medication with another.

In some embodiments, at least one of a medical records dashboard and a Medication Management chart/tool of the present principles enables a medical care provider to document why a medication was started or stopped or if there has been a reaction to the medication. For instance, if the medication has been stopped because the patient is allergic or cannot afford it, or if it did not work. Such reasons can be input into the medical management tool by selecting the choice by any means such as a drop-down menu or through voice recognition software or any means. The information can then be displayed on a bar or line graph of that particular medication and either be permanently displayed or accessed via an icon or other access point.

In various embodiments of a medical records dashboard having a medication management tool (such as displayed in FIG. 30), in addition to a laboratory or clinical finding, there can be included an option to input information regarding procedures performed on a patient. For example, for a particular patient, a surgical procedure might be the reason there has been a sudden change in the well-being of the patient. For instance, there are some glaucoma pressure surgeries which will reduce the pressure and have the same effect as a medication or a laser surgery that might cause a pressure to be lower. It is important that a medical care provider have the option to view what procedure were performed on a patient to determine if a procedure might also have had an effect on the clinical finding, symptoms or disease progression on which the medication can also have an impact. Perhaps it is not the medication that is working, maybe it is the surgery.

Embodiments of the present principles are fully adjustable for all types of conditions, such as high blood pressure, diabetes, rheumatological diseases, and all types of cancer. All of these conditions have certain laboratories and clinical measurements that are taken either at the patient's home or from a testing center or on each visit with a medical care provider (i.e., doctors often record weight and blood pressure of the patient, etc.). In addition, a medical care provider can be enabled via at least on of a medical records dashboard and a Medication Management chart of the present principles to now E-prescribe or place an order for a new medication or cancel a drug. As such, by ordering a next medication, a medical care provider can instantly visualize what is being ordered as the new order can be displayed as a future medication. In such embodiments, a new column or row can appear with, for instance, a new bar graph because the medical care provider is now ordering a new medication.

A Data Command Center of the present principles enables a medical care provider to determine if incompatible medications or procedures have been ordered and/or scheduled. For example, in some embodiments, upon the visual display of ordered medications and/or procedures in at least one of a medical records dashboard and a Medication Management chart of the present principles, a medical care provider, by looking at the display, can visually determine through his/her experience and training that incompatible medications and/or procedures have been ordered or scheduled. Alternatively or in addition, in some embodiments a Rules module, such as the Rules module 004 of the Data Command center 001 of FIG. 1, can be programmed to recognize incompatible medications and/or procedures. As such, when patient-related data/information containing incompatible medications and/or procedures is received or when incompatible medications or procedures have been ordered or scheduled by, for example, a medical care provider using for example at least one of a medical records dashboard and a Medication Management chart of the present principles, the Rules module 004 can cause an alert to be displayed by, for example, the Display module 006, the alert intended to bring to a user's attention that incompatible medications and/or procedures exist. In some embodiments, if such a condition exists, a pop-up can appear to enable a medical care provider to re-do their order and make sure the order is corrected.

In some embodiments, multiple medication graphs can be shown independently or on for example, at least one of a medical records dashboard and a Medication Management chart of the present principles, such that a user is able to compare different reporting of the same medications. For example, in some instances patient-related data from an EMR can be inaccurate. However, it is advantageous for a medical care provider to know what has been documented, even if inaccurate. Embodiments of a medication management tool of the present principles can display two graphs, a first displaying what is actually documented in the EMR and a second displaying patient-related data that has been corrected by a medical care provider. In such embodiments, a medical care provider is enabled to check patient-related information from an EMR for accuracy.

In the medical field, medical care providers, such as doctors, use drug categories according to the affects they have on the human body. Many types of categories can be classified on the basis of chemical nature of the drug. The term of the drug or medication is used for diagnosing, curing, or treating a disease. Drugs classification can include but are not limited to a Chemical nature of the drug, Symptoms or diseases for which they are used (i.e., antihypertensive drugs), Organ system affected, Generations of drugs, such as antimicrobials or oral hypoglycemic agents, Receptor theory, Duration of action, and method of administration. Embodiments of a medical management tool in accordance with the present principles enable medical care providers to display all of a patient's medications by classification by, in some embodiments, selecting from a menu whatever classification method is most intuitive to the medical care provider as the medical care provider is treating the patient. By way of example, in the case of a subspecialist, like an ophthalmologist, the doctor might just want to know all medications of the eye, so the organ system affected is the eye. For instance, in the eyes category of disease can be glaucoma, which includes pressure control in the eyes. For glaucoma, there is a group of medications that control pressure in the eyes. Currently, there are eight classifications. In addition, there is macular degeneration disease or diabetic macular edema disease and there are classifications for those diseases as well. A medical care provider can decide to display, on a single display, either all of the ocular medications that the patient is taking singly or in categories. Alternatively or in addition, a medical care provider can select to display medication by symptoms of the disease, such as the anti-hypertensive medications.

It can also be helpful to a medical care provider to know if a patient is taking an originally prescribed brand of the medication or if the patient is taking a generic medication. Embodiments of a medication management tool of the present principles provide a means for listing whether a patient is taking an originally prescribed brand of the medication or if the patient is taking a generic brand. A difference between the two brands of medication is that one might cost a significant amount more than the other and some can work a little differently and not be as affective. Medical care providers need to know whether the patient is taking a brand name or a generic. Some insurance companies will only pay for certain brands or generics, and mandate that medication be taken. Some medications will have a copay by the patient and the patient has to pay additional money. It can be critical that medical care providers also note cost to patients and to the insurance companies, so that medical care providers can control health care dollars.

In some embodiments of a Medication Management tool of a Data Command Center of the present principles, the Medication Management tool can make suggestions in regards to using a less expensive generic medication and in some embodiments can compare medication and procedure recommendations made by a user/medical care provider against what a patient's insurance will allow. For example, in some embodiments information regarding generic medications that can be substituted for brand name medications can be stored in a storage means accessible to, for example, the Rules module 004 of the Data Command center 001 of FIG. 1. As such, when a user/medical care provider prescribes a medication using the Medication Management tool and/or a medical records dashboard of the present principles, the Rules module 004, via for example the Display module 006, can cause a display of suggested generic medications, in some embodiments in a pop-up window, that can be prescribed to a patient in place of the brand name medication. Similarly, in some embodiments information regarding what medications and procedures can be authorized by a patient's insurance company can be stored in a storage means accessible by, for example, the Rules module 004 of the Data Command center 001 of FIG. 1. As such, when a user/medical care provider prescribes a medication or schedules a procedure using the Medication Management tool and/or a medical records dashboard of the present principles, the Rules module 004 can compare the information regarding what a patient's insurance company will allow and what medication the user/medical care provider has prescribed or what procedure was scheduled to determine if the patient's insurance company will allow the medication and/or procedure. If the Rules module 004 determines that a prescribed medication and/or scheduled procedure is not allowed by a patient's insurance company, the Rules module 004, via for example the Display module 006, can cause a display of an alert to the user/medical care provider to alert the user/medical care provider that a prescribed medication and/or scheduled procedure is not allowed by the patient's insurance company. In some embodiments, information regarding what medications and procedures can be authorized by a patient's insurance company can be stored in a storage means accessible to the Rules module 004 of the Data Command center 001 of FIG. 1.

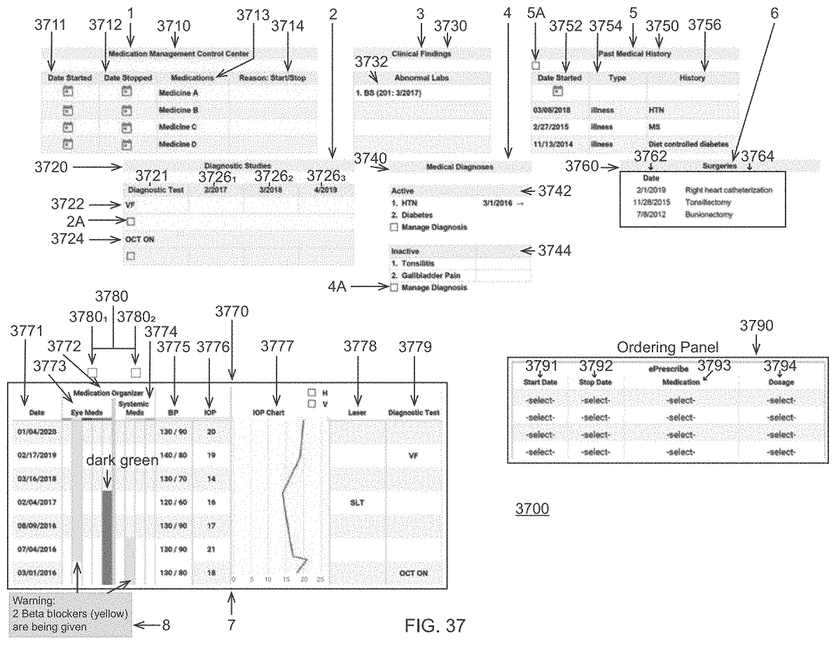

FIG. 37 depicts an exemplary embodiment of a Medications Management chart/tool 3700 which does not use rows or columns in accordance with an alternate embodiment of the present principles. Block 1 of the Medications Management chart/tool 3700 of FIG. 37 depicts a control panel 3710, which can be used to configure the bar graphs of block 7 and 8 described in greater detail below. The control panel 3710 of FIG. 37 illustratively comprises a date started column 3711, a date stopped column 3712, a medications column 3713 illustratively listing medicines A, B C and D, and a start/stop reasons column 3714.

Block 2 of the Medications Management chart/tool 3700 of FIG. 37 depicts a diagnostic studies menu 3720, which can be used to list diagnostic studies performed on a patient. The diagnostic studies menu 3720 of FIG. 37 illustratively comprises a diagnostic test column 3721, including a VF row 3722 and an OCT ON row 3724, and three date columns 37261, 37262 and 37263. In the diagnostic studies menu 3720 of FIG. 37, by hitting 2A, a user can pull up an individual test or get thumbnails of the tests performed on a patient. Block 3 of the Medications Management chart/tool 3700 of FIG. 37 depicts a clinical findings menu 3730, which can be used to list clinical findings on a patient. The clinical findings menu 3730 illustratively comprises an abnormal labs column 3732 for listing abnormal laboratory findings for a patient.

Block 4 of the Medications Management chart/tool 3700 of FIG. 37 depicts a medical diagnosis menu 3740, which can be used to list medical diagnosis made by a user for a patient. As depicted in the embodiment of FIG. 37, the medical diagnosis menu 3740 can be divided into active 3742 and inactive 3744 diseases. In the embodiment of FIG. 37, the various diagnoses or conditions of the patient can be managed on the screen by clicking 4A. Block 5 of the Medications Management chart/tool 3700 of FIG. 37 depicts a past medical history menu 3750, which can be used to list conditions that affect the well-being of a patient. As depicted in the embodiment of FIG. 37, the past medical history menu 3750 illustratively includes a date started column 3752, a type column 3754 and a history column 3756. In the embodiment of the past medical history menu 3750 of FIG. 37, by hitting 5A, a user is able to edit any of the information in the past medical history menu 3750.

Block 6 of the Medications Management chart/tool 3700 of FIG. 37 depicts a surgeries menu 3760, which can be used to list surgeries performed on a patient. As depicted in the embodiment of FIG. 37, the past medical history menu 3750 illustratively includes a date started column 3752, a type column 3754 and a history column 3756.

Block 7 of the Medications Management chart/tool 3700 of FIG. 37 depicts a dashboard 3770. The Dashboard 3770 of the Medications Management chart/tool 3700 of FIG. 37 illustratively comprises a date column 3771, a medication organizer column 3772 including an eye medications column 3773 and a systemic medications column 3774, a blood pressure (BP) column 3775, an intraocular pressure (IOP) column 3776, an IOP chart/graph column 3777, a laser column 3778, and a Diagnostic test column 3779. The Dashboard 3770 of the Medications Management chart/tool 3700 of FIG. 37 illustratively further comprises a respective ordering panel selection block 37801, 37802 for each of the eye medication column 3773 and the systemic medications column 3774. When a user selects either of the ordering panel selection blocks 37801, 37802, an ordering panel 3790 such as an E-prescribed panel is displayed that enables the user to place an order, which can include prescribing a medicine, and comes up in a way that does not block the entire view. The ordering panel 3790 illustratively comprises a start date column 3791, a stop date column 3792, a medication column 3793, and a dosage column 3794.

In the Dashboard 3770 of the Medications Management chart/tool 3700 of FIG. 37, the eye medication column 3773 and the systemic medications column 3774 include bar graph representations of medications associated with the treatment of a patient's eye. Illustratively, in the Medications Management chart/tool 3700 of FIG. 37, a user is being warned in block 8 that two beta blockers, depicted as yellow bars, are being given to the patient. Since there is a relationship between the two, the user needs to know.

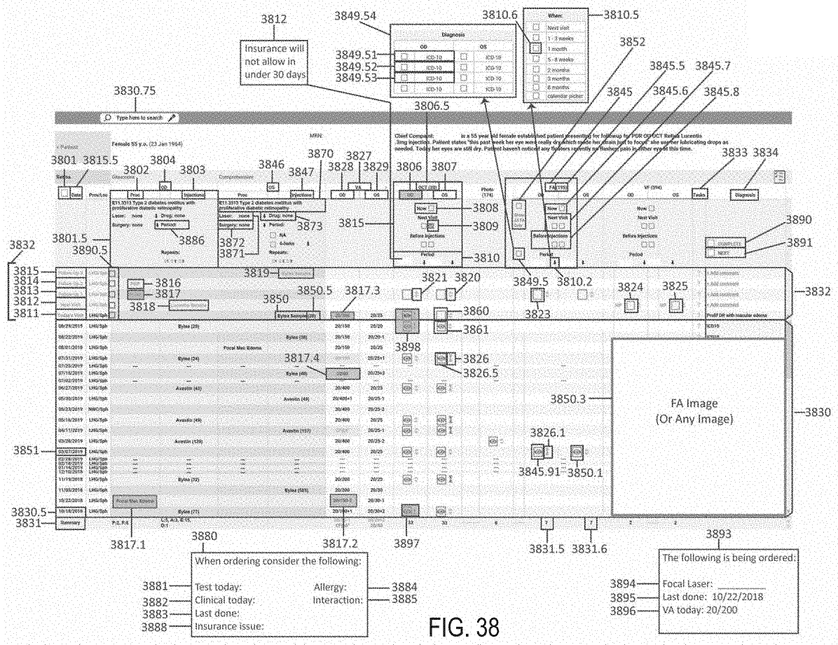

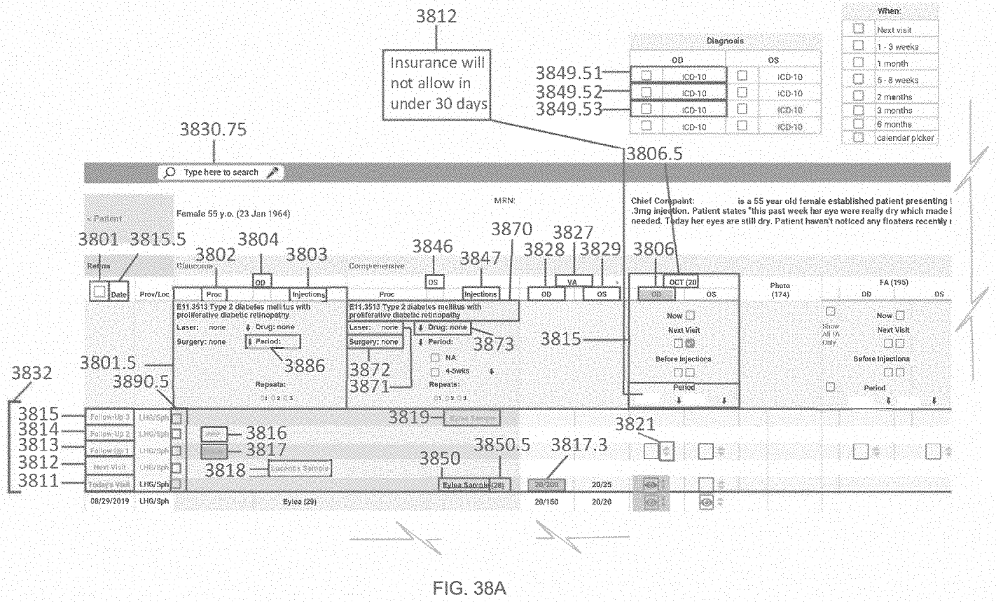

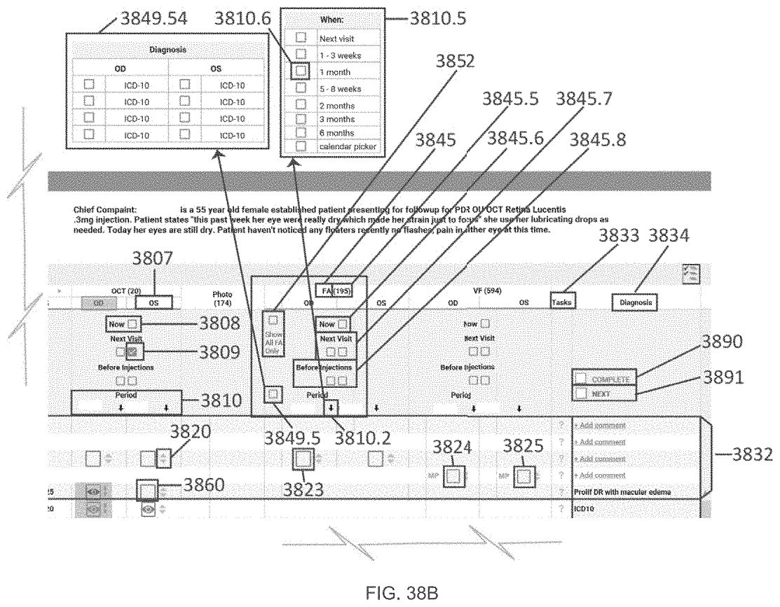

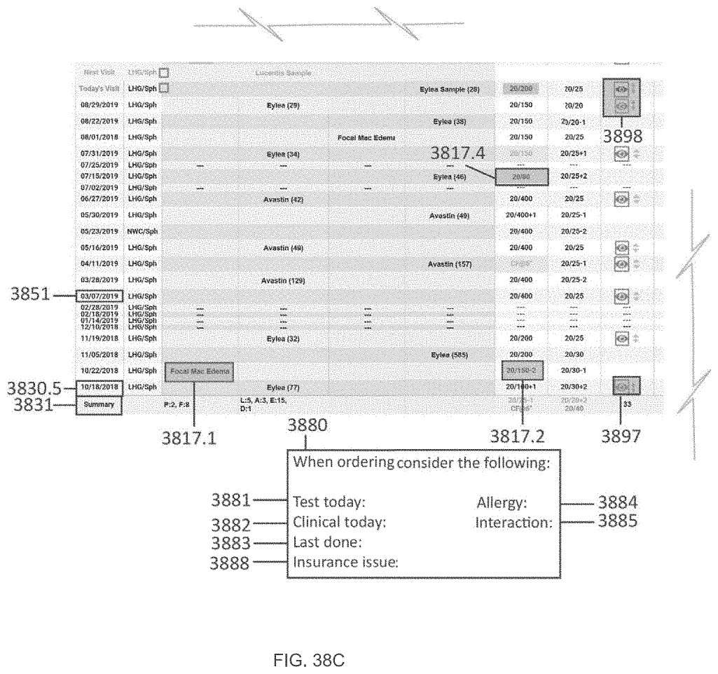

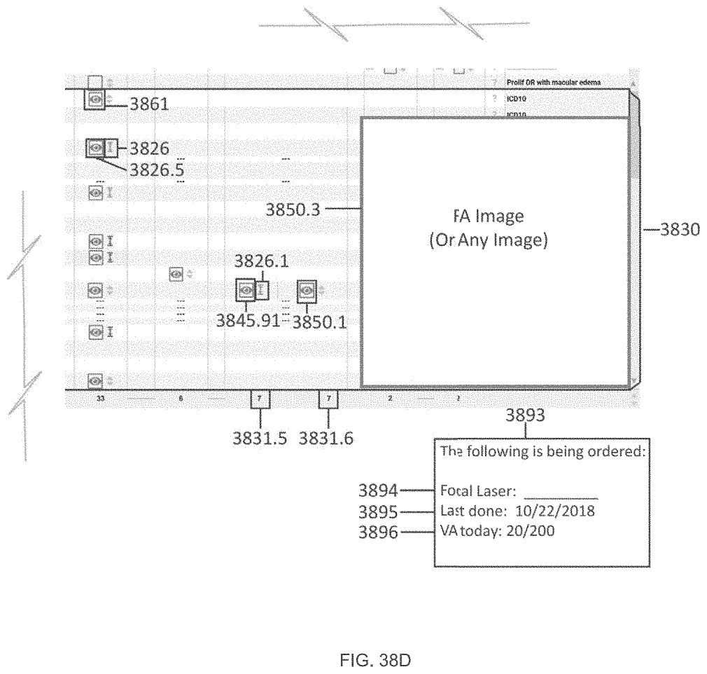

FIG. 38 depicts an embodiment of a medical records dashboard of a Data Command Center in which a user/medical care provider is enabled to place orders in context with other relevant patient data/information, so as to enable the user/medical care provider to see the future orders in context and confirm that the orders submitted are in fact what the user/medical care provider intends in accordance with the present principles. The details of FIG. 38 are being presented as FIGS. 38A-38D (collectively referred to as FIG. 38 below) to enable more clear visualization of the features of the embodiment of FIG. 38. In some embodiments, a column of the medical records dashboard can be expanded by selecting the column. For example, in FIG. 38, the column 3801 is expanded as depicted by window 3801.5. In some embodiments, the window 3801.5 can comprise a pop-up panel for placing orders. In FIG. 38 cells 3804, 3806.5, and 3845 depict examples of cells displayed in the medical records dashboard that are in the line and above corresponding columns that identify that orders have been made and/or enable the placement of new orders. For example, cell 3804 corresponds to a panel that can be used for placing orders for a right eye (OD) and is located directly above procedures performed in the past for the right eye (OD) identified in cells 3802 and 3803. Another example is the ordering panel 3845 which is above the FA column. Illustratively, in the FA column, a user can identify when the last time something was performed, enabling a user/medical care provider to determine if it is time to order a new procedure. From the medical records dashboard of FIG. 38 it can be determined from column 3851 that the last FA was done (Mar. 7, 2019) and the FA in the header cell 3845 depicts that the last FA, was 195 days ago as depicted in cell 3845.5. In the embodiment of the medical records dashboard of FIG. 38, a user is enabled place an order while visualizing a particular CPT codes (diagnostic test, procedures, office visit, etc.) ordered in the past and can visualize how often it was performed, when the last time it was performed. In FIG. 38, an illustrated FA, row 3831 reports the total number of times the item to be re-ordered was previously performed. In the example of an FA shown in cell 3831.5 of FIG. 38, in the right eye (OD) cell 3831.6 depict that an FA was performed seven times in the past.

As described above, in the embodiment of FIG. 38, expansion of an ordering panel can occur in both in height and in width. In some embodiments, to enable the expansion of an ordering panel, columns that are considered by a user/medical care provider as unnecessary can be collapsed to enable viewing expanded ordering panels in context with information deemed necessary. For example, a clinical measurement, such as vision measurements in columns 3827, 3828, 3829 can be collapsed if a user determines such information is not currently needed, enabling horizontal expansion of ordering panels. In some embodiments, the ordering panels (3845, 3806.5, 3804) can widen when the user/medical care provider clicks on them to then place an order to enable a user/medical care provider to simultaneously visualize, using a single display, data relevant to the newly placed orders. In accordance with embodiments of the present principles, the display 3830 remains interactive during the display of the ordering panels to enable a user to scroll down to see past FA performed, for example, prior to the Oct. 18, 2018 row (3830.5). Cell 3830.75 of FIG. 38 depicts a search mechanism enabling the user to type in or ask any questions and whatever rows with the relevant data would be the rows visualized with other rows collapsed or hidden. For instance, Cell 3831.5 depicts that seven FA were done yet only 3851 is displayed in this single view, but all seven dates of service when 3845 were performed, the tool would display those rows for instance clicking on cell 3831.5, which may be important as a user is ordering a new FA. In this way, as the user orders, for example, an FA, the user is able to visualize what was done in the past.

In the embodiment of FIG. 38, an FA can be ordered by activating ordering panel 3845 to expand the panel. The user could then decide if what the users want displayed in that column, 3845 are just the most recent FAs, in FIG. 38 depicted by cells 3845.91 and 3850.1 in row 3851. A user, alternatively, could scroll down and find the other FA's for the earlier dates or by clicking on cells 3831.5 or 3831.6. Embodiments of the present principles enable a user to search as depicted in cell 3830.75 or to scroll to display the seven FA rows. In some embodiments, all of the rows and dates of service can be collapsed to make room to display today's visit in, for example, cell 3811. That is, because an action is being performed by a user, a current row can remain visible. A next visit then can be displayed in a follow up cell 3812 and a future order cell 3813 can become visible, as the user places orders for different future dates of service with row popping-up as user places orders for each future visit. Alternatively, in the embodiment of FIG. 38, a user can prioritize the visualization of rows/cells depicting when FA was performed and collapse other rows/cells by clicking on icon 3852, which enables a collapsing of all rows except the rows when an FA was performed.

In the embodiment of the medical records dashboard of FIG. 38, if a user/medical care provider wants to double check if an order placed is proper and wants to see a related study itself, the user/medical care provider can select cells 3845.91 or 3850.1 and a respective image can be displayed so the ordered study can be interpreted in context of all other information being presented in the medical records dashboard. The user/medical care provider can view directly, an image or even choose multiple icon images of, for example, the FA. The ordering panels that are displayed when selected (i.e., 3801 or 3845) can be customized by specialty, for example in FIG. 38 for a retina specialist. In the embodiment of FIG. 38, a retina specialist can perform injections on a patient, as such in accordance with some embodiments of the present principles, the retina specialist can be presented with an option to perform the FA before an injection, 3845.8. In such embodiments, the injections are not hidden and can be seen in column 3847. The scheduling for the test (e.g., FA 3845) can then be accomplished by activating cell 3810.2, at which point an option for selection can be displayed (i.e., 3810.5) and the user can select form a pull down menu how far in the future (illustratively one month 3810.6) to order the study.

In some embodiments, a Rules module, such as the Rules module 004 of the Data Command center 001 of the embodiment of FIG. 1, can be configured to determine if a patient's insurance company will disapprove of ordered studies and can further be programmed to determine if a patient has an aversion to an ordered study and can cause a display, for example via the Display module 006, of an alert or information window on the medical records dashboard to inform a user/medical care provider of such instances.

In the embodiment of FIG. 38, cell 3806.5 can be used to order an OCT test. For example, cell 3807 can be selected by a user to select a left eye then OCT (OS), cell 3809 selects a next visit, and cell 3810 can be selected for choosing a time period. In some embodiments, a Rules module, such as the Rules module 004 of the Data Command center 001 of the embodiment of FIG. 1, can have access to a storage means containing rules for scheduling tests (i.e., certain tests have rules for how often the tests can be performed on a patient) and the Rules module 004 be configured to determine if tests/studies have been improperly ordered. In such instances, the Rules module 004 can cause a display, for example via the Display module 006, of an alert or information window on the medical records dashboard to inform a user/medical care provider that perhaps a test/study has been improperly ordered via, for example, a pop-up window 3812.

In the embodiment of FIG. 38, a user/medical care provider is enabled by the medical records dashboard to select a reason for ordering a test or procedure. In the embodiment of FIG. 38, cell 3849.5 can provide a menu providing options for a user to select for inputting reasons for ordering a test or procedure. In some embodiments, such options provided to a user in cell 3849.5 can be pre-programmed. Alternatively or in addition, a Rules module, such as the Rules module 004 of the Data Command center 001 of FIG. 1, can be programmed to monitor data/information related to a patient including, but not limited to, previous diagnosis made, previous tests ordered, previous procedures ordered and respective reasons for ordering the tests and procedures, and the Rules module 004 can be configured to learn, for example, through machine learning and/or artificial intelligence means to determine at least a best reason for ordering tests and procedures depending on relevant patient information. In such embodiments, the Rules module 004 can cause the display, for example via the Display module 006, of most logical reasons for ordering a test or procedure in, for example, a drop down menu provided by cell 3849.5 of the medical records dashboard of FIG. 38. For example, the Rules module 004 can be aware of what CPT codes can be associated with ICDs for a particular patient for which test and/or procedures are being ordered and the most logical diagnostic codes can be presented, for example in cells 3849.51, 3849.52,3849. In the embodiment of FIG. 38, if a user/medical care provider is unsatisfied with the reasons for ordering provided in, for example, a drop down menu provided by cell 3849.5, the user/medical care provider can select cell 3849.54 to see more options or to insert a reason for ordering.

In the embodiment of the medical records dashboard of FIG. 38, a user/medical care provider can select using for example cell 3806, for which eye a test/study/procedure is to be ordered. A diagnosis and information regarding what is ordered is displayed in cells 3811, 3812, 3813, 3814, and 3815 depending on when the order is scheduled. The user can visualize the order, then by any means, confirm it is correct, by selecting cell 3807. The user/medical care provider is able to confirm everything in a row displayed is correct as visualized and confirm the order for that entire future date of service by selecting cell 3890.5. In the embodiment of FIG. 38, a user can be informed of what is being ordered by displaying in a corresponding row, an empty icon or empty box, for example 3813, 3823. If the doctor wants to also order an OCT in the right eye, cell 3806 can be selected and the process repeated.

In the embodiment of the medical records dashboard of FIG. 38, cell 3830 shows all past encounters of relevance in which a user can view all of the information by scrolling or viewing on a single display. Cell 3830 keeps track of every encounter and a date and/or time of the encounter, any medical service, ICD 10 with diagnosis or clinical information or procedural information. Cell 3831 includes a summary of how often orders have been placed in any period of time. Row 3811 depicts information regarding “today's visit.” Today's visit can be live and in real time in some embodiments. Clinical information, i.e. in this example vision (VA), can be displayed as it is input in corresponding columns 3827, 3828, 3829. Column 3847 depicts what is to be done today and in the embodiment of FIG. 38 depicts an injection with medication 3850, “Eylea sample.” Cell 3850.5 of FIG. 38 depicts that the procedure was to be performed 28 days ago, which, as described above, can be checked by the medical records dashboard for compliance.

In the embodiment of FIG. 38, row 3811 shows under column 3807 an OCT and an empty box 3860. Such configuration can indicate to a user/medical care provider that the ordered procedure/test/study has not yet been performed because in the embodiment of FIG. 38 the order was scheduled in “today's visit,” meaning that the user/medical care provider placed the order today. In comparison, cell 3861 is filled in because on the last visit the test had been performed.

In some embodiment of the present principles, an appearance of the cells of the medical records dashboard can be altered to distinguish/highlight the information in the cells. For example, in the embodiment of FIG. 38, cells 3822, 3823, 3824, 3825, 3817, 3818, 3816, 3819 are examples of cells containing future orders. In some embodiments, cells can be made lighter or darker to differentiate past versus future actions/orders. In addition and for example, row 3811 of “today's visit” can be made blue. Even further, in some embodiments of the present principles, icons or markers can be included in cells/rows/columns of the medical records dashboard to enable a user to make a determination of the information included in a cell just by looking at the icon/marker. In some embodiments, the icons/markers can also include color to further distinguish between information represented by the icon/marker. For example, icons 3898, 3897 can be shown as colored indicators to indicate a status of the condition of a user's eye described in cell 3826.

In the embodiment of the medical records dashboard of FIG. 38, related cells can be highlighted to call a user's attention to relevant patient data when placing an order. For example, cell 3871 enables a user to order a laser. Cell 3817 depicts that a focal laser is to be ordered in the future. In conjunction, cell 3817.1 can be highlighted to alert the user/medical care provider of the last time a similar focal laser was done. In addition, cell 3817.2 can be highlighted to alert a user what the vision of the patient was at the time of the last laser performed Oct. 22, 2018, which is displayed in cell 3830.5. As such, a user/medical care provider can take into account related patient data as they place an order for a focal laser in cell 3802 as displayed in cell 3817 for a follow up row 3813, as scheduled by any means, by way of example, within the pop-up window 3802 or 3810.6. By noting a previous condition of the vision of a patient in accordance with the present principles, a user can identify if a patient's condition is getting better, worse or remaining the same. For example, in the embodiment of FIG. 38, icons 3898 and 3897 show red indicators to indicate a worsening of a condition of a patient's eye.

In another example of placing orders, as described above a medical records dashboard of the present principles, via for example a Rules module, can be aware of what the most common ICD10 might be (i.e., via cell 3849.54) when ordering. Cell 3845.7 depicts a user selecting a box and an order can be directly linked to the box the user selects, which can be displayed in a pop-up window as depicted in cell 3823. The future encounter can be selected and confirmed in cell 3890 and the next encounter ordered in cell 3891, which in this embodiment means another date of service in the future is to be ordered and displayed, and the process starts again. This functionality enables users/medical care providers to confirm future orders by reviewing available patient related data being simultaneously displayed in the medical records dashboard.

As depicted in the embodiment of FIG. 38, the medical records dashboard can include panel 3880 for assisting a user/medical care provider in placing an order. That is, in some embodiments, when a user/medical care provider is placing an order, panel 3880 can be presented to the user/medical care provider to present to the user/medical care provider a list of things that the user/medical care provider should take into considerations when placing an order. In the embodiment of FIG. 38, the panel 3880 includes considerations such as 3881 a diagnostic test that was done today or on a previous visit, 3882 clinical findings found today, 3883 a last time the same or similar test/study/procedure was done, 3888 insurance issues, 3884 allergy concerns, and 3885 possible interactions with other tests and/or medications. A Rules module, such as the Rules module 004 of the Data Command center 001 of FIG. 1, can be configured to monitor such considerations and alert a user/medical care provider if a problem is determined. Although the panel 3880 of FIG. 38 depicts a specific listing of considerations in panel 3880, in alternate embodiments, the considerations listed in panel 3880 can change dependent upon what is being ordered.

As depicted in the embodiment of FIG. 38, the medical records dashboard can include panel 3893 for assisting a user/medical care provider in placing orders. That is, in some embodiments, when a user/medical care provider is placing an order, panel 3893 can be presented to the user/medical care provider to present to the user/medical care provider an order summary. In the embodiment of FIG. 38, the panel 3893 includes a listing of 3894 what is being ordered, 3895 a last date the same procedure was performed on the patient, and 3896 any relevant clinical information. Although the panel 3893 of FIG. 38 depicts a specific listing of related order information in panel 3893, in alternate embodiments, the order related information listed in panel 3893 can change dependent upon what is being ordered.

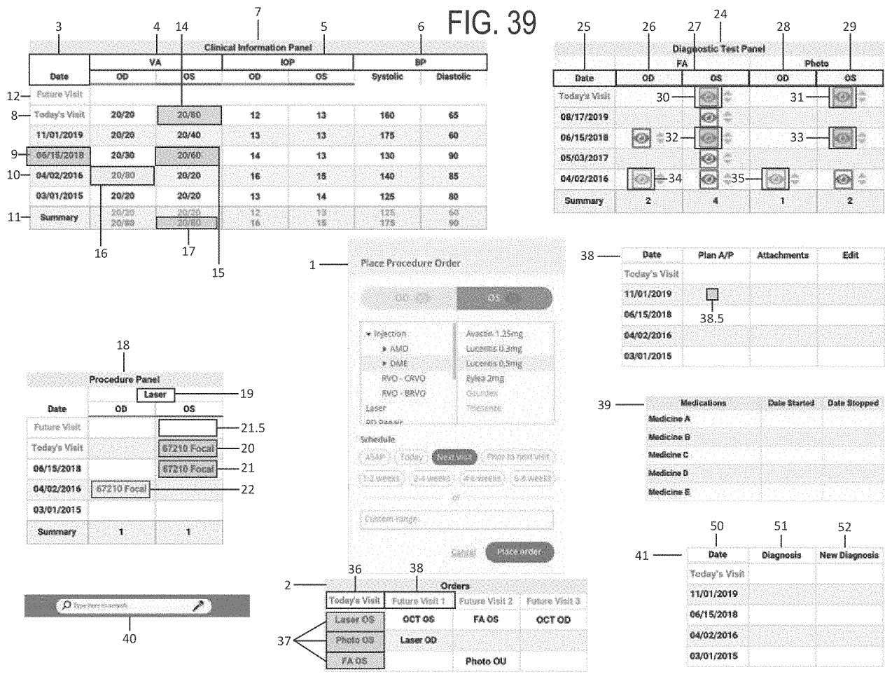

FIG. 39 depicts an embodiment of a medical records dashboard of a Data Command Center in which a user/medical care provider is enabled to place orders in context with other relevant patient data/information, so as to enable the user/medical care provider to see the future orders in context in an embodiment not using rows and columns in accordance with the present principles. Some doctors or EMR companies may prefer not all relevant data that's related being on one row or column on a screen or dashboard. The invention allows for other options where on a screen there can be multiple areas on that screen that display different data sets that could be grouped into multiple panels, multiple dashboards, or just lists and not in rows and columns. Zooming and scrolling functions are enabled so user can see information that they want to see while always able to see the bird's eye view, so as not to lose overall focus. In FIG. 39, element 7 depicts a clinical information panel which could also be examination elements for just today or over a period of time. Window/panel 18 shows procedures, which most commonly are CPT codes, which could be performed in an office setting or operating room and can be individualized and divided in any way the user would best be able to interact with the data. This could include wishes separating right from left, up or down when different parts of the body could be confused. Panel 24 depicts diagnostic tests of which there can be just one or many which can include any type of medical service most commonly represented by a CPT code including biopsies, chemistries, angiogram's, photographs, x-rays. More than just one panel for diagnostic tests can be on the screen as a user would need for a patient or several patients. Panel 39 can list all the medicines, including start and stop dates. Another panel could be where a doctor can view a plan and notes can be entered or past plans over time are seen and could, in some embodiments, plans created, edited, and in some embodiments populated elsewhere into the chart.

Unique to this embodiment is the fact that if the user wants any more information, data in the panels can be selected in one embodiment with direct one click access or hovering and pop-up more information. The search the database mechanism Panel 40 of FIG. 39 can be typed or through voice recognition can search all the data in the tool or EMR or PM system and display only or light up the information in each of the relevant panels that answer the question of the search. If the panels are in date order only the dates of the encounters that are related to the question were displayed in each of the panels 7, 18, 24, 38, 39, and 41. If, For instance, a question is asked, relevant information can be displayed and highlighted for instance if the question is asked ‘show me when the last time or all times that a focal laser was done in the right eye’ and all relevant information, number 16 in panel 7 number 34, 35 in panel 24, number 22, in panel number 18 also lights up. All that are highlighted as occurring the last time a laser was performed along with the diagnostic tests and clinical findings on that day. The doctor can also choose to show the immediate previous visit and a visit afterwards to see the effect of the treatments all on one screen.

In the embodiment of FIG. 39, if the order today is for a focal laser it appears in 37 or it can appear in 20, which is today's column for that and the procedure panel 18 (which can list in columns all procedures that specialist does, but then when laser is ordered by any method, the laser procedure columns come up automatically and focal laser column 19 can light up, but extremely important also presented would be number 21 showing the last time that procedure had been done, and today's clinical relevant information number 14 showing decreased vision 20/80 in this case and the tests that are related lighting up 30 and 31 all being shown. But, on 21, the last time the laser was done Jun. 15, 2018, also perhaps in another color, but lighting up number 9 Jun. 15, 2018, The clinical information is presented number 15 showing 20/60 vision and 32, 33 can light up so user can realize what had occurred when they last did a focal and user can readily select 30 and 32, and the underlying images is displayed for comparison, so user can compare before order is confirmed or treatment performed. All of this information the doctor will quickly be able to understand, but also the embodiment can guide some embodiments since that procedure is being done in the left eye and highlighted can also be the last time that procedure might have been done in the other right eye. Consideration number 16, 2, 3, 4, 35 can show the details of when it was done in the right eye even though the order now was been placed in the left eye but the data may well be relevant.

In some embodiments, the Data Command center 001 enables the medical records dashboard to intelligently expand, collapse, display, and/or hide columns, rows and/or any other portion of the medical records dashboard to show precisely what a user wishes to display. For example, in one embodiment, a Flowsheet including patient treatment and health information can be accessed from an EHR system using, in some embodiments, an icon/button, keystroke, or series of keystrokes associated with at least one of the Data Command center 001 and the medical records dashboard. Upon accessing the Flowsheet, a set of Rules and Configurations associated with, for example, the Rules module 004 of the Data Command center 001, can be evaluated to determine which data from the Flowsheet is to be displayed in the medical records dashboard. For example, in some embodiments, the Rules module 004 can include information on what data to display, and in turn what portions of the medical records dashboard to display, based on, including but not limited to, at least one of an identity of a medical care provider, an identity of a patient, a medical care provider's specialty, conditions of a patient, patient procedures, risk factors, diagnostic results, future orders, future appointments, values recorded, values not recorded, calculated values, and absolute values for display.

For example, in some embodiments in accordance with the present principles, Rules and Configurations can be predetermined and stored, for example, in the Rules module 004, for determining which data of a Flowsheet and, as such, which portions of the medical records dashboard to display or hide. Alternatively or in addition, in some embodiments, a user can self-configure the medical records dashboard to display only certain portions or to hide certain data of a Flowsheet and, as such, which portions of the medical records dashboard to display or to hide using, for example, a user interface (not shown) associated with the medical records dashboard. Alternatively or in addition, data of the Flowsheet can contain an indicator (e.g., a flag) that can be identified by, for example, the Rules module 004, for determining when and if a piece of data should be displayed or hidden.

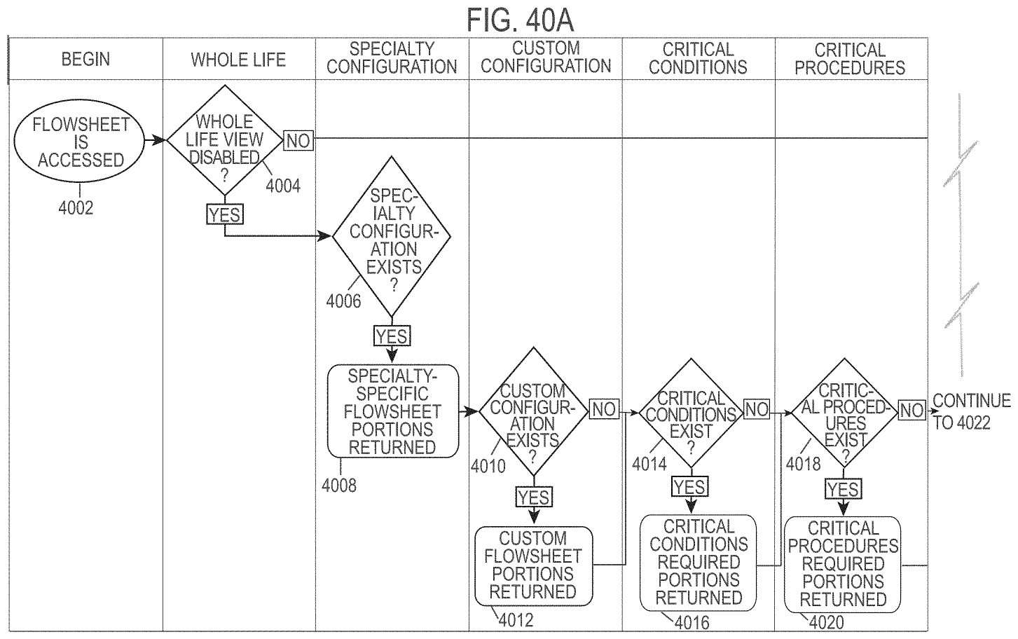

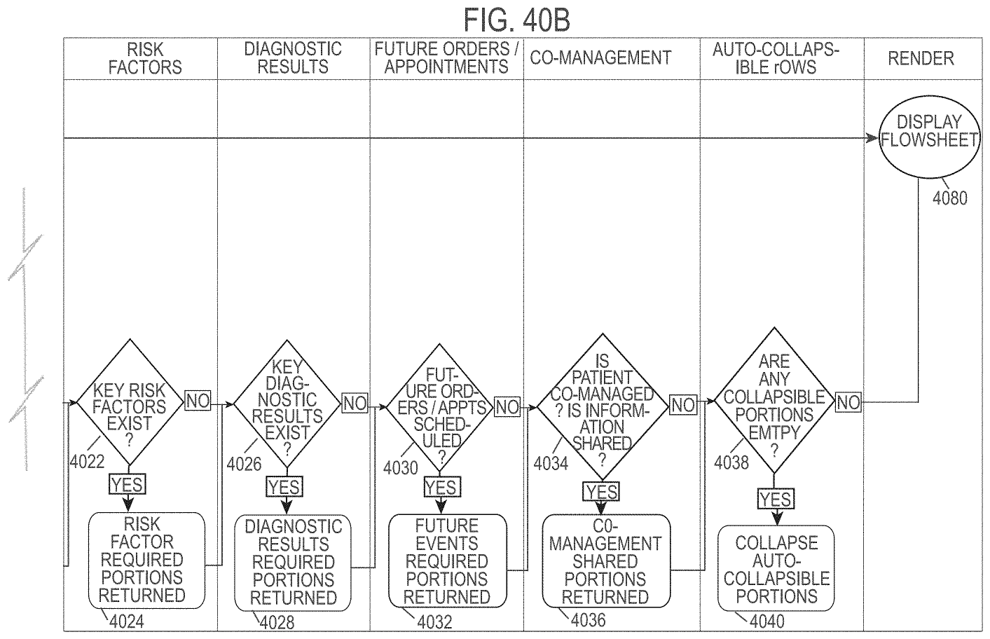

FIGS. 40A and 40B (referred to collectively herein as FIG. 40) depict a workflow diagram of a process for intelligently expanding, collapsing, displaying, and/or hiding columns, rows and/or any other portion of the medical records dashboard in accordance with an embodiment of the present principles. In the embodiment depicted in FIG. 40 the process begins at 4002 during which a Flowsheet including patient treatment and health information is accessed from, for example, an EHR system. The process illustratively proceeds to 4004. At 4004, it is determined if, what the inventors refer to as a “Whole Life View”, is disabled. More specifically, At 4004 it is determined if all the data in the Flowsheet should be displayed in the medical records dashboard. If Whole Life View is disabled, the process proceeds to 4080 during which all of the data from the Flowsheet is displayed in the medical records dashboard. If not, the process illustratively proceeds to 4006.

At 4006, it is determined if at least one Specialty Configuration exists. For example, in some embodiments a Specialty Configuration can include a configuration based on the specialty of a medical care provider. If so, the process proceeds to 4008 during which all Specialty Configurations are identified such that the data from the Flowsheet can be filtered to only display data associated with identified Specialty Configurations. For example, as previously described, in some embodiments information associated with medical care provider specialties and data to be displayed and hidden in the medical records dashboard dependent on the specialties can be predetermined and stored in the Rules module 004. In accordance with the present principles, Specialty Configurations can require certain portions, columns, and/or rows of the medical records dashboard to be displayed or hidden. After the Specialty Configurations are identified and/or if it is determined that a Specialty Configuration does not exist, the process illustratively proceeds to 4010. In accordance with the present principles, data from the Flowsheet to be displayed in or hidden from the medical records dashboard can be filtered using the identified Specialty Configurations.

At 4010, it is determined if at least one Custom Configuration exists. If so, the process proceeds to 4012 during which all Custom Configurations are identified such that the data from the Flowsheet is filtered to only display data or hide data associated with the identified Custom Configurations. For example, in some embodiments custom configurations and data to be displayed in or hidden from the medical records dashboard dependent on the custom configurations can be predetermined and stored in the Rules module 004. Alternatively or in addition, in some embodiments, a user can use a user interface associated with the medical records dashboard to create and/or identify custom configurations. In accordance with the present principles, Custom Configurations can require certain portions, columns, and/or rows of the medical records dashboard to be displayed or hidden. After the Custom Configurations are identified and/or if it is determined that a Custom Configuration does not exist, the process illustratively proceeds to 4014. In accordance with the present principles, data from the Flowsheet to be displayed in or hidden from the medical records dashboard can be filtered using the identified Custom Configurations.

At 4014, it is determined if at least one Critical Condition exists. That is, in some embodiments, critical conditions can be identified that, no matter what rules indicate that certain data should not be displayed or hidden, the identified critical conditions are to be displayed in at least one location of the medical records dashboard 400. In some embodiments, Critical Conditions can be identified and stored in the Rules module 004. Alternatively or in addition, a user can identify Critical Conditions using a user interface associated with the medical records dashboard 400. If it is determined that at least one Critical Condition exists, the process proceeds to 4016 during which the Critical Conditions are identified such that any data from the Flowsheet identified as a Critical Condition can be displayed in at least one portion of the medical records dashboard 400. In accordance with the present principles, Critical Conditions can require certain portions, columns, and/or rows of the medical records dashboard to be displayed or hidden. After the Critical Conditions are identified or if it is determined that a Critical Condition does not exist, the process illustratively proceeds to 4018.

At 4018, it is determined if at least one Critical Procedure exists. That is, in some embodiments, critical procedures can be identified that, no matter what rules indicate that certain data should not be displayed or hidden, data associated with the identified critical procedures are to be displayed in at least one location of the medical records dashboard 400. In some embodiments, Critical Procedures can be identified and stored in the Rules module 004. Alternatively or in addition, a user can identify Critical Procedures using a user interface associated with the medical records dashboard 400. If it is determined that at least one Critical Procedure exists, the process proceeds to 4020 during which data associated the Critical Procedures are identified such that any data from the Flowsheet identified as being associated with a Critical Procedure can be displayed in at least one portion of the medical records dashboard 400. In accordance with the present principles, Critical Procedures can require certain portions, columns, and/or rows of the medical records dashboard to be displayed or hidden. After the Critical Procedures are identified or if it is determined that a Critical Procedure does not exist, the process illustratively proceeds to 4022.

At 4022, it is determined if at least one Risk Factor exists. That is, in some embodiments, Risk Factors can be identified that, no matter what rules indicate that certain data should not be displayed or hidden, the identified Risk Factors are to be displayed in at least one location of the medical records dashboard 400. In accordance with the present principles, Risk Factors can require certain portions, columns, and/or rows of the medical records dashboard to be displayed or hidden. For example, a smoker with high blood pressure, and diabetes having an identified Risk Factor for a heart attack can require a visual field column with an alert to be displayed in at least a portion of the medical records dashboard 400. In some embodiments, Risk Factors can be identified and stored in the Rules module 004. Alternatively or in addition, a user can identify Risk Factors using a user interface associated with the medical records dashboard 400. If it is determined that at least one Risk Factor exists, the process proceeds to 4024 during which the Risk Factors are identified such that any data from the Flowsheet identified as identifying a Risk Factor can be displayed in at least one portion of the medical records dashboard 400. After the Risk Factors are identified or if it is determined that a Risk Factor does not exist, the process illustratively proceeds to 4026.

At 4026, it is determined if at least one Key Diagnostic Result exists. That is, in some embodiments, Diagnostic Results that are considered Key can be identified that, no matter what rules indicate that certain data should not be displayed or should be hidden, data associated with the identified Key Diagnostic Results are to be displayed in at least one location of the medical records dashboard 400. In accordance with the present principles, Key Diagnostic Results can require certain portions, columns, and/or rows of the medical records dashboard to be displayed or hidden. For example, if a lab returns a positive infectious disease test, data associated with that Key Diagnostic Result can be caused to be displayed in at least a portion of the medical records dashboard 400. In some embodiments, Key Diagnostic Results can be identified and stored in the Rules module 004. Alternatively or in addition, a user can identify Key Diagnostic Results using a user interface associated with the medical records dashboard 400. If it is determined that at least one Key Diagnostic Results exists, the process proceeds to 4028 during which the Key Diagnostic Results are identified such that any data from the Flowsheet identified as being associated with a Key Diagnostic Results can be displayed in at least one portion of the medical records dashboard 400. After the Key Diagnostic Results are identified or if it is determined that a Key Diagnostic Results does not exist, the process illustratively proceeds to 4030.

At 4030 of the embodiment of FIG. 40, it is determined if at least one Future Order/Appointment exists. That is, in some embodiments, Future Orders/Appointments can be identified that, no matter what rules indicate that certain data should not be displayed or should be hidden, data associated with the identified Future Order/Appointment are to be displayed in at least one location of the medical records dashboard 400. In accordance with the present principles, Future Orders/Appointments can require certain portions, columns, and/or rows of the medical records dashboard to be displayed or hidden. For example, if an Open-heart surgery is scheduled for the future, it can be desirable for all medical care providers to see the scheduled Open-heart surgery in at least a portion of the medical records dashboard regardless of a medical care provider's specialty. In some embodiments, Future Orders/Appointments can be identified and stored in the Rules module 004. Alternatively or in addition, a user can identify Future Orders/Appointments using a user interface associated with the medical records dashboard 400. If it is determined that at least one Future Order/Appointment exists, the process proceeds to 4032 during which the Future Orders/Appointments are identified such that any data from the Flowsheet identified as being associated with a Future Order/Appointment can be displayed in at least one portion of the medical records dashboard 400. After the Future Orders/Appointments are identified or if it is determined that a Future Order/Appointment does not exist, the process illustratively proceeds to 4034.

At 4034, it is determined if Co-Management of at least one patient is allowed and if patient information sharing is allowed. That is, in some embodiments, Co-Management of patients can require certain portions, columns, and/or rows of the medical records dashboard to be shared or hidden amongst different users/medical care providers. For example, if a medical records dashboard in accordance with the present principles is being used by multiple medical care providers to care for a patient, the patient's primary care physician is able to see lab results from a specialist if the specialist has shared at least the relevant portions of a medical records dashboard. In some embodiments, patient data/information to be shared and, as such, portions of a medical records dashboard to be shared can be identified and stored in the Rules module 004. Alternatively or in addition, a user can identify patient data/information to be shared and, as such, portions of a medical records dashboard to be shared using a user interface associated with the medical records dashboard. If it is determined that Co-Management of at least one patient exists and if patient information sharing is allowed, the process proceeds to 4036 during which the existence of Co-Management of at least one patient and patient information sharing is identified such that any data from the Flowsheet identified as being associated with Co-Management and patient information sharing can be displayed in at least one portion of the medical records dashboard 400. After the Co-Management and patient information sharing is identified or if it is determined that Co-Management and patient information sharing does not exist, the process illustratively proceeds to 4038.

In the embodiment of FIG. 40, at 4038, it is determined if any of the collapsible portions, columns, and/or rows of the medical records dashboard contain no respective values (i.e., are empty). If it is determined that collapsible portions, columns, and/or rows of the medical records dashboard contain no respective values, the process proceeds to 4040 during which the collapsible portions, columns, and/or rows of the medical records dashboard 400 containing no respective values can be collapsed or hidden from display on a least a portion of the medical records dashboard. After all of the display configurations have been determined as described above, at 4080 the data of the Flowsheet to be displayed, as determined by the process of FIG. 40 described above, is displayed in the medical records dashboard 400. The process can then be exited.

In accordance with the present principles and as described above, in some embodiments, rules determine portions, columns, and/or rows of the medical records dashboard to expand or display based on predefined criteria, and also determine portions, columns, and/or rows of the medical records dashboard to collapse or hide based on the predefined criteria, and can also determine portions, columns, and/or rows of the medical records dashboard to flag or highlight based on the predefined criteria. For example, in some embodiments, the entirety of a patient's accessible records can be viewed. In some embodiments, the entirety of a patient's accessible records are evaluated against specialty and user-specific configuration criteria (e.g., Rules), actively collapsing or hiding portions, columns, and/or rows of the medical records dashboard deemed unnecessary for a user or specialty and actively enabling the display of portions, columns, and/or rows of the medical records dashboard deemed relevant to the user or specialty. In some embodiments, an intelligent Rules system actively determines which portions, columns, and/or rows of the medical records dashboard to display based on a user, a user's specialty, a patient, a patient conditions, a patient procedures, risk factors, diagnostic results, future orders, future appointments, values recorded, values not recorded, calculated values, and absolute values for display. In another embodiment, shared portions, columns, and/or rows of the medical records dashboard between medical care providers and facilities can be added or expanded based on preconfigured or point-of-sharing decisions made by the sharing medical care providers.

Although the embodiment of the process for intelligently expanding, collapsing, displaying, and/or hiding columns, rows and/or any other portion of the medical records dashboard of the present principles described with reference to FIG. 40 illustratively comprises specific Rules-based configurations, other embodiments of the process in accordance with the present principles can comprise any combination of some or all of the described Rules-based configurations and can also comprise other Rules-based configurations. Even further, those skilled in the art will appreciate that the order of operations denoted in the process above with reference to FIG. 40 can be non-linear and optimized based on usage and workflow. That is, order, inclusion, and omission can be intelligently determined based on accessibility of data, predefined configurations, real-time user selection, custom configurations, preferred practice patterns, and/or workflow.

In addition, although in the embodiment of the process for intelligently expanding, collapsing, displaying, and/or hiding columns, rows and/or any other portion of the medical records dashboard of the present principles described with reference to FIG. 40 the Rules are described as being stored in the Rules module 004, those skilled in the art will appreciate that rules and configurations of a process of the present principles can be stored in tables, accessed remotely via API or other digital communications technology, or generated on-the-fly as the result of calculations during the operations. Rules and configurations can be stored within the application or reference outside data sources. Rules and configurations can be altered by the user, in some embodiments, by the application, in some embodiments, and/or by outside resources.

In addition, although in the embodiment of the process for intelligently expanding, collapsing, displaying, and/or hiding columns, rows and/or any other portion of the medical records dashboard of the present principles described with reference to FIG. 40 it is described that upon rendering the Flowsheet, data populates within the columns specified, in some embodiments, further rules and configurations can apply post-rendering, based on data returned and/or calculated within columns. In addition, in some embodiments, manual manipulation allows for human interaction with the finally determined dataset. As such, a user can acknowledge and remove portions, columns, and/or rows of the medical records dashboard once they have been rendered. Removal of such portions, columns, and/or rows of the medical records dashboard can be one-time, or permanent unless a subsequent event retriggers the rendering of those portions, columns, and/or rows of the medical records dashboard, and such rendering can be patient-specific, provider-specific, location-specific, or otherwise tied to an event, condition, or trigger.

In one example of the process of the present principles, a dentist can access a Flowsheet for a patient with a rare blood disorder. As a dentist, the returned set of data to be displayed in accordance with a process of the present principles would ordinarily include data germane to dentistry, collapsing or hiding certain portions, columns, and/or rows of the medical records dashboard with no values present and/or deemed unnecessary. The dentist can have also chosen not to view certain portions, columns, and/or rows of the medical records dashboard as a matter of practice. In accordance with embodiments of the present principles, as a patient with a rare blood disorder, additional portions, columns, and/or rows of the medical records dashboard could be added to the display to reflect the patient's condition of the rare blood disorder and such information could be highlighted/flagged to alert a user as to the importance of the information being displayed.

In another example, an ophthalmologist sees a diabetic patient with no diagnostic testing for a chronic illness. As an ophthalmologist, the patient data ordinarily returned for display by a process of the present principles would ordinarily include data germane to ophthalmology, collapsing or hiding certain portions, columns, and/or rows of the medical records dashboard with no values present or data deemed unnecessary for display by the process. In some embodiments, the ophthalmologist can have also chosen not to view certain columns as a matter of practice. As a patient with a lapse in testing and underlying condition requiring testing, portions, columns, and/or rows of the medical records dashboard having no value present which would normally be collapsed/hidden, could now be expanded/displayed, and highlighted or flagged to draw the attention of a user to the lack of testing having been performed on the patient.

In a third example, a primary care physician (PCP) may wish to view an entire patient history. The patient history can consist of patient care provided by the PCP, patient care provided by doctors in the same office as the PCP, and patient care provided by specialists outside the practice that co-manage the patient and have shared data with the PCP. In this arrangement, the entire dataset is provided for viewing on the medical records dashboard for care provided by the PCP and doctors within the same practice, and a shared dataset can be provided for viewing on the medical records dashboard for care provided by the specialists. Columns with no values can be collapsed or hidden if no value exists as described above.

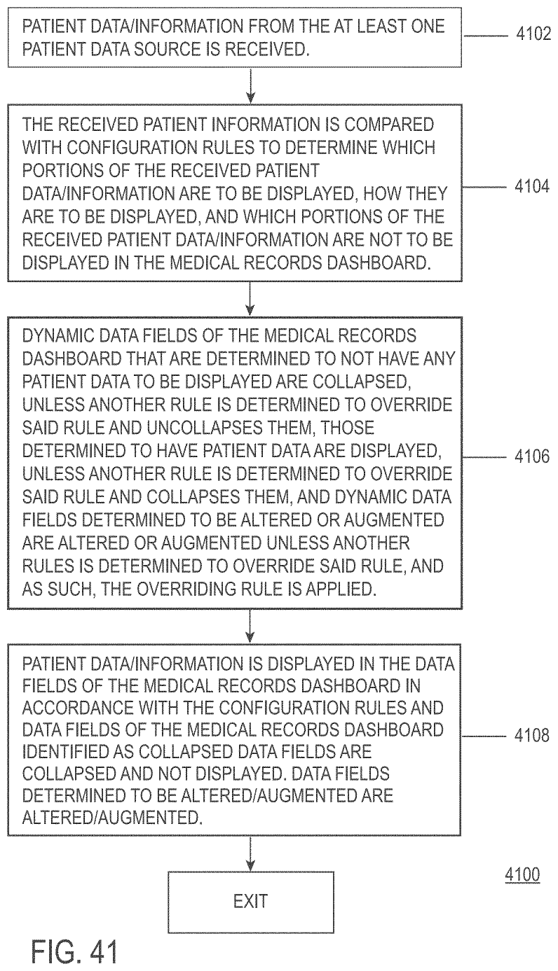

FIG. 41 depicts a flow diagram 4100 of a method for rules-based data display in a data command center comprising a medical records dashboard including one or more windows including information received or derived from at least one patient database, the medical records dashboard comprising a display on a screen, using the one or more windows, of at least one of medical services, clinical data, examination findings, diagnostic tests, and the procedures performed on one or more patients, the one or more windows comprising a plurality of collapsible data entry fields for displaying the information received or derived from the at least one patient database, wherein the at least one of the medical services, the clinical data, the examination findings, the diagnostic tests, and the procedures are arranged in rows or columns on the screen according to at least one of a time and a date that the medical services, the clinical data, the examination findings, the diagnostic tests and the procedures were performed on the one or more patients, the method beginning at 4102 during which patient data/information from the at least one patient database is received. The method 4100 can proceed to 4104.

At 4104, the received patient information is compared with configuration rules to determine which portions of the received patient data/information are to be displayed and which portions of the received patient data/information is not to be displayed in the medical records dashboard. The method 4100 can proceed to 4106.

At 4106, collapsible data entry fields of the medical records dashboard that are determined to not have any patient data to display are identified as collapsed data entry fields. The method 4100 can proceed to 4108.

At 4108, patient data/information is displayed in the data entry fields of the medical records dashboard in accordance with the configuration rules and data entry fields of the medical records dashboard identified as collapsed data entry fields are collapsed and not displayed. The method 4100 can then be exited.

In some embodiments the collapsible data entry fields identified as collapsed data entry fields comprise at least one of a column and a row of the medical records dashboard.

In some embodiments, the Data Command Center of the present principles, such as the Data Command center 001 of FIG. 1, provides a user(s) with the ability to collate data and visualize the correlation between different, related datapoints, each with their own distinct visualizations (considered by the inventors as a Corelative Line Graph display). Novel to customizable visualizations is to display an array of customized visualizations correlated on a comparative axis or axes. In some embodiments of the preset principles, the customized, correlative display consists of one or more visualizations of patient data and other data related to the Data Command Center data, horizontally, vertically, on a Z axis, or on multiple axes displaying multiple events, results, and/or calculations. In some embodiments, the Customizable, Correlative Line Graph display can be launched from within a medical records dashboard of the present principles using an icon/button, keystroke, or series of keystrokes.

Upon launch, the Customizable, Correlative Line Graph can display as a pop-up window, popover window, pop-out window, or other display format that enables the simultaneous accessibility of the Correlative Line Graph and the medical records dashboard of the present principles. The Graph may overlay or adjoin an underlying medical records dashboard in opaque or transparent states, be pinned to the medical records dashboard, and/or may hover over or aside the medical records dashboard.

Upon initiating the Customizable, Correlative Graph, a series of actions are performed to determine data and format of data displayed. Preconfigured CCG displays may be stored in tables or generated on-the-fly based on key considerations such as those laid out in Collapsible Columns and Rows, and those laid out in Guiding Actions in a Dashboard.

In one embodiment, relevant data is visualized graphically, as a series of events graphed against a timeline, correlated with a series of results, a series of actions, and a series of contributing factors. Any number of relevant details may be correlated as needed.

Data visualization is achieved with a series of configurations to determine what and how to display. In one embodiment, Source Data consists of a Value, an Inclusion/Exclusion Rule, and a Visual Representation Configuration. The data may consist of one type, a series of data points collected, values captured, validated for inclusion, and visualized across 2 intervals, correlated against a second type, a series of separate data points collected, values captured, validated for inclusion, and visualized across the same 2 intervals, correlated with a third type, a series of data points collected, values captured, validated for inclusion, and visualized across the same 2 intervals.

Rendered Customizable, Correlative Graphs may be interacted with in such ways as to turn on or off represented values in a similar manner to manually expanding/collapsing of columns and rows, i.e. turning on or off subsections of data, individual visualizations categorized by rows or columns, or selecting key elements to only display, selecting key icons within the display, and/or moving elements between positions to achieve a different view.

Those skilled in the art will appreciate additional visualizations may be added, additional flags derived, and a series of rules explained through this patent to manifest in the final rendering. Those skilled in the art will appreciate that the above described algorithm may be non-linear, may be automated in whole or in individual or groups of steps, and algorithms may intelligently update, flag, or otherwise override certain steps of the rendering process. Those skilled in the art will appreciate that single axis representation in the above description does not preclude multi-dimensional representations with multiple parallel representations as well as multiple perpendicular, or otherwise non-parallel representations.

The Customizable, Correlative Graph reaches its logical end at which point all data is rendered, processing of rendered data has occurred, and any/all necessary actions have been taken based on the processed data, including, but not limited to, Flags, Alerts, Clinical Decision Support, and Auto-Tasks. Auto-updates to patient data may initiate refactoring of the Customizable, Correlative Graph.

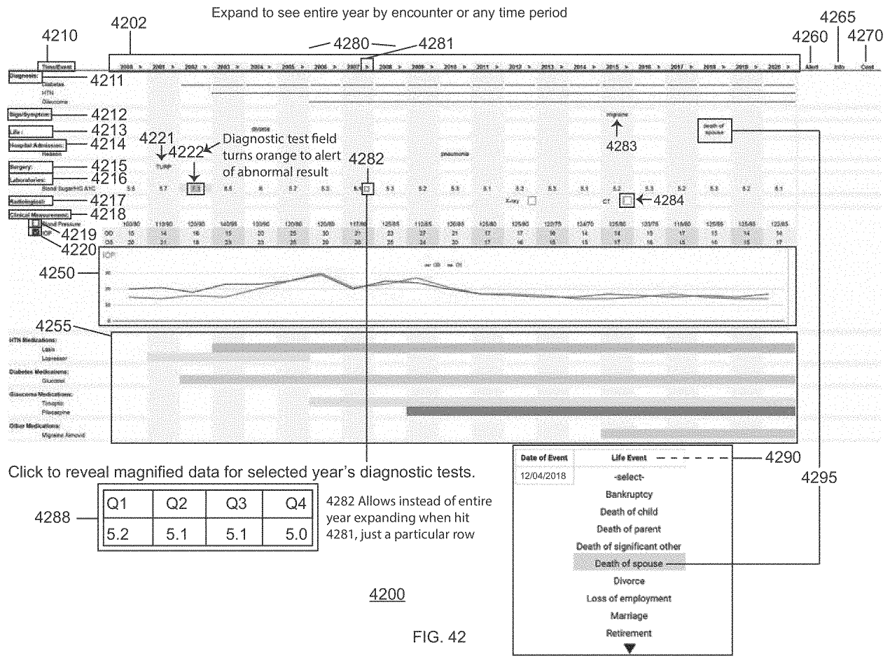

In some embodiments, the Data Command Center of the present principles, such as the Data Command center 001 of FIG. 1 enables, either as part of a medical records dashboard of the present principles or individually as a Whole Life tool, a user/medical care provider to graphically view, in a single display, a patient's entire medical history. For example, FIG. 42 depicts a graphical view of the entire medical history of a patient as a Whole Life tool in accordance with an embodiment of the present principles. In the embodiment of FIG. 42, the Whole Life tool 4200 illustratively lists dates, in one year incremented columns, across a top row 4202 of the Whole Life tool for a period of 20 years from 2000 through 2020. Although in the embodiment of FIG. 42 the time increments are illustratively one year increments, in other embodiments the time increments can be substantially any time increments chosen by the user/medical care provider.

In the embodiment of FIG. 42, the Whole Life tool 4200 in a first column 4210 lists a series of life events that occurred in a patient's life including diagnosis 4211 given to the patient, signs and symptoms 4212 the patient has had, major life events 4213 of the patient, hospital admissions 4214, surgeries 4215 the patient has had, laboratories 4216 performed on the patient, radiological procedures 4217 performed on the patient, and clinical measurements 4218 made on the patient. The Whole Life tool 4200 of FIG. 42 further illustratively includes an IOP section 4250 graphically displaying the intraocular pressure of a patient's right eye (OD) and the patient's left eye (OS) as a line graph spanning the 20 depicted years of the patient's medical history. In the embodiment of FIG. 42, the line graph of the IOP of a patient's right eye (OD) is color-coded red and the line graph of the IOP of the patient's left eye is color-coded blue for easier distinction. In the embodiment of the Whole Life tool 4200 of FIG. 42, a lower section 4260 graphically displays a medication history for the patient. In FIG. 42, horizontal bar graphs depict a history of the medication taken by and/or prescribed to a patient spanning the 20 depicted years of the patient's medical history. In the embodiment of FIG. 42, the various medication bar graphs can be color-coded to more easily distinguish between medications. In some embodiments, color standards, such as defined by the American Academy of Ophthalmology, can be used for color coding the medications. Alternatively or in addition, in some embodiment custom colors can be used.

In the Whole Life tool 4200 of FIG. 42 any column, 4281, can be selected 4280 and expanded to take up the entire page, or a partial part of the page, or a navigation template 4290 may be used to navigate the timeline by date range or to zoom in on specific results for that time increment 4282. For example, if a user/medical provider selects the year 2007, that particular year can expand so that instead of displaying one full year as depicted in FIG. 42, the Whole Life tool 4200 can display 12 months in the year in one month increments or quarterly or in any other increments, for example, for every medical encounter the patient has had. In some embodiments, a user/medical care provider is enabled to select whether to display all the encounters that the patient has had with any medical care providers or just particular medical care providers. In some embodiments, a zoom view of a particular time span can be displayed on another monitor such that a user/medical care provider is able to view the zoomed time increment simultaneously with the whole life view.

In the embodiment of the Whole Life tool 4200 of FIG. 42, the patient illustratively had three major disease states, diabetes, hypertension, and glaucoma, as listed in the diagnosis row 4211. The Whole Life tool 4200 enables a user/medical care provider to select any of the identified major disease states to find out more detailed data regarding the selected disease state and update start/stop dates or activate/inactive a diagnosis. As depicted in FIG. 42, a user/medical care provider is able to determine when the disease exactly occurred by referring to the Whole Life tool 4200. In the embodiment of FIG. 42, the diabetes occurred in 2002, 2003 was hypertension, and 2006 was glaucoma. These are chronic diseases, and these are the dates of onset. In some embodiments, the Whole Life tool can include a bar graph that can continue along a horizontal date line displaying the time period that the patient had that diagnosis, and if for some reason they no longer had that diagnosis, the bar graph could stop.

The Whole Life tool 4200 of FIG. 42 displays for a user/medical care provider in row 4212 when a patient developed a symptom and identify the symptom 4283. Similarly, the Whole Life tool 4200 of FIG. 42 is able to display for a user/medical care provider in row 4213 when a major life event that can affect the well-being of a patient occurred such as a divorce or the loss of a loved one, etc. As previously described, in row 4214, the Life tool 4200 of FIG. 42 is able to display for a user/medical care provider hospital admissions the patient had over the 20 years spanning the patient's recorded medical history. In the embodiment of FIG. 42, in 2010, the patient was hospitalized for pneumonia. As depicted in row 4215 of the Whole Life tool 4200 of FIG. 42, the patient had a surgery, transurethral resection of the prostate, in 2001. In addition, row 4216 of the Whole Life tool 4200 of FIG. 42 depicts that the patient has had laboratories, illustratively, blood sugars labs were performed, like hemoglobin A1C and update start/stop dates or activate/inactive a diagnosis. It should be noted that in the embodiment of the Whole Life tool 4200 of FIG. 42, a valued displayed in some rows and/or columns can be an average value of a measured parameter for the time increment depicted by the column. That is, in some embodiments each row and/or column can be a smart row or column and if a laboratory was taken four times in a year, the Whole Life tool 4200 can be configured to display an average of all values measured during the time increment. In some embodiments, by selecting a value in a row, patient data/information can be displayed in a window or other display means depicting all of the values measured and/or laboratories for the time increment. Even further, by selecting a particular measured value or laboratory, further detailed information for that particular value or laboratory can be displayed to a user/medical provider. Although the embodiment of FIG. 42 is described as displaying an average value, in some embodiments a high, low or other particular value can be selected by a user to be displayed 4222 represents an alert for an abnormal result.

In row 4217 of the Whole Life tool 4200 of FIG. 42, radiological procedures performed on the patient are displayed. For example, in FIG. 42, a CT scan was performed on the patient in 2015. In accordance with the present principles, by selectin the indicator in row 4217 of the year 2105, the image of the CT scan can be displayed to the user/medical care provider. In row 4217 of the Whole Life tool 4200 of FIG. 42, clinical measurement taken on the patient can be displayed. Such clinical measurement can include blood pressures taken at each doctor's visit. In some embodiments, the results can be displayed as a number. Alternative or in addition, in some embodiments, by selecting an icon associated with the clinical measurements, a graph representing the clinical measurements over time can be displayed. 4219 and 4220 represent radiological procedures and show how they may be toggled between one, many, or all. Images may be directly accessed and viewed within context by selecting them 4284.

In accordance with the present principles, in the Whole Life tool 4200 of FIG. 42, substantially any portion of a time increment or presentation of patient-related data/information can be selected to cause a display of a more detailed view of the selected time period/value.

In the Medications section (4255) of the Whole Life tool 4200 of FIG. 42, start dates and stop dates for each of the medications are displayed and may be interacted with in accordance with Medication Management protocols described herein.

The Whole Life tool 4200 of FIG. 42 illustratively comprises three optional columns; an alert column 4260, an info column 4265 and a cost column 4270. The alert column 4260 can be used to alert a user/medical care provider of an issue that requires further attention. In some embodiment alerts are automatically created by, for example a Rules module (described in greater detail below), and alternatively or in addition, alerts can be input by the user/medical providers with access to the Whole Life tool 4200.

Whole Life view may be interacted with whereby a doctor may choose to update an event, such as a life event (4290) by selecting said event and the event will auto-populate on the whole life view (4295).

The info column 4265 of the Whole Life tool 4200 of FIG. 42 can be used to provide information for a user/medical care provider. For example, in some embodiments, links can be provided to direct a user/medical care provider to sources of additional information, such as PUBMED, if the user/medical care provider is interested in learning about medications. Alternatively or in addition, the info column 4265 can be used by users/medical care providers to provide information to other users/medical care providers.

The cost column 4270 of the Whole Life tool 4200 of FIG. 42 can be used to display to a user/medical care provider information associated with cost in providing medical care a patient. For example, in some embodiments, the cost column 4270 can be used to provide to a user/medical care provider information regarding what a patient's insurance company will authorize. Alternatively or in addition, in some embodiments that cost column 4270 of the Whole Life tool 4200 can display to a user/medical care provider information regarding bills, paid or unpaid, associated with a patient.

In some embodiments, a user/medical care provider can input patient-related data/information into a Whole Life tool of the present principles. Alternatively or in addition, a Rules module can auto-populate patient-related data/information into a Whole Life tool of the present principles. For example, in some embodiments, an integration module of the present principles, such as the integration module 002 of the Data Command Center 001 of FIG. 1, can collect patient data/information from outside sources (e.g., an EMR system). The patient data/information is made accessible, for example via a storage means, to a Rules module of the present principles, such as the Rules module 004 of the Data Command Center of FIG. 1. In addition to having access to the data/information collected by the Integration module 002, the Rules module 004 can have access to all information input by a user/medical care provider via, for example, a medical records dashboard or any other user interface. Alternatively or in addition, in some embodiments, the Rules module 004 is configured to further have access to patient related information and general medical knowledge including but not limited to medical information regarding health conditions and treatments, symptoms and side effects, procedures, images and diagnosis, and other related medical information. As such, in some embodiments, the Rules module 004 can auto-populate at least portions of a Whole Life tool of the present principles. The Rules module 004 can then, via for example a Display module, such as the Display module 006 of the Data Command center 001 of FIG. 1, can cause the display of any portion or zoomed-in portion of a Whole Life tool of the present principles.

In some embodiments, the Data Command Center of the present principles, such as the Data Command center 001 of FIG. 1, can provide, either via a medical records dashboard of the present principles or individually, a Medical Guidance tool to assist users/medical care providers to plan and schedule health services for patients. In some embodiments, the medical guidance tool of the present principles enables a scheduling of patients with automated methodology by, for example, prioritizing the risks of symptoms and diseases, and associating these with past procedures, diagnostic tests and other critical items that need to be evaluated. With such methodology, a medical guidance tool of the present principles guides users/medical care providers in determining, which patients needs the timeliest interventions, appointments and follow up. In some embodiment, the medical guidance tool is configured to examine patient records and information to determine if medications ordered, procedures ordered, follow up visits ordered and if a plan of treatment determined for the patient by a user/medical care provider are accurate or contain any errors.

Alternatively or in addition, in some embodiments a medical guidance tool of the present principles can determine if a patient has missed an appointment and, in response, can alert a user/medical care provider to the fact that a patient has missed an appointment and/or can schedule a task for a user to at least contact the patient to schedule another appointment. In some embodiments, in addition to determining that the patient has missed an appointment, a medical guidance tool of the present principles can determine a level of risk presented to the patient's health by that patient missing the appointment. As such, patient's whose health is at a high risk by missing the appointment can be identified and contacted in an urgent manner to reschedule the missed appointment. In addition, the number of missed appointments can be tracked, whether the patient cancels or the practice cancels, and a pattern identified for the user/medical care provider.

In some embodiments of a medical guidance tool of the present principles, tasks can be generated for different users (e.g., doctors, staff, schedulers, etc) and such tasks can be presented to different users depending on a determined level of risk or urgency to a patient. For example, doctors typically do no schedule follow up appointments for patients. Such task is usually performed by a scheduler. As such, typically scheduling tasks generated by a medical guidance tool of the present principles are generally directed to an identified scheduler. In some embodiments however, if a patient misses an appointment and the a medical guidance tool of the present principles determines that missing the appointment presents an elevated risk to a patient's health, the medical guidance tool of the present principles can generate a rescheduling task that is now directed to the doctor. Alternatively or in addition, the medical guidance tool of the present principles can generate an alert to be present to a user/medical care provider that the missed appointment presents an elevated risk to the health of the patient.

For example, in a scheduling embodiment, an integration module of the present principles, such as the integration module 002 of the Data Command Center 001 of FIG. 1, can collect patient data/information from outside sources (e.g., an EMR system). The patient data/information is made accessible, for example via a storage means, to a Rules module of the present principles, such as the Rules module 004 of the Data Command Center of FIG. 1. In addition to having access to the data/information collected by the Integration module 002, the Rules module 004 has access to all information input by a user/medical care provider via, for example, a medical records dashboard, such as the medical records dashboard 400. In some embodiments, the Rules module 004 is configured to further have access to patient related information and general medical knowledge including but not limited to medical information regarding health conditions and treatments, symptoms and side effects, procedures, images and diagnosis, and other related medical information. As such, in some embodiments, the Rules module 004 can monitor patient data/information and can be configured to monitor patient scheduling. As such, when a Rules module 004 determines that a patient has missed a scheduled appointment, by for example determining if a user/medical care provider has interacted with the patient that day or not by determining if any information has been entered into a medical records dashboard or other user system for that patient that day, a Rules module 004 can determine if a patient has missed a scheduled appointment. If the Rules module 004 determines that a patient has missed a scheduled appointment, the Rules module 004, via for example a Display module of the present principles, such as the Display module 006 of the Data Command center 001 of FIG. 1, can cause a display of an alert, to call to the attention of a user/medical care provider that the patient has missed a scheduled appointment. Alternatively or in addition, the Rules module 004 can cause the scheduling of a task to be presented to a user/medical care provider such that a new appointment can be scheduled for the patient.

In some embodiments, having information regarding at least patient medical conditions, general and specific treatments and procedures, patient scheduling and other patient-related data/information, the Rules module 004 is able to determine if missing the scheduled appointment place the patient's health at an elevated risk. If so, the Rules module 004 can cause, for example via the Display module 006, a display of an alert, to call to the attention of a user/medical care provider that the patient's missed appointment results in an elevated risk to the patient's health. As described above, the determination of the elevated risk can cause the alert to be directed to a higher-level user such as a doctor instead of an administrator. In some embodiments of the present principles, the display of the alert itself can change and can be caused to be presented in a different color than usual or with other visual attributes, such as blinking or appear large on a display.

In some embodiments, a Medical Guidance tool of the present principles can assist in the scheduling of an appointment for a patient. For example, in an embodiment in which a scheduler is inputting patient data/information into an electronic system/spreadsheet/form, the Rules module 004 of the present principles can be configured to monitor such input patient data/information. Using the monitored input data/information and medical information known to the Rules module 004, the Rules module 004 can cause a display of a suggested appointment date to a user. For example, if a patient is known to have had a procedure performed and such procedure has a post-operative appointment typically scheduled for 30 days, the Rules module 004 can cause a display of a suggestion to a user that an appointment be scheduled for 30 days after the procedure was performed. In some embodiments, for suggesting an appointment, the Rules module 004 can further consider parameters such as time since a last procedure, symptoms since the last procedure, the doctor that performed the last procedure, medical history of the patient, the patient's disease state, and the like. In some embodiments, for new patients, the Rules module 004 can even take into account, who is referring the patient. If a patient referral comes from a doctor in a subspecialty that clearly would know what is an emergency, like another eye doctor, the Rules module 004 might suggest that an early appointment date must be made. In some embodiments, the Rules module 004 can create tasks for a user to make appointments on a suggested date or alternatively or in addition can schedule appointments without the need for a user intervention.

In some embodiments of the present principles, a Medical Guidance tool can assist in scheduling a patient to see a different doctor than the patient came to see. By way of example, in ophthalmology there may be in one office a general ophthalmologist, an optometrist, a retina surgeon and a glaucoma surgeon. A patient with diabetes and glaucoma may need to see the retina surgeon four times a year and the glaucoma surgeon three times a year. A scheduler for the practice or even the patient themselves can get confused as to which doctor to see. For instance, if the glaucoma doctor sees the patient and does not schedule the patient to return to the retina doctor, who handles another type of disease, not infrequently, patients can be totally lost and the wrong provider is assigned to give care. While one provider may be taking care of one disease state, (i.e. glaucoma), the other states (i.e., diabetic eye disease or macular degeneration), can be inadvertently neglected. The same can be true in a multispecialty practice of internists, cardiologists and pulmonologists. For example, an internist can have a good working relationship with the patient and sees the patient on a regular basis, however, the internist may not realize that the patient did not keep or ever get scheduled for an appointment with a cardiologist.

In some embodiments in accordance with the present principles, the Rules module 004, having knowledge of a patient's entire medical history, conditions, current procedures and treatments and having general knowledge of medicine and specifically the relationship between treatments and procedures of internists and cardiologists, can cause a display, for example via the display module 006, of at least one of an alert, suggestion and/or a task that causes the patient to be scheduled for an appointment with a cardiologist. A Medical Guidance tool of the present principles is able to determine when a patient is supposed to return for an appointment, what the high-risk scenarios exist, whether there was a procedure performed on a patient that requires a follow up with a particular doctor, whether a follow up appointment is kept by the patient, and is able to suggest or remind a user/medical care provider that they should consider sending a patient to another doctor. In some embodiments, not only are there indicators and alerts sent to the doctor who sees the patient, but indicator and alerts can be sent to an original doctor whose appointment has been missed, to a practice manager, or to anyone else in the practice to be able to determine whether a particular patient should be seeing a particular doctor or if the patient has been lost in the shuffle of so many visits. Such mistakes can happen in health systems and hospitals in which a patient who is not knowledgeable about medicine makes the assumption that each doctor talks to one another and shares records and therefore the patient assumes that if one doctor does not suggest that the patient sees another doctor, that the original doctor will be taking care of everything and the patient does not need follow-up care from a different doctor.

In some embodiments, a Medical Guidance tool of the present principles can monitor patient-related information intended to be reviewed by at least one user/medical care provider to determine if that information has been reviewed by the at least one user. For example, in some embodiments, the Medical Guidance tool, for example via the Rules module 004 can identify if results from tests ordered or notes from other medical care providers or any other “attachments” sent to for example a medical records dashboard, have been reviewed by all intended users/medical care providers for which they were intended. If the patient-related data/information intended for review by users/medical care providers has not been reviewed, the Medical Guidance tool can cause a display of an alert to the users/medical care providers that have not reviewed the patient-related data/information and for which the patient-related data/information was intended. Alternatively or in addition, the Medical Guidance tool can create a task for at least one of users/medical care providers for which the patient-related data/information was intended and whom have not reviewed the patient-related data.

For example, in a multispecialty practice, if the pathology results of a biopsy of a skin lesion was received and a family doctor sees the results, but the dermatologist who ordered the biopsy does not see the results, an alert can be sent out to either one or both of the doctors or alternatively or in addition, a task can be created for one or both of the doctors to view the results of the biopsy.

In some embodiments, a Medical Guidance tool of the present principles enables the pre-analysis of current and future patent visits. Such functionality enables users/medical are providers to prepare for patient visits and review scheduling of patients and test/procedures to determine if any errors exist. A user/medical care provider can review tests/procedures scheduled for a patient, when the patient last had similar tests, what patient's disease states are, what the likelihood is that the patient might need additional tests or another type of procedure, and even whether or not something might have been scheduled in error because information from the previous visit doesn't match up. For example, if a patient treatment plan indicates that an injection is to be done in the left eye but the schedule says injection in the right eye, the Medical Guidance tool via, for example, the Rules module 004, can discover the discrepancy and cause an alert to be displayed to a user/medical care provider to warn of the discrepancy.

In some embodiments, a Medical Guidance tool of the present principles enables the post-analysis of patent visits. Such functionality enables users/medical care providers to pull up patient data/information related to visits of any past patients seen in any office or a particular patient seen with certain disease states or procedures or diagnostic tests and see what was done on any given time period or visit. Such functionality can be especially useful if a user/medical care provider references, for example, a medical records dashboard on the same day of a visit or shortly thereafter when the memory of patients are fresh in their memory. During such review, a user/medical care provider can review to determine if their examinations were filled out correctly and that any diagnostic and/or procedural matters were performed and performed correctly and determine if any tests or orders were missed.

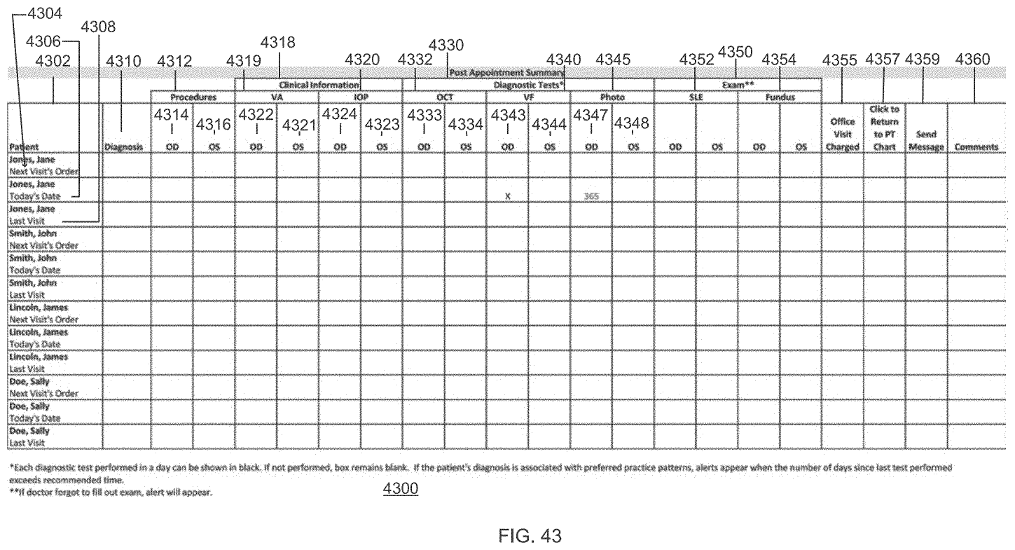

FIG. 43 depicts a post appointment summary chart 4300 of a Medical Guidance tool in accordance with an embodiment of the present principles. The post appointment summary chart 4300 illustratively includes a first column 4302 listing a next visit's order row 4304, a today's date visit row 4306, and a last visit's row 4308. The post appointment summary chart 4300 has a plurality of other columns including a diagnosis column 4310, a Procedures column 4312 including a right eye column 4314 and a left eye column 4316, a Clinical information column 4318, including a VA column 4319 and an IOP column 4320, each having respective right eye columns 4322, 4324, and respective left eye columns 4321, 4323, and a Diagnosis Tests column 4330, including an OCT column 4332, a VF column 4340 and a Photo column 4345, each including respective right eye columns 4333, 4343, 4347, and respective left eye columns 4334, 4344, 4348.

The appointment summary chart 4300 of FIG. 43 further illustratively includes an Exam column 4350 including an SLE column 4352 and a Fundus column 4354, an Office Visit charged column 4355, a Click to return to PT chart column 4357, a Send message column 4359, and a Comments column 4360. Although in the embodiment of FIG. 43 the appointment summary chart 4300 illustratively includes specific columns and rows for providing the illustrated patient related data/information, in some other embodiments different patent related data/information can comprise the appointment summary chart 4300. In addition, although in the embodiment of FIG. 43 the appointment summary chart 4300 illustratively comprises a post appointment summary chart of the present principles, the appointment summary chart 4300 can comprise a pre appointment summary chart in accordance with the present principles. In some embodiments, at least some of the rows and columns of the appointment summary chart 4300 can be auto-populated.

In some embodiments, a Medical Guidance tool of the present principles enables users/medical care providers to create a preferred practice method. For example, in some embodiments, a Rules module 004 is programmed with a preferred practice method of a user/medical care provider. The Rules module 004 can then provide services, such as assisting in the creation of appointments and determining if patients kept their scheduled appointments in accordance with the preferred practice method of the user/medical care provider.

In some embodiments, a Medical Guidance tool of the present principles enables a tracking of payments in accordance with the present principles. Current EMR systems require user driven reports to be run manually to identify items that have not been paid or are rejected by insurance carriers. Most insurance companies send payment for hundreds of separate patient claims on the same electronic check that is then posted automatically to many different patient accounts without inspection or review by a billing or staff member. This electronic process was developed to reduce workloads on staff who before were required to read the explanation of benefits and apply the payment manually to each individual claim item in the billing system, which allowed for greater oversight of incorrect payments and rejections.

In some embodiments of a Medical Guidance tool of the present principles, a Rules module, such as the Rules module 004 of the Data Command center 001 of FIG. 1, can be configured to monitor individual CPT codes in, for example in some embodiments, a medical records dashboard of the present principles, to identify when bills are not completely paid or are rejected. In some embodiments, if it is determined that a bill is not completely paid or rejected, the Rules module 004 can cause, for example via the Display module 006, a display of an alert to alert a user/medical care provider that a bill was not completely paid. Alternatively or in addition, a task can be created for a user/medical care provider to correct the unpaid bill. In such embodiments, the Rules module 004 can have access to such information as specific insurance payors information, patients with high deductible plans, amount of billing, and the like. All pertinent data can be analyzed by, for example, the Rules module 004 and an indicator or a task can be created to alert the appropriate staff members and physicians enabling the users to make corrections rapidly. In some embodiments, based on user preferences, fully automated queries can generate indicators that can be viewed live while a patient is being treated.

In some embodiments of the present principles, a Medical Guidance tool of the present principles provides an electronic patient interface. For example, when a patient calls for an appointment or to ask questions or emails to schedule an appointment or ask questions, a user interface enables a patient to ask and answer questions, enter information, refill medications and the like. The reality is doctors often do not have the time to communicate with each and every single patient. In some embodiments, a Rules module of the present principles, such as the Rules module 004 of the Data Command center 001 of FIG. 1, having knowledge of all patient related data/information is also provided access to all information provided by a patient via the caller or email user interface. The Rules module 004 can evaluate every patient query in light of the information available to the Rules module 004. In some embodiments, the Rules module 004 determines if the patient is a current patient and if so, if the patient h had procedures or a risky diagnosis so that the Rules module 004 can present to a user/medical care provider a most complete picture of a patient as possible including which patients might be more problematic and urgent based on patient's symptoms, diagnosis, past procedures and other patient history In some embodiments the Rules module 004 can generate an alert directed to a user/medical care provider, the alert including the details of the patient developed by the Rules module 004. Alternatively or in addition, the Rules module 004 can create a task for a user/medical care provider, the task including the details of the patient developed by the Rules module 004. If the alert/task is not responded to within a certain amount of time by the user/medical care provider, the Rules module 004 can generate another alert and/or task directed to another user/medical care provider to attempt to elicit a response for the patient.

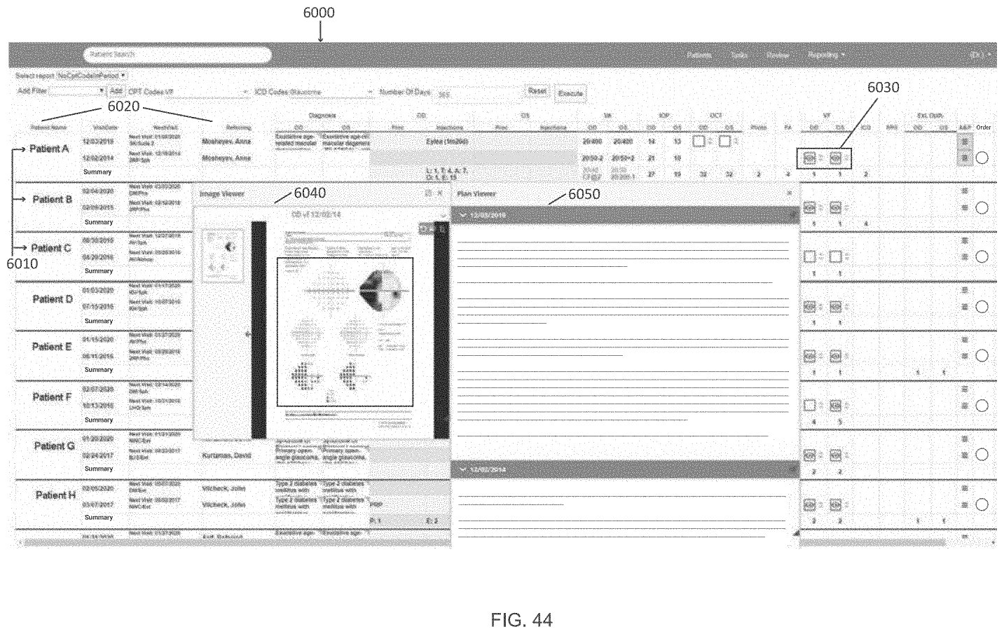

In some embodiments of the present principles, a Data Command Center of the present principles enables an organized view derived from disparate data sources in accordance with user-defined parameters, of a specific report type, executable on a single or group of patients. For example, FIG. 44 depicts an Evaluative Clinical Reporting (ECR) interface 6000 that provides an organized view derived from disparate data sources in accordance with user-defined parameters, of a specific report type, executable on a single or group of patients. The ECR interface 6000 displays only necessary and relevant data based on individual patient information in rows 6010 and columns 6020 that allows Clinical Professionals the ability to evaluate many data points and make informed clinical decisions, which would not otherwise be possible in a single display. While reviewing the data, the Clinical Professional can execute multiple actions, including but not limited to the following:

First, the Clinical Professional can, with a single click, view additional relevant data by clicking on icons 6030. An example of this would be viewing diagnostic imaging in a separate, overlaid panel 6040, from the relevant visit date. It is important to note that while viewing the diagnostic imaging, the Clinical Professional can manipulate the underlying flowsheet while manipulating the diagnostic image.

An additional example of this functionality would be viewing the plan 6050 that was used for that specified visit. As with the previous example, the underlying flowsheet can still be manipulated when viewing the plan 6050. It is important to note that N number of additional data panels can be simultaneously opened and overlaid on the reporting results.

A clinical professional can also use a single click on an icon 6030 to load a specified letter for viewing in a separate, overlaid panel, while still able to view the flowsheet in context. As with the previous examples, the Clinical Professional is able to manipulate the flowsheet and also view additional relevant data panels by clicking an icon on the flowsheet while viewing the letter.

After the Clinical Professional has reviewed the patient, there may be some action to take in order to rectify whatever is causing the patient to be included in the report. The Clinical Professional may choose to create a task for a support staff member to take some action. The Clinical Professional may also choose to create an order for a patient while viewing the report results. An ordering panel may be launched within context by clicking an icon. The panel is overlaid with the reporting results flowsheet. As described, the panel provides a mechanism by which the Clinical Professional can select the type of order, input the relevant parameters and then create the order. A Clinical Professional may also choose to create a letter to a Referring Provider in order to satisfy regulatory requirements or provide guidance to the referring provider in a co-management situation. The Clinical Professional would launch the create letters interface over the top of reporting results flowsheet allowing them to write a letter in context of the flowsheet data on screen. However, it is possible that the Clinical Professional does not need to take any action and simply chooses to evaluate the many data points in a concise view of rows and columns of relevant data.

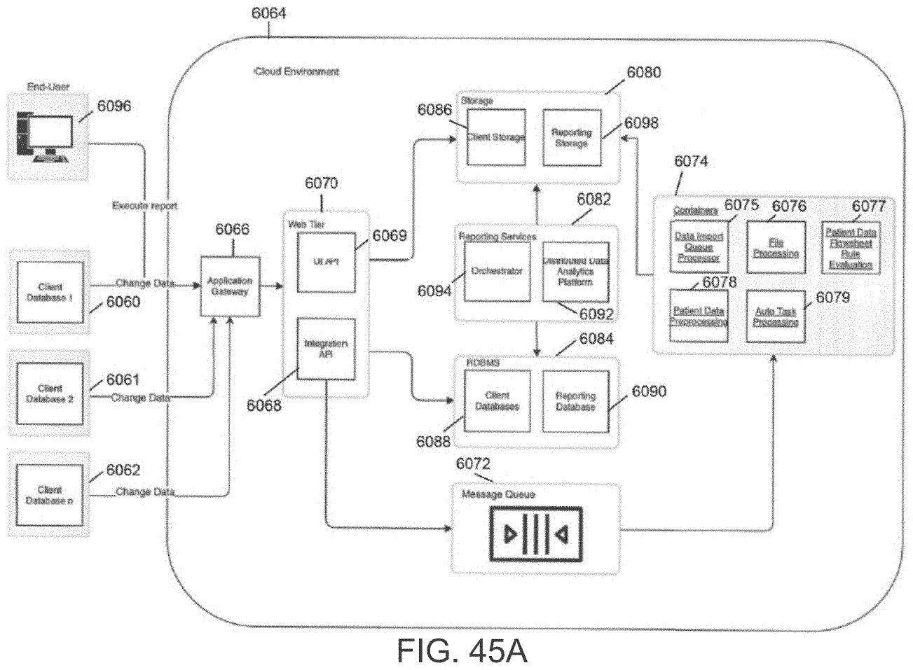

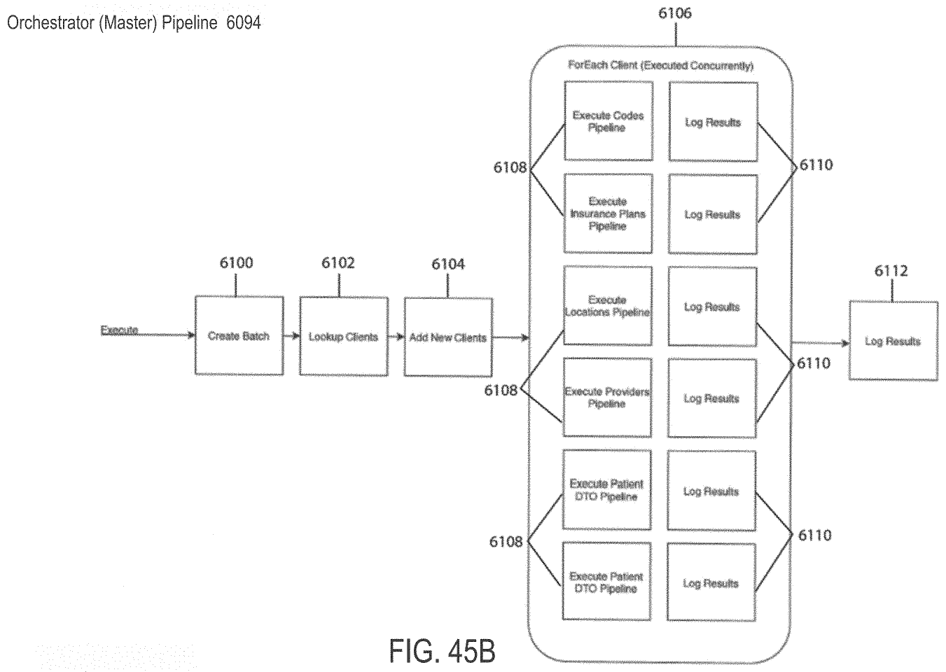

FIG. 45A depicts a reporting architecture of Data Command Center in accordance with an embodiment of the present principles. As data changes on a remote client's EHR 6060, Practice Management 6061, Diagnostic Equipment 6062, or other system, it is pushed to the cloud environment 6064 via an application gateway 6066 to an Integration API server 6068 in web tier 6070. Once the Integration API server 6068 receives the HTTP request with the updated data, it will then insert a message into the appropriate message queue 6072. Five separate application containers 6074 are connected to the message queue 6072 waiting to process information as it is received. A container is defined as a method to package an application, all its dependencies, and runs in isolation from other processes.

The first application container, Data Import Queue Processor 6075, processes changed data and loads it into the appropriate client database 6080. During the processing, the data is mapped to the correct database entities by reporting services 6082 and inserted into the Client Relational Database Management System (RDBMS) 6084. Once the entities are inserted into the client database 6086, a message is added to a Patient Data Preprocessing queue. Placing this message into the queue will cause the Patient Data Preprocessing Queue to begin processing for the patients whose records were updated.

The second application container, File Processing 6076, loads all new diagnostic imaging into Client Storage 6086. Additionally, a record will be added to the Client Database 6088 which contains a pointer that allows for the UI API 6069 to retrieve the correct image for the patient.

The third application container, Patient Data Flowsheet Rule Evaluation 6077, queries the client database 6086 for the latest snapshot information, including, but not limited to, Demographic Information, Billing History, Diagnostic Imaging, and Medical Data. Once the relevant information has been loaded into memory, a series of rules are executed in order to transform those data into a JSON object optimized for displaying Medical Information, Diagnostic Testing results, etc. in rows and columns of the flowsheet. Once all rules have been run, the Flowsheet Encounter Data is then written to the Client Storage File System 6086.

The fourth application container, Patient Data Preprocessing 6078, will generate an object that contains a historical snapshot of everything that has happened to the patient including, but not limited to, Demographic Information, Billing History, Diagnostic Imaging, and Medical Data. This snapshot, Patient Data Transfer Object (Patient DTO), is stored in a JSON object and written to the Reporting File System 6090 which will be used during the Extract, Transform, and Load (ETL) process to insert rows into the Reporting Database. The ETL process consists of multiple steps to Extract the data from the client RDBMS 6084, Transform it with logical commands executed by the Data-Analytics platform 6092 which is distributed to multiple worker nodes by Orchestrator 6094, and Load the data into the Reporting Database 6090.

The Reporting Database 6090 includes a series of Facts and Dimensions tables which can be extended to include new tables as new use-cases are encountered. Those skilled in the art will appreciate that the data may be architected to use a Star Schema in order to ensure that data is able to be returned in an acceptable time. All the data is stored in a single database. In order to ensure that data is kept appropriately segregated per client, all tables contain a Customer Id Unique Identifier column. The defined process to generate new reports mandates that Report Developers write all queries to include a Customer ID parameter in the WHERE clause, ensuring that data will only be returned for the customer that is specified. In order to be able to view the report execution interface, the user must complete the login, or authentication, process. The authentication process requires a practice identifier, a user, and a password be passed as parameters to the authentication controller. Once authentication has completed, a Secure, HTTP Only, Same Site cookie is generated by the Integration API server 6068 which contains a claim that ties the current authenticated user, to a customer ID. When the report execution HTTP request is made, the cookie is passed in the request, the customer ID is extracted from the cookie and passed automatically as a parameter to the stored procedure execution.

Finally, the Auto Task processing container 6079 runs a set of defined rules to evaluate for certain conditions and to generate a task for a patient once certain conditions are met. If it is found that a certain condition has exceeded a threshold, a task will automatically be sent to the appropriate user to handle the task. For example, if a patient has a diagnosis that requires a certain diagnostic test every 365 days and has exceeded that threshold of 365 days, a task will be sent to the computer 6096 of the Front Desk Staff to schedule that diagnostic test. Once the patient has the required diagnostic test, change data will trigger the reprocessing of the auto task rules, and will automatically resolve the task for the relevant patient.

In order to move the newly generated files from Client Storage 6086 to Reporting Storage 6098, a time-based job executes on a scheduled interval (cron job) on the order of minutes, to find all new and/or modified Patient DTOs in the Client File System since the last time the job was run, and then places a replica of the newly modified file in the Reporting File System of the Reporting Storage 6098. These jobs will continually copy new or modified files to the Reporting File System as the Patient DTOs are generated.

During the day, Patient DTOs will be moved from Client Storage 6086 to Reporting Storage 6098, waiting for its data to be loaded into the reporting database 6090. In order to start the ETL process, another cron job starts the execution of the Orchestrator Master Pipeline which contains all its child pipelines. The interval for execution is generally set to each night. The master pipelines specify the order in which the child pipelines should execute, error handling, and other logical functions. The pipelines will load the base changed data from the clients' database to create a batch at FIG. 45B 6100. Base changed data consists of, but is not limited to, patient demographics, patient insurances, customers, providers, practice locations, etc. The orchestrator pipeline allows the addition, or removal, of pipelines at 6102, 6104 to extend the ability to transform and load new facts and dimensions. While the base data is loaded, an additional pipeline 6106 executes concurrently which will process the Patient DTO data. Each child pipeline 6108 is processed by calling separate transformation jobs which contain the necessary logic to transform the Patient DTO data for insertion to the relevant Facts and Dimensions tables at 6110. The transformation logic is applied using the distributed data-analytics platform 6092. After the Orchestrator Pipeline 6094 has finished processing, the data is logged at 6112 and is available to be reported on.

Once the ETL process begins, the Patient DTO data is staged to be transformed and loaded to the reporting database. To begin the Transformation process, the most recent JSON schema for the Patient DTO object is loaded into memory. The schema is then used to read the DTO object from reporting storage into a proprietary table common for the data-analytics platform 6092. This object allows, but is not limited to, filtering out certain values, adding new columns, dropping unnecessary columns, and flattening child arrays. Due to the nature of the data coming from various source systems, with their own constraints for data-integrity, it is expected that some of the data will not be in a state where it can be loaded into the reporting storage. These data, dirty data, must be scrubbed and cleaned, or be discarded for later analysis. If the data has been discarded, an alert is sent, within the reporting system that data has been dropped and inserted into the dropped data table and requires investigation for the reporting developers. If the data can be scrubbed, then it is transformed in order to meet the minimum constraints of the reporting database. Once the data is sufficiently clean, rows in the proprietary table object which contain child data in arrays, must be exploded, or flattened, to create additional rows to be inserted.

Consider the following:

A table is defined to have two columns, X and Y

The table contains one row with values of [{I X:1, Y: [1,2]}]

The table is then flattened, specifying Y as the column to flatten

The output of the table is now two rows [{X:1, Y:1}, {X:1, Y:2}].

If the table contains multiple arrays, it may be necessary to flatten the table multiple times. Once the table has been appropriately flattened, columns may be Renamed, Dropped if they are not needed and/or Added with static values including, but not limited to, batch ID, customer ID, etc. or with dynamic lookup values.

After the data has been transformed, it is then inserted into the reporting database 6090 to be used in the report execution process which queries two sources, the reporting database 6090 and the client storage 6086 which contains the most recently cached flowsheet encounter data in order to display the results in the UI on the end-user computer 6096, for example.

During use, an Interface a user is enabled to select a report from a dropdown list. Once the report has been selected, the UI will make an HTTP request for the available parameters for the report specified from the UI API. FIG. Examples of the parameters which can be used include, but are not limited to, procedure codes, diagnosis codes, number of days since a procedure occurred, clinical data elements, and next appointment status. After the UI receives the response to the HTTP request, a list of parameters is then displayed. This list allows the end-user to dynamically add parameters for the specified report. Those skilled in the art will appreciate the ability to select multiple parameters using Boolean logic to specify whether all the parameters must match (AND Logic) or only some of the parameters must match (OR Logic). It is known that not all the parameters are required but there will always be at least one required parameter. Until all the required parameters have been added and set by the end-user, the report cannot be executed.

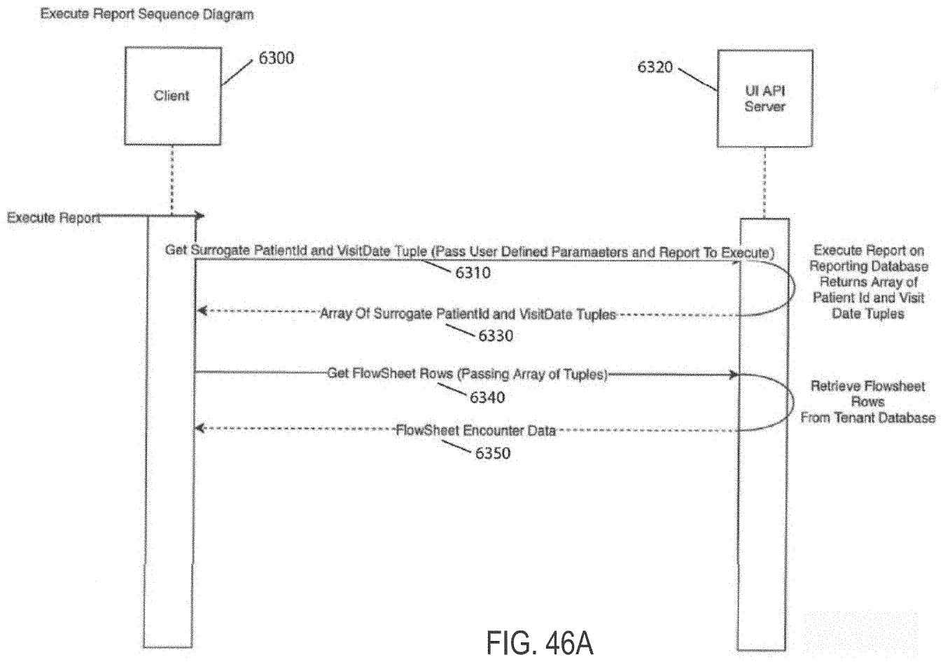

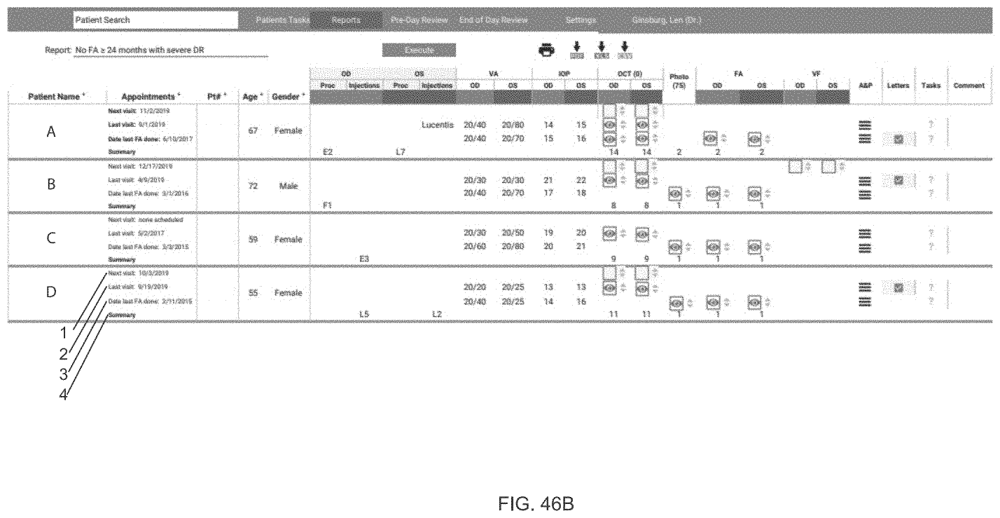

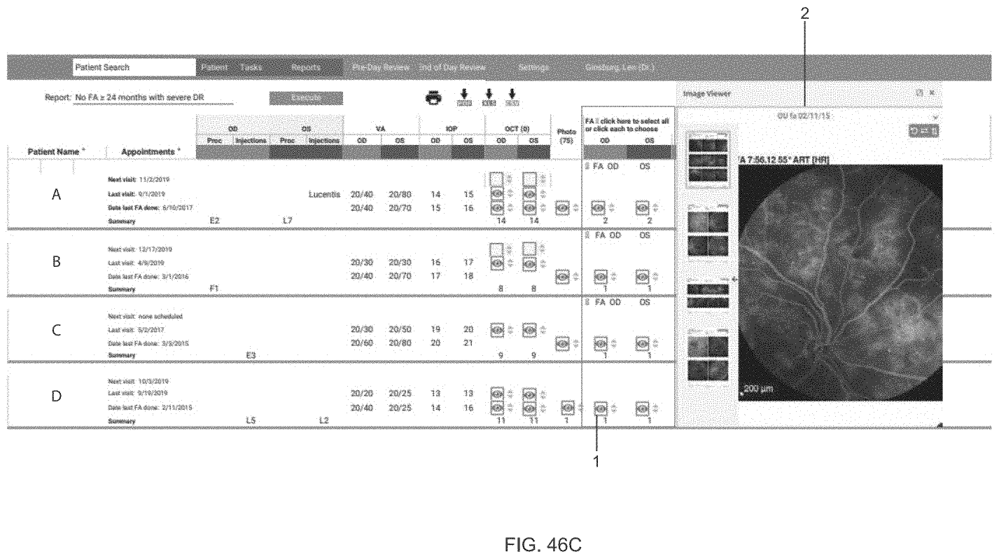

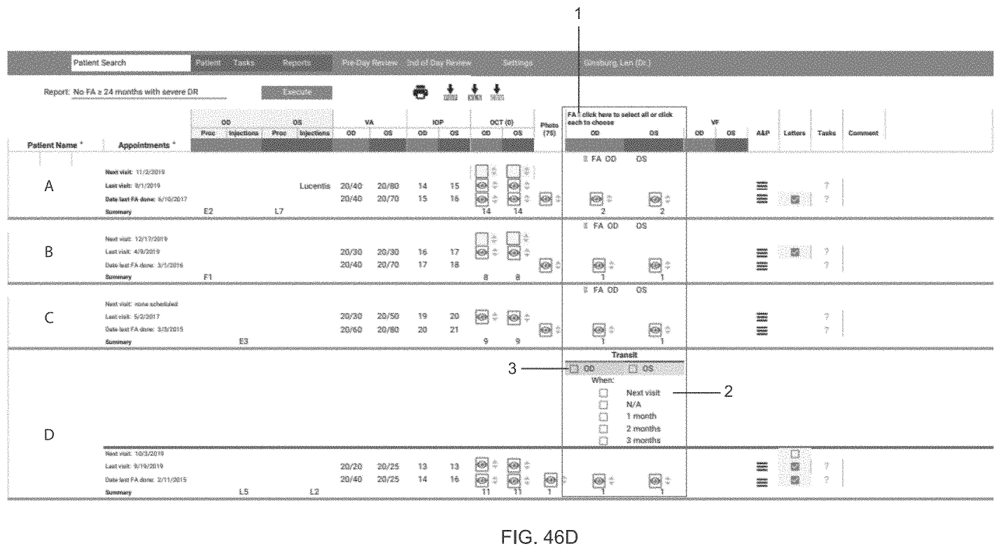

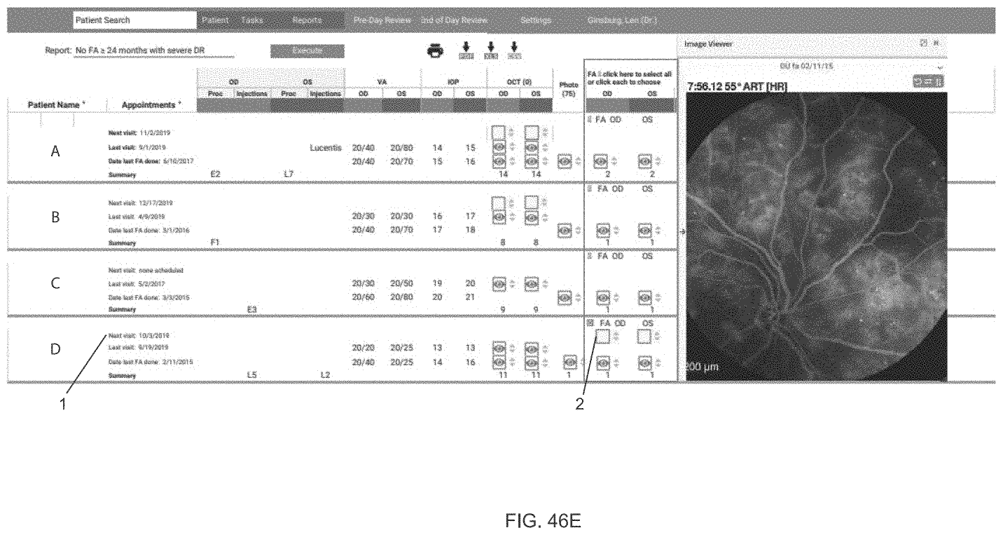

FIG. 46A depicts a sequence diagram for executing a report in accordance with an embodiment of the present principles. Once all the required parameters have been added, the end-user (client 6300) may then click a button to generate, or execute, the report HTTP request with the user-defined parameters. After the button to execute the report has been clicked, the UI app of the client 6300 will make an HTTP request 6310 to a controller in the UI API server 6320. The controller is responsible for setting the correct customer ID context, ensuring that the current user has access to the specified report, and calling the correct Stored Procedure in the Reporting RDBMS 6084 of FIG. 45A. The Stored procedure executes a series of queries and returns an array of Visit Date and Patient ID tuples 6330. Once the Stored Procedure has finished execution, the array of Visit Date and Patient ID Tuples is returned to the UI App. The UI App then makes another HTTP request 6340 to another controller in the UI API passing the list of Visit Date and Patient ID Tuples. The controller then executes a stored procedure on the Client Database which returns Flowsheet Encounter Data filenames 6350 for the specified visit dates. Once the list of filenames are returned to the controller, the application will loop through all of the unique patient IDs in the array of tuples that was passed as a parameter, and load the encounter data using the specified filenames retrieved from the stored procedure. Once the encounter data has been loaded from the filesystem, a summary row is generated which contains summary information for all columns specified in the flowsheet. An example of summary information could be the number of injections, by type, listed in the procedure column. Another example of summary row information could be for a patient's Visual Acuity (VA).

It is often helpful to know what a patient's best VA or worst VA over the course of care. The summary row would analyze all the historical data for a patient and insert the patient's worst VA and best VA into the summary row allowing the provider to view and evaluate years' worth of data in seconds. In order to evaluate years' worth of data without a summary row, a Clinical Professional would need to undertake the time consuming task of reviewing all Vision Tests, or Refractions, a patient has had, often having multiple refractions per visit, sometimes over the course of 10+ years of treatment. Finally, the API will then return an array of flowsheet data to the UI app which represents the group of patients which match the report type and user-defined parameters.

The UI app will then process the flowsheet data and render the relevant data in the appropriate rows and columns as shown, for example, in FIG. 44. The columns that are displayed are configurable based on the type of procedures, diagnoses, next appointment status or date, etc., selected as parameters for a report. Based on the parameters specified, an HTTP request will be made to the Flowsheet configuration controller, which will return the flowsheet configuration which contains the columns to display. This allows the Clinical Professional the ability to only see the most relevant data for the group of patients that they may be reviewing.

Ordering:

YOU can order the next visit or today's visit hit complete then you go to the next visit hit complete then the next follow-up hit complete or you can order all four and then hit confirmed at the end because as we order, a new roll pops up populating instantly as the doctor orders so if there's a mistake they can correct which incidentally you need in the ordering a reverse because what if the doctor accidentally hits the wrong drug and wants to change their mind.

FIG. 46B, 46C, 46D includes a mockup of one example of how the clinical actionable report could function.

FIG. 46B shows, is an actionable report in regards to taking the most important rows, which each report before its created, the creator would have to identify which rows of a patient makes sense. In this case, the doctor needs to know when the next visit is (1), what the last visit was (2), and then most importantly the row that identifies the last time (3) that particular visit the diagnostic test was done. Then, the fourth is a summary row (4), so in context the doctor has all of the information that they need to decide what to do with that patient. As always, the plan can be pulled up and, so could images.

FIG. 46C shows an example of how an image is now pulled up (2). The doctor is able to look at the fluorescein (1) from Feb. 1, 2015, on this patient, and decide if they want another fluorescein, the subject of this particular report.

FIG. 46D shows, an ordering mechanism (1) now that the doctor has made decisions on what to do on an individual patient since on all of these patients have a common theme could order for all of them. Now in context, the doctor can to order the diagnostic test to be repeated “next visit” (2) or another time. The doctor can actually take action from the report without leaving the screen.

FIG. 46D shows an example of when the doctor clicks on the fluorescein and the rows expand giving room to make the order of next visit, or as soon as possible, or any time period and which eye in this particular case is the first eye photographed, right (3) or left.

FIG. 46E how's the doctor choosing the test and then suddenly, since the next visit (1) has already been scheduled in this patient's case, an empty box (2) representing a fluorescein will now be done on that day. When the patient shows up on that next visit, a fluorescein will be done.

This is but one mechanism of how this can work. But, for the first time, doctors can view reports in context, make medical decisions and then actually order right without leaving the screen. Naturally, if doctors want more information, by hitting the patient's name it will go back into that patient's tool, so the entire history of that patient is known.

FIGS. 46B, 46C, 46D, 46E only considers a retina Doctor, but every specialty in medicine dentistry and Veterinary medicine will have different tests and the tool will show the Doctor in the column, row, panel, pop up or any other method in context what makes medical sense for that particular laboratory, pathology, radiological chemistry testing and procedures and injections of every kind.

Incidentally, the same type of mechanism is true for tasks. Not needed for billing tasks, because the billing department, as I understand, would not bother to look at those rows anyway. But, clinical tasks, whether they are auto generated or otherwise, chief technicians can at least benefit and understand what the issue is on a task, as could doctors. For instance, if it is a task that says a diabetes letter has not been sent, by the entire row shows it is missing, and in that case, it should also be the last time a letter was sent. Those two rows would come up, the doctor can see the task and then decide if they want to send the letter or if it is a case of where a patient misses a postoperative visit in a high-risk procedure, showing the missed postoperative visit the day that the injection or surgery was performed, that row also showing. Suddenly surgical schedulers can know how risky this patient is and schedule them appropriately, and in context understand the situation.

So, to doctors could also decide how important it is. So, these same mechanisms are for both tasks and reports as an option.

Currently when a doctor or practice want to generate a report, they are usually canned reports and the way data is stored, it is very specific and limited in what can be reported, especially clinical reports. You cannot ask a complex question like, “Report all patients in the practice with severe diabetes who had a laser or injection, but did not have an angiogram in two years.” There is no system that can create such a report. We can do this easily.

Let's go one step further. Reports traditionally give the name of the patient, their medical record, gender and may list when the last test that is being searched in the report was done. But doctors do not use those reports, usually managers look at it, because who has time to go into the chart and try to figure out what is going on? Our actionable report brings the chart to the doctor and gives enough rows of visits to allow the doctor to make all decisions. Which rows (date of service) to present to the physician in the report, is defined before the report is generated.

We then go to an even further step. Now that the doctor knows what they want to do, why should they have to go into multiple screens to place an order. Now a doctor can order in context! Also, instead of going to each individual patient, you can do multiple patients who all meet a certain criterion.

FIG. 46B represents the important dates of service and what happened that day taken from the individual tool, we already have created a summary row of all that has transpired, are presented to the doctor.

FIG. 46C shows even images (2) and other information can be pulled up (1) for each patient, so a decision can be made by the doctor without leaving the screen.

FIG. 46D shows one method where now that a doctor has made a decision to order a test (1), which has not been performed in two years (that was the subject of the report). Now in context, the doctor can order the diagnostic test to be performed “next visit” or another time. The doctor can actually take action from the report without leaving the screen. The rows expand giving room to allow the doctor to order next visit or any time period and to select which eye is to be photographed.

FIG. 46E shows the doctor after ordering the test, an empty box (2) representing a fluorescein will now be done on that next visit.

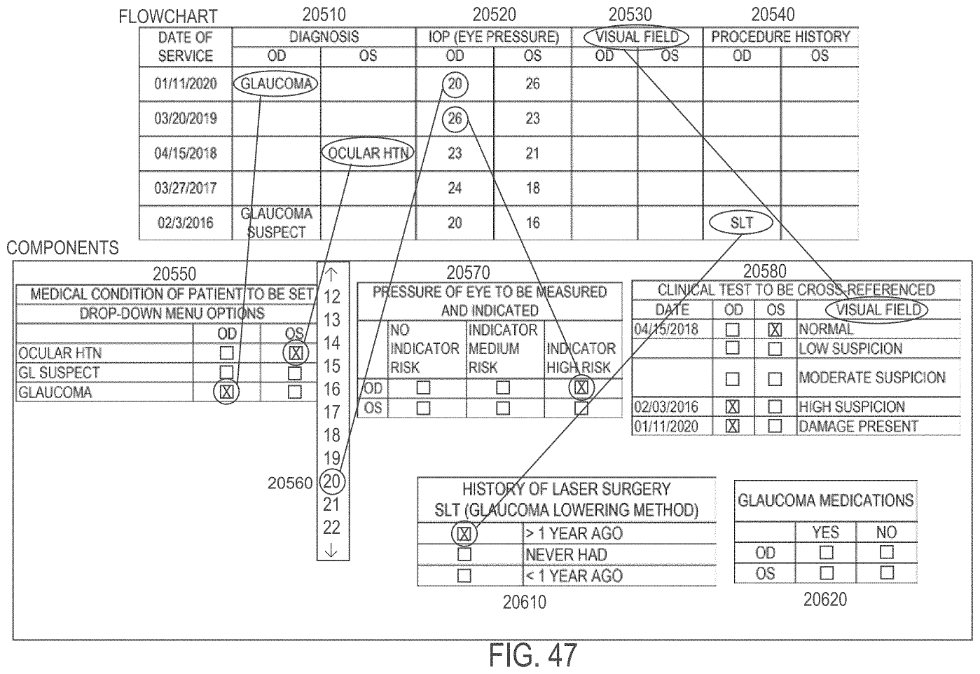

In some embodiments, a Data Command Center of the present principles enables the configuring of alerts in, for example, a medical records dashboard of the present principles. FIG. 47 depicts an embodiment of a medical records dashboard of a Data Command Center in which alerts and tasks can be performed in accordance with an embodiment of the present principles. Indicators such as diagnosis 20510, key results 20520, diagnostic tests 20530, and procedures 20540 are all key factors which may be used to trigger an alert or to send an automated task. In the diagnosis column 20510, glaucoma is indicated for the right eye (OD) and Ocular HTN is indicated for the left eye (OS) as required within the medical conditions dialog box 20550 as triggers. Thresholds are selected from a dropdown menu for key results 20560, for the results column 20520. Visual indicators may be selected 20570 to alert users of a threshold being met, exceeded, or falling below the specified level. A task panel 20590 may be utilized to set parameters at which an automated task will be sent and may involve triggers and time delays as to when the task will be sent 20600. Clinical tests may be validated and cross-referenced 20580 against a variety of conditions 20550, thresholds 20560, events 20610, and relevant factors 20620 to intelligently determine if an alert or task would be created.

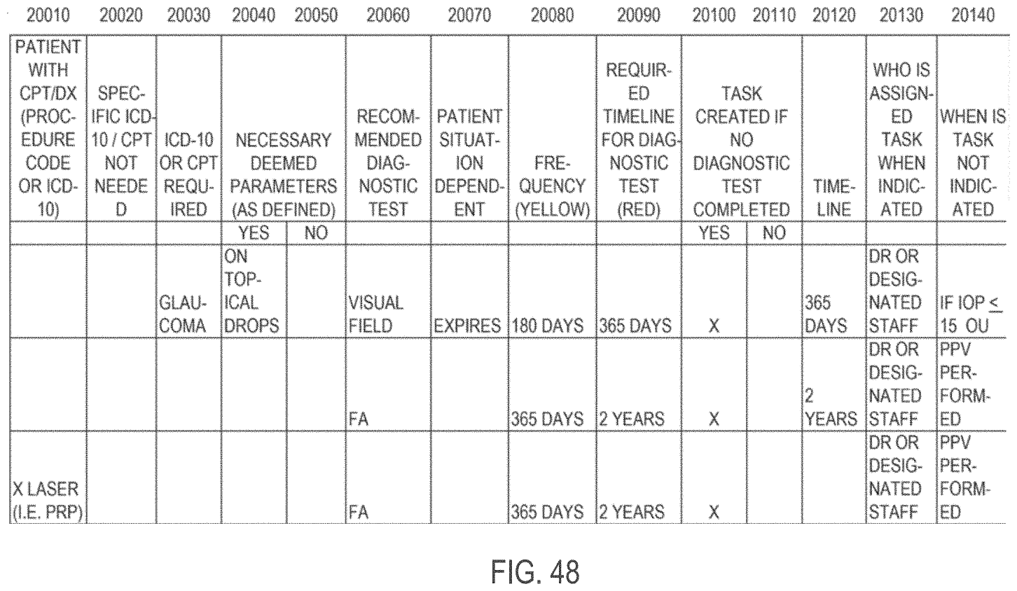

FIG. 48 depicts a menu for pre-configuring alerts in accordance with the present principles. That is, in some embodiments, alerts can be configured ahead of time or at point of care for individual or groups of patients. An example of configuring an alert is shown in FIG. 48. Parameters are defined such as including specific diagnostic or CPT codes 20010, ignoring specific diagnostic or CPT codes 20020, required specific diagnosis or CPT codes 20030, parameters deemed necessary which may be necessarily true 20040 or false 20050, recommended diagnostic tests 20060, patient dependent situations 20070, defined by frequency in yellow 20080, have a time interval within which to be performed in red 20090, tasks may trigger if required diagnostic test is not completed 20100/20110, be aligned to a timeline 20120, have tasks auto-generated to a specific person or group 20130, and exceptions which prevent tasks from sending 20140.

In this example, a Laser or Injection can be a trigger in the first column 20010. Glaucoma and Glaucoma Suspect would be required parameters in the third column 20030. The patient being on topical drops is a requirement deemed necessary in the first half of the fourth column 20040. Visual field, photo, and FAs are recommended tests in the fifth column 20060. Expiry and expiry based on seeing a retina specialist are examples of patient situation dependencies 20070. Frequency may be defined in yellow for any time increment 20080. Required Timeline may also be specified in red for any time increment (20090). Tasks are specified to be created or not based on completion of said diagnostic test 20100. Timeline may be specified in any time increment (20120). An individual or group is chosen as assignee 20130. Finally, the task trigger may be ignored based on specific criteria such as an IOP threshold or action taken 20140.

In accordance with the present principles, substantially any portion of a display, such as a medical records dashboard of the present principles, can be configured for an alert, including, but not limited to column headers and summaries, individual values within rows and/or columns, whole rows and/or columns, module headers, values displayed within modules, whole modules, and general application alerts outside of rows, columns, and/or modules. In some embodiments, alerts can be configured in accordance with Clinical Trials. Clinical trials exist throughout medicine often to determine efficacy of treatment and can include any number of other validations. As defined, alerts and auto-tasks can be configured for individual patients or groups of patients. In the specific case of clinical trials, a group of patients can be defined with specific criteria. As alerts can be configured by condition, procedure, risk factors, demographics, and a variety of other reasons, alerts can be used to isolate a group of patients deemed eligible for clinical trials.

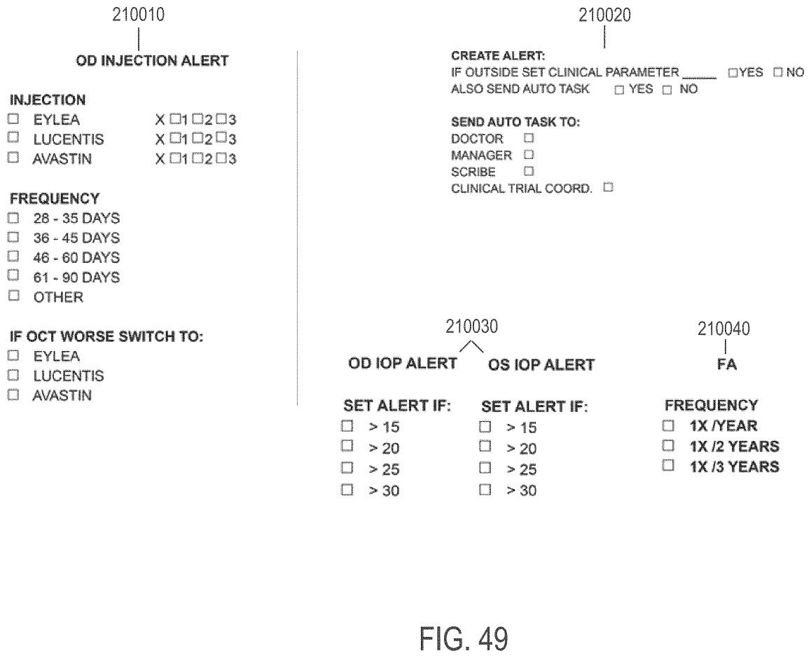

Often, clinical trials are based on medication efficacy. FIG. 49 depicts an embodiment of a medical records dashboard in which alerts are configured based on medication. As seen in FIG. 49, alerts can be configured based on medication, instance count of medication, frequency of medication, and can also have secondary or tertiary responses to the outcome 210010. Alerts can be configured based on defined parameters for clinical trials and may send automated tasks in the case of a patient or patients falling outside defined parameters 210020. Results can have predefined thresholds to trigger alerts and/or automated tasks 210030 and procedures, diagnostics, or lack thereof, can also trigger an alert or automated task. Additionally, required actions such as a diagnostic test 210040 can be required within a specific amount of time. One skilled in the art will appreciate that the ability to preconfigure rules, alerts, notifications, and tasks, in accordance with clinical trials is an example which can be applied across multiple areas of research and healthcare. The inclusion of financial decision support also expands this functionality to be of interest to pharmacological, insurance, and other entities and organization to denote key requirements for said entities, whether for a single patient, group of patients, group or groups of patients within specific parameters.

In some embodiments, if a patient misses an appointment, an auto task can be generated to alert a user/medical care provider schedule that the patient missed the appointment so that another appointment can be scheduled. or to the Clinical trial coordinator if it is part of a research protocol. Even parameters of when to create the task such as two missed appointments in a row can be set. This enables automatic tracking and a user/medical care provider can set it knowing the unique individual issues with a patient and can determine how important a missed appointment might be for a particular patient at a glance by showing previous data projected in the background or through one user interface on another monitor the user/medical care provider can cross check and individualize the alerts and tasks since a single missed appointment may be serious for one patient, but not so serious for another. Even parameters of when to create the task such as two missed appointments in a row can be set.

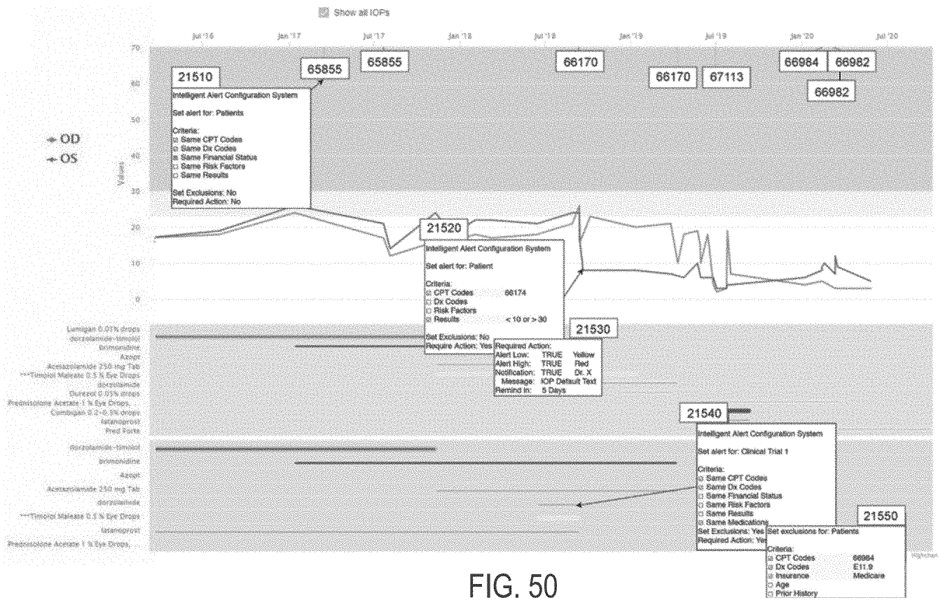

An intelligent alert configuration system, in one embodiment, can be represented in multiple ways, based on several criteria. For example, FIG. 50 depicts three different representations of an intelligent alert configuration system overlayed upon several different aspects of an application in accordance with an embodiment of the present principles. The alert configuration system of FIG. 50 can, in the case of a procedure 21510, display parameters associated with procedures, patients with like procedures, correlations to diagnoses, financial status, risk factors, results, or other relevant criteria, while enabling for exclusion criteria and actions to be taken, while also being able to be assigned to a patient, group of patients, all patients, or a patient or group of patients with specified criteria. In the case of launching the intelligent alert configuration from a result 21520, a different set of parameters can be specified relevant to that result, patients with similar results, correlations to diagnoses, financial status, risk factors, results, or other relevant criteria, while enabling for exclusion criteria and actions to be taken, while also being able to be assigned to a patient, group of patients, all patients, or a patient or group of patients with specified criteria. An example of required actions 21530 denotes several key actions which can occur upon triggering the alert. These include, but are not limited to, a visual or audio alert, tiers of alerts based on values, notifications to be sent and to whom, reminders are to be sent. In the case of launching the intelligent alert configuration from a medication 21540, a different set of parameters can be specified relevant to medications, patients with similar medications, correlations to diagnoses, financial status, risk factors, results, or other relevant criteria, while allowing for exclusion criteria and actions to be taken, while also being able to be assigned to a patient, group of patients, all patients, or a patient or group of patients with specified criteria. An example of exclusions 21550 denotes several key actions which can exclude a patient or patients from an alert.

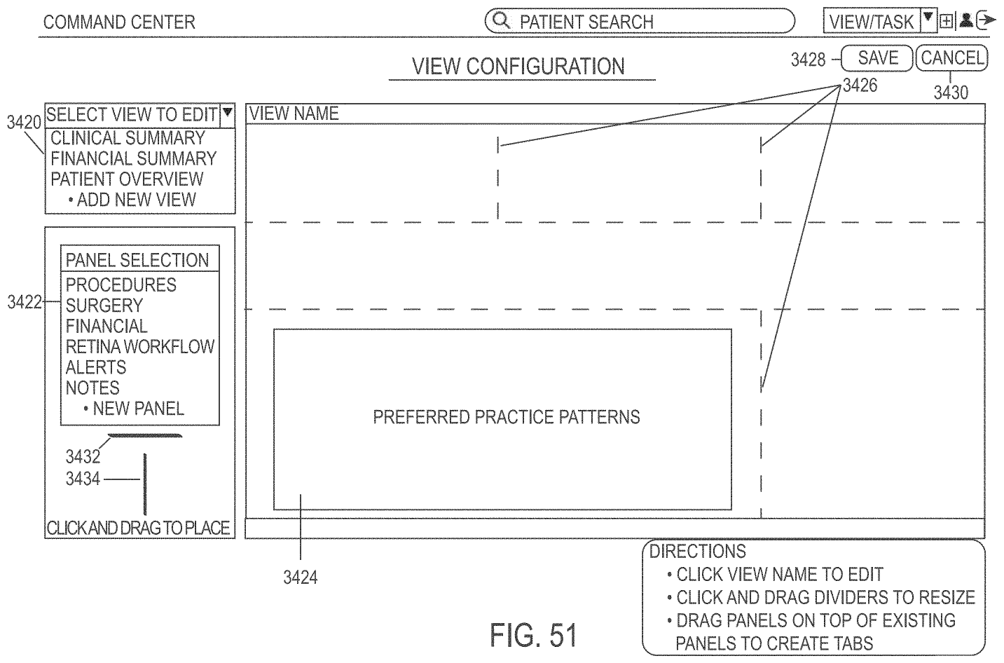

FIG. 51 depicts a view configuration page in accordance with an embodiment of the present principles. The View Configuration page presented in FIG. 51 provides the user a mechanism to create or modify dynamic dashboards to accommodate their unique requirements. These dashboards are referred to herein as Command Center Views. The user identifies the view to be created or modified by selecting the appropriate option from the drop down menu 3420. Once the view has been selected, the user will select a panel to place in the view from the list 3422. Once the panel has been anchored in the View 3424, the dimensions of the panel may be adjusted by means for manipulating the panel frame(s) 3426. When the user is finished creating or modifying the view, the user may save (3428) or cancel (3430) their actions by selecting the associated buttons. Additional horizontal dividers 3432 and vertical dividers 3434 can be dragged and dropped onto the View in order to create new display panels. The dividers also can be removed by clicking on the desired divider and dragging it off the View.

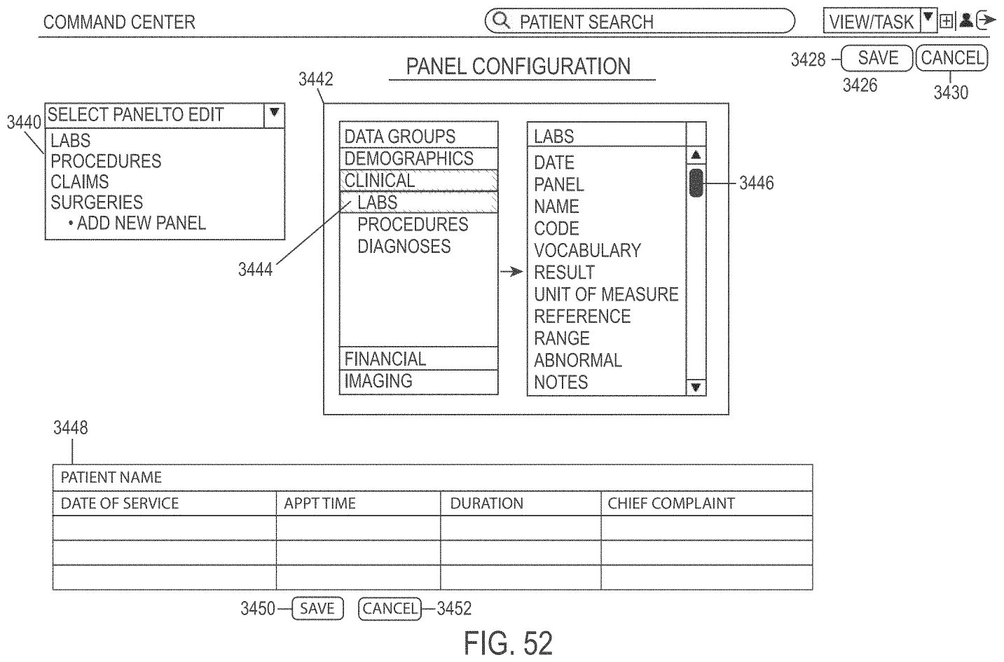

FIG. 52 depicts a view configuration page in accordance with another embodiment of the present principles. The Panel Configuration page presented in FIG. 52 provides the user a mechanism to create or modify display panels to use when populating medical records dashboards of the present principles. The user identifies the panel to be created or modified by selecting the appropriate option from drop down menu 3440. The user then selects data to populate the panel from the Data Selector 3442. Available data 3444 is selected and migrated to the panel's Column List 3446. Users may also add custom fields allowing them to track any data relevant to the user. A preview of the panel 3448 is presented and updated as the user makes changes. When the user is finished creating or modifying the panel, the user may save 3450 or cancel 3452 their actions by clicking the associated buttons. The panels from the drop-down menu 3440 can provide a template for creating a customized Data Command Center in which all information desired by the user can be accessed without leaving the Data Command Center.

In some embodiments, the Data Command Center medical records dashboard can be characterized by an ability to present large volumes of dynamic data in a single display interface whereby the data can be navigated without leaving the screen. For example, the data is either available in a display window, behind a tab, or available via a pop-up window and is thus accessible without leaving the display. In some embodiments, to enable such processing to occur without unacceptable delay in data presentation, and to enable the display panels and flowsheet panels to be reconfigured as described, a Data Command Center architecture provides for flexibility in storage and presentation of dynamic data as well as dynamic caching techniques that allow for prompt presentation. Two-way auto-population techniques can be implemented whereby changes made in the Data Command Center are not only reflected in the display but also the associated electronic medical record is auto-populated as well without leaving the Data Command Center display screen.

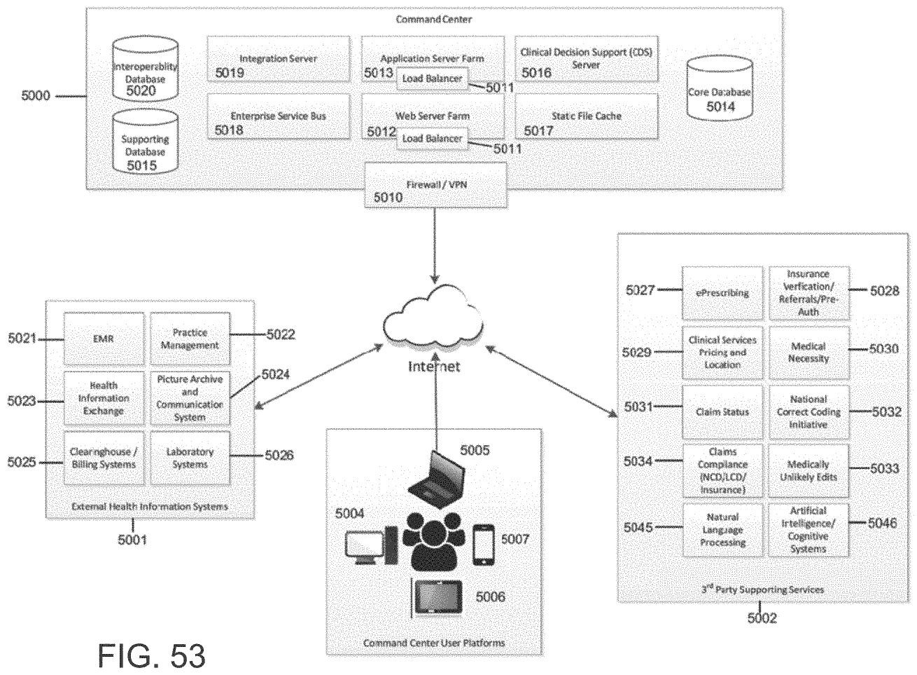

FIG. 53 depicts a Data Command Center architecture 5000 and connectivity to external Health Information Technology systems 5001 and third party services 5002 in accordance with an embodiment of the present principles. The Command Center architecture 5000 is a multi-tenant cloud-based web application. As known by those skilled in the art, multi-tenant means that the application is deployed once and all customers access the same server. Data is segregated by the application so that customers can only access their own data. The Data Command Center is accessible over the Internet via user platforms 5003 that implement a modern web browser (e.g. Internet Explorer 10, Google Chrome) on a desktop computer 5004, laptop 5005, tablet 5006, or a smartphone 5007 with internet connectivity.

The Command Center architecture 5000 illustrated in FIG. 53 is one embodiment of how the system can be configured to support a large user base. For security purposes, access to the cloud platform containing the Command Center is governed by a firewall and Virtual Private Networks (VPNs) 5010. Additionally, end to end encryption is an inherent part of the Command Center architecture 5000 as the Command Center architecture 5000 is optimized to meet the highest privacy and security expectations. Each component allows for both the application of current high strength encryption (ex. AES 256) as well as continuous implementation of evolving data protection and security best practices. The Command Center architecture 5000 configuration supports proven high-availability and disaster recovery practices, including fully encrypted off-site backup and redundant, geographically dispersed operations. Load balancers 5011 distribute the client requests to the most available web server in the web server farm 5012 to optimize response time. The web servers in the web server farm 5012 pass requests through the load balancer 5011 to the application server farm 5013 to continue processing the client requests. The application servers of the application server farm 5013 process the requests and access data from the core database 5014 and supporting database(s) 5015. Depending on the client request, the application servers may interact with the Clinical Decision Support Server 5016 where the primary business logic resides. Once processed, the web server returns the results of the client request back to the client for display. Static files are optimized for retrieval by residing on a dedicated server (static file cache) 5017 where they are cached and optimized for this purpose.

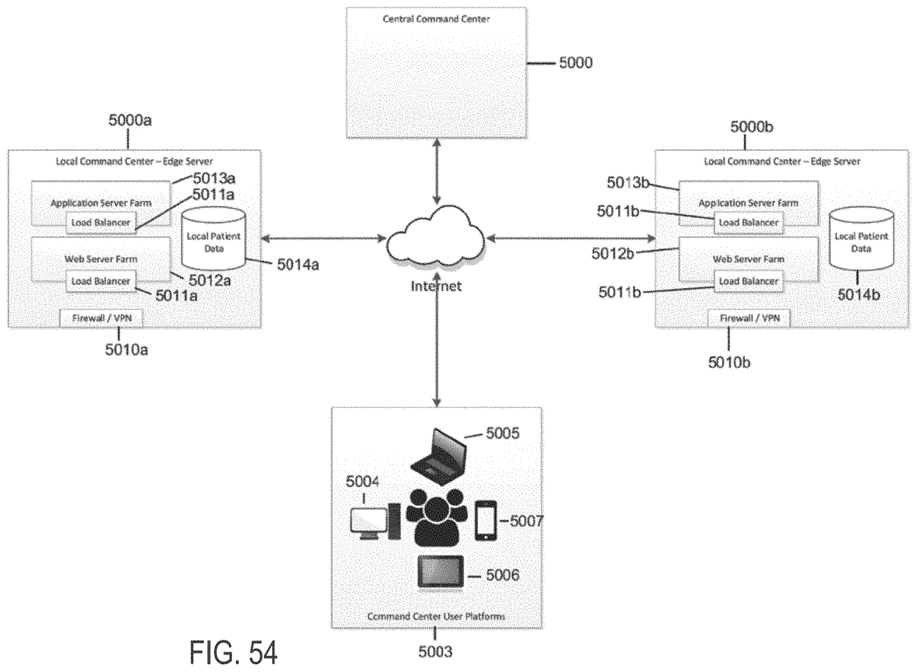

The exchange of information between external health information technology (HIT) systems 5001 and the Command Center 5000 is managed by dedicated servers designed to optimize system throughput and secure communications. All communications take place through a secure VPN 5010 connection. If the integration method is message-based, the sending HIT system 5001 will transmit messages to the Enterprise Service Bus 5018. However, if communication is based on an API standard, the sending HIT system 5001 will communicate with the Integration Server 5019. Both of these servers 5018, 5019 communicate directly with the Interoperability database 5020 and, in turn, with the core and supporting databases 5014, 5015. While communications with third party services 5002 are largely outbound from the Command Center 5000 to the services, it is possible to receive inbound communications. These third party communications will also be handled through the Enterprise Service Bus 5018 and the Integration Server 5019. As illustrated in FIG. 54, examples of HIT systems 5001 include Electronic Medical Records (EMRs) 5021, Practice Management Systems 5022, Health Information Exchanges (HIEs) 5023, Picture archiving and communication systems (PACS) 5024, claims-based systems such as Clearinghouses and Insurance Companies billing systems 5025, and Laboratory Systems 5026.

In exemplary embodiments, many third party supporting services 5002 are integrated to provide feedback and advice. Examples of these services include ePrescribing 5027, Insurance verification including referrals and pre-authorizations 5028, clinical pricing and location services 5029 used to find the best value on purchasing medications, procedures and imaging services, medical necessity checking 5030 to verify a procedure or medication is associated with a correct ICD10 code supporting its use, claim status checking 5031, services in support of the National Correct Coding Initiative 5032, Medically Unlikely Edits 5033 provided by Center of Medicare and Medicaid Services (CMS) to proactively ensure claims are coded correctly to prevent issues in billing, and claims compliance services 5034 which evaluate claims against CMS National Coverage Determination (NCD) and Local Coverage Determination (LCD) guidelines as well as local insurance regulations all in an effort to establish and document medical necessity and to document same in support of streamlined billing. Natural language processing program 5045 and artificial intelligence/cognitive systems 5046 may also be provided to, for example, provide clinical decision support features. In exemplary embodiments, the NCD and LCD guidance is programmed into the Command Center 5000 so that alerts may be generated when a physician attempts to follow a treatment protocol that is non-compliant with the NCD and LCD guidance.

Those skilled in the art will appreciate that data latency may be improved by storing the data in the static file cache 5017 in the server of the distributed network of servers that is closest to the geographic location of the patient's appointment. In an exemplary embodiment, the server closest to the geographic location of the patient's appointment could contain data only for today's patients so that there is less data to query, thus improving the access speed for the data. Also, any data that is not stored locally may be cached locally after it has been accessed for the first time as it is more likely to be accessed a second time, thereby speeding up the data access. This architecture implements Proximity Request storage whereby data accessed most frequently is stored geographically closer to the user to reduce the time it takes to travel over the Internet. This approach is used by Netflix and others when hosting large movie files. In the present case, the most relevant patient data is stored within proximity of where it is stored. Relevant patient data is for patients that have been accessed in the past few days and any patients with an upcoming appointment. Having a smaller local subset of data makes the whole network operate more efficiently.

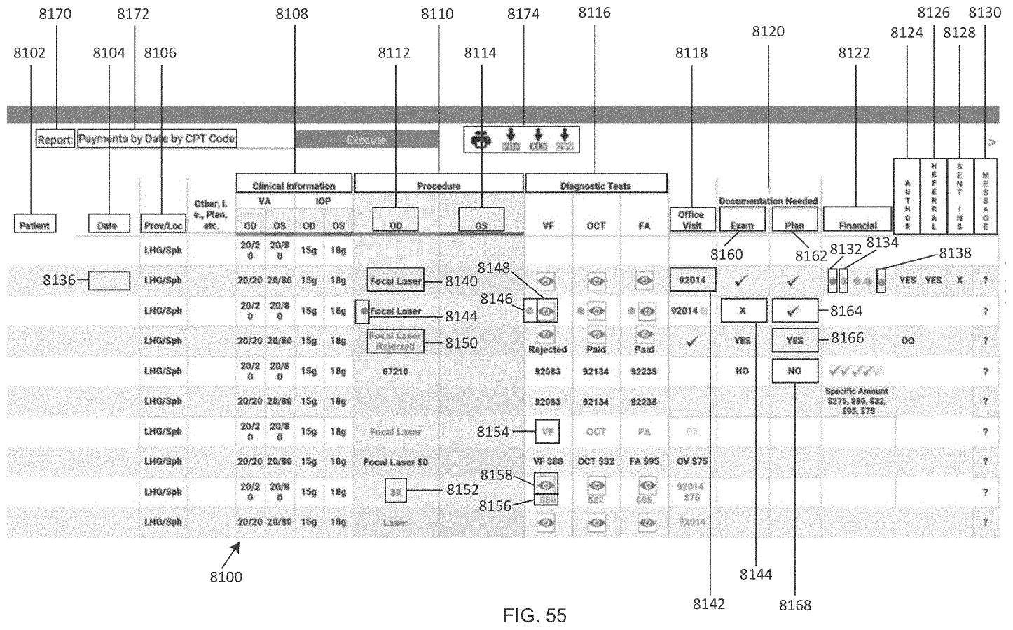

FIG. 55 depicts a medical records dashboard of a Data Command Center of the present principles that enables a healthcare provider while delivering medical care to a patient to participate in revenue cycle management in accordance with an embodiment of the present principles. Without interrupting medical care, the healthcare provider may understand what things cost, whether they have been authorized to do something like a procedure, and whether the previous claims were rejected or paid. Critical compliance issues of properly following the laws can now be often confirmed because if the healthcare provider was paid for something and the services was not performed, the failure to perform the service would be detected. The billers know when charges are paid or rejected and the corresponding diagnosis and CPT codes, but this embodiment correlates the financial with clinical as the doctor is seeing the patient without interrupting the patient flow.

The display 8100 shows a particular patient's name or the name of a number of patients at 8102. The date of a particular encounter is shown at 8104. The patient data can be seen over many encounters listed in the different rows. The provider who delivered the service as well as the location of the provider who saw the patient is provided at 8106. The clinical information that can be displayed simultaneously for the doctors to be able to care for the patient is provided at 8108. This clinical information is different for all medical specialties. Eye doctors might look at vision. Family doctors might look at blood pressure or blood sugar.

A column of procedures 8110 that were performed are defined that need to be measured by specialty. Every specialty might have different procedures and the tool can have multiple columns for different procedures. 8112 and 8114 demonstrate that a procedure can be on different sides of the body. 8112 represents the right (in eye care—OD) side while 8114 represents the left side of the body (in eye care—OS). In orthopedics, procedures of the right knee and left knee could be populated.

8116 provides an example three types of different diagnostic tests usually performed and billed in an eye doctor's office (e.g., VF, OCT, and FA) but any diagnostic test or other CPT code could be in these columns. 8118 is the office visit that was charged as there are different levels of office visits that a provider can charge at different times. 8120 illustrates whether proper documentation was needed or not needed to be able to bill any of the procedures, diagnostic tests, or office visits.

A financial column 8122 may include an authorization column 8124 where providers as they are seeing patients can actually know whether or not the insurance company has authorized the procedure. A referral column 8126 may indicate whether the proper referral from a family doctor to a specialist was received. A sent insurance column 8128 may be used to indicate whether insurance information has been received for the patient, while message column 8130 may provide messages or tasks that manually or automatically can be created by the user or anyone in the practice to communicate to the user. Financial column 8122 may include many different ways of indicating to a doctor or provider the financial information. For instance, indicator 8132 could be red which could mean rejected, or green for paid, or yellow for partially paid. Another indicator 8134 that is associated with one of the other columns representing something performed and charged by the user that particular date 8136 may also be provided. A third indicator 8138 could be red, yellow or green to represent payments or express costs and corresponding to another of the charges for date 8136. 8140 demonstrates that a focal laser type surgery was performed on a particular encounter date. Indicator 8132 could be associated with the focal laser type surgery 8140 and represents, if red, that the insurance company rejected payment and nothing was paid. Indicator 8134 could relate to whether another item performed on date 8136 was paid. Similarly, indicator 8138 could correlate with whether office visit 8142 was paid.

Instead of all indicators of payment being on one row together in column 8122, the indicators could instead be in the column next to the medical service charged for. In addition, an indicator 8144 may show that there was rejection right next to the procedure, in this case the focal laser, as there is a red dot 8144 which correlates to zero payment. This type of indicator can instead of being all in one column 8122 also can be in each column associated directly with what was done as at indicator 8146 where icon 8148 shows that a VF was performed. Indicator 8150 shows the procedure focal itself can be red or even written ‘rejected’. Similarly, a zero 8152 may be used to show rejected payments. Indicator 8154 demonstrates that a VF was performed but could be highlighted green meaning it was fully paid or red if it was rejected. On the other hand, the amount paid 8156 could in the same cell next to, above or below what was performed, in this case $80.

The healthcare provider may also confirm that something was done or if they want to see the actual item being billed or to interpret it. For example, icon 8158 would allow the healthcare provider to click on it and actually the image itself comes up and an interpretation could be seen. If no image is attached, they might discover that it was not done and billed incorrectly, so the healthcare provider could elect to return the money to the insurance company. This decision may be made rapidly while examining a patient. Additionally, a lack of documentation may be indicated in column 8120. Because doctors often use scribes or assistants, they may have inadvertently not completed the chart. Column 8160 is an exam column, while column 8162 is a plan column. Perhaps they are completed or not, but instantly doctors can see whether a check mark 8164 has been provided to indicate that documentation has been completed. Alternatively, the word ‘yes’ 8166 or any other appropriate word or indicator may indicate that the documentation has been provided, while the word ‘no’ 8168 or other appropriate word or indicator may indicate that the documentation has not been completed. 8144 could be a ‘X’ noting that the indicated documentation was not completed. In this fashion, doctors instantly can be informed and take action to complete the chart.

Doctors may have an interest in running a report on any of the elements, columns or rows to identify payments in one patient or a group of similar patients. 8170 depicts the ability to run a report. The type of report that is requested may be indicated at 8172, where patient list 8102 may list a whole group of patients, perhaps sharing a common insurance company, but doctors can quickly make decisions because they have at their access not only the exact financial information, but in context for each individual patient can understand what was really done that day, how the patient's care was performed, what tests were or were not completed and whether they were paid properly or improperly or, from a compliance point of view not documented correctly as indicated in column 8120. The user may elect to export the data at 8174.

In sample embodiments, the indicators 8132, 8134, or 8138 could be selected to bring up a ledger that matches each CPT code individually with payments. Here the exact row of the date of service with all the CPT codes one or more done that day and payments can all be brought up.

In some embodiments, a method for rules-based data display in a data command center including a medical records dashboard including one or more windows including information received or derived from at least one patient database, the medical records dashboard comprising a display on a screen, using the one or more windows, of at least one of medical services, clinical data, examination findings, diagnostic tests, and the procedures performed on one or more patients, the one or more windows comprising a plurality of data entry fields, including at least one collapsible data entry field, for displaying the information received or derived from the at least one patient database, wherein the at least one of the medical services, the clinical data, the examination findings, the diagnostic tests, and the procedures are arranged in rows or columns on the screen according to at least one of a time and a date that the medical services, the clinical data, the examination findings, the diagnostic tests and the procedures were performed on the one or more patients, the method includes receiving patient-related data from the at least one patient database, comparing the received patient-related data with configuration rules to determine which portions of the received patient-related data are to be displayed in data entry fields of the medical records dashboard, identifying collapsible data entry fields of the at least one collapsible data entry field of the medical records dashboard that are determined to not have any patient-related data to display as collapsed data entry fields, displaying patient-related data in the data entry fields of the medical records dashboard in accordance with the configuration rules and collapsing data entry fields of the medical records dashboard identified as collapsed data entry fields.

In some embodiments, a data command center visual display system that displays data on a display screen includes a computing device comprising at least one processor, a non-transitory computer-readable medium, having stored thereon, software instructions that when executed by the at least one processor of the computing device, cause the computing device to perform operations comprising at least, linking to and receiving patient related medical records including patient data from at least one patient data source, and displaying a medical records dashboard including one or more windows, the medical record dashboard capable of displaying, using the one or more windows, patient data from at least one patient data source including at least one of medical services, clinical data, examination findings, diagnostic tests, and the procedures performed on one or more patients, the one or more windows comprising a plurality of data entry fields, including at least one collapsible data entry field, for displaying the information received or derived from the at least one patient database, wherein the at least one of the medical services, the clinical data, the examination findings, the diagnostic tests, and the procedures are arranged in rows or columns on the screen according to at least one of a time and a date that the medical services, the clinical data, the examination findings, the diagnostic tests and the procedures were performed on the one or more patients, wherein a display of patient data in the medical records dashboard is determined by: comparing the patient data with configuration rules to determine which portions of the patient data are to be displayed in the data entry fields of the medical records dashboard, identifying collapsible data entry fields of the at least one collapsible data entry field of the medical records dashboard that are determined to not have patient data to display as collapsed data entry fields, and displaying patient data in the data entry fields of the medical records dashboard in accordance with the configuration rules and collapsing data entry fields of the medical records dashboard identified as collapsed data entry fields.

In some embodiments, a method for unique patient identification of a subject patient in a data command center including patient-related data received or derived from at least one patient database includes collecting patient-related data having different data classifications from the at least one patient database, assigning a level of accuracy score for each of the patient-related data of the different classifications, adding, the level of accuracy scores for each of the patient-related data of the different classifications, comparing a total of the added level of accuracy scores to a previously determined matching threshold, if the total of the added level of accuracy scores exceeds the matching threshold, establishing an identification of the subject patient, and if the total of the added level of accuracy scores does not exceed the matching threshold, collecting additional patient-related data and returning to the assigning phase.

In some embodiments, a data command center visual display system for determining a unique patient identification includes a computing device comprising at least one processor, a non-transitory computer-readable medium, having stored thereon, software instructions that when executed by the at least one processor of the computing device, cause the computing device to perform operations comprising at least: linking to and receiving patient related medical records including patient data from at least one patient data source, collecting patient-related data having different data classifications from the at least one patient database, assigning a level of accuracy score for each of the patient-related data of the different classifications, adding, the level of accuracy scores for each of the patient-related data of the different classifications, comparing a total of the added level of accuracy scores to a previously determined matching threshold, if the total of the added level of accuracy scores exceeds the matching threshold, establishing an identification of the subject patient, and if the total of the added level of accuracy scores does not exceed the matching threshold, collecting additional patient-related data and returning to the assigning.

In some embodiments, a method for medication management and display in a data command center comprising one or more windows for display and including information received or derived from at least one patient database, the data command center displaying on a screen, using the one or more windows, at least one of medical services, clinical data, examination findings, diagnostic tests, and procedures performed on one or more patients, the one or more windows comprising a plurality of data entry fields for displaying the information received or derived from the at least one patient database, wherein the at least one of the medical services, the clinical data, the examination findings, the diagnostic tests, and the procedures are arranged in on the screen according to at least one of a time and a date that the medical services, the clinical data, the examination findings, the diagnostic tests and the procedures were performed on the one or more patients, includes determining, from at least one of the information received or derived from the at least one patient database and the at least one of the medical services, the clinical data, the examination findings, the diagnostic tests, and the procedures, medications administered to the one or more patients, generating a respective graphical representation for each of the determined medications administered to the one or more patients, and displaying at least one generated, respective graphical representation of at least one medication administered to a patient in the at least one or more windows in context with at least one of the information received or derived from the at least one patient database and the at least one of the medical services, the clinical data, the examination findings, the diagnostic tests, and the procedures, wherein the at least one generated, respective graphical representation of the at least one medication administered to the patient is arranged in on the screen according to at least one of the times and the dates that the at least one medication was being administered to the patient.

In some embodiments, a data command center visual display system that displays data on a display screen includes a computing device comprising at least one processor, a non-transitory computer-readable medium, having stored thereon, software instructions that when executed by the at least one processor of the computing device, cause the computing device to perform operations including at least, linking to and receiving patient related medical records including patient data from at least one patient data source, wherein the patient data includes at least one of medical services, clinical data, examination findings, diagnostic tests, and procedures performed on one or more patients, determining, from at least one of the patient data and the at least one of the medical services, the clinical data, the examination findings, the diagnostic tests, and the procedures, medications administered to the one or more patients, generating a respective graphical representation for each of the determined medications administered to the one or more patients, and displaying using the one or more windows, at least one of medical services, clinical data, examination findings, diagnostic tests, and procedures performed on one or more patients and at least one generated, respective graphical representation of at least one medication administered to a patient in context with at least one of the patient data and the at least one of the medical services, the clinical data, the examination findings, the diagnostic tests, and the procedures, wherein the at least one of the medical services, the clinical data, the examination findings, the diagnostic tests, and the procedures are arranged on the screen according to at least one of a time and a date that the medical services, the clinical data, the examination findings, the diagnostic tests and the procedures were performed on the one or more patients, and wherein the at least one generated, respective graphical representation of the at least one medication administered to the patient is arranged on the screen according to at least one of the times and the dates that the at least one medication was being administered to the patient.

In some embodiments, a method for a display of a graphical representation of complete medical history of a patient in a data command center comprising one or more windows for display and including patient-related data received or derived from at least one patient database, the method includes determining, from the patient-related data, a complete medical history of at least one patient including at least one of medical services, clinical data, examination findings, diagnostic tests, medications administered to and procedures performed on a patient, generating a graphical representation of the determined complete medical history of the patient including the at least one of medical services, clinical data, examination findings, diagnostic tests, medications administered to and procedures performed on the patient, and displaying the generated graphical representation in the at least one or more windows according to at least one of a time and a date that the at least one of the medical services, the clinical data, the examination findings, the diagnostic tests, and the procedures the medical services, the clinical data, the examination findings, the diagnostic tests and the procedures were performed on the one or more patients and at least one of the times and the dates that the medications were being administered to the patient, wherein a user is enabled to select a location in the displayed graphical representation and details regarding the at least one of medical services, clinical data, examination findings, diagnostic tests, medications administered to and procedures performed on the patient related to that selected location are presented to the user.

In some embodiments, the Data Command Center of the present principles, such as the Data Command center 001 of FIG. 1, can provide an in-context image management system configured to display at least one image as a result of a user action in the context of at least one patient's medical data. In accordance with the present principles, one or multiple images can be displayed in the context of one or multiple patients' data. Images can be tagged, interpreted, highlighted, defaulted, prioritized, enhanced, edited, resized, rotated, or otherwise altered and saved or not saved in the images' altered state. In some embodiments, images can be displayed and accessed directly from within rows and columns of patient data. Images can be displayed and accessed directly from thumbnails, either displayed within rows and columns of patient data, or displayed within modules within the context of other modules within the application. Additionally, rows, columns, images, and/or modules can be interacted with through a single interface displaying and enabling the editing of images and/or a patient or multiple patients' data.

In some embodiments of the present principles, in-context display of images and patient related information can begin with a retrieval of patient related information from at least one patient database or server. At least one medical record dashboard can then be displayed including one or more windows for displaying, using a single display interface, patient related information retrieved from or derived from the at least one patient database or server. In some embodiments, the patient related information can include at least one of medical services, clinical data, examination findings, diagnostic tests, and procedures performed on one or more patients. In some embodiments, the one or more windows can include a plurality of data entry fields for displaying the patient related information received or derived from the at least one patient database, such that the at least one of the medical services, the clinical data, the examination findings, the diagnostic tests, and the procedures can be arranged on a display according to at least one of a time and a date that the medical services, the clinical data, the examination findings, the diagnostic tests and the procedures were performed on the one or more patients. In such embodiments, at least one visual representation of at least one image related to the at least one of the medical services, the clinical data, the examination findings, the diagnostic tests, and the procedures performed on the one or more patients can be generated. That is, in some embodiments visual representations of images of at least tests and procedures performed on a patient are generated. In some embodiments, such visual representations can include icons providing links to an available image such that the icons can be displayed in the medical records dashboard and when selected provide links to the available images which can be displayed in a pop-up window concurrently with the displayed patient related information.

Alternatively or in addition, in some embodiments, the visual representations of the images can include thumbnails of the images represented. In such embodiments, a user is able to determine the contents of an underlying image from the displayed thumbnail which assists a user in selecting an appropriate image to view in greater detail or larger scale in, for example, a pop-up window. That is, in accordance with some embodiments, the generated visual representations of available images related to patient care can be displayed in at least one of the plurality of data entry fields, such that when a displayed visual representation is selected, a respective image is displayed concurrently with the patient related information on the display.

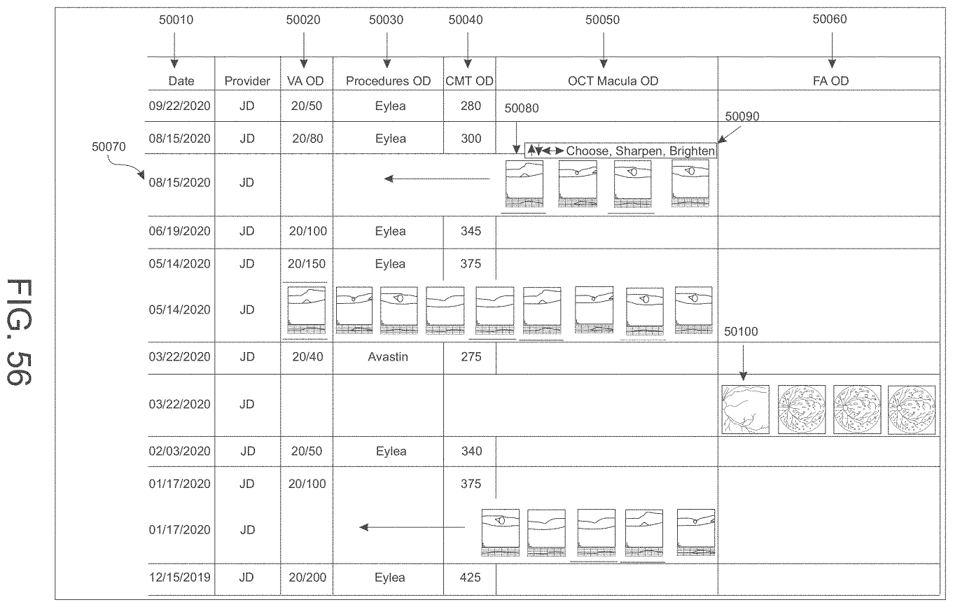

For example, FIG. 56 depicts an embodiment of a display of an in-context image management system displaying thumbnailed images in rows and columns in accordance with an embodiment of the present principles. In the embodiment of FIG. 56, the in-context image management system displays one image within the context of one patient. Patient data, consisting of, but not limited to, date of service, physician, and location 50010, key results 50020, procedures 50030, and additional results 50040, can be displayed alongside thumbnails of images of a single type 50050, or multiple image types 50060. Images can be displayed in rows comprising patient visits on a particular date of service 50070 or on multiple rows for multiple dates of service. Thumbnailed images can consist of one or multiple images for a single date of service 50080 and can enable for interaction with image editing tools to allow the image to be tagged, interpreted, highlighted, defaulted, prioritized, enhanced, edited, resized, rotated, or otherwise altered and saved or not saved in said images' altered state. Multiple image types can be displayed in subsequent columns 50100, and, in some embodiments, the rows and columns can expand to show a larger thumbnail or contract to show a smaller thumbnail. Thumbnails can also be directly accessed and enlarged for more concise review and/or editing.

In the embodiment of FIG. 56, the date of service 50010, provider (not labeled), visual acuity, procedures 50030, and central macular thickness 50040, are displayed alongside an OCT of the macula 50050 and fluorescein angiography 50060, although any combination of data and image can be substituted. In FIG. 56, the date of service 08/15/2020 50070 displays multiple OCT images 50080. The images can be altered through an image editing widget 50090 to flip, resize, sharpen, brighten, or otherwise alter the image or directly accessed for more editing options. For the date of service occurring on 03/22/2020, fluorescein angiography 50100 can be viewed alongside other images. In some embodiments, images of any displayable type can be accessed individually or in groups. When the existence of multiple images of the same type occurs on the same date of service, images can be configured to be displayed in a predetermined or selected order or priority.

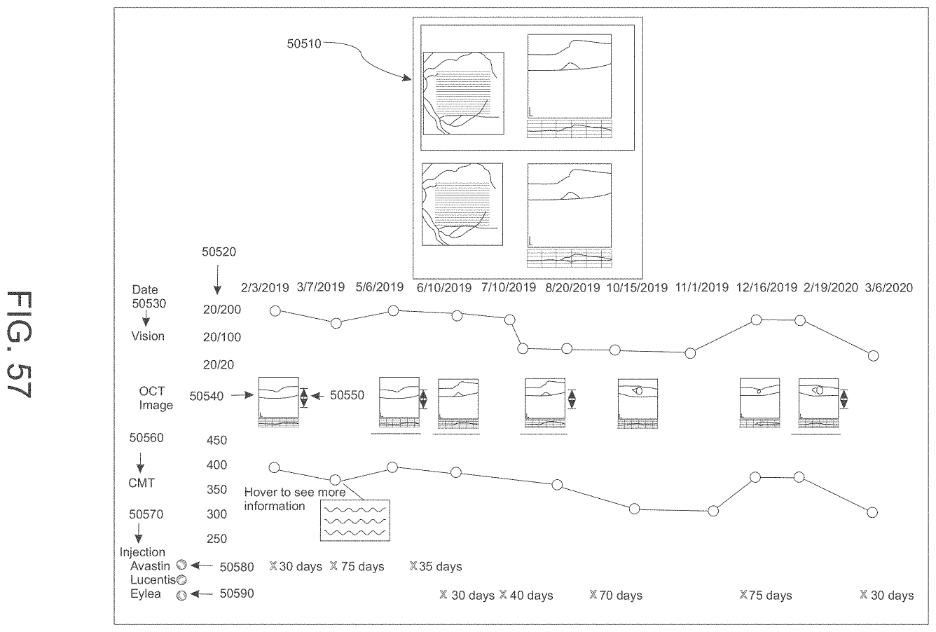

FIG. 57 depicts another embodiment of a display of an in-context image management system displaying images in the context of graphically visualized medical data modules in accordance with an embodiment of the present principles. In the in-context image management system of FIG. 57, 50510 can be displayed within the context of graphical representations of patient data. Large or thumbnailed versions of images can be display and edited and/or otherwise altered. In FIG. 57, within the image viewing module 50510 one or multiple images can be interacted with at the same time, not limiting the user to accessing and editing a single image before moving on to the next.

In the embodiment of FIG. 57, date of service 50520 is displayed horizontally instead of vertically. Those skilled in the art will appreciate that relevant axes may be horizontal, vertical, along a z-axis, or any other axis as deemed appropriate. In the embodiment of FIG. 57, visual acuity 50530 is now displayed horizontally and graphically. Those skilled in the art will appreciate that the representation of underlying data can be displayed as text, lists of text, formatted or unformatted text, as a graphical symbol or icon, plotted on a graph, or otherwise graphically represented. OCT diagnostic images 50540 can be displayed horizontally within the context of relevant data. OCT diagnostic images can also be tagged 50550 for ease of assessment in such a manner as to enable a medical care provider to later save the step of actually viewing a detailed version of the image by denoting if the image was an improvement or deterioration from the previous study. In some embodiments, algorithms can be implemented to automatically denote an increase or decrease in key results returned within image metadata or by other digital means. Additional results, such as central macular thickness 50560, can also be displayed horizontally in accordance with some embodiments. Key results, correlated against events, actions, and diagnostic imaging is an example of in-context image management. In some embodiments, key procedures 50570 can also display alongside images and results. Procedures, results, images, and other patient data may trigger rules to highlight, flag, or otherwise draw attention to their relevant impact on the study or compliance. In the embodiment of FIG. 57, an Avastin injection 50580 is highlighted, for example, in red to denote an issue with compliance, impact on relevant results, or other problem or deficiency related to the injection. In the embodiment of FIG. 57, an Eylea injection 50590 is highlighted, for example, in green to denote proper compliance, and improvement in results, or other improvement or positive result related to the injection. Key indicators expressed throughout the in-context image management system offer caregivers quick access to relevant information while maintaining the ability to view and edit images.

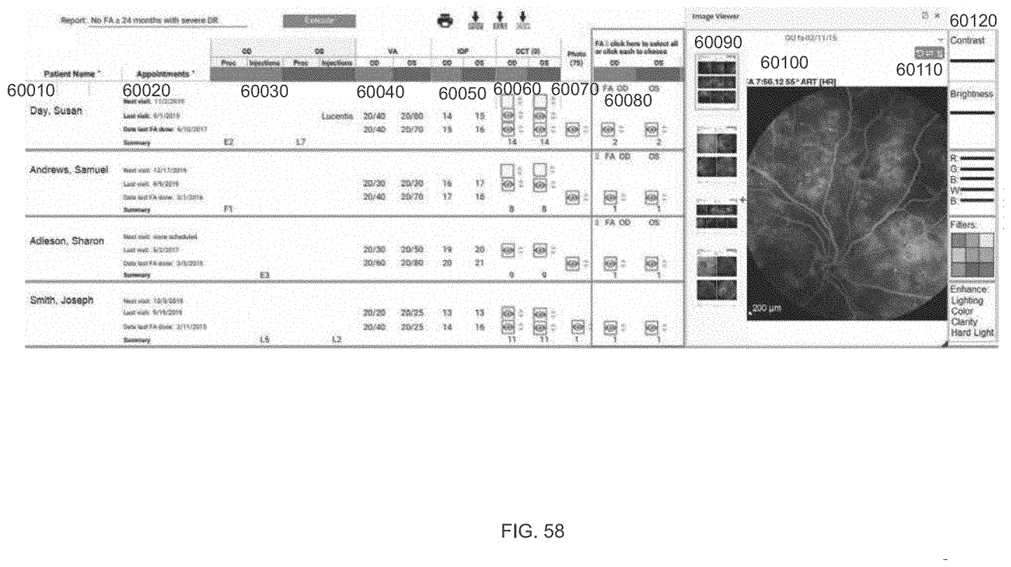

FIG. 58 depicts an embodiment of a display of an in-context image management system displaying multiple patients and multiple images in accordance with an embodiment of the present principles. In the embodiment of FIG. 58 a column denoting individual patients 60010 lists one or more patients with relevant patient data in subsequent columns. A summary of patient information is displayed within a column 60020, and/or individual columns can display precise patient data such as procedures and injections 60030, visual acuity 60040, and intraocular pressure 60050. In the embodiment of FIG. 58, patient data is displayed alongside diagnostic images such as an OCT 60060, fundus photos 60070, and fluorescein angiography 60080. In the embodiment of FIG. 58, an icon or graphical representation depicts the presence of an image. One skilled in the art will appreciate that an icon or graphical representation, such as displayed in columns 60060, 60070, and 60080, can be substituted for a thumbnail as denoted in 50080 of FIG. 56.

In some embodiments, a series of thumbnailed images can be displayed in a column or row 60090 adjacent to a full-sized or enlarged image 60100. Such images can be configured to default or prioritize in a specified order. In some embodiments, images can be edited using buttons, icons, or other graphical interfaces to enable editing such as resizing, rotating, and/or flipping horizontally or vertically 60110, as well as more in depth editing tools 60120 such as adjusting contrast, brightness, individual color channels, applying filters, or individual attribute enhancements.

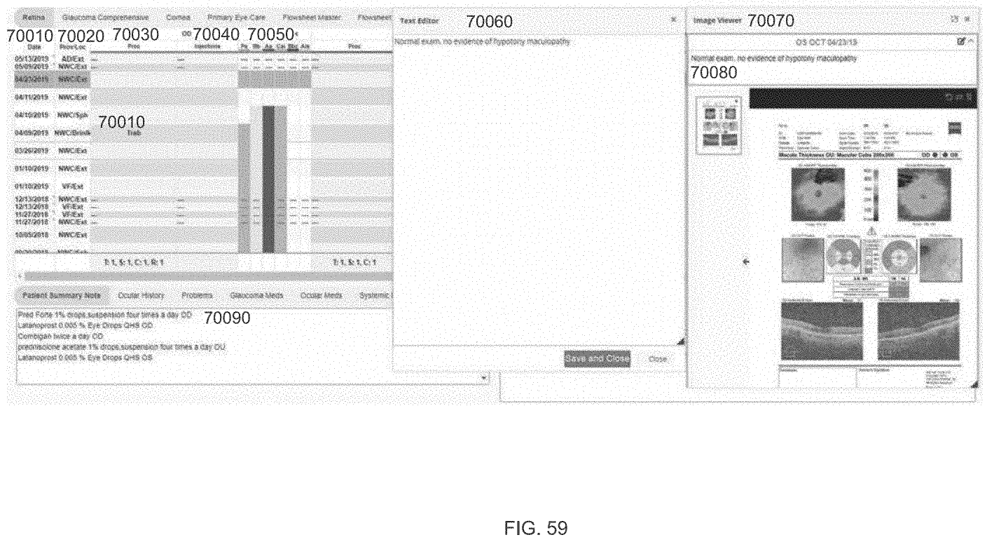

FIG. 59 depicts an embodiment of a display of an in-context image management system enabling a direct edit of image information in accordance with an embodiment of the present principles. In the embodiment of FIG. 59, date of service 70010, provider and location 70020, procedures 70030, injections 70040, and medications 70050 illustratively comprise patient data. In FIG. 59, a text editor 70060 is launched in context of both patient data and relevant imagery 70070. Edited text inserted by a user can update and/or overwrite existing interpretation text 70080 upon completion, by, for example, selecting Save and/or Close. In some embodiments, images can be selected using icons or thumbnails present in any row or column indicating that an image is present. In some embodiments, the icons and/or thumbnails can include relevant information about the image to enable rapid identification of which image or images a user wishes to select. As depicted in FIG. 59, additional modules can be selected and edited while viewing the image through the in-context image management system 70070, such as recording a patient summary note 70090. Those skilled in the art will appreciate that any available module which allows for editing may be substituted for patient summary note in this example. Embodiments of the present principles enable the editing of patient data while viewing the image in-context, and conversely the editing of any image while viewing patient data in-context.

In accordance with the present principles, a user/medical care provider is enabled, by an in-context image management system of the present principles, to view/manipulate/edit images in context of patient data via an ability to select icons/thumbnails of images, for viewing/manipulating/editing of images while having access to (i.e., viewing) patient data. In some embodiments of the present principles, portions of images and/or image viewers can be transparent such that underlying patient data can still be viewable/selectable while viewing an image.

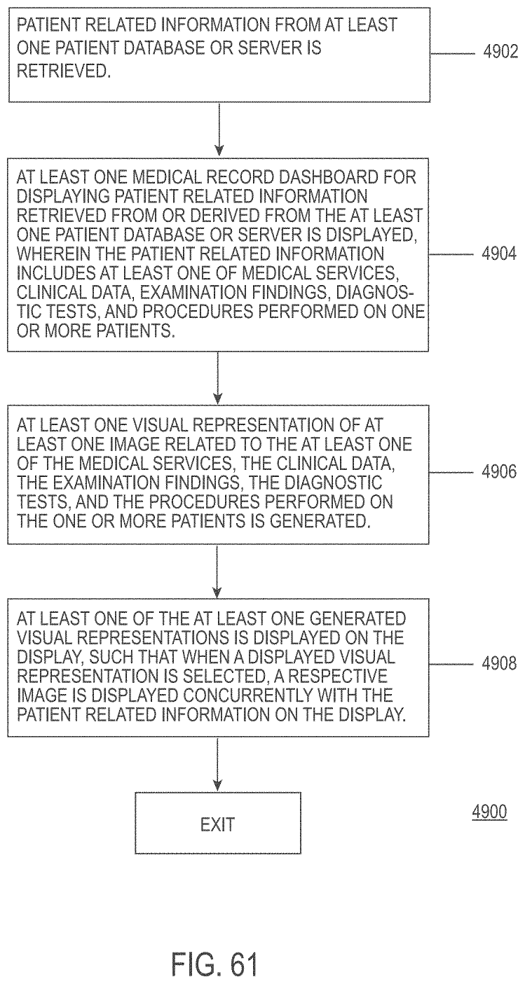

FIG. 61 depicts a flow diagram of a method 4900 for in-context display of images and patient related information in accordance with an embodiment of the present principles. The method 4900 can begin at 4902 during which patient related information from at least one patient database or server is retrieved. The method 4900 can proceed to 4904.

At 4904, at least one medical record dashboard including one or more windows for displaying, using a single display interface, patient related information retrieved from or derived from the at least one patient database or server including at least one of medical services, clinical data, examination findings, diagnostic tests, and procedures performed on one or more patients can be displayed. In some embodiment, the one or more windows can include a plurality of data entry fields for displaying the patient related information received or derived from the at least one patient database, wherein the at least one of the medical services, the clinical data, the examination findings, the diagnostic tests, and the procedures are arranged on a display according to at least one of a time and a date that the medical services, the clinical data, the examination findings, the diagnostic tests and the procedures were performed on the one or more patients. The method 4900 can proceed to 4906.

At 4906, at least one visual representation of at least one image related to the at least one of the medical services, the clinical data, the examination findings, the diagnostic tests, and the procedures performed on the one or more patients is generated. The method 4900 can proceed to 4908.

At 4908, at least one of the at least one generated visual representations is displayed on the display in at least one of the plurality of data entry fields, such that when a displayed visual representation is selected, a respective image is displayed concurrently with the patient related information on the display. The method 4900 can then be exited.

In some embodiments, a visual representation of a patient-related image in accordance with the present principles can include at least one thumbnail representation of an image.

In some embodiments, a user of an in-context image management system in accordance with the present principles is able to select an image to view by reviewing the thumbnail representations of the available patient-related images.

In some embodiments, a selection of a displayed visual representation of patient-related images can include clicking on the displayed visual representation of patient-related images using a pointing device.

In some embodiments, a selection of a displayed visual representation of patient-related images can include hovering over a displayed visual representation of patient-related images using a pointing device.

In some embodiment of the present principles, a system for in-context display of images and patient related information include a computing device comprising at least one processor and a non-transitory computer-readable medium, having stored thereon, software instructions. In such embodiment, when the software instructions are executed by the at least one processor of the computing device, the system is configured to perform operations including at least retrieving patient related information from at least one patient database or server, displaying at least one medical record dashboard including one or more windows for displaying, using a single display interface, patient related information retrieved from or derived from the at least one patient database or server including at least one of medical services, clinical data, examination findings, diagnostic tests, and procedures performed on one or more patients, where the one or more windows include a plurality of data entry fields for displaying the patient related information received or derived from the at least one patient database, and where the at least one of the medical services, the clinical data, the examination findings, the diagnostic tests, and the procedures are arranged on a display according to at least one of a time and a date that the medical services, the clinical data, the examination findings, the diagnostic tests and the procedures were performed on the one or more patients. In such embodiments the system is further configured to perform operations including generating at least one visual representation of at least one image related to the at least one of the medical services, the clinical data, the examination findings, the diagnostic tests, and the procedures performed on the one or more patients, and displaying at least one of the at least one generated graphical representation on the display in at least one of the plurality of data entry fields, such that when a displayed graphical representation is selected, a respective image is displayed concurrently with the patient related information on the display.

Some additional features of a medical records dashboard of the present principles, such as the medical records dashboard 2100 of FIG. 15, include displaying at least one visual indication of a payment for services provided. Further, the user can be provided with access to a detailed ledger comprising financial information related to one or more procedures. For example and as depicted in FIG. 15, the medical records dashboard 2100 can comprise a payment indicator column 2200 including one or more indicator and/or access icons. For example, in some embodiments, the payment indicator column 2200 can comprise a column 2205a that can be populated with one or more indicator icons. In other embodiments, the column 2205b can be provided with one or more indicator or access icons. In some embodiments, the one or more indicator or access icons can comprise icons of color such as red, yellow, or green to indicate a status of payment from an insurer, varying colors to account for patient payments such as gray for items of no concern, blue for a middling balance or low balance that is past due, or black for a high balance or middling balance that is past due. Other indicator or access icons can display messages such as denial code descriptions or useful messaging, and the whole field may be highlighted in cases where indicator or access icons require additional notification. The payment indicator column 2200 can be located anywhere on the of the medical records dashboard 2100. In the embodiment of FIG. 15, the payment indicator column 2200 is positioned between the procedure column 2110, illustrating or providing access to information detailing one or more procedures performed on the patient and information related to the medical care provider, and the provider column 2130, that can display the location where the procedure was performed, and office visit information. The payment indicator column 2200 may also be flagged or alerted when key parameters are met.

In some embodiments, one or more of the icons of the payment indicator column 2200 can be accessed by the user to initiate the display of more detailed financial information. For example, FIG. 16 depicts a ledger window 2300 accessible from the medical records dashboard 2100 of FIG. 15 in accordance with an embodiment of the present principles. In some embodiments, the Data Command Center of the present principles can display the ledger window 2300 overlaid onto the medical records dashboard 2100. In other embodiments, the ledger window 2300 can be displayed in place of the medical records dashboard 2100. In other embodiments, the ledger window 2300 can be displayed with the medical records dashboard 2100. In some embodiments, the ledger window 2300 can include information processed by the Data Command Center, which includes information related to the date of procedure, description of the procedure, dates entered, date of last transaction, a charge type, modifiers, charge status, etc. and important line items may be highlighted through color changes, alerts, flags, or other digital or textual notifications to draw attention to important details and/or trends. For example, and as depicted in the embodiment of FIG. 16, the ledger window 2300 can include the service to column 2310, entered column 2320, line column 2330, type column 2340, and description column 2350. Further, in some embodiments, the ledger window 2300 can include information related to payments and billing. For example, in some embodiments, the ledger window 2300 can include a display of a charge column 2360, payment column 2370, write-off column 2380, adjustment column 2390, and a balance column 2400, although any relevant financial data may be included. In some embodiments, the user can close the ledger window 2300 and return to the medical records dashboard 2100 at any time. In other embodiments, more than one ledger window 2300 can be displayed based on selections made by the user in the medical records dashboard 2100.

FIG. 17 depicts an embodiment of a Data Command Center menu 2500 including a medical records dashboard 2530 implemented as a data interface to a medical record system in accordance with an embodiment of the present principles. The Data Command Center menu 2500 in the embodiment of FIG. 17 is designed to interact with a conventional EMR system although, as noted above, the Data Command Center menu 2500 of FIG. 17 can be used with other large data systems, such as a Practice Management System, to present data to users in a meaningful way. In addition, the exemplary embodiment illustrates a Data Command Center menu 2500 for implementation in an Ophthalmology practice. Those skilled in the art will appreciate that the interface can be readily configured for other medical specialties. The Data Command Center menu 2500 is able to display data from multiple data sources in multiple different panels on a single interface. In the exemplary embodiment of FIG. 17, the Data Command Center menu 2500 provides a comprehensive overview of the patient's clinical and financial history as well as providing a means to quickly order tests while retaining the ability to see previous medical and financial history. Clinical, insurance, and regulatory guidelines, as well as preferred practice and billing patterns, can be quickly accessible based on the patient's conditions, medications and procedures so that a medical care provider/user can readily provide optimal care and be compliant with medical and billing requirements. The medical care provider thus becomes a part of revenue cycle management for each patient in the medical care provider's practice. Through preprogrammed alerts, notifications, and tasks, or alerts, notifications, and tasks programmed at point of care, the medical care provider has a simplified means of being instructed when key aspects of revenue cycle management diverge from expected results.

The Patient Information Bar 2512 illustrated in FIG. 17 displays high level information about the patient. The user can click on the Patient Information Bar 2512 and the Patient Information Panel 2610 shown in FIG. 20 is displayed underneath the Patient Information Bar 2512. Clicking the bar a second time or anywhere else on the page will close the Patient Information Panel 2610. The Patient Information Panel 2610 contains the patient's date of birth 2612, race 2614, phone number 2616, the date when the patient was first seen 2618, the referring physician 2620, and interesting facts to remember 2622. Information in the Patient Information Panel 2610 can be edited in place by clicking on the item to be changed. When clicked, the field will change to a text field allowing the value to be changed with a Save button displayed next to it (not depicted) or allow for auto-save functionality upon leaving the field. Clicking Save will save the data and/or auto-saving may be enabled to allow saving the data upon exiting the field. Clicking a Cancel button (not shown) will not save the data leaving the value unchanged and the control will revert to static text when auto-saving is not enabled on the field. Below these fields is a Revenue Summary 2624 for the patient. The Revenue Summary 2624 displays patient totals for each year 2626 as well as grand totals 2628 for the total amount billed 2630, amount paid by insurance 2632, amount paid by the patient 2634, amount written off 2636, and the adjustments 2638 for each year. Any or all of these values may be alerted or tasks may be generated based on preconfigured rules or rules configured within the context of the panel.

To the right of the Patient Information Bar 2512 in FIG. 17 is the Patient Insurance Bar 2518. The user can click on the bar and the Patient Insurance Panel 2640 illustrated FIG. 21 is displayed underneath the Patient Insurance Bar 2518. Clicking the Patient Insurance Bar 2518 a second time or anywhere else on the page will close the Patient Insurance Panel 2640. The Patient Insurance Panel 2640 contains information about the patient's insurance 2642 including the type 2644, name 2646, group number 2648, insurance ID number 2650, and phone number 2652 for each insurance company. A link to the patient's benefit document 2654 is provided as well as an overview of the patient's in-network benefits 2656 and out of network benefits 2658 as provided by the patient's insurance company. The values illustrated as 2656 and 2658 are provided for example purposes and will vary based on the data provided by insurance companies. The patient's employer's address and contact information 2660 is also displayed for convenience. Item 2662 displays an alternative embodiment of the Patient Insurance Bar 2520. In this case, the patient has a high deductible plan and this fact is displayed at 2664 in red in the Patient Insurance Bar 2520 to be sure the physician is aware that, in this case, the patient has a high deductible plan. The intelligent alert configuration system illustrated in FIG. 200 may be launched to configure rules, alerts, notifications, and tasks based on clinical and/or financial parameters.

The Financial Flowsheet 2532 is illustrated in FIG. 60. The Financial Flowsheet 2532 contains a dynamic table 3030 of financial activity for the patient, as opposed to the physician's practice, as is usually the case with conventional EMR systems. As illustrated, an exemplary embodiment of the Financial Flowsheet 2532 includes the following fields: the Service Date 3031, Bill Date 3032, Service Description 3033, Relative Value Units (RVUs) 3034 that determine how a physician is paid in accordance with work effort, Gross Charges 3035, the Amount Due from the Patient 3036, the Amount Due from Insurance 3037, any Adjustments 3038, Write Offs 3039, and the outstanding balance 3040. An intelligent alert configuration system can be launched to configure rules, alerts, notifications, and tasks based on clinical and/or financial parameters denoted in FIG. 60. The rules, alerts, notifications, and tasks may be applied to one or a group of patients. Selection of the Encounter expansion control 3047 produces a second dynamic table 3041 within the first dynamic table 3030. The second table 3041 presents individual financial transactions associated with the encounter. In an exemplary embodiment, the second dynamic table 3041 contains: the Service Date 3042, Date Entered 3043, the Type of financial activity entered 3044, the Service Description 3033, the Charge or Amount Billed 3045, Payments 3046, Adjustments 3038, Write Offs 3039, and the outstanding balance 3040. The intelligent alert configuration system illustrated may be launched to configure rules, alerts, notifications, and tasks based on clinical and/or financial parameters at a more granular level, which may then be applied to one or a group of patients.

The methods and processes described herein may be implemented in software, hardware, or a combination thereof, in different embodiments. In addition, the order of methods can be changed, and various elements can be added, reordered, combined, omitted or otherwise modified. All examples described herein are presented in a non-limiting manner. Various modifications and changes can be made as would be obvious to a person skilled in the art having benefit of this disclosure. Realizations in accordance with embodiments have been described in the context of particular embodiments. These embodiments are meant to be illustrative and not limiting. Many variations, modifications, additions, and improvements are possible. Accordingly, plural instances can be provided for components described herein as a single instance. Boundaries between various components, operations and data stores are somewhat arbitrary, and particular operations are illustrated in the context of specific illustrative configurations. Other allocations of functionality are envisioned and can fall within the scope of claims that follow. Structures and functionality presented as discrete components in the example configurations can be implemented as a combined structure or component. These and other variations, modifications, additions, and improvements can fall within the scope of embodiments as defined in the claims that follow.

In the foregoing description, numerous specific details, examples, and scenarios are set forth in order to provide a more thorough understanding of the present disclosure. It will be appreciated, however, that embodiments of the disclosure can be practiced without such specific details. Further, such examples and scenarios are provided for illustration, and are not intended to limit the disclosure in any way. Those of ordinary skill in the art, with the included descriptions, should be able to implement appropriate functionality without undue experimentation.

References in the specification to “an embodiment,” etc., indicate that the embodiment described can include a particular feature, structure, or characteristic, but every embodiment may not necessarily include the particular feature, structure, or characteristic. Such phrases are not necessarily referring to the same embodiment. Further, when a particular feature, structure, or characteristic is described in connection with an embodiment, it is believed to be within the knowledge of one skilled in the art to affect such feature, structure, or characteristic in connection with other embodiments whether or not explicitly indicated.

Embodiments in accordance with the disclosure can be implemented in hardware, firmware, software, or any combination thereof. Embodiments can also be implemented as instructions stored using one or more machine-readable media, which may be read and executed by one or more processors. A machine-readable medium can include any mechanism for storing or transmitting information in a form readable by a machine (e.g., a computing device or a “virtual machine” running on one or more computing devices). For example, a machine-readable medium can include any suitable form of volatile or non-volatile memory.

Modules, data structures, and the like defined herein are defined as such for ease of discussion and are not intended to imply that any specific implementation details are required. For example, any of the described modules and/or data structures can be combined or divided into sub-modules, sub-processes or other units of computer code or data as can be required by a particular design or implementation.

In the drawings, specific arrangements or orderings of schematic elements can be shown for ease of description. However, the specific ordering or arrangement of such elements is not meant to imply that a particular order or sequence of processing, or separation of processes, is required in all embodiments. In general, schematic elements used to represent instruction blocks or modules can be implemented using any suitable form of machine-readable instruction, and each such instruction can be implemented using any suitable programming language, library, application-programming interface (API), and/or other software development tools or frameworks. Similarly, schematic elements used to represent data or information can be implemented using any suitable electronic arrangement or data structure. Further, some connections, relationships or associations between elements can be simplified or not shown in the drawings so as not to obscure the disclosure.

This disclosure is to be considered as exemplary and not restrictive in character, and all changes and modifications that come within the guidelines of the disclosure are desired to be protected.

1. A method for in-context display of images and patient related information, comprising:
retrieving patient related information from at least one patient database or server;
displaying at least one medical record dashboard comprising one or more windows for displaying, using a single display interface, patient related information retrieved from or derived from the at least one patient database or server including at least one of medical services, clinical data, examination findings, diagnostic tests, and procedures performed on one or more patients, the one or more windows comprising a plurality of data entry fields for displaying the patient related information received or derived from the at least one patient database, wherein the at least one of the medical services, the clinical data, the examination findings, the diagnostic tests, and the procedures are arranged on a display according to at least one of a time and a date that the medical services, the clinical data, the examination findings, the diagnostic tests and the procedures were performed on the one or more patients;
generating at least one visual representation of at least one image related to the at least one of the medical services, the clinical data, the examination findings, the diagnostic tests, and the procedures performed on the one or more patients; and
displaying at least one of the at least one generated visual representations on the display in at least one of the plurality of data entry fields, such that when a displayed visual representation is selected, a respective image is displayed concurrently with the patient related information on the display.

2. The method of claim 1, wherein the at least one visual representation comprises at least one thumbnail representation of the at least one image.

3. The method of claim 2, wherein a user is able to select an image to view by viewing the at least one thumbnail representation of the at least one image.

4. The method of claim 1, wherein the selection of a displayed visual representation comprises clicking on the displayed visual representation using a pointing device.

5. The method of claim 1, wherein the selection of a displayed visual representation comprises hovering over the displayed visual representation using a pointing device.

6. A system for in-context display of images and patient related information, comprising:
a computing device comprising at least one processor;
a non-transitory computer-readable medium, having stored thereon, software instructions that when executed by the at least one processor of the computing device, cause the system to perform operations comprising at least:
retrieving patient related information from at least one patient database or server;
displaying at least one medical record dashboard comprising one or more windows for displaying, using a single display interface, patient related information retrieved from or derived from the at least one patient database or server including at least one of medical services, clinical data, examination findings, diagnostic tests, and procedures performed on one or more patients, the one or more windows comprising a plurality of data entry fields for displaying the patient related information received or derived from the at least one patient database, wherein the at least one of the medical services, the clinical data, the examination findings, the diagnostic tests, and the procedures are arranged on a display according to at least one of a time and a date that the medical services, the clinical data, the examination findings, the diagnostic tests and the procedures were performed on the one or more patients;
generating at least one visual representation of at least one image related to the at least one of the medical services, the clinical data, the examination findings, the diagnostic tests, and the procedures performed on the one or more patients; and
displaying at least one of the at least one generated graphical representation on the display in at least one of the plurality of data entry fields, such that when a displayed graphical representation is selected, a respective image is displayed concurrently with the patient related information on the display.

7. A method for a display of a graphical representation of complete medical history of a patient in a data command center comprising one or more windows for display and including patient-related data received or derived from at least one patient database, the method comprising:
determining, from the patient-related data, a complete medical history of at least one patient including at least one of medical services, clinical data, examination findings, diagnostic tests, medications administered to and procedures performed on a patient;
generating a graphical representation of the determined complete medical history of the patient including the at least one of medical services, clinical data, examination findings, diagnostic tests, medications administered to and procedures performed on the patient; and
displaying the generated graphical representation in the at least one or more windows according to at least one of a time and a date that the at least one of the medical services, the clinical data, the examination findings, the diagnostic tests, and the procedures the medical services, the clinical data, the examination findings, the diagnostic tests and the procedures were performed on the one or more patients and at least one of the times and the dates that the medications were being administered to the patient;
wherein a user is enabled to select a location in the displayed graphical representation and details regarding the at least one of medical services, clinical data, examination findings, diagnostic tests, medications administered to and procedures performed on the patient related to that selected location are presented to the user.
